SEQUENCE LISTING (273 sequences)

SEQ ID NO: 1 - protein, 5 aa, synthetic construct
  REGION 1..5: note = TLA0015-HCDR1
  VARIANT 1: note = Xaa = Ser, Thr or Tyr
XYGVD

SEQ ID NO: 2 - protein, 5 aa, synthetic construct
  REGION 1..5: note = TLA0015-HCDR1-1
SYGVD

SEQ ID NO: 3 - protein, 5 aa, synthetic construct
  REGION 1..5: note = TLA0015-HCDR1-2
YYGVD

SEQ ID NO: 4 - protein, 5 aa, synthetic construct
  REGION 1..5: note = TLA0015-HCDR1-3
TYGVD

SEQ ID NO: 5 - protein, 16 aa, synthetic construct
  REGION 1..16: note = TLA0015-HCDR2
  VARIANT 1: note = Xaa = Arg or Val
  VARIANT 7: note = Xaa = Gly or Lys
  VARIANT 9: note = Xaa = Asp, His or Asn
  VARIANT 11: note = Xaa = Asn or Gln
  VARIANT 12: note = Xaa = Ala, Pro or Ser
XIWGFGXTXYXXALKS

SEQ ID NO: 6 - protein, 16 aa, synthetic construct
  REGION 1..16: note = TLA0015-HCDR2-1
VIWGFGGTNYNSALKS

SEQ ID NO: 7 - protein, 16 aa, synthetic construct
  REGION 1..16: note = TLA0015-HCDR2-2
VIWGFGGTNYQSALKS

SEQ ID NO: 8 - protein, 16 aa, synthetic construct
  REGION 1..16: note = TLA0015-HCDR2-3
VIWGFGGTNYNAALKS

SEQ ID NO: 9 - protein, 16 aa, synthetic construct
  REGION 1..16: note = TLA0015-HCDR2-4
VIWGFGGTNYNPALKS

SEQ ID NO: 10 - protein, 16 aa, synthetic construct
  REGION 1..16: note = TLA0015-HCDR2-5
VIWGFGGTHYNSALKS

SEQ ID NO: 11 - protein, 16 aa, synthetic construct
  REGION 1..16: note = TLA0015-HCDR2-6
RIWGFGGTNYNSALKS

SEQ ID NO: 12 - protein, 16 aa, synthetic construct
  REGION 1..16: note = TLA0015-HCDR2-7
VIWGFGKTNYNSALKS

SEQ ID NO: 13 - protein, 16 aa, synthetic construct
  REGION 1..16: note = TLA0015-HCDR2-8
VIWGFGGTDYNSALKS

SEQ ID NO: 14 - protein, 8 aa, synthetic construct
  REGION 1..8: note = TLA0015-HCDR3
  VARIANT 2: note = Xaa = Asp, Leu or Asn
  VARIANT 3: note = Xaa = Phe or Tyr
GXXDAMDY

SEQ ID NO: 15 - protein, 8 aa, synthetic construct
  REGION 1..8: note = TLA0015-HCDR3-1
GNFDAMDY

SEQ ID NO: 16 - protein, 8 aa, synthetic construct
  REGION 1..8: note = TLA0015-HCDR3-2
GNYDAMDY

SEQ ID NO: 17 - protein, 8 aa, synthetic construct
  REGION 1..8: note = TLA0015-HCDR3-3
GDFDAMDY

SEQ ID NO: 18 - protein, 8 aa, synthetic construct
  REGION 1..8: note = TLA0015-HCDR3-4
GLFDAMDY

SEQ ID NO: 19 - protein, 30 aa, synthetic construct
  REGION 1..30: note = TLA0015-HFR1
  VARIANT 2: note = Xaa = Ile or Val
  VARIANT 5: note = Xaa = Leu or Val
  VARIANT 12: note = Xaa = Ile or Val
  VARIANT 20: note = Xaa = Ile or Leu
  VARIANT 28: note = Xaa = Asp or Ser
  VARIANT 30: note = Xaa = Ile, Lys or Ser
EXQLXESGGGLXQPGGSLRXSCAVSGFXLX

SEQ ID NO: 20 - protein, 30 aa, synthetic construct
  REGION 1..30: note = TLA0015-HFR1-1
EIQLLESGGGLVQPGGSLRLSCAVSGFSLS

SEQ ID NO: 21 - protein, 30 aa, synthetic construct
  REGION 1..30: note = TLA0015-HFR1-2
EIQLLESGGGLVQPGGSLRISCAVSGFSLS

SEQ ID NO: 22 - protein, 30 aa, synthetic construct
  REGION 1..30: note = TLA0015-HFR1-3
EIQLVESGGGLVQPGGSLRISCAVSGFSLS

SEQ ID NO: 23 - protein, 30 aa, synthetic construct
  REGION 1..30: note = TLA0015-HFR1-4
EVQLLESGGGLVQPGGSLRLSCAVSGFSLS

SEQ ID NO: 24 - protein, 30 aa, synthetic construct
  REGION 1..30: note = TLA0015-HFR1-5
EIQLVESGGGLIQPGGSLRLSCAVSGFSLS

SEQ ID NO: 25 - protein, 30 aa, synthetic construct
  REGION 1..30: note = TLA0015-HFR1-6
EIQLVESGGGLIQPGGSLRISCAVSGFSLS

SEQ ID NO: 26 - protein, 30 aa, synthetic construct
  REGION 1..30: note = TLA0015-HFR1-7
EIQLVESGGGLIQPGGSLRISCAVSGFSLI

SEQ ID NO: 27 - protein, 30 aa, synthetic construct
  REGION 1..30: note = TLA0015-HFR1-8
EIQLVESGGGLIQPGGSLRISCAVSGFSLK

SEQ ID NO: 28 - protein, 30 aa, synthetic construct
  REGION 1..30: note = TLA0015-HFR1-9
EIQLVESGGGLIQPGGSLRISCAVSGFDLS

SEQ ID NO: 29 - protein, 14 aa, synthetic construct
  REGION 1..14: note = TLA0015-HFR2
  VARIANT 5: note = Xaa = Ala or Ser
  VARIANT 13: note = Xaa = Leu or Val
WVRQXPGKGLEWXG

SEQ ID NO: 30 - protein, 14 aa, synthetic construct
  REGION 1..14: note = TLA0015-HFR2-1/TLA-0238-HFR2-3
WVRQAPGKGLEWVG

SEQ ID NO: 31 - protein, 14 aa, synthetic construct
  REGION 1..14: note = TLA0015-HFR2-2
WVRQAPGKGLEWLG

SEQ ID NO: 32 - protein, 14 aa, synthetic construct
  REGION 1..14: note = TLA0015-HFR2-3
WVRQSPGKGLEWLG

SEQ ID NO: 33 - protein, 32 aa, synthetic construct
  REGION 1..32: note = TLA0015-HFR3
  VARIANT 2: note = Xaa = Phe or Leu
  VARIANT 6: note = Xaa = Ala, Lys, Arg or Val
  VARIANT 13: note = Xaa = Leu or Val
  VARIANT 32: note = Xaa = Arg or Ser
RXTISXDNSKNTXYLQMNSLRAEDTAVYYCAX

SEQ ID NO: 34 - protein, 32 aa, synthetic construct
  REGION 1..32: note = TLA0015-HFR3-1
RLTISRDNSKNTLYLQMNSLRAEDTAVYYCAS

SEQ ID NO: 35 - protein, 32 aa, synthetic construct
  REGION 1..32: note = TLA0015-HFR3-2
RLTISKDNSKNTVYLQMNSLRAEDTAVYYCAS

SEQ ID NO: 36 - protein, 32 aa, synthetic construct
  REGION 1..32: note = TLA0015-HFR3-3
RFTISRDNSKNTVYLQMNSLRAEDTAVYYCAS

SEQ ID NO: 37 - protein, 32 aa, synthetic construct
  REGION 1..32: note = TLA0015-HFR3-4
RLTISRDNSKNTVYLQMNSLRAEDTAVYYCAS

SEQ ID NO: 38 - protein, 32 aa, synthetic construct
  REGION 1..32: note = TLA0015-HFR3-5
RLTISRDNSKNTVYLQMNSLRAEDTAVYYCAR

SEQ ID NO: 39 - protein, 32 aa, synthetic construct
  REGION 1..32: note = TLA0015-HFR3-6
RLTISVDNSKNTVYLQMNSLRAEDTAVYYCAS

SEQ ID NO: 40 - protein, 32 aa, synthetic construct
  REGION 1..32: note = TLA0015-HFR3-7
RLTISADNSKNTVYLQMNSLRAEDTAVYYCAS

SEQ ID NO: 41 - protein, 11 aa, synthetic construct
  REGION 1..11: note = TLA0015-HFR4-1/TLA-0238-HFR4-2
WGQGTLVTVSS

SEQ ID NO: 42 - protein, 11 aa, synthetic construct
  REGION 1..11: note = TLA0015-LCDR1-1
RASQDISNYLN

SEQ ID NO: 43 - protein, 7 aa, synthetic construct
  REGION 1..7: note = TL0015-LCDR2-1
YTSRLHS

SEQ ID NO: 44 - protein, 9 aa, synthetic construct
  REGION 1..9: note = TL0015-LCDR3-1
QQGNTLPFT

SEQ ID NO: 45 - protein, 23 aa, synthetic construct
  REGION 1..23: note = TL0015-LFR1
  VARIANT 7: note = Xaa = Ser or Thr
  VARIANT 8: note = Xaa = Pro or Thr
  VARIANT 22: note = Xaa = Ser or Thr
DIQMTQXXSSLSASVGDRVTIXC

SEQ ID NO: 46 - protein, 23 aa, synthetic construct
  REGION 1..23: note = TL0015-LFR1-1
DIQMTQTTSSLSASLGDRVTISC

SEQ ID NO: 47 - protein, 23 aa, synthetic construct
  REGION 1..23: note = TL0015-LFR1-2/TLA0241-LFR1-1
DIQMTQSPSSLSASVGDRVTITC

SEQ ID NO: 48 - protein, 15 aa, synthetic construct
  REGION 1..15: note = TL0015-LFR2
  VARIANT 9: note = Xaa = Ala or Val
  VARIANT 10: note = Xaa = Pro or Val
WYQQKPGKXXKLLIY

SEQ ID NO: 49 - protein, 15 aa, synthetic construct
  REGION 1..15: note = TL0015-LFR2-1
WYQQKPDGTVKLLIY

SEQ ID NO: 50 - protein, 15 aa, synthetic construct
  REGION 1..15: note = TL0015-LFR2-2
WYQQKPGKVVKLLIY

SEQ ID NO: 51 - protein, 15 aa, synthetic construct
  REGION 1..15: note = TL0015-LFR2-3
WYQQKPGKAVKLLIY

SEQ ID NO: 52 - protein, 15 aa, synthetic construct
  REGION 1..15: note = TL0015-LFR2-4/TLA0241-LFR2-1
WYQQKPGKAPKLLIY

SEQ ID NO: 53 - protein, 32 aa, synthetic construct
  REGION 1..32: note = TL0015-LFR3
  VARIANT 15: note = Xaa = Phe or Tyr
  VARIANT 27: note = Xaa = Phe or Val
  VARIANT 31: note = Xaa = Phe or Tyr
GVPSRFSGSGSGTDXTLTISSLQPEDXATYXC

SEQ ID NO: 54 - protein, 32 aa, synthetic construct
  REGION 1..32: note = TL0015-LFR3-1
GVPSRFSGSGSGTDYSLTISNLEQEDIATYFC

SEQ ID NO: 55 - protein, 32 aa, synthetic construct
  REGION 1..32: note = TL0015-LFR3-2
GVPSRFSGSGSGTDYTLTISSLQPEDVATYFC

SEQ ID NO: 56 - protein, 32 aa, synthetic construct
  REGION 1..32: note = TL0015-LFR3-3
GVPSRFSGSGSGTDYTLTISSLQPEDFATYFC

SEQ ID NO: 57 - protein, 32 aa, synthetic construct
  REGION 1..32: note = TL0015-LFR3-4/TLA0238-LFR3-4/TLA0241-LFR3-2
GVPSRFSGSGSGTDFTLTISSLQPEDFATYYC

SEQ ID NO: 58 - protein, 10 aa, synthetic construct
  REGION 1..10: note = TL0015-LFR4
  VARIANT 3: note = Xaa = Pro or Gln
FGXGTKLEIK

SEQ ID NO: 59 - protein, 10 aa, synthetic construct
  REGION 1..10: note = TL0015-LFR4-1
FGSGTKLEIK

SEQ ID NO: 60 - protein, 10 aa, synthetic construct
  REGION 1..10: note = TL0015-LFR4-2
FGPGTKLEIK

SEQ ID NO: 61 - protein, 10 aa, synthetic construct
  REGION 1..10: note = TL0015-LFR4-3/TLA0238-LFR4-3
FGQGTKLEIK

SEQ ID NO: 62 - protein, 116 aa, synthetic construct
  REGION 1..116: note = TLA0015-VH-1
QIQLKESGPGLVAPSQSLSITCTVSGFSLSSYGVDWVRQSPGKGLEWLGVIWGFGGTNYNSALKSRLSISKDNSKSQVFLKMNSLQSDDSAIYYCASGNFDAMDYWGQGTSVTVSS

SEQ ID NO: 63 - protein, 116 aa, synthetic construct
  REGION 1..116: note = TLA0015-VH-2
EIQLLESGGGLVQPGGSLRLSCAVSGFSLSSYGVDWVRQAPGKGLEWVGVIWGFGGTNYNSALKSRLTISRDNSKNTLYLQMNSLRAEDTAVYYCASGNFDAMDYWGQGTLVTVSS

SEQ ID NO: 64 - protein, 116 aa, synthetic construct
  REGION 1..116: note = TLA0015-VH-3
EIQLLESGGGLVQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 65 - protein, 116 aa, synthetic construct
  REGION 1..116: note = TLA0015-VH-4
EIQLVESGGGLVQPGGSLRISCAVSGFSLSSYGVDWVRQSPGKGLEWLGVIWGFGGTNYNSALKSRLTISKDNSKNTVYLQMNSLRAEDTAVYYCASGNFDAMDYWGQGTLVTVSS

SEQ ID NO: 66 - protein, 116 aa, synthetic construct
  REGION 1..116: note = TLA0015-VH-5
EVQLLESGGGLVQPGGSLRLSCAVSGFSLSSYGVDWVRQAPGKGLEWLGVIWGFGGTNYNSALKSRFTISRDNSKNTVYLQMNSLRAEDTAVYYCASGNFDAMDYWGQGTLVTVSS

SEQ ID NO: 67 - protein, 116 aa, synthetic construct
  REGION 1..116: note = TLA0015-VH-6
EVQLLESGGGLVQPGGSLRLSCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 68 - protein, 116 aa, synthetic construct
  REGION 1..116: note = TLA0015-VH-7
EVQLLESGGGLVQPGGSLRLSCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 69 - protein, 116 aa, synthetic construct
  REGION 1..116: note = TLA0015-VH-8
EIQLVESGGGLIQPGGSLRLSCAVSGFSLSSYGVDWVRQAPGKGLEWLGVIWGFGGTNYNSALKSRLTISRDNSKNTVYLQMNSLRAEDTAVYYCARGNFDAMDYWGQGTLVTVSS

SEQ ID NO: 70 - protein, 116 aa, synthetic construct
  REGION 1..116: note = TLA0015-VH-9
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 71 - protein, 116 aa, synthetic construct
  REGION 1..116: note = TLA0015-VH-10
EIQLVESGGGLIQPGGSLRISCAVSGFSLSSYGVDWVRQAPGKGLEWLGVIWGFGGTNYNSALKSRLTISRDNSKNTVYLQMNSLRAEDTAVYYCASGNFDAMDYWGQGTLVTVSS

SEQ ID NO: 72 - protein, 116 aa, synthetic construct
  REGION 1..116: note = TLA0015-VH-11
EIQLVESGGGLIQPGGSLRISCAVSGFSLSSYGVDWVRQAPGKGLEWLGVIWGFGGTNYNSALKSRLTISVDNSKNTVYLQMNSLRAEDTAVYYCASGNFDAMDYWGQGTLVTVSS

SEQ ID NO: 73 - protein, 116 aa, synthetic construct
  REGION 1..116: note = TLA0015-VH-12
EIQLVESGGGLIQPGGSLRISCAVSGFSLSSYGVDWVRQAPGKGLEWLGVIWGFGGTNYQSALKSRLTISKDNSKNTVYLQMNSLRAEDTAVYYCASGNFDAMDYWGQGTLVTVSS

SEQ ID NO: 74 - protein, 116 aa, synthetic construct
  REGION 1..116: note = TLA0015-VH-13
EIQLVESGGGLIQPGGSLRISCAVSGFSLSSYGVDWVRQAPGKGLEWLGVIWGFGGTNYNAALKSRLTISKDNSKNTVYLQMNSLRAEDTAVYYCASGNFDAMDYWGQGTLVTVSS

SEQ ID NO: 75 - protein, 116 aa, synthetic construct
  REGION 1..116: note = TLA0015-VH-14
EIQLVESGGGLIQPGGSLRISCAVSGFSLSSYGVDWVRQAPGKGLEWLGVIWGFGGTNYNSALKSRLTISADNSKNTVYLQMNSLRAEDTAVYYCASGNFDAMDYWGQGTLVTVSS

SEQ ID NO: 76 - protein, 116 aa, synthetic construct
  REGION 1..116: note = TLA0015-VH-15
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 77 - protein, 116 aa, synthetic construct
  REGION 1..116: note = TLA0015-VH-16
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 78 - protein, 116 aa, synthetic construct
  REGION 1..116: note = TLA0015-VH-17
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 79 - protein, 116 aa, synthetic construct
  REGION 1..116: note = TLA0015-VH-18
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGLFDAMDYWGQGTLVTVSS

SEQ ID NO: 80 - protein, 116 aa, synthetic construct
  REGION 1..116: note = TLA0015-VH-19
EIQLVESGGGLIQPGGSLRISCAVSGFSLSTYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 81 - protein, 116 aa, synthetic construct
  REGION 1..116: note = TLA0015-VH-20
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 82 - protein, 116 aa, synthetic construct
  REGION 1..116: note = TLA0015-VH-21
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 83 - protein, 116 aa, synthetic construct
  REGION 1..116: note = TLA0015-VH-22
EIQLVESGGGLIQPGGSLRISCAVSGFSLKSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 84 - protein, 116 aa, synthetic construct
  REGION 1..116: note = TLA0015-VH-23
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 85 - protein, 116 aa, synthetic construct
  REGION 1..116: note = TLA0015-VH-24
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 86 - protein, 116 aa, synthetic construct
  REGION 1..116: note = TLA0015-VH-25
EIQLVESGGGLIQPGGSLRISCAVSGFDLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 87 - protein, 116 aa, synthetic construct
  REGION 1..116: note = TLA0015-VH-26
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNFDAMDYWGQGTLVTVSS

SEQ ID NO: 88 - protein, 116 aa, synthetic construct
  REGION 1..116: note = TLA0015-VH-27
EIQLVESGGGLIQPGGSLRISCAVSGFSLSSYGVDWVRQAPGKGLEWLGVIWGFGGTDYNSALKSRLTISKDNSKNTVYLQMNSLRAEDTAVYYCASGNFDAMDYWGQGTLVTVSS

SEQ ID NO: 89 - protein, 116 aa, synthetic construct
  REGION 1..116: note = TLA0015-VH-28
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 90 - protein, 116 aa, synthetic construct
  REGION 1..116: note = TLA0015-VH-29
EIQLVESGGGLIQPGGSLRISCAVSGFSLSYYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 91 - protein, 116 aa, synthetic construct
  REGION 1..116: note = TLA0015-VH-30
EIQLVESGGGLIQPGGSLRISCAVSGFSLSYYGVDWVRQAPGKGLEWLGVIWGFGGTNYNPALKSRLTISKDNSKNTVYLQMNSLRAEDTAVYYCASGNFDAMDYWGQGTLVTVSS

SEQ ID NO: 92 - protein, 116 aa, synthetic construct
  REGION 1..116: note = TLA0015-VH-31
EIQLVESGGGLIQPGGSLRISCAVSGFSLSYYGVDWVRQAPGKGLEWLGVIWGFGGTHYNSALKSRLTISKDNSKNTVYLQMNSLRAEDTAVYYCASGNFDAMDYWGQGTLVTVSS

SEQ ID NO: 93 - protein, 116 aa, synthetic construct
  REGION 1..116: note = TLA0015-VH-32
EIQLVESGGGLIQPGGSLRISCAVSGFSLSYYGVDWVRQAPGKGLEWLGRIWGFGGTNYNSALKSRLTISKDNSKNTVYLQMNSLRAEDTAVYYCASGNFDAMDYWGQGTLVTVSS

SEQ ID NO: 94 - protein, 116 aa, synthetic construct
  REGION 1..116: note = TLA0015-VH-33
EIQLVESGGGLIQPGGSLRISCAVSGFDLSYYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 95 - protein, 116 aa, synthetic construct
  REGION 1..116: note = TLA0015-VH-34
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGNYDAMDYWGQGTLVTVSS

SEQ ID NO: 96 - protein, 116 aa, synthetic construct
  REGION 1..116: note = TLA0015-VH-35
EIQLVESGGGLIQPGGSLRISCAVSGFSLSSYGVDWVRQAPGKGLEWLGVIWGFGGTNYNPALKSRLTISKDNSKNTVYLQMNSLRAEDTAVYYCASGNYDAMDYWGQGTLVTVSS

SEQ ID NO: 97 - protein, 116 aa, synthetic construct
  REGION 1..116: note = TLA0015-VH-36
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGNYDAMDYWGQGTLVTVSS

SEQ ID NO: 98 - protein, 116 aa, synthetic construct
  REGION 1..116: note = TLA0015-VH-37
EIQLVESGGGLIQPGGSLRISCAVSGFSLSSYGVDWVRQAPGKGLEWLGRIWGFGGTNYNSALKSRLTISKDNSKNTVYLQMNSLRAEDTAVYYCASGNYDAMDYWGQGTLVTVSS

SEQ ID NO: 99 - protein, 116 aa, synthetic construct
  REGION 1..116: note = TLA0015-VH-38
EIQLVESGGGLIQPGGSLRISCAVSGFSLSSYGVDWVRQAPGKGLEWLGVIWGFGKTNYNSALKSRLTISKDNSKNTVYLQMNSLRAEDTAVYYCASGNYDAMDYWGQGTLVTVSS

SEQ ID NO: 100 - protein, 116 aa, synthetic construct
  REGION 1..116: note = TLA0015-VH-39
EIQLVESGGGLIQPGGSLRISCAVSGFSLISYGVDWVRQAPGKGLEWLGVIWGFGGTNYNSALKSRLTISKDNSKNTVYLQMNSLRAEDTAVYYCASGDFDAMDYWGQGTLVTVSS

SEQ ID NO: 101 - protein, 116 aa, synthetic construct
  REGION 1..116: note = TLA0015-VH-40
EIQLVESGGGLIQPGGSLRISCAVSGFSLSSYGVDWVRQAPGKGLEWLGVIWGFGGTHYNSALKSRLTISKDNSKNTVYLQMNSLRAEDTAVYYCASGDFDAMDYWGQGTLVTVSS

SEQ ID NO: 102 - protein, 107 aa, synthetic construct
  REGION 1..107: note = TLA0015-VL-1
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 103 - protein, 107 aa, synthetic construct
  REGION 1..107: note = TLA0015-VL-2
DIQMTQSPSSLSASVGDRVTITCRASQDISNYLNWYQQKPGKVVKLLIYYTSRLHSGVPSRFSGSGSGTDYTLTISSLQPEDVATYFCQQGNTLPFTFGPGTKLEIK

SEQ ID NO: 104 - protein, 107 aa, synthetic construct
  REGION 1..107: note = TLA0015-VL-3
DIQMTQSPSSLSASVGDRVTITCRASQDISNYLNWYQQKPGKAPKLLIYYTSRLHSGVPSRFSGSGSGTDFTLTISSLQPEDFATYYCQQGNTLPFTFGQGTKLEIK

SEQ ID NO: 105 - protein, 107 aa, synthetic construct
  REGION 1..107: note = TLA0015-VL-4
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPFTFGQGTKLEIK

SEQ ID NO: 106 - protein, 107 aa, synthetic construct
  REGION 1..107: note = TLA0015-VL-5
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 107 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 108 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 109 - protein, 180 aa, synthetic construct
  REGION 1..180: note = Cyno sTL1A (L72-L251)
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 110 - protein, 192 aa, synthetic construct
  REGION 1..192: note = Mouse sTL1A (A61-L252)
AGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 111 - protein, 192 aa, synthetic construct
  REGION 1..192: note = Rat sTL1A (T61-I252)
TGQLRIPGKDCMFPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 112 - protein, 175 aa, synthetic construct
  REGION 1..175: note = Human DR3-ECD (Q25-Q199)
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 113 - skipped

SEQ ID NO: 114 - protein, 5 aa, synthetic construct
  REGION 1..5: note = 18E5-HCDR-1-1
RYLIH

SEQ ID NO: 115 - protein, 5 aa, synthetic construct
  REGION 1..5: note = 18E5-HCDR-1-2
RYLMH

SEQ ID NO: 116 - protein, 5 aa, synthetic construct
  REGION 1..5: note = 18E5-HCDR-1-3
RYLMN

SEQ ID NO: 117 - protein, 17 aa, synthetic construct
  REGION 1..17: note = 18E5-HCDR-2
  VARIANT 7: note = Xaa = Asp or Glu
  VARIANT 8: note = Xaa = Ala or Gly
  VARIANT 12: note = Xaa = Ala or Asn
  VARIANT 13: note = Xaa = Ala, Glu or Gln
  VARIANT 14: note = Xaa = Lys or Pro
  VARIANT 15: note = Xaa = Phe or Val
  VARIANT 16: note = Xaa = Lys or Gln
YINPYNXXTKYXXXXXG

SEQ ID NO: 118 - protein, 17 aa, synthetic construct
  REGION 1..17: note = 18E5-HCDR-2-1
YINPYNDGTKYNEKFKG

SEQ ID NO: 119 - protein, 17 aa, synthetic construct
  REGION 1..17: note = 18E5-HCDR-2-2
YINPYNDGTKYAQKFQG

SEQ ID NO: 120 - protein, 17 aa, synthetic construct
  REGION 1..17: note = 18E5-HCDR-2-3
YINPYNDGTKYAAPVKG

SEQ ID NO: 121 - protein, 17 aa, synthetic construct
  REGION 1..17: note = 18E5-HCDR-2-4
YINPYNDGTKYNQKFQG

SEQ ID NO: 122 - protein, 17 aa, synthetic construct
  REGION 1..17: note = 18E5-HCDR-2-5
YINPYNDATKYNEKFKG

SEQ ID NO: 123 - protein, 17 aa, synthetic construct
  REGION 1..17: note = 18E5-HCDR-2-6
YINPYNEGTKYNEKFKG

SEQ ID NO: 124 - protein, 6 aa, synthetic construct
  REGION 1..6: note = 18E5-HCDR-3
NWDLDY

SEQ ID NO: 125 - protein, 11 aa, synthetic construct
  REGION 1..11: note = 18E5-LCDR-1
RASQSISDYLY

SEQ ID NO: 126 - protein, 7 aa, synthetic construct
  REGION 1..7: note = 18E5-LCDR-2
FASQSIS

SEQ ID NO: 127 - protein, 8 aa, synthetic construct
  REGION 1..8: note = 18E5-LCDR-3
  VARIANT 1: note = Xaa = Asn or Gln
  VARIANT 2: note = Xaa = Ala or Gly
XXHSFPFT

SEQ ID NO: 128 - protein, 8 aa, synthetic construct
  REGION 1..8: note = 18E5-LCDR-3-1
NGHSFPFT

SEQ ID NO: 129 - protein, 8 aa, synthetic construct
  REGION 1..8: note = 18E5-LCDR-3-2
QGHSFPFT

SEQ ID NO: 130 - protein, 8 aa, synthetic construct
  REGION 1..8: note = 18E5-LCDR-3-3
NAHSFPFT

SEQ ID NO: 131 - protein, 243 aa, synthetic construct
  REGION 1..243: note = scTLA0015-11 VHVL
EIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIK

SEQ ID NO: 132 - protein, 243 aa, synthetic construct
  REGION 1..243: note = scTLA0015-11 VLVH
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGQGTKLEIKGGGGSGGGGSGGGGSGGGGSEIQLVESGGGLIQPGGSLRISCAVSGFSLSSYGVDWVRQAPGKGLEWLGVIWGFGGTNYNSALKSRLTISKDNSKNTVYLQMNSLRAEDTAVYYCASGNFDAMDYWGQGTLVTVSS

SEQ ID NO: 133 - protein, 243 aa, synthetic construct
  REGION 1..243: note = scTLA0015-11cc VHVL
EIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 134 - protein, 243 aa, synthetic construct
  REGION 1..243: note = scTLA0015-11cc VLVH
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSS

SEQ ID NO: 135 - protein, 115 aa, synthetic construct
  REGION 1..115: note = 16003-18E5VH
EVQLQQSGPELVKPGAAVKMSCKASGYTFTRYLIHWVKQKPGQGLEWIGYINPYNDGTKYNEKFKGKATLTSDKSSSTAYMELSSLTSEDSAVYYCTSNWDLDYWGQGTTLTVSS

SEQ ID NO: 136 - protein, 115 aa, synthetic construct
  REGION 1..115: note = 23A0144VH
QVQLVQSGAEVKKPGSSVKVSCKASGYTFTRYLIHWVRQAPGQGLEWIGYINPYNDGTKYAQKFQGRVTITADESTSTAYMELSSLRSEDTAVYYCTSNWDLDYWGQGTLVTVSS

SEQ ID NO: 137 - protein, 115 aa, synthetic construct
  REGION 1..115: note = 23A0148VH
EVQLVESGGGLVKPGGSLRLSCAASGYTFTRYLIHWVRQAPGKGLEWIGYINPYNDGTKYAAPVKGRFTISRDDSKNTLYLQMNSLKTEDTAVYYCTSNWDLDYWGQGTLVTVSS

SEQ ID NO: 138 - protein, 115 aa, synthetic construct
  REGION 1..115: note = 23A0149VH
EVQLQQSGPELVKPGASVKVSCKASGYTFTRYLMHWVRQAPGQGLEWIGYINPYNDGTKYNQKFQGRVTITADKSTSTAYMELSSLTSEDTAVYYCTSNWDLDYWGQGTLVTVSS

SEQ ID NO: 139 - protein, 115 aa, synthetic construct
  REGION 1..115: note = 23A0150VH
EVQLQQSGPEVKKPGASVKVSCKASGYTFTRYLIHWVRQAPGQGLEWIGYINPYNDGTKYNQKFQGRVTITADKSTSTAYMELSSLRSEDTAVYYCTSNWDLDYWGQGTTLTVSS

SEQ ID NO: 140 - protein, 115 aa, synthetic construct
  REGION 1..115: note = 23A0151VH
QVQLQQSGSELKKPGASVKVSCKASGYTFTRYLMHWVRQAPGKGLEWIGYINPYNDGTKYNQKFQGRVTITADKSTSTAYMELSSLRSEDTAVYYCTSNWDLDYWGQGTLVTVSS

SEQ ID NO: 141 - protein, 115 aa, synthetic construct
  REGION 1..115: note = 23A0152VH
EIQLQQSGSELKKPGASVKVSCKASGYTFTRYLMNWVRQAPGKGLEWIGYINPYNDGTKYNQKFQGRVTITADKSTSTAYMELSSLRSEDTAVYYCTSNWDLDYWGQGTLVTVSS

SEQ ID NO: 142 - protein, 115 aa, synthetic construct
  REGION 1..115: note = 23A0153VH
EVQLQQSGPEVKKPGASVKVSCKASGYTFTRYLMHWVRQPPGQGLEWIGYINPYNDGTKYNQKFQGRVTITADKSTSTAYMELSSLRSEDTAVYYCTSNWDLDYWGQGTTLTVSS

SEQ ID NO: 143 - protein, 115 aa, synthetic construct
  REGION 1..115: note = 23A0178VH
QVQLVQSGAEVKKPGASVKVSCKASGYTFTRYLIHWVRQAPGQGLEWMGYINPYNDGTKYNEKFKGRVTMTRDTSTSTVYMELSSLRSEDTAVYYCASNWDLDYWGQGTLVTVSS

SEQ ID NO: 144 - protein, 115 aa, synthetic construct
  REGION 1..115: note = 23A0182VH
QVQLVQSGAEVKKPGASVKVSCKASGYTFTRYLIHWVRQAPGQGLEWIGYINPYNDGTKYNEKFKGRVTMTSDKSTSTAYMELSSLRSEDTAVYYCTSNWDLDYWGQGTLVTVSS

SEQ ID NO: 145 - protein, 115 aa, synthetic construct
  REGION 1..115: note = 23A0186-3VH
QVQLVQSGAEVVKPGASVKMSCKASGYTFTRYLIHWVKQAPGQGLEWIGYINPYNDATKYNEKFKGRATLTSDKSTSTAYMELSSLRSEDTAVYYCTSNWDLDYWGQGTLVTVSS

SEQ ID NO: 146 - protein, 115 aa, synthetic construct
  REGION 1..115: note = 23A0186-5VH
QVQLVQSGAEVVKPGASVKMSCKASGYTFTRYLIHWVKQAPGQGLEWIGYINPYNDGTKYNEKFKGRATLTSDKSTSTAYMELSSLRSEDTAVYYCTSNWDLDYWGQGTLVTVSS

SEQ ID NO: 147 - protein, 115 aa, synthetic construct
  REGION 1..115: note = 23A0186-6VH
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSS

SEQ ID NO: 148 - protein, 126 aa, synthetic construct
  REGION 1..126: note = 23A0198 (A5M21)VH
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSS

SEQ ID NO: 149 - protein, 107 aa, synthetic construct
  REGION 1..107: note = 16003-18E5VL
DIVMTQSPATLSVTPGDRVSLSCRASQSISDYLYWYQQKSHESPRLLIKFASQSISGIPSRFTGSGSGSDFTLSIDSVEPDDVGVFFCQNGHSFPFTFGGGTKLEIK

SEQ ID NO: 150 - protein, 107 aa, synthetic construct
  REGION 1..107: note = 23A0144VL
AIQMTQSPSSLSASVGDRVTITCRASQSISDYLYWYQQKPGKAPKLLIKFASQSISGVPSRFSGSGSGTDFTLTISSLQPEDFATYYCQNGHSFPFTFGPGTKVDIK

SEQ ID NO: 151 - protein, 107 aa, synthetic construct
  REGION 1..107: note = 23A0145VL
DIQMTQSPSSLSASVGDRVTITCRASQSISDYLYWFQQKPGKAPKLLIKFASQSISGVPSRFSGSGSGTDFTLTISSLQPEDFATYYCQNGHSFPFTFGQGTRLEIK

SEQ ID NO: 152 - protein, 107 aa, synthetic construct
  REGION 1..107: note = 23A0146VL
NIQMTQSPSAMSASVGDRVTITCRASQSISDYLYWFQQKPGKVPKLLIKFASQSISGVPSRFSGSGSGTEFTLTISSLQPEDFATYYCQNGHSFPFTFGPGTKVDIK

SEQ ID NO: 153 - protein, 107 aa, synthetic construct
  REGION 1..107: note = 23A0147VL
DIQMTQSPSTLSASVGDRVTITCRASQSISDYLYWYQQKPGKAPKLLIKFASQSISGVPSRFSGSGSGTEFTLTISSLQPDDFATYYCQNGHSFPFTFGPGTKVDIK

SEQ ID NO: 154 - protein, 107 aa, synthetic construct
  REGION 1..107: note = 23A0148VL
AIQMTQSPSSLSASVGDRVTITCRASQSISDYLYWYQQKPGKAPKLLIKFASQSISGVPSRFSGSGSGTDFTLTISSLQPEDFATYYCQNGHSFPFTFGQGTKVEIK

SEQ ID NO: 155 - protein, 107 aa, synthetic construct
  REGION 1..107: note = 23A0149VL
AIVMTQSPATLSVSPGERVTLSCRASQSISDYLYWYQQKPDQSPKLLIKFASQSISGVPSRFSGSGSGRDFTLTISSLQPEDFAVYYCQNGHSFPFTFGGGTKLEIK

SEQ ID NO: 156 - protein, 107 aa, synthetic construct
  REGION 1..107: note = 23A0150VL
NIVMTQSPATLSVSPGERATLSCRASQSISDYLYWYQQKPDQSPKLLIKFASQSISGVPSRFSGSGSGRDFTLTISSLEPEDFAVYYCQNGHSFPFTFGGGTKLEIK

SEQ ID NO: 157 - protein, 107 aa, synthetic construct
  REGION 1..107: note = 23A0151VL
EIVMTQSPATLSLSPGERATLSCRASQSISDYLYWYQQKPDQSPKLLIKFASQSISGVPSRFSGSGSGTEFTLTISSLQPEDFAVYYCQNGHSFPFTFGGGTKVEIK

SEQ ID NO: 158 - protein, 107 aa, synthetic construct
  REGION 1..107: note = 23A0152VL
DIVMTQSPATLSVSPGERVTLSCRASQSISDYLYWYQQKPDQSPKLLIKFASQSISGVPSRFSGSGSGSDFTLTISSLQPEDFATYYCQNGHSFPFTFGGGTKLEIK

SEQ ID NO: 159 - protein, 107 aa, synthetic construct
  REGION 1..107: note = 23A0153VL
DIVMTQSPATLSVSPGERVTLSCRASQSISDYLYWYQQKPDQSPRLLIKFASQSISGVPSRFSGSGSGSDFTLKISSVEAEDVGVYYCQNGHSFPFTFGGGTKVEIK

SEQ ID NO: 160 - protein, 107 aa, synthetic construct
  REGION 1..107: note = 23A0183VL
DIQMTQSPSSLSASVGDRVTITCRASQSISDYLYWYQQKPGKAPKLLIYFASQSISGVPSRFSGSGSGTDFTLTISSLQPEDFATYYCQNGHSFPFTFGGGTKVEIK

SEQ ID NO: 161 - protein, 107 aa, synthetic construct
  REGION 1..107: note = 23A0184VL
DIQMTQSPSSLSASVGDRVTITCRASQSISDYLYWYQQKPGKAPKLLIKFASQSISGIPSRFSGSGSGSDFTLTISSLQPEDFATFYCQNGHSFPFTFGGGTKVEIK

SEQ ID NO: 162 - protein, 107 aa, synthetic construct
  REGION 1..107: note = 23A0185VL
DIVMTQSPSSLSASVGDRVTLTCRASQSISDYLYWYQQKPGKAPKLLIKFASQSISGIPSRFSGSGSGSDFTLTISSVQPEDFATFFCQNGHSFPFTFGGGTKVEIK

SEQ ID NO: 163 - protein, 107 aa, synthetic construct
  REGION 1..107: note = 23A0186-4VL
EIVMTQSPATLSVSPGERVTLSCRASQSISDYLYWYQQKPGQAPRLLIKFASQSISGIPARFSGSGSGSEFTLTISSVQSEDFAVFFCQNAHSFPFTFGGGTKVEIK

SEQ ID NO: 164 - protein, 107 aa, synthetic construct
  REGION 1..107: note = 23A0186-5VL
EIVMTQSPATLSVSPGERVTLSCRASQSISDYLYWYQQKPGQAPRLLIKFASQSISGIPARFSGSGSGSEFTLTISSVQSEDFAVFFCQQGHSFPFTFGGGTKVEIK

SEQ ID NO: 165 - protein, 107 aa, synthetic construct
  REGION 1..107: note = 23A0186-6VL
EIVMTQSPATLSVSPGERVTLSCRASQSISDYLYWYQQKPGQAPRLLIKFASQSISGIPARFSGSGSGSEFTLTISSVQSEDFAVFFCQNGHSFPFTFGGGTKVEIK

SEQ ID NO: 166 - protein, 111 aa, synthetic construct
  REGION 1..111: note = 23A0198 (A5M21)VL
DIVMTQSPDSLAVSLGERATINCKASKKVTIYGSTSALHWYQQKPGQPPKLIYNLAKPESGVPDRFSGSGSGTDFTLTISSLQAEDVAVYYCLQNKEVPYTFGGGTKVEIK

SEQ ID NO: 167 - protein, 107 aa, synthetic construct [sequence omitted]

SEQ ID NO: 168 - protein, 103 aa, synthetic construct
  REGION 1..103: note = CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 169 - protein, 227 aa, synthetic construct
  REGION 1..227: note = Fc(LALA+YTE)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 227 aa, synthetic construct
  REGION 1..227: note = Fc(LALA+LS)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 171 - protein, 227 aa, synthetic construct
  REGION 1..227: note = Fc-Knob (LALA+YTE)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 172 - protein, 227 aa, synthetic construct
  REGION 1..227: note = Fc-Hole (LALA+YTE)
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 173 - protein, 227 aa, synthetic construct
  REGION 1..227: note = Fc-Knob (LALA+LS)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 174 - protein, 227 aa, synthetic construct
  REGION 1..227: note = Fc-Hole (LALA+LS)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRFTQKSLSLSPGK

SEQ ID NO: 175 - protein, 214 aa, synthetic construct
  REGION 1..214: note = light chain 1
AIVMTQSPATLSVSPGERVTLSCRASQSISDYLYWYQQKPDQSPKLLIKFASQSISGVPSRFSGSGSGRDFTLTISSLQPEDFAVYYCQNGHSFPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 214 aa, synthetic construct
  REGION 1..214: note = light chain 2
EIVMTQSPATLSLSPGERATLSCRASQSISDYLYWYQQKPDQSPKLLIKFASQSISGVPSRFSGSGSGTEFTLTISSLQPEDFAVYYCQNGHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 214 aa, synthetic construct
  REGION 1..214: note = light chain 3
DIVMTQSPATLSVSPGERVTLSCRASQSISDYLYWYQQKPDQSPKLLIKFASQSISGVPSRFSGSGSGSDFTLTISSLQPEDFATYYCQNGHSFPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 214 aa, synthetic construct
  REGION 1..214: note = light chain 4
EIVMTQSPATLSVSPGERVTLSCRASQSISDYLYWYQQKPGQAPRLLIKFASQSISGIPARFSGSGSGSEFTLTISSVQSEDFAVFFCQQGHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 214 aa, synthetic construct
  REGION 1..214: note = light chain 5
EIVMTQSPATLSVSPGERVTLSCRASQSISDYLYWYQQKPGQAPRLLIKFASQSISGIPARFSGSGSGSEFTLTISSVQSEDFAVFFCQNGHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 214 aa, synthetic construct
  REGION 1..214: note = light chain 6
EIVLTQSPATLSLSPGERATLSCRASQSVSSNYLAWYQQKPGQAPRLLIYYASRRATGVPARFSGSGSGTDFTLTISSLEPEDFAYYCQQTSNTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 218 aa, synthetic construct
  REGION 1..218: note = light chain 7
EIVLTQSPATLSLSPGERATLSCRASKSVRFSDYFYMHWYQQKPGQAPRLLIYLASNLESGIPARFSGSGSGTDFTLTISSLEPEDFAVYYCQNARELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 218 aa, synthetic construct
  REGION 1..218: note = light chain 8
DIVMTQSPDSLAVSLGERATINCKASKKVTIYGSTSALHWYQQKPGQPPKLIYNLAKPESGVPDRFSGSGSGTDFTLTISSLQAEDVAVYYCLQNKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 445 aa, synthetic construct
  REGION 1..445: note = TLb0090 heavy chain1
EVQLQQSGPELVKPGASVKVSCKASGYTFTRYLMHWVRQAPGQGLEWIGYINPYNDGTKYNQKFQGRVTITADKSTSTAYMELSSLTSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 445 aa, synthetic construct
  REGION 1..445: note = TLb0091 heavy chain1
QVQLQQSGSELKKPGASVKVSCKASGYTFTRYLMHWVRQAPGKGLEWIGYINPYNDGTKYNQKFQGRVTITADKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 445 aa, synthetic construct
  REGION 1..445: note = TLb0092 heavy chain1
EIQLQQSGSELKKPGASVKVSCKASGYTFTRYLMNWVRQAPGKGLEWIGYINPYNDGTKYNQKFQGRVTITADKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 445 aa, synthetic construct
  REGION 1..445: note = TLb0100 heavy chain1
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 445 aa, synthetic construct; identical to SEQ ID NO: 186
  REGION 1..445: note = TLb0102 heavy chain1

SEQ ID NO: 188 - protein, 445 aa, synthetic construct
  REGION 1..445: note = TLb0102-1 heavy chain1
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 189 - protein, 456 aa, synthetic construct
  REGION 1..456: note = TLb0109 heavy chain1
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 190 - protein, 456 aa, synthetic construct
  REGION 1..456: note = TLb0109-1 heavy chain1
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 191 - protein, 475 aa, synthetic construct
  REGION 1..475: note = TLb0090 heavy chain2
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSEPKSADKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 192 - protein, 475 aa, synthetic construct; identical to SEQ ID NO: 191
  REGION 1..475: note = TLb0091 heavy chain2

SEQ ID NO: 193 - protein, 475 aa, synthetic construct; identical to SEQ ID NO: 191
  REGION 1..475: note = TLb0092 heavy chain2

SEQ ID NO: 194 - protein, 475 aa, synthetic construct; identical to SEQ ID NO: 191
  REGION 1..475: note = TLb0100 heavy chain2

SEQ ID NO: 195 - protein, 475 aa, synthetic construct; identical to SEQ ID NO: 191
  REGION 1..475: note = TLb0102 heavy chain2

SEQ ID NO: 196 - protein, 475 aa, synthetic construct
  REGION 1..475: note = TLb0102-1 heavy chain2
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVLHEALHSRFTQKSLSLSPGK

SEQ ID NO: 197 - protein, 475 aa, synthetic construct; identical to SEQ ID NO: 191
  REGION 1..475: note = TLb0109 heavy chain2

SEQ ID NO: 198 - protein, 475 aa, synthetic construct; identical to SEQ ID NO: 196
  REGION 1..475: note = TLb0109-1 heavy chain2

SEQ ID NO: 199 - protein, 708 aa, synthetic construct
  REGION 1..708: note = TLb0103 heavy chain
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 200 - protein, 708 aa, synthetic construct
  REGION 1..708: note = TLb0103-1 heavy chain
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 201 - protein, 698 aa, synthetic construct
  REGION 1..698: note = TLb0103-2 heavy chain
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 202 - protein, 703 aa, synthetic construct
  REGION 1..703: note = TLb0103-3 heavy chain
QVQLVQSGAEVVKPGASVKMSCKASGYTFTRYLIHWVKQAPGQGLEWIGYINPYNEGTKYNEKFKGRATLTSDKSTSTAYMELSSLRSEDTAVYYCTSNWDLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 203 - protein, 711 aa, synthetic construct
  REGION 1..711: note = TLb0107 heavy chain
QVQLVQSGAEVKKPGSSVKVSCKASGGTFSSNYISWVRQAPGQGLEWMGAYAQKFQGRVTITADESTSTAYMELSSLRSEDTAVYYCARSKKGMYGGWTYPLMMFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 204 - protein, 712 aa, synthetic construct
  REGION 1..712: note = TLb0108 heavy chain
QVQLVQSGAEVVKPGSSVKVSCKASGYTFTDYYMNWVRQAPGQGLEWIGDFNHNNDVITYNPKFKGRVTLTSDKSTSTAYMELSSLRSEDTAVYYCARGLRGYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 205 - protein, 719 aa, synthetic construct
  REGION 1..719: note = TLb0110 heavy chain
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 206 - protein, 719 aa, synthetic construct
  REGION 1..719: note = TLb0110-1 heavy chain
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 207 - protein, 709 aa, synthetic construct
  REGION 1..709: note = TLb0110-2 heavy chain
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 208 - protein, 714 aa, synthetic construct
  REGION 1..714: note = TLb0110-3 heavy chain
QVQLVQSGAEVKKPGSSVKVSCKASGYTFTSYGITWVRQAPGQGLEWMGENYPRSGNTYYNEKFKGRVTITADKSTSTAYMELSSLRSEDTAVYYCARAEFISTVVAPYYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGGGSGGGGSGGGGSEIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSSGGGGSGGGGSGGGGSGGGGSDIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 209 - protein, 30 aa, synthetic construct
  REGION 1..30: note = HFR1-1
QVQLVQSGAEVKKPGSSVKVSCKASGYTFT

SEQ ID NO: 210 - protein, 30 aa, synthetic construct
  REGION 1..30: note = HFR1-2
EVQLVESGGGLVKPGGSLRLSCAASGYTFT

SEQ ID NO: 211 - protein, 30 aa, synthetic construct
  REGION 1..30: note = HFR1-3
EVQLQQSGPELVKPGASVKVSCKASGYTFT

SEQ ID NO: 212 - protein, 30 aa, synthetic construct
  REGION 1..30: note = HFR1-4
EVQLQQSGPEVKKPGASVKVSCKASGYTFT

SEQ ID NO: 213 - protein, 30 aa, synthetic construct
  REGION 1..30: note = HFR1-5
QVQLQQSGSELKKPGASVKVSCKASGYTFT

SEQ ID NO: 214 - protein, 30 aa, synthetic construct
  REGION 1..30: note = HFR1-6
EIQLQQSGSELKKPGASVKVSCKASGYTFT

SEQ ID NO: 215 - protein, 30 aa, synthetic construct
  REGION 1..30: note = HFR1-7
QVQLVQSGAEVKKPGASVKVSCKASGYTFT

SEQ ID NO: 216 - protein, 30 aa, synthetic construct
  REGION 1..30: note = HFR1-8
QVQLVQSGAEVVKPGASVKMSCKASGYTFT

SEQ ID NO: 217 - protein, 14 aa, synthetic construct
  REGION 1..14: note = HFR2-1
WVRQAPGQGLEWIG

SEQ ID NO: 218 - protein, 14 aa, synthetic construct
  REGION 1..14: note = HFR2-2
WVRQAPGKGLEWIG

SEQ ID NO: 219 - protein, 14 aa, synthetic construct
  REGION 1..14: note = HFR2-3
WVRQPPGQGLEWIG

SEQ ID NO: 220 - protein, 14 aa, synthetic construct
  REGION 1..14: note = HFR2-4
WVRQAPGQGLEWMG

SEQ ID NO: 221 - protein, 14 aa, synthetic construct
  REGION 1..14: note = HFR2-5
WVKQAPGQGLEWIG

SEQ ID NO: 222 - protein, 32 aa, synthetic construct
  REGION 1..32: note = HFR3-1
RVTITADESTSTAYMELSSLRSEDTAVYYCTS

SEQ ID NO: 223 - protein, 32 aa, synthetic construct
  REGION 1..32: note = HFR3-2
RFTISRDDSKNTLYLQMNSLKTEDTAVYYCTS

SEQ ID NO: 224 - protein, 32 aa, synthetic construct
  REGION 1..32: note = HFR3-3
RVTITADKSTSTAYMELSSLTSEDTAVYYCTS

SEQ ID NO: 225 - protein, 32 aa, synthetic construct
  REGION 1..32: note = HFR3-4
RVTITADKSTSTAYMELSSLRSEDTAVYYCTS

SEQ ID NO: 226 - protein, 32 aa, synthetic construct
  REGION 1..32: note = HFR3-5
RVTMTRDTSTSTVYMELSSLRSEDTAVYYCAS

SEQ ID NO: 227 - protein, 32 aa, synthetic construct
  REGION 1..32: note = HFR3-6
RVTMTSDKSTSTAYMELSSLRSEDTAVYYCTS

SEQ ID NO: 228 - protein, 32 aa, synthetic construct
  REGION 1..32: note = HFR3-7
RATLTSDKSTSTAYMELSSLRSEDTAVYYCTS

SEQ ID NO: 229 - protein, 32 aa, synthetic construct
RVTITADKSTSTAYMELSSLRSEDTAVYYCAR

SEQ ID NO: 230 - protein, 11 aa, synthetic construct
  REGION 1..11: note = HFR4-2
WGQGTTLTVSS

SEQ ID NO: 231 - protein, 23 aa, synthetic construct
  REGION 1..23: note = LFR1-1
AIQMTQSPSSLSASVGDRVTITC

SEQ ID NO: 232 - protein, 23 aa, synthetic construct
DIVMTQSPDSLAVSLGERATINC

SEQ ID NO: 233 - protein, 23 aa, synthetic construct
  REGION 1..23: note = LFR1-3
NIQMTQSPSAMSASVGDRVTITC

SEQ ID NO: 234 - protein, 23 aa, synthetic construct
  REGION 1..23: note = LFR1-4
DIQMTQSPSTLSASVGDRVTITC

SEQ ID NO: 235 - protein, 23 aa, synthetic construct
  REGION 1..23: note = LFR1-5
AIVMTQSPATLSVSPGERVTLSC

SEQ ID NO: 236 - protein, 23 aa, synthetic construct
  REGION 1..23: note = LFR1-6
NIVMTQSPATLSVSPGERATLSC

SEQ ID NO: 237 - protein, 23 aa, synthetic construct
  REGION 1..23: note = LFR1-7
EIVMTQSPATLSLSPGERATLSC

SEQ ID NO: 238 - protein, 23 aa, synthetic construct
  REGION 1..23: note = LFR1-8
DIVMTQSPATLSVSPGERVTLSC

SEQ ID NO: 239 - protein, 23 aa, synthetic construct
  REGION 1..23: note = LFR1-9
DIVMTQSPSSLSASVGDRVTLTC

SEQ ID NO: 240 - protein, 23 aa, synthetic construct
  REGION 1..23: note = LFR1-10
EIVMTQSPATLSVSPGERVTLSC

SEQ ID NO: 241 - protein, 15 aa, synthetic construct
  REGION 1..15: note = LFR2-1
WYQQKPGKAPKLLIK

SEQ ID NO: 242 - protein, 15 aa, synthetic construct
  REGION 1..15: note = LFR2-2
WFQQKPGKAPKLLIK

SEQ ID NO: 243 - protein, 15 aa, synthetic construct
  REGION 1..15: note = LFR2-3
WFQQKPGKVPKLLIK

SEQ ID NO: 244 - protein, 15 aa, synthetic construct
  REGION 1..15: note = LFR2-4
WYQQKPDQSPKLLIK

SEQ ID NO: 245 - protein, 15 aa, synthetic construct
  REGION 1..15: note = LFR2-5
WYQQKPDQSPRLLIK

SEQ ID NO: 246 - protein, 14 aa, synthetic construct
WYQQKPGQPPKLIY

SEQ ID NO: 247 - protein, 15 aa, synthetic construct
  REGION 1..15: note = LFR2-7
WYQQKPGQAPRLLIK

SEQ ID NO: 248 - protein, 33 aa, synthetic construct
  REGION 1..33: note = LFR3-1
GVPSRFSGSGSGTDFTLTISSLQPEDFATYYCQ

SEQ ID NO: 249 - protein, 33 aa, synthetic construct
  REGION 1..33: note = LFR3-2
GVPSRFSGSGSGTEFTLTISSLQPEDFATYYCQ

SEQ ID NO: 250 - protein, 33 aa, synthetic construct
  REGION 1..33: note = LFR3-3
GVPSRFSGSGSGTEFTLTISSLQPDDFATYYCQ

SEQ ID NO: 251 - protein, 33 aa, synthetic construct
  REGION 1..33: note = LFR3-4
GVPSRFSGSGSGRDFTLTISSLQPEDFAVYYCQ

SEQ ID NO: 252 - protein, 33 aa, synthetic construct
  REGION 1..33: note = LFR3-5
GVPSRFSGSGSGRDFTLTISSLEPEDFAVYYCQ

SEQ ID NO: 253 - protein, 33 aa, synthetic construct
  REGION 1..33: note = LFR3-6
GVPSRFSGSGSGTEFTLTISSLQPEDFAVYYCQ

SEQ ID NO: 254 - protein, 33 aa, synthetic construct
  REGION 1..33: note = LFR3-7
GVPSRFSGSGSGSDFTLTISSLQPEDFATYYCQ

SEQ ID NO: 255 - protein, 33 aa, synthetic construct
  REGION 1..33: note = LFR3-8
GVPSRFSGSGSGSDFTLKISSVEAEDVGVYYCQ

SEQ ID NO: 256 - protein, 33 aa, synthetic construct
  REGION 1..33: note = LFR3-9
GIPSRFSGSGSGSDFTLTISSLQPEDFATFYCQ

SEQ ID NO: 257 - protein, 33 aa, synthetic construct
  REGION 1..33: note = LFR3-10
GIPSRFSGSGSGSDFTLTISSVQPEDFATFFCQ

SEQ ID NO: 258 - protein, 33 aa, synthetic construct
  REGION 1..33: note = LFR3-11
GIPARFSGSGSGSEFTLTISSVQSEDFAVFFCQ

SEQ ID NO: 259 - protein, 10 aa, synthetic construct
  REGION 1..10: note = LFR4-1
FGPGTKVDIK

SEQ ID NO: 260 - protein, 10 aa, synthetic construct
  REGION 1..10: note = LFR4-2
FGQGTRLEIK

SEQ ID NO: 261 - protein, 10 aa, synthetic construct
  REGION 1..10: note = LFR4-3
FGQGTKVEIK

SEQ ID NO: 262 - protein, 10 aa, synthetic construct
  REGION 1..10: note = LFR4-4
FGGGTKLEIK

SEQ ID NO: 263 - protein, 10 aa, synthetic construct
  REGION 1..10: note = LFR4-5
FGGGTKVEIK

SEQ ID NO: 264 - protein, 5 aa, synthetic construct
  REGION 1..5: note = 23A0198 HCDR1
SYGIT

SEQ ID NO: 265 - protein, 17 aa, synthetic construct
  REGION 1..17: note = 23A0198 HCDR2
ENYPRSGNTYYNEKFKG

SEQ ID NO: 266 - protein, 17 aa, synthetic construct
  REGION 1..17: note = 23A0198 HCDR3
AEFISTVVAPYYYALDY

SEQ ID NO: 267 - protein, 16 aa, synthetic construct
  REGION 1..16: note = 23A0198 LCDR1
KASKKVTIYGSTSALH

SEQ ID NO: 268 - protein, 7 aa, synthetic construct
  REGION 1..7: note = 23A0198 LCDR2
NLAKPES

SEQ ID NO: 269 - protein, 9 aa, synthetic construct
  REGION 1..9: note = 23A0198 LCDR3
LQNKEVPYT

SEQ ID NO: 270 - protein, 116 aa, synthetic construct
EIQLVESGGGLIQPGGSLRISCAVSGFSLSSYGVDWVRQAPGKCLEWLGVIWGFGGTNYNSALKSRLTISKDNSKNTVYLQMNSLRAEDTAVYYCASGNFDAMDYWGQGTLVTVSS

SEQ ID NO: 271 - protein, 107 aa, synthetic construct
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPFTFGCGTKLEIK

SEQ ID NO: 272 - protein, 11 aa, synthetic construct
WGQGTLVTVSS

SEQ ID NO: 273 - protein, 32 aa, synthetic construct
GVPDRFSGSGSGTDFTLTISSLQAEDVAVYYC

(19) United States
(12) Patent Application Publication
(10) Pub. No.: US 2026/0062472 A1
(43) Pub. Date: Mar. 5, 2026

(54) BISPECIFIC ANTIBODY AND USE THEREOF

(71) Applicant: EARENDIL LABS INC., Wilmington, DE (US)

(72) Inventors: Hao RAN, Suzhou, Jiangsu (CN); Jian HUANG, Suzhou, Jiangsu (CN); Zhenping ZHU, Suzhou, Jiangsu (CN); Jian PENG, Suzhou, Jiangsu (CN); Liang TIAN, Suzhou, Jiangsu (CN)

(21) Appl. No.: 19/312,684

(22) Filed: Aug. 28, 2025

(30) Foreign Application Priority Data
Aug. 28, 2024 (CN) 202411195305.2
Jan. 27, 2025 (CN) 202510127798.4
Feb. 5, 2025 (CN) 202510130736.9
May 30, 2025 (CN) 202510725012.9

(51) Int. Cl. C07K 16/24; A61K 38/00; A61P 29/00; C12N 15/11

(52) U.S. Cl. CPC C07K 16/241; A61K 38/00; A61P 29/00; C07K 16/244; C12N 15/11; C07K 2317/31; C07K 2317/565; C07K 2317/622; C07K 2317/76; C07K 2317/92

(57) ABSTRACT

The present application provides an isolated antigen-binding protein, comprising: a first binding domain and a second binding domain, wherein the first binding domain specifically binds to TNF-like protein A (TL1A), and the first binding domain and the second binding domain are different.

### RELATED APPLICATIONS

This application claims priority to Chinese Patent Application Nos. 202510725012.9, filed May 30, 2025, 202510130736.9, filed Feb. 5, 2025, 202510127798.4, filed Jan. 27, 2025, and 202411195305.2, filed Aug. 28, 2024, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Sep. 2, 2025, is named 769250_SA9-944_ST26.xml and is 379,157 bytes in size.

### FIELD OF THE INVENTION

The present application relates to the field of biomedicine, and specifically to an antigen binding protein of TL1A and IL-23p19.

### BACKGROUND OF THE INVENTION

The immune system can identify and eliminate foreign substances to maintain the homeostasis of the immune system. Under normal circumstances, the immune system only responds to foreign substances, but not to self-tissue antigens. However, under the influence of some unknown reasons, the immune system functions abnormally and responds to self-tissue antigens, producing antibodies or immune cells to attack self-cells or tissues. Such a reaction is called an autoimmune reaction, which can lead to inflammation and tissue damage. Such diseases are called autoimmune diseases. There are many types of autoimmune diseases, and more than 100 autoimmune diseases have been discovered.

Inflammatory bowel disease is a chronic, progressive, recurrent or remitted intestinal disease that can affect the colon and small intestine. Types include ulcerative colitis (UC) and Crohn's disease (CD). For most IBD patients, traditional treatments such as hormones, immunosuppressants, and surgical treatment are the main treatments, which enable patients to achieve clinical remission and reduce disease recurrence. As research on IBD deepens, biological agents are also developing rapidly. Currently, the drugs approved for marketing include anti-TNF-α monoclonal antibody drugs, anti-α4β7 monoclonal antibody drugs, anti-IL-12 monoclonal antibody drugs, anti-IL-23 monoclonal antibody drugs, and anti-TL1A monoclonal antibody drugs in the clinical stage, which have shown positive therapeutic effects in inflammatory bowel disease, but only some patients have achieved clinical benefits in clinical practice, and the overall efficacy needs to be further improved.

In the past 20 years, biological macromolecule preparations have achieved significant therapeutic effects in the treatment of ulcerative colitis, promoting new breakthroughs in the clinical remission rate of ulcerative colitis. However, most current treatments have a 1-year clinical remission rate between 30% and 50%, and more than 50% of patients still have insufficient clinical response, indicating that the upper limit of treatment may have been reached by using a single drug. With this in mind, the concept of combined treatment with two biological agents may be a promising strategy for patients with refractory ulcerative colitis, patients at high risk of complications, or patients with uncontrolled immune-mediated inflammatory diseases. This strategy has been applied to actual treatment and has achieved good results. A systematic review and meta-analysis of combination therapy published in 2022 showed that among the 279 patients who received combination therapy in a total of 30 studies, the clinical remission rate and endoscopic remission rate reached 59% and 34%, respectively, which were significantly higher than the treatment effect of a single drug. Among them, 81% of patients with refractory ulcerative colitis and 12% of patients with ulcerative colitis complicated by extraintestinal manifestations or rheumatism. The most commonly used combinations in treatment regimens include anti-integrin monoclonal antibody+anti-TNF-α monoclonal antibody (accounting for 48%) and anti-integrin monoclonal antibody+ustekinumab (accounting for 19%). Therefore, the combination of multiple targets will be an important development direction for the future development and clinical treatment of IBD and other autoimmune diseases.

The present invention relates to antigen binding proteins, in particular antibodies that bind to TL1A and interleukin 23 (IL-23) and neutralize their activity, polynucleotides encoding such antigen binding proteins, pharmaceutical preparations containing the antigen binding proteins, and the use of such antigen binding proteins for the treatment and/or prevention of diseases associated with inflammation. Other aspects, objects and advantages of the invention will become apparent from the following description.

### SUMMARY OF THE INVENTION

In one aspect, the present application provides a separated antigen binding protein, comprising: a first binding domain and a second binding domain, wherein the first binding domain specifically binds to TNF-like protein A (TL1A), and the first binding domain and the second binding domain are different.

In some embodiments, the second binding domain specifically binds to IL-23, preferably, binds to IL-23p19.

In some embodiments, the KD of the antigen binding protein for IL-23 is less than or equal to about 5E-08M.

In some embodiments, the KD of the antigen binding protein for monomeric TL1A and/or trimeric TL1A is less than or equal to about 5E-08M.

In some embodiments, the antigen binding protein inhibits the production of IL-17 or IL-22 in cells.

In some embodiments, the antigen binding protein inhibits STAT3 phosphorylation in cells.

In some embodiments, the antigen binding protein comprises one or more polypeptides, wherein one polypeptide comprises at least a first binding domain and at least one second binding domain.

In some embodiments, the antigen binding protein includes a first polypeptide and a second polypeptide, wherein the first polypeptide includes a first binding domain and a second binding domain, and the second polypeptide includes a first binding domain or a second binding domain, selected from one or more of the following situations
(1) the first binding domain of the first polypeptide is located at the C-terminus of the first polypeptide, and the first binding domain of the second polypeptide is located at the C-terminus of the first polypeptide;
(2) the first binding domain of the first polypeptide is located at the N-terminus of the first polypeptide, and the first binding domain of the second polypeptide is located at the N-terminus of the first polypeptide;
(3) the second binding domain of the first polypeptide is located at the N-terminus of the first polypeptide, and the second binding domain of the second polypeptide is located at the N-terminus of the second polypeptide;
(3) the second binding domain of the first polypeptide is located at the C-terminus of the second polypeptide, and the second binding domain of the second polypeptide is located at the C-terminus of the second polypeptide.

In some embodiments, the antigen binding protein includes a first polypeptide and a second polypeptide, wherein the first polypeptide includes a first binding domain and a second binding domain, and the second polypeptide includes a first binding domain and a second binding domain, and one or more of the following situations are selected
(1) the first binding domain of the first polypeptide is located at the C-terminus of the first polypeptide, and the first binding domain of the second polypeptide is located at the C-terminus of the first polypeptide;
(2) the first binding domain of the first polypeptide is located at the N-terminus of the first polypeptide, and the first binding domain of the second polypeptide is located at the N-terminus of the first polypeptide;
(3) the second binding domain of the first polypeptide is located at the N-terminus of the first polypeptide, and the second binding domain of the second polypeptide is located at the N-terminus of the second polypeptide;
(3) the second binding domain of the first polypeptide is located at the C-terminus of the second polypeptide, and the second binding domain of the second polypeptide is located at the C-terminus of the second polypeptide.

In some embodiments, it includes a first polypeptide and a second polypeptide, wherein the first polypeptide includes the first binding domain and does not include the second binding domain, and the second polypeptide includes the second binding domain and does not include the first binding domain.

In some embodiments, the first binding domain or the second binding domain comprises one of the following or any combination thereof:

Fab, Fab′, F(ab′)2, Fd and Fv fragments, disulfide-stabilized Fv fragments (dsFv), (dsFv)2, bispecific dsFv (dsFv-dsFv′), disulfide-stabilized diabody (dsdiabody), single-chain antibody molecule (scFv), single domain antibody (sdab), scFv dimer (bivalent diabody), single domain antibody or nanobody, domain antibody (dAb), shark variable IgNAR domain, camelized VH domain, chicken heavy chain antibody domain, VHH domain, minimal recognition unit composed of CDRs of mimicking antibodies, alternative scaffolds for binding to antigens, bivalent domain antibodies, pre-designed ankyrin repeat proteins (DARPins), multispecific proteins (comprising antigen-binding fragments or any other antibody fragments that bind to antigens, but not comprising complete antibody structures).

In some embodiments, the first binding domain comprises a heavy chain variable region (VH), wherein the VH comprises the HCDR3 comprising an amino acid sequence of SEQ ID NO: 14.

In some embodiments, wherein the HCDR3 comprises an amino acid sequence of any one of SEQ ID NOs: 15-18.

In some embodiments, the VH comprises the HCDR2 comprising an amino acid sequence of SEQ ID NO: 5.

In some embodiments, the HCDR2 comprises an amino acid sequence of any one of SEQ ID NOs: 6-13.

In some embodiments, the VH comprises the HCDR1 comprising an amino acid sequence of SEQ ID NO: 1.

In some embodiments, the HCDR1 comprises an amino acid sequence of any one of SEQ ID NOs: 2-4.

In some embodiments, the VH of the first binding domain comprises HCDR1 as shown in SEQ ID NO: 2, the HCDR2 as shown in SEQ ID NO: 7, and the HCDR3 as shown in SEQ ID NO: 15.

In some embodiments, the VH of the first binding domain comprises the HCDR1 as shown in SEQ ID NO:2, the HCDR2 as shown in SEQ ID NO:8, and the HCDR3 as shown in SEQ ID NO:15.

In some embodiments, the VH of the first binding domain comprises the HCDR1 as shown in SEQ ID NO:2, the HCDR2 as shown in SEQ ID NO:6, and the HCDR3 as shown in SEQ ID NO:18.

In some embodiments, the VH of the first binding domain comprises the HCDR1 as shown in SEQ ID NO:4, the HCDR2 as shown in SEQ ID NO:6, and the HCDR3 as shown in SEQ ID NO:15.

In some embodiments, the VH of the first binding domain comprises the HCDR1 as shown in SEQ ID NO:2, the HCDR2 as shown in SEQ ID NO:9, and the HCDR3 as shown in SEQ ID NO:15.

In some embodiments, the VH of the first binding domain comprises the HCDR1 as shown in SEQ ID NO:2, the HCDR2 as shown in SEQ ID NO:10, and the HCDR3 as shown in SEQ ID NO:15.

In some embodiments, the VH of the first binding domain comprises the HCDR1 as shown in SEQ ID NO:2, the HCDR2 as shown in SEQ ID NO:11, and the HCDR3 as shown in SEQ ID NO:15.

In some embodiments, the VH of the first binding domain comprises the HCDR1 as shown in SEQ ID NO:2, the HCDR2 as shown in SEQ ID NO:6, and the HCDR3 as shown in SEQ ID NO:15.

In some embodiments, the VH of the first binding domain comprises the HCDR1 as shown in SEQ ID NO:2, the HCDR2 as shown in SEQ ID NO:12, and the HCDR3 as shown in SEQ ID NO:15.

In some embodiments, the VH of the first binding domain comprises the HCDR1 as shown in SEQ ID NO:2, the HCDR2 as shown in SEQ ID NO:13, and the HCDR3 as shown in SEQ ID NO:15.

In some embodiments, the VH of the first binding domain comprises the HCDR1 as shown in SEQ ID NO:3, the HCDR2 as shown in SEQ ID NO:6, and the HCDR3 as shown in SEQ ID NO:16.

In some embodiments, the VH of the first binding domain comprises the HCDR1 as shown in SEQ ID NO:3, the HCDR2 as shown in SEQ ID NO:6 and the HCDR3 as shown in SEQ ID NO:17.

In some embodiments, the VH of the first binding domain comprises the HCDR1 as shown in SEQ ID NO:3, the HCDR2 as shown in SEQ ID NO:9, and the HCDR3 as shown in SEQ ID NO:15.

In some embodiments, the VH of the first binding domain comprises the HCDR1 as shown in SEQ ID NO:3, the HCDR2 as shown in SEQ ID NO:10, and the HCDR3 as shown in SEQ ID NO:15.

In some embodiments, the VH of the first binding domain comprises the HCDR1 as shown in SEQ ID NO:3, the HCDR2 as shown in SEQ ID NO:11, and the HCDR3 as shown in SEQ ID NO:15.

In some embodiments, the VH of the first binding domain comprises the HCDR14 as shown in SEQ ID NO:3, the HCDR2 as shown in SEQ ID NO:6, and the HCDR3 as shown in SEQ ID NO:15.

In some embodiments, the VH of the first binding domain comprises the HCDR1 as shown in SEQ ID NO:2, the HCDR2 as shown in SEQ ID NO:6, and the HCDR3 as shown in SEQ ID NO:16.

In some embodiments, the VH of the first binding domain comprises the HCDR1 as shown in SEQ ID NO:2, the HCDR2 as shown in SEQ ID NO:9, and the HCDR3 as shown in SEQ ID NO:16.

In some embodiments, the VH of the first binding domain comprises the HCDR1 as shown in SEQ ID NO:2, the HCDR2 as shown in SEQ ID NO:10, and the HCDR3 as shown in SEQ ID NO:16.

In some embodiments, the VH of the first binding domain comprises the HCDR1 as shown in SEQ ID NO:2, the HCDR2 as shown in SEQ ID NO:11, and the HCDR3 as shown in SEQ ID NO:16.

In some embodiments, the VH of the first binding domain comprises the HCDR1 as shown in SEQ ID NO:2, the HCDR2 as shown in SEQ ID NO:12, and the HCDR3 as shown in SEQ ID NO:16.

In some embodiments, the VH of the first binding domain comprises the HCDR1 as shown in SEQ ID NO:2, the HCDR2 as shown in SEQ ID NO:6, and the HCDR3 as shown in SEQ ID NO:17.

In some embodiments, the VH of the first binding domain comprises the HCDR1 as shown in SEQ ID NO:2, the HCDR2 as shown in SEQ ID NO:10, and the HCDR3 as shown in SEQ ID NO:17.

In some embodiments, the VH comprises HFR1, and the HFR1 comprises the amino acid sequence of SEQ ID NO:19.

In some embodiments, the HFR1 is selected from the amino acid sequence shown in any one of SEQ ID NO:20-28.

In some embodiments, the VH comprises HFR2, and the HFR2 comprises the amino acid sequence of SEQ ID NO:29.

In some embodiments, the HFR2 is selected from the amino acid sequence shown in any one of SEQ ID NO:30-32.

In some embodiments, the VH comprises HFR3 comprising the amino acid sequence of SEQ ID NO:33.

In some embodiments, the HFR3 is selected from the amino acid sequence shown in any one of SEQ ID NO:34-40.

In some embodiments, the VH comprises HFR4 comprising the amino acid sequence shown in SEQ ID NO:41.

In some embodiments, the VH of the first binding domain comprises HFR1 as shown in SEQ ID NO:20, HFR2 as shown in SEQ ID NO:30, HFR3 as shown in SEQ ID NO:34, and HFR4 as shown in SEQ ID NO:41.

In some embodiments, the VH of the first binding domain comprises HFR1 as shown in SEQ ID NO:21, HFR2 as shown in SEQ ID NO:31, HFR3 as shown in SEQ ID NO:35, and HFR4 as shown in SEQ ID NO:41.

In some embodiments, the VH of the first binding domain comprises HFR1 as shown in SEQ ID NO:22, HFR2 as shown in SEQ ID NO:32, HFR3 as shown in SEQ ID NO:35, and HFR4 as shown in SEQ ID NO:41.

In some embodiments, the VH of the first binding domain comprises HFR1 as shown in SEQ ID NO:23, HFR2 as shown in SEQ ID NO:31, HFR3 as shown in SEQ ID NO:35, and HFR4 as shown in SEQ ID NO:41.

In some embodiments, the VH of the first binding domain comprises HFR1 as shown in SEQ ID NO:24, HFR2 as shown in SEQ ID NO:31, HFR3 as shown in SEQ ID NO:38, and HFR4 as shown in SEQ ID NO:41.

In some embodiments, the VH of the first binding domain comprises HFR1 as shown in SEQ ID NO:23, HFR2 as shown in SEQ ID NO:31, HFR3 as shown in SEQ ID NO:37, and HFR4 as shown in SEQ ID NO:41.

In some embodiments, the VH of the first binding domain comprises HFR1 as shown in SEQ ID NO:23, HFR2 as shown in SEQ ID NO:31, HFR3 as shown in SEQ ID NO:36, and HFR4 as shown in SEQ ID NO:41.

In some embodiments, the VH of the first binding domain comprises HFR1 as shown in SEQ ID NO:25, HFR2 as shown in SEQ ID NO:31, HFR3 as shown in SEQ ID NO:37, and HFR4 as shown in SEQ ID NO:41.

In some embodiments, the VH of the first binding domain comprises HFR1 as shown in SEQ ID NO:25, HFR2 as shown in SEQ ID NO:31, HFR3 as shown in SEQ ID NO:39, and HFR4 as shown in SEQ ID NO:41.

In some embodiments, the VH of the first binding domain comprises HFR1 as shown in SEQ ID NO:25, HFR2 as shown in SEQ ID NO:31, HFR3 as shown in SEQ ID NO:40, and HFR4 as shown in SEQ ID NO:41.

In some embodiments, the VH of the first binding domain comprises HFR1 as shown in SEQ ID NO:27, HFR2 as shown in SEQ ID NO:31, HFR3 as shown in SEQ ID NO:35, and HFR4 as shown in SEQ ID NO:41.

In some embodiments, the VH of the first binding domain comprises HFR1 as shown in SEQ ID NO:28, HFR2 as shown in SEQ ID NO:31, HFR3 as shown in SEQ ID NO:35, and HFR4 as shown in SEQ ID NO:41.

In some embodiments, the VH of the first binding domain comprises HFR1 as shown in SEQ ID NO:26, HFR2 as shown in SEQ ID NO:31, HFR3 as shown in SEQ ID NO:35, and HFR4 as shown in SEQ ID NO:41.

In some embodiments, the VH of the first binding domain comprises HFR1 as shown in SEQ ID NO:25, HFR2 as shown in SEQ ID NO:31, HFR3 as shown in SEQ ID NO:35, and HFR4 as shown in SEQ ID NO:41.

In some embodiments, the first binding domain further comprises a light chain variable region (VL), and the VL of the first binding domain comprises at least one light chain complementary determining region (LCDR) comprising an amino acid sequence selected from the amino acid sequences shown in SEQ ID NO:42, SEQ ID NO:43 and SEQ ID NO:44. In some embodiments, the VL comprises the LCDR3 comprising the amino acid sequence of SEQ ID NO:44.

In some embodiments, the VL comprises the LCDR2 comprising the amino acid sequence of SEQ ID NO:43.

In some embodiments, the VL comprises the LCDR1 comprising the amino acid sequence of SEQ ID NO:42.

In some embodiments, the VL comprises LFR1 comprising the amino acid sequence of SEQ ID NO:45.

In some embodiments, the LFR1 comprises an amino acid sequence as shown in any one of SEQ ID NOs: 46-47.

In some embodiments, the VL comprises LFR2 comprising an amino acid sequence as shown in any one of SEQ ID NOs: 48.

In some embodiments, the LFR2 comprises an amino acid sequence as shown in any one of SEQ ID NOs: 49-52.

In some embodiments, the VL comprises LFR3 comprising an amino acid sequence as shown in any one of SEQ ID NOs: 53.

In some embodiments, the LFR3 comprises an amino acid sequence as shown in any one of SEQ ID NOs: 54-57.

In some embodiments, the VL comprises LFR4 comprising an amino acid sequence as shown in any one of SEQ ID NOs: 58.

In some embodiments, the LFR4 comprises an amino acid sequence as shown in any one of SEQ ID NOs: 59-61.

In some embodiments, the VL of the first binding domain comprises LFR1 as shown in SEQ ID NO: 47, LFR2 as shown in SEQ ID NO: 50, LFR3 as shown in SEQ ID NO: 55, and LFR4 as shown in SEQ ID NO: 60.

In some embodiments, the VL of the first binding domain comprises LFR1 as shown in SEQ ID NO:47, LFR2 as shown in SEQ ID NO:51, LFR3 as shown in SEQ ID NO:56, and LFR4 as shown in SEQ ID NO:61.

In some embodiments, the VL of the first binding domain comprises LFR1 as shown in SEQ ID NO:47, LFR2 as shown in SEQ ID NO:52, LFR3 as shown in SEQ ID NO:57, and LFR4 as shown in SEQ ID NO:61.

In some embodiments, the VL of the first binding domain comprises LFR1 as shown in SEQ ID NO:47, LFR2 as shown in SEQ ID NO:52, LFR3 as shown in SEQ ID NO:56, and LFR4 as shown in SEQ ID NO:61.

In some embodiments, the VH of the second binding domain comprises the HCDR1 as shown in SEQ ID NO: 113, the HCDR2 as shown in SEQ ID NO: 117, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 118, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 119, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 120, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 116, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 115, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 122, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 123, and the HCDR3 as shown in SEQ ID NO: 124.

In some embodiments, the VL of the second binding domain comprises the LCDR1 as shown in SEQ ID NO: 125, the LCDR2 as shown in SEQ ID NO: 126, and the LCDR3 as shown in SEQ ID NO: 127.

In some embodiments, the VL of the second binding domain comprises the LCDR1 as shown in SEQ ID NO: 125, the LCDR2 as shown in SEQ ID NO: 126, and the LCDR3 as shown in SEQ ID NO: 130.

In some embodiments, the VL of the second binding domain comprises the LCDR1 as shown in SEQ ID NO: 125, the LCDR2 as shown in SEQ ID NO: 126, and the LCDR3 as shown in SEQ ID NO: 129.

In some embodiments, the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 119, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 120, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 116, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 115, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 122, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 130.

In some embodiments, the VH of the second binding domain comprises the HCDR1 as shown in SEQ ID NO: 114, the HCDR2 as shown in SEQ ID NO: 118, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 130.

In some embodiments, the first binding domain is a scFv or Fab, including VL and VH of the first binding domain.

In some embodiments, in the scFv of the first binding domain, the C-terminus of the VL of the first binding domain is directly or through a linker connected to the N-terminus of the VH of the first binding domain, or the N-terminus of the VL of the first binding domain is directly or through a linker connected to the C-terminus of the VH of the first binding domain.

In some embodiments, the scFv of the first binding domain includes a sequence as shown in any one of SEQ ID NOs: 131-134.

In some embodiments, the second binding domain is a scFv or Fab, and the scFv includes VL and VH of the second binding domain.

In some embodiments, in the scFv of the second binding domain, the C-terminus of the VL of the second binding domain is directly or through a linker connected to the N-terminus of the VH of the second binding domain, or the N-terminus of the VL of the second binding domain is directly or through a linker connected to the C-terminus of the VH of the second binding domain.

In some embodiments, it also comprises a heavy chain constant region of IgG and/or a light chain constant region of a human antibody.

In some embodiments, it comprises a heavy chain constant region of IgG1, IgG2, IgG3 or IgG4.

In some embodiments, it comprises a Kappa or Lambda light chain constant region.

In some embodiments, it is IgG, IgM, IgA, IgD or IgE.

In some embodiments, wherein the antigen binding protein comprises one or more first heavy chains and one or more first light chains, the first heavy chain comprises the VH of the first binding domain and/or the VH of the second binding domain and/or the scFv of the first binding domain and/or the scFv of the second binding domain; the first light chain comprises the VL of the first binding domain or the VL of the second binding domain.

In some embodiments, wherein the first heavy chain comprises the VH of the first binding domain and the scFv of the second binding domain, or the first heavy chain comprises the VH of the second binding domain and the scFv of the first binding domain.

In some embodiments, the antigen binding protein comprises two dimers, each dimer comprising a first heavy chain and a first light chain.

In some embodiments, the N-terminus of the scFv of the first binding domain or the second binding domain is directly or through a linker connected to the C-terminus of the first heavy chain, or the C-terminus of the scFv of the first binding domain or the second binding domain is directly or through a linker connected to the N-terminus of the first heavy chain. In some embodiments, one of the following situations is selected:
(1) the C-terminus of the VH of the first binding domain is directly or through a linker connected to the N-terminus of the first heavy chain, and the N-terminus of the scFv of the second binding domain is directly or through a linker connected to the C-terminus of the first heavy chain; the C-terminus of the VL of the first binding domain is connected to the N-terminus of the light chain constant region;
(2) the C-terminus of the VH of the second binding domain is directly or through a linker connected to the N-terminus of the first heavy chain, and the N-terminus of the scFv of the first binding domain is directly or through a linker connected to the C-terminus of the first heavy chain; the C-terminus of the VL of the second binding domain is connected to the N-terminus of the light chain constant region;
(3) the C-terminus of the scFv of the first binding domain is directly or through a linker connected to the N-terminus of the VH of the second binding domain, and the C-terminus of the VL of the second binding domain is connected to the N-terminus of the light chain constant region;
(4) the C-terminus of the scFv of the second binding domain is directly or through a linker connected to the N-terminus of the VH of the first binding domain, and the C-terminus of the VL of the first binding domain is connected to the N-terminus of the light chain constant region. In some embodiments, the first heavy chain is selected from a sequence shown in any one of SEQ ID NOs: 183-208, and the first light chain is selected from a sequence shown in any one of SEQ ID NOs: 175-182.

In some embodiments, the antigen binding protein is selected from the following combination:
1) a light chain as shown in SEQ ID NO: 179 and a heavy chain as shown in SEQ ID NO: 199;
2) a light chain as shown in SEQ ID NO: 179 and a heavy chain as shown in SEQ ID NO: 200;
3) a light chain as shown in SEQ ID NO: 179 and a heavy chain as shown in SEQ ID NO: 201;
4) a light chain as shown in SEQ ID NO: 179 and a heavy chain as shown in SEQ ID NO: 202;
5) a light chain as shown in SEQ ID NO: 180 and a heavy chain as shown in SEQ ID NO: 203;
6) a light chain as shown in SEQ ID NO: 181 and a heavy chain as shown in SEQ ID NO: 204;
7) a light chain as shown in SEQ ID NO: 182 and a heavy chain as shown in SEQ ID NO: 205;
8) a light chain as shown in SEQ ID NO: 182 and a heavy chain as shown in SEQ ID NO: 206;
9) a light chain as shown in SEQ ID NO: 182 and a heavy chain as shown in SEQ ID NO: 207;
10) a light chain as shown in SEQ ID NO: 182 and a heavy chain as shown in SEQ ID NO: 208.

In some embodiments, the antigen binding protein comprises two light chains and two heavy chains.

In some embodiments, the antigen binding protein comprises one or more first light chains, one or more third heavy chains, and one or more fourth heavy chains, wherein:
1) the third heavy chain comprises the VH of the first binding domain or the second binding domain, and the fourth heavy chain comprises the scFv of the first or second binding domain, or;
2) the third heavy chain comprises the scFv of the first binding domain or the second binding domain, and the fourth heavy chain comprises the VH of the first or second binding domain.

In some embodiments, the N-terminus of the VH of the first binding domain or the second binding domain is directly or through a linker connected to the C-terminus of the third heavy chain, or the N-terminus of the scFv of the first or second binding domain is directly or through a linker connected to the C-terminus of the fourth heavy chain.

In some embodiments, wherein
1) the third heavy chain comprises the VH of the first binding domain, and the fourth heavy chain comprises the scFv of the second binding domain, or;
2) the third heavy chain comprises the scFv of the first binding domain, and the fourth heavy chain comprises the VH of the second binding domain, or;
3) the third heavy chain comprises the VH of the second binding domain, and the fourth heavy chain comprises the scFv of the first binding domain, or
4) the third heavy chain comprises the scFv of the second binding domain, and the fourth heavy chain comprises the VH of the first binding domain.

In some embodiments, the antigen binding protein is selected from the following combination:
1) a light chain as shown in SEQ ID NO: 175, a third heavy chain as shown in SEQ ID NO: 183, and a fourth heavy chain as shown in SEQ ID NO: 191;
2) a light chain as shown in SEQ ID NO: 176, a third heavy chain as shown in SEQ ID NO: 184, and a fourth heavy chain as shown in SEQ ID NO: 192;
3) a light chain as shown in SEQ ID NO: 177, a third heavy chain as shown in SEQ ID NO: 185, and a fourth heavy chain as shown in SEQ ID NO: 193;
4) a light chain as shown in SEQ ID NO: 178, a third heavy chain as shown in SEQ ID NO: 186, and a fourth heavy chain as shown in SEQ ID NO: 194;
5) a light chain as shown in SEQ ID NO: 179, a third heavy chain as shown in SEQ ID NO: 187, and a fourth heavy chain as shown in SEQ ID NO: 195;
6) a light chain as shown in SEQ ID NO: 179, a third heavy chain as shown in SEQ ID NO: 188, and a fourth heavy chain as shown in SEQ ID NO: 196;
7) the light chain as shown in SEQ ID NO: 182, the third heavy chain as shown in SEQ ID NO: 189 and a fourth heavy chain as shown in SEQ ID NO: 197;
8) the light chain as shown in SEQ ID NO: 182, the third heavy chain as shown in SEQ ID NO: 190 and a fourth heavy chain as shown in SEQ ID NO: 198.

In another aspect, the present application provides an isolated antigen-binding protein targeting IL-23p19, wherein the VH comprises the HCDR1 as shown in SEQ ID NO: 113, the HCDR2 as shown in SEQ ID NO: 117, and the HCDR3 as shown in SEQ ID NO: 124.

In some embodiments, the VH comprises the HCDR1 as shown in SEQ ID NO: 114, the HCDR2 as shown in SEQ ID NO: 118, and the HCDR3 as shown in SEQ ID NO: 124.

In some embodiments, the VH comprises the HCDR1 as shown in SEQ ID NO: 114, the HCDR2 as shown in SEQ ID NO: 119, and the HCDR3 as shown in SEQ ID NO: 124.

In some embodiments, the VH comprises the HCDR1 as shown in SEQ ID NO: 114, the HCDR2 as shown in SEQ ID NO: 120, and the HCDR3 as shown in SEQ ID NO: 124.

In some embodiments, the VH comprises the HCDR1 as shown in SEQ ID NO: 114, the HCDR2 as shown in SEQ ID NO: 121, and the HCDR3 as shown in SEQ ID NO: 124.

In some embodiments, the VH comprises the HCDR1 as shown in SEQ ID NO: 116, the HCDR2 as shown in SEQ ID NO: 121, and the HCDR3 as shown in SEQ ID NO: 124.

In some embodiments, the VH comprises the HCDR1 as shown in SEQ ID NO: 115, the HCDR2 as shown in SEQ ID NO: 121, and the HCDR3 as shown in SEQ ID NO: 124.

In some embodiments, the VH comprises the HCDR1 as shown in SEQ ID NO: 114, the HCDR2 as shown in SEQ ID NO: 122, and the HCDR3 as shown in SEQ ID NO: 124.

In some embodiments, the VH comprises the HCDR1 as shown in SEQ ID NO: 114, the HCDR2 as shown in SEQ ID NO: 123, and the HCDR3 as shown in SEQ ID NO: 124.

In some embodiments, the VL comprises the LCDR1 as shown in SEQ ID NO: 125, the LCDR2 as shown in SEQ ID NO: 126, and the LCDR3 as shown in SEQ ID NO: 127.

In some embodiments, the VL comprises the LCDR1 as shown in SEQ ID NO: 125, the LCDR2 as shown in SEQ ID NO: 126, and the LCDR3 as shown in SEQ ID NO: 130.

In some embodiments, the VL comprises the LCDR1 as shown in SEQ ID NO: 125, the LCDR2 as shown in SEQ ID NO: 126, and the LCDR3 as shown in SEQ ID NO: 129.

In some embodiments, the VL of the second binding domain includes the LCDR1 as shown in SEQ ID NO: 125, the LCDR2 as shown in SEQ ID NO: 126, and the LCDR3 as shown in SEQ ID NO: 128.

In some embodiments, the VH of the second binding domain includes the sequence shown in SEQ ID NO: 136-147, and the VH of the second binding domain includes the sequence shown in SEQ ID NO: 149-165.

In some embodiments, the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and the second antigen-binding domain comprises HCDR1 as set forth in SEQ ID NO: 264, HCDR2 as set forth in SEQ ID NO: 265, HCDR3 as set forth in SEQ ID NO: 266, LCDR1 as set forth in SEQ ID NO: 267, LCDR2 as set forth in SEQ ID NO: 268, and LCDR3 as set forth in SEQ ID NO: 269.

In some embodiments, the first antigen-binding domain comprises a VH as set forth in SEQ ID NO: 70 and a VL as set forth in SEQ ID NO: 106; and the second antigen-binding domain comprises a VH as set forth in SEQ ID NO: 148 and a VL as set forth in SEQ ID NO: 166.

In some embodiments, the first antigen-binding domain comprises an scFv as set forth in SEQ ID NO: 133; and the second antigen-binding domain comprises the VH as set forth in SEQ ID NO: 148 and the VL as set forth in SEQ ID NO: 166.

In some embodiments, the isolated antigen-binding protein includes a light chain as shown in SEQ ID NO: 182 and heavy chain as shown in SEQ ID NO: 207, or includes a light chain as shown in SEQ ID NO: 182 and heavy chain as shown in SEQ ID NO: 208.

In another aspect, the present application provides an isolated nucleic acid molecule encoding the isolated antigen-binding protein.

In another aspect, the present application provides an expression vector comprising the nucleic acid.

In another aspect, the present application provides a host cell comprising the isolated nucleic acid and/or the expression vector.

In another aspect, the present application provides a composition comprising the isolated antigen-binding protein, and optionally a pharmaceutically acceptable carrier.

A bispecific antibody, comprising: a first binding domain and a second binding domain, wherein the first binding domain specifically binds to TNF-like protein A (TL1A), and the second binding domain specifically binds to IL-23p19, wherein the first binding domain comprises a heavy chain variable region (VH), and the VH comprises an HCDR3 comprising an amino acid sequence of SEQ ID NO: 14.

In some embodiments, the HCDR3 of the VH of the first binding domain of the bispecific antibody comprises an amino acid sequence of any one of SEQ ID NOs: 15-18.

In some embodiments, the VH of the first binding domain of the bispecific antibody comprises an HCDR2 comprising an amino acid sequence of SEQ ID NO: 5.

In some embodiments, the HCDR2 of the VH first binding domain of the bispecific antibody comprises an amino acid sequence of any one of SEQ ID NOs: 6-13.

In some embodiments, the VH of the first binding domain of the bispecific antibody comprises an HCDR1 comprising the amino acid sequence of SEQ ID NO:1.

In some embodiments, the HCDR1 of the VH of the first binding domain of the bispecific antibody comprises an amino acid sequence of any one of SEQ ID NOs:2-4.

In some embodiments;
(i) the VH of the first binding domain of the bispecific antibody comprises the HCDR1 as set forth in SEQ ID NO:2, the HCDR2 as set forth in SEQ ID NO:7, and the HCDR3 as set forth in SEQ ID NO:15;
(ii) the VH of the first binding domain of the bispecific antibody comprises the HCDR1 as set forth in SEQ ID NO:2, the HCDR2 as set forth in SEQ ID NO:8, and the HCDR3 as set forth in SEQ ID NO:15;
(iii) the VH of the first binding domain of the bispecific antibody comprises the HCDR1 as set forth in SEQ ID NO:2, the HCDR2 as set forth in SEQ ID NO:6, and the HCDR3 as set forth in SEQ ID NO: 18;
(iv) the VH of the first binding domain of the bispecific antibody comprises the HCDR1 as set forth in SEQ ID NO:4, the HCDR2 as set forth in SEQ ID NO:6, and the HCDR3 as set forth in SEQ ID NO: 15;
(v) the VH of the first binding domain of the bispecific antibody comprises the HCDR1 as set forth in SEQ ID NO:2, the HCDR2 as set forth in SEQ ID NO:9, and the HCDR3 as set forth in SEQ ID NO:15;
(vi) the VH of the first binding domain of the bispecific antibody comprises the HCDR1 as set forth in SEQ ID NO:2, the HCDR2 as set forth in SEQ ID NO:10, and the HCDR3 as set forth in SEQ ID NO: 15;
(vii) the VH of the first binding domain of the bispecific antibody comprises the HCDR1 as set forth in SEQ ID NO:2, the HCDR2 as set forth in SEQ ID NO:11, and the HCDR3 as set forth in SEQ ID NO: 15;
(viii) the VH of the first binding domain of the bispecific antibody comprises the HCDR1 as set forth in SEQ ID NO:2, the HCDR2 as set forth in SEQ ID NO:6, and the HCDR3 as set forth in SEQ ID NO: 15;
(ix) the VH of the first binding domain of the bispecific antibody comprises the HCDR1 as set forth in SEQ ID NO:2, the HCDR2 as set forth in SEQ ID NO:12, and the HCDR3 as set forth in SEQ ID NO: 15;
(x) the VH of the first binding domain of the bispecific antibody comprises the HCDR1 as set forth in SEQ ID NO:2, the HCDR2 as set forth in SEQ ID NO:13, and the HCDR3 as set forth in SEQ ID NO:15;
(xi) the VH of the first binding domain of the bispecific antibody comprises the HCDR1 as set forth in SEQ ID NO:3, the HCDR2 as set forth in SEQ ID NO:6, and the HCDR3 as set forth in SEQ ID NO:16;
(xii) the VH of the first binding domain of the bispecific antibody comprises the HCDR1 as set forth in SEQ ID NO:3, the HCDR2 as set forth in SEQ ID NO:6, and the HCDR3 as set forth in SEQ ID NO:17;
(xiii) the VH of the first binding domain of the bispecific antibody comprises the HCDR1 as set forth in SEQ ID NO:3, the HCDR2 as set forth in SEQ ID NO:9, and the HCDR3 as set forth in SEQ ID NO:15;
(xiv) the VH of the first binding domain of the bispecific antibody comprises the HCDR1 as set forth in SEQ ID NO:3, the HCDR2 as set forth in SEQ ID NO:10, and the HCDR3 as set forth in SEQ ID NO:15;
(xv) the VH of the first binding domain of the bispecific antibody comprises the HCDR1 as set forth in SEQ ID NO:3, the HCDR2 as set forth in SEQ ID NO:11, and the HCDR3 as set forth in SEQ ID NO:15;
(xvi) the VH of the first binding domain of the bispecific antibody comprises the HCDR1 as set forth in SEQ ID NO:3, the HCDR2 as set forth in SEQ ID NO:6, and the HCDR3 as set forth in SEQ ID NO:15;
(xvii) VH of the first binding domain of the bispecific antibody comprises the HCDR1 as set forth in SEQ ID NO:2, the HCDR2 as set forth in SEQ ID NO:6, and the HCDR3 as set forth in SEQ ID NO:16;
(xviii) the VH of the first binding domain of the bispecific antibody comprises the HCDR1 as set forth in SEQ ID NO:2, the HCDR2 as set forth in SEQ ID NO:9, and the HCDR3 as set forth in SEQ ID NO: 16;
(xix) the VH of the first binding domain of the bispecific antibody comprises the HCDR1 as set forth in SEQ ID NO: 2, the HCDR2 as set forth in SEQ ID NO:10, and the HCDR3 as set forth in SEQ ID NO:16;
(xx) the VH of the first binding domain of the bispecific antibody comprises the HCDR1 as set forth in SEQ ID NO:2, the HCDR2 as set forth in SEQ ID NO:11, and the HCDR3 as set forth in SEQ ID NO:16;
(xxi) the VH of the first binding domain of the bispecific antibody comprises the HCDR1 as set forth in SEQ ID NO:2, the HCDR2 as set forth in SEQ ID NO:12, and the HCDR3 as set forth in SEQ ID NO:16;
(xxii) the VH of the first binding domain of the bispecific antibody comprises the HCDR1 as set forth in SEQ ID NO:2, the HCDR2 as set forth in SEQ ID NO:6, and the HCDR3 as set forth in SEQ ID NO:17; or
(xxiii) the VH of the first binding domain of the bispecific antibody comprises the HCDR1 as set forth in SEQ ID NO:2, the HCDR2 as set forth in SEQ ID NO:10, and the HCDR3 as set forth in SEQ ID NO:17.

In some embodiments, the first binding domain of the bispecific antibody further comprises a light chain variable region (VL), and the VL of the first binding domain comprising at least one light chain complementary determining region (LCDR), which comprises an amino acid sequence selected from the amino acid sequences shown in SEQ ID NO:42, SEQ ID NO:43 and SEQ ID NO:44.

In some embodiments, the first binding domain of the bispecific antibody comprises the VH as set forth in SEQ ID NO:270.

In some embodiments, the first binding domain of the bispecific antibody comprises the VL as set forth in SEQ ID NO:271.

In some embodiments, the VH of the second binding domain of the bispecific antibody comprises the HCDR1 as set forth in SEQ ID NO: 264, the HCDR2 as set forth in SEQ ID NO: 265, and the HCDR3 as set forth in SEQ ID NO: 266.

In some embodiments, the VL of the second binding domain of the bispecific antibody comprises the LCDR1 as set forth in SEQ ID NO: 267, the LCDR2 as set forth in SEQ ID NO: 268, and the LCDR3 as set forth in SEQ ID NO: 269.

In some embodiments, the second antigen binding domain of the bispecific antibody comprises a VL as set forth in SEQ ID NO: 166.

In some embodiments, the bispecific antibody comprises a Fab, Fab′, Fv fragment, F(ab)′₂, scFv,

di-scFv and/or dAb.

In some embodiments, the bispecific antibody comprises the scFv of the first antigen binding domain or the scFv of the second antigen binding domain.

In some embodiments, the scFv of the first antigen-binding domain of the bispecific antibody comprises a sequence as set forth in any one of SEQ ID NOs: 131-134.

In some embodiments, the scFv of the first antigen-binding domain of the bispecific antibody comprises a sequence as set forth in SEQ ID NO: 133.

In some embodiments, the VL and VH of the second binding domain are selected from the following combination:
VH as set forth in SEQ ID NO:135 and VL as set forth in SEQ ID NO:149;
VH as set forth in SEQ ID NO:136 and VL as set forth in SEQ ID NO:150;
VH as set forth in SEQ ID NO:137 and VL as set forth in SEQ ID NO:151;
VH as set forth in SEQ ID NO:137 and VL as set forth in SEQ ID NO:152;
VH as set forth in SEQ ID NO:137 and VL as set forth in SEQ ID NO:153;
VH as set forth in SEQ ID NO:137 and VL as set forth in SEQ ID NO:154;
VH as set forth in SEQ ID NO:138 and VL as set forth in SEQ ID NO:155;
VH as set forth in SEQ ID NO:139 and VL as set forth in SEQ ID NO: 156;
VH as set forth in SEQ ID NO:140 and VL as set forth in SEQ ID NO: 157;
VH as set forth in SEQ ID NO:141 and VL as set forth in SEQ ID NO:158;
VH as set forth in SEQ ID NO:142 and VL as set forth in SEQ ID NO:159;
VH as set forth in SEQ ID NO:143 and VL as set forth in SEQ ID NO:160;
VH as set forth in SEQ ID NO:143 and VL as set forth in SEQ ID NO:161;
VH as set forth in SEQ ID NO:143 and VL as set forth in SEQ ID NO:162;
VH as set forth in SEQ ID NO:143 and VL as set forth in SEQ ID NO:165;
VH as set forth in SEQ ID NO:144 and VL as set forth in SEQ ID NO:160;
VH as set forth in SEQ ID NO:144 and VL as set forth in SEQ ID NO:161;
VH as set forth in SEQ ID NO:144 and VL as set forth in SEQ ID NO:162;
VH as set forth in SEQ ID NO:144 and VL as set forth in SEQ ID NO:165;
VH as set forth in SEQ ID NO:146 and VL as set forth in SEQ ID NO:160;
VH as set forth in SEQ ID NO:146 and VL as set forth in SEQ ID NO:161;
VH as set forth in SEQ ID NO:146 and VL as set forth in SEQ ID NO:162;
VH as set forth in SEQ ID NO:146 and VL as set forth in SEQ ID NO:165;
VH as set forth in SEQ ID NO:145 and VL as set forth in SEQ ID NO:164;
VH as set forth in SEQ ID NO:147 and VL as set forth in SEQ ID NO:164;
VH as set forth in SEQ ID NO:145 and VL as set forth in SEQ ID NO:163;
VH as set forth in SEQ ID NO:147 and VL as set forth in SEQ ID NO:163;
VH as set forth in SEQ ID NO:146 and VL as set forth in SEQ ID NO:164;
VH as set forth in SEQ ID NO:147 and VL as set forth in SEQ ID NO:165;
VH as set forth in SEQ ID NO:148 and VL as set forth in SEQ ID NO:166.

In some embodiments, the heavy chain of the first antigen-binding domain is selected from the sequence shown in any one of SEQ ID NOs: 183-208 and/or the light chain of the first antigen-binding domain is selected from the sequence shown in any one of SEQ ID NOs: 175-182.

In some embodiments, the first antigen-binding domain of the bispecific antibody is selected from the following combination:
1) a light chain as set forth in SEQ ID NO: 179 and a heavy chain as set forth in SEQ ID NO: 199;
2) a light chain as set forth in SEQ ID NO: 179 and a heavy chain as set forth in SEQ ID NO: 200;
3) a light chain as set forth in SEQ ID NO: 179 and a heavy chain as set forth in SEQ ID NO: 201;
4) a light chain as set forth in SEQ ID NO: 179 and a heavy chain as set forth in SEQ ID NO: 202;
5) a light chain as set forth in SEQ ID NO: 180 and a heavy chain as set forth in SEQ ID NO: 203;
6) a light chain as set forth in SEQ ID NO: 181 and a heavy chain as set forth in SEQ ID NO: 204;
7) a light chain as set forth in SEQ ID NO: 182 and a heavy chain as set forth in SEQ ID NO: 205;
8) a light chain as set forth in SEQ ID NO: 182 and a heavy chain as set forth in SEQ ID NO: 206;
9) a light chain as set forth in SEQ ID NO: 182 and a heavy chain as set forth in SEQ ID NO: 207;
10) a light chain as set forth in SEQ ID NO: 182 and a heavy chain as set forth in SEQ ID NO: 208.

In some embodiments, the first antigen-binding domain of the bispecific antibody comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and the second antigen-binding domain of the bispecific antibody comprises HCDR1 as set forth in SEQ ID NO: 264, HCDR2 as set forth in SEQ ID NO: 265, HCDR3 as set forth in SEQ ID NO: 266, LCDR1 as set forth in SEQ ID NO: 267, LCDR2 as set forth in SEQ ID NO: 268, and LCDR3 as set forth in SEQ ID NO: 269.

In some embodiments, the first antigen-binding domain of the bispecific antibody comprises a VH as set forth in SEQ ID NO: 270 and a VL as set forth in SEQ ID NO: 271; and the second antigen-binding domain of the bispecific antibody comprises a VH as set forth in SEQ ID NO: 148 and a VL as set forth in SEQ ID NO: 166.

In some embodiments, the first antigen-binding domain of the bispecific antibody comprises an scFv as set forth in SEQ ID NO: 133; and the second antigen-binding domain of the bispecific antibody comprises the VH as set forth in SEQ ID NO: 148 and the VL as set forth in SEQ ID NO: 166.

In some embodiments, the bispecific antibody comprises a light chain as shown in SEQ ID NO: 182 and heavy chain as shown in SEQ ID NO: 207.

In another aspect, the present application provides an isolated nucleic acid molecule encoding a bispecific antibody as described herein.

In another aspect, the present application provides an expression vector comprising a nucleic acid encoding a bispecific antibody as described herein.

In another aspect, the present application provides a host cell comprising an isolated nucleic acid and/or an expression vector as described herein.

In another aspect, the present application provides a composition comprising a bispecific antibody as described herein and optionally a pharmaceutically acceptable carrier.

In another aspect, the present application provides a method for preparing a bispecific antibody as described herein, comprising culturing the host cell described herein, and recovering the isolated antigen-binding protein.

In another aspect, the present application provides a method for treating a disease or condition, comprising administering a bispecific antibody, an isolated nucleic acid, an expression vector, a host cell or a composition as described herein.

In another aspect, the present application provides a bispecific antibody as described herein for use in the treatment of a disease or condition.

In another aspect, the present application provides an isolated nucleic acid molecule, an expression vector, a host cell or a composition as described herein for use in the treatment of a disease or condition.

In another aspect, the present application provides an isolated nucleic acid molecule, an Expression vector, a host cell or a composition as described herein for use in the treatment of a disease or condition.

In another aspect, the present application provides a bispecific antibody, an isolated nucleic acid molecule, an expression vector, a host cell or a composition as described herein for the treatment of disease or condition or in the preparation of a drug for the treatment of disease or condition.

In some embodiments, the disease or the condition is a TL1A-mediated disease or condition.

In some embodiments, the TL1A or IL-23 mediated disease or condition is inflammatory disease, optionally wherein the TL1A or IL-23 mediated inflammatory disease is selected from one of the following: allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the TL1A or IL-23 mediated disease or condition is autoimmune disease,

optionally wherein the TL1A or IL-23 mediated autoimmune diseases are selected from one of the following: achalasia, Addison's disease, Adult Stil's disease, agammaglobulinemia, alopecia areata, amyloidosis, ankylosing spondylitis, anti-GBM/anti-TBM nephritis, antiphospholipid syndrome, autoimmune angioedema, autoimmune familial autonomic dysfunction, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Barlow disease (Balodisease), Behcet's disease, benign mucous membrane pemphigoid, bullous pemphigoid, Castleman's disease (Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatous disease (EGPA), cicatricial pemphigoid, and Cogan's syndrome), cold agglutinin disease, congenital heart block, coxsackienew myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, primary mixed cryoglobulinemia, Evans syndrome, fibromyalgia, fibrotic alveolitis, giant cell arteritis (temporal arteritis), giant cell myocarditis, glomerulonephritis, Goodpasture's syndrome, granulomatosis with polyangiitis, Graves' disease, Guillain-Barre syndrome syndrome), Hashimoto's thyroiditis, hemolytic anemia, Henoch-Schonlein purpura (HSP), herpes gravilens or pemphigoid gestationum (PG), hidradenitis suppurativa (HS) (paradoxical acne), hypogammaglobulinemia, IgA nephropathy, IgG4-associated sclerotic disease, immune thrombocytopenic purpura (ITP), Inclusion body myositis (IBM), interstitial cystitis (IC), juvenile arthritis, juvenile diabetes mellitus (type 1 diabetes), juvenile myositis (JM), Kawasaki disease, Lambert-Eaton syndrome, leukocytoclastic vasculitis, lichen planus, lichen sclerosus, lignoconjunctivitis, linear IgA disease (LAD), lupus, chronic Lyme disease (Lyme disease chronic), Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, recurrent rheumatism (PR), PandAS, paraneoplastic cerebellar degeneration (PCD), paroxysmal nocturnal hemoglobinuria (PNH), Parry Romberg syndrome, ciliary planitis (peripheral uveitis), Parsonage-Tumor syndrome), pemphigus, peripheral neuropathy, perivenous encephalomyelitis, pernicious anemia (PA), POEMS syndrome, polyarteritis nodosa, polyglandular syndrome type I, II, type III, polymyalgia rheumatica, polymyositis, post-myocardial infarction syndrome, postpericardiotomy syndrome, primary biliary cirrhosis, primary sclerosing cholangitis, progesterone dermatitis, psoriasis, psoriatic arthritis, pure red blood cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff-person syndrome (SPS), subacute bacterial endocarditis (SBE), Susac's syndrome syndrome), sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes mellitus, ulcerative colitis (UC), undifferentiated connective tissue disease (UCTD), uveitis, vasculitis, Vitiligo and Vogt-Koyanagi-Harada disease.

In some embodiments, the TL1A or IL-23 mediated disease or condition is cancer, optionally

wherein the TL1A or IL-23 mediated cancers are selected from one or more of the following: non-restrictive examples of cancer include adenoid cystic carcinoma, adrenal carcinoma, amyloidosis, anal cancer, ataxia-telangiectasia, atypical nevus syndrome, basal cell carcinoma, cholangiocarcinoma, Birt Hogg Dube syndrome, bladder cancer, bone cancer, brain tumor, breast cancer, male breast cancer, carcinoid tumor, cervical cancer, colorectal cancer, ductal cancer, Endometrial Cancer, Esophageal Cancer, Gastric Cancer, Gastrointestinal Stromal Tumor (GIST), HER2-Positive Breast Cancer, Pancreatic Islet Cell Tumor, Juvenile Polyposis Syndrome, Kidney Cancer, Laryngeal Cancer, Leukemia-Acute Lymphoblastic Leukemia, Acute Lymphoblastic Leukemia (ALL), Acute Myeloid Leukemia AML, Adult Leukemia, Childhood Leukemia, Chronic Lymphocytic Leukemia (CLL), Chronic Myeloid Leukemia (CML), Liver Cancer, Lobular Carcinoma, Lung Cancer, Small Cell Lung Cancer (SCLC), Non-small cell lung cancer (NSCLC), Hodgkin's lymphoma (Lymphoma-Hodgkin's), non-Hodgkin's lymphoma (Lymphoma-Non-Hodgkin's), malignant glioma, melanoma, meningioma, multiple myeloma, myelodysplastic syndrome (MD), nasopharyngeal carcinoma, neuroendocrine tumor, oral cancer, osteosarcoma, ovarian cancer, pancreatic cancer, pancreatic neuroendocrine tumor, parathyroid carcinoma, penile cancer, peritoneal cancer, Peutz-Jeghers syndrome, pituitary tumors, polycythemia vera, prostate cancer, renal cell carcinoma, retinoblastoma, salivary gland carcinoma, sarcoma, Sarcoma-Kaposi, skin cancer, small bowel cancer, stomach cancer, testicular cancer, thymoma, thyroid cancer, uterine (endometrial) cancer, vaginal cancer, and Wilms' Tumor.

Other aspects and advantages of the present application will become readily apparent to those skilled in the art from the following detailed description. Only exemplary embodiments of the present application are shown and described in the following detailed description. As will be recognized by those skilled in the art, the contents of the present application enable those skilled in the art to make modifications to the specific embodiments disclosed without departing from the spirit and scope of the present application to which the present application pertains. Accordingly, the drawings and description herein are to be regarded as illustrative in nature, and not as restrictive.

### BRIEF DESCRIPTION OF THE DRAWINGS

Specific features of the present application to which the present application relates are as set forth in the appended claims. The features and advantages of the present application to which the present application relates will be better understood by reference to the exemplary embodiments and drawings described in detail below. A brief description of the drawings is as follows:

FIG. 1 shows a schematic diagram of the configuration of the anti-TL1A+IL-23p19 bispecific antibody described in the present application;

FIGS. 2A-2C respectively show the binding and dissociation curves of the anti-IL-23 monoclonal antibodies Risankizumab, 23A0186-6, and 23A0198 described in the present application with human IL-23 protein;

FIGS. 3A-3C respectively show the binding and dissociation curves of the anti-IL-23 monoclonal antibodies Risankizumab, 23A0186-6, and 23A0198 described in the present application with monkey IL-23 protein;

FIGS. 4A-4C respectively show the binding and dissociation curves of the anti-TL1A+IL-23p19 bispecific antibodies TLb0103-3, TLb0110-2, and TLb0110-3 described in the present application with human IL-23 protein;

FIGS. 5A-5C respectively show the binding and dissociation curves of the anti-TL1A+Binding and dissociation curves of IL-23p19 bispecific antibodies TLb0103-3, TLb0110-2, TLb0110-3 and monkey IL-23 protein;

FIGS. 6A-6D respectively show the binding and dissociation curves of the anti-TL1A monoclonal antibody TLA0015-11 and anti-TL1A+IL-23p19 bispecific antibodies TLb0103-3, TLb0110-2, TLb0110-3 described in this application and human TL1A-his protein;

FIGS. 7A-7D respectively show the binding and dissociation curves of the anti-TL1A monoclonal antibody TLA0015-11 and anti-TL1A+IL-23p19 bispecific antibodies TLb0103-3, TLb0110-2, TLb0110-3 described in this application and monkey TL1A-his protein;

FIG. 8 shows the binding and dissociation curves of the anti-TL1A+IL-23p19 bispecific antibody blocks the binding of TL1A-his protein to 293-DR3 cells;

FIG. 9 shows the effect of the anti-TL1A+IL-23p19 bispecific antibody described in the present application on inhibiting Caspase3/7 activated by TL1A;

FIGS. 10A-10B show the effect of the anti-TL1A monoclonal antibody and anti-TL1A+IL-23p19 bispecific antibody described in the present application on inhibiting NF-κB activated by TL1A-his protein;

FIGS. 11A-11B show the effect of the anti-IL-23p19 monoclonal antibody and anti-TL1A+IL-23p19 bispecific antibody described in the present application on inhibiting STAT3 activated by IL-23;

FIGS. 12A-12C show the effect of the anti-IL-23p19 monoclonal antibody and anti-TL1A+The effect of IL-23p19 bispecific antibody in inhibiting the secretion of mIL-17 in mouse spleen cells;

FIGS. 13A-13B show the anti-IL-23p19 monoclonal antibody and anti-TL1A+The effect of IL-23p19 bispecific antibody on inhibiting the secretion of mIL-22 in mouse spleen cells;

FIG. 14A-FIG. 14B show the weight change curves of each group of mice in the DSS-induced hTL1A/hIL-23 mouse enteritis model;

FIG. 15 shows the daily scores of each group of mice in the DSS-induced hTL1A/hIL-23 mouse enteritis model;

FIG. 16A shows the changes in the length of the colorectum of each group of mice in the DSS-induced hTL1A/hIL-23 mouse enteritis model;

FIG. 16B shows the changes in the weight of the colorectum of each group of mice in the DSS-induced hTL1A/hIL-23 mouse enteritis model;

FIG. 16C shows the ratio of colorectal weight to length of each group of mice in the DSS-induced hTL1A/hIL-23 mouse enteritis model;

FIG. 17A-FIG. 17B show the weight change curves of each group of mice in the DSS-induced hTL1A/hIL-23 mouse enteritis model;

FIG. 18 shows Daily scores of mice in each group in the DSS-induced hTL1A/hIL-23 mouse enteritis model;

FIGS. 19A-19B show the changes in colorectal length of mice in each group in the DSS-induced hTL1A/hIL-23 mouse enteritis model;

FIGS. 20A-20B show the changes in colorectal weight of mice in each group in the DSS-induced hTL1A/hIL-23 mouse enteritis model;

FIGS. 21A-21B show the weight change curves of mice in each group in the IMQ-induced hTL1A/hIL-23 transgenic mouse psoriasis model;

FIG. 22 shows the skin thickness change curves of the modeling site of mice in each group in the IMQ-induced hTL1A/hIL-23 transgenic mouse psoriasis model;

FIG. 23 shows the PASI score results of mice in each group in the IMQ-induced hTL1A/hIL-23 transgenic mouse psoriasis model;

FIG. 24A shows the IMQ-induced hTL1A/hIL-23 transgenic mouse psoriasis model. Daily scoring of the degree of scaling in the modeling area of each group of mice in the genetic mouse psoriasis model;

FIG. 24B shows the daily scoring of erythema in the modeling area of each group of mice in the IMQ-induced hTL1A/hIL-23 transgenic mouse psoriasis model;

FIG. 24C shows the daily scoring of the degree of skin damage in the modeling area of each group of mice in the IMQ-induced hTL1A/hIL-23 transgenic mouse psoriasis model;

FIG. 24D shows the daily scoring of the degree of infiltration in the modeling area of each group of mice in the IMQ-induced hTL1A/hIL-23 transgenic mouse psoriasis model;

FIG. 25A shows the spleen weight results of each group of mice in the IMQ-induced hTL1A/hIL-23 transgenic mouse psoriasis model;

FIG. 25B shows the ratio of spleen weight to body weight of each group of mice in the IMQ-induced hTL1A/hIL-23 transgenic mouse psoriasis model;

FIG. 26A shows the results of H&E staining of the skin in the back modeling area of mice;

FIG. 26B shows the results of H&E staining epidermal thickness results;

FIG. 26C shows the H&E staining pathological scoring results;

FIG. 27A shows the weight change curve of rats in the rat psoriasis model induced by IL-23+TL1A;

FIG. 27B shows the weight change ratio curve of rats in the rat psoriasis model induced by IL-23+TL1A;

FIG. 28 shows the thickness change curve of rat ear skin in the rat psoriasis model induced by IL-23+TL1A;

FIG. 29 shows the PASI scoring results of rats in the rat psoriasis model induced by IL-23+TL1A;

FIG. 30A shows the daily scoring of the scale degree of rat modeling area in the rat psoriasis model induced by IL-23+TL1A;

FIG. 30B shows the daily scoring of erythema in the rat modeling area in the rat psoriasis model induced by IL-23+TL1A;

FIG. 30C shows the daily scoring of the infiltration degree of rat modeling area in the rat psoriasis model induced by IL-23+TL1A

### DETAILED DESCRIPTION OF THE INVENTION

The embodiments of the invention of the present application are illustrated by particular examples. Other advantages and effects of the present application will become readily apparent to those skilled in the art from disclosure of the present specification.

### DEFINITION OF TERMS

In the present application, the term “isolated” generally refers to a product obtained from a natural state by artificial means. If an “isolated” substance or component occurs in nature, it may be altered from its natural environment, or the substance may be isolated from its natural environment, or both. For example, an unisolated polynucleotide or polypeptide naturally occurs in a living animal and the same polynucleotide or polypeptide isolated from its natural state in high purity is the to be isolated. The term “isolated” does not exclude the admixture of artificial or synthetic substances, nor the presence of other impure substances which do not affect the activity of the substance.

In the present application, the term “isolated antigen-binding protein” generally refers to a protein with antigen-binding ability obtained by artificial means from a natural state. The “isolated antigen-binding protein” may include a portion that binds to an antigen and, optionally, a framework or framework portion that allows the antigen-binding portion to adopt a conformation that promotes the antigen-binding portion to bind to an antigen. The antigen-binding protein may include, for example, a protein framework region (FR) derived from an antibody or an alternative protein framework region or an artificial framework region having a transplanted CDR or CDR derivative. Such frameworks include, but are not limited to, a framework region derived from an antibody that includes a mutation introduced, for example, to stabilize the three-dimensional structure of the antigen-binding protein and a fully synthetic framework region including, for example, a biocompatible polymer. See, for example, Korndorfer et al., 2003, Proteins: Structure, Function, and Bioinformatics, 53 (1): 121-129 (2003); Roque et al., Biotechnol. Prog. 20: 639-654 (2004). Examples of antigen binding proteins include, but are not limited to, human antibodies, humanized antibodies; chimeric antibodies; recombinant antibodies; single-chain antibodies; bifunctional antibodies; trifunctional antibodies; tetrafunctional antibodies; Fab, Fab′, Fv fragment, F(ab′)2, F(ab)2, scFv, di-scFv, dAb, IgD antibody; IgE antibody; IgM antibody; IgG1 antibody; IgG2 antibody; IgG3 antibody; or IgG4 antibody and fragments thereof.

In the present application, the separated antigen binding protein may comprise more than one antigen binding domain. In the present application, the antigen binding domain may target different antigens. In the present application, the antigen binding domain may target different epitopes of the same antigen. For example, the separated antigen binding protein may comprise a first binding domain and a second binding domain. For example, the first binding domain may target TL1A protein, and the second binding domain may target IL-23 protein, for example.

In the present application, the term “variable domain” is used interchangeably with “variable region” and generally refers to a portion of an antibody heavy chain and/or light chain. The variable domains of the heavy and light chains may be referred to as “VH” and “VL”, respectively (or “VH” and “VL”, respectively). These domains are generally the most variable parts of an antibody (relative to other antibodies of the same type) and contain antigen binding sites.

In the present application, the term “variable” generally refers to the fact that certain segments of the variable domain may differ greatly in sequence between antibodies. The variable domain mediates antigen binding and determines the specificity of a particular antibody for its particular antigen. However, the variability is not evenly distributed throughout the variable domain. It is usually concentrated in three segments called hypervariable regions (CDRs or HVRs) in the light chain and heavy chain variable domains. The more highly conserved parts of the variable domains are called framework regions (FRs). The variable domains of natural heavy and light chains each contain four FR regions, most of which adopt a β-folded configuration, connected by three CDRs, which form a ring connection and form a part of the β-folded structure in some cases. The CDRs in each chain are kept together in close proximity by the FR region, and the CDRs from the other chain together promote the formation of the antigen binding site of the antibody (see Kabat et al, Sequences of Immunological Interest, Fifth Edition, National Institute of Health, Bethesda, Md. (1991)). The CDRs described in this application are defined according to the Kabat coding system.

In the present application, the term “antibody” generally refers to an immunoglobulin or its fragment or derivative thereof, covering any polypeptide including an antigen binding site, whether it is produced in vitro or in vivo. The term includes, but is not limited to, polyclonal, monoclonal, monospecific, multispecific, nonspecific, humanized, single-chain, chimeric, synthetic, recombinant, hybrid, mutated and transplanted antibodies. Unless otherwise modified by the term “complete”, as in “complete antibody”, for the purposes of the present invention, the term “antibody” also includes antibody fragments, such as Fab, F (ab′) 2, Fv, scFv, Fd, dAb and other antibody fragments that retain antigen binding function (e.g., specifically binds to TIGIT). Typically, such fragments should include an antigen binding domain. The basic 4-chain antibody unit is a heterotetrameric glycoprotein composed of two identical light (L) chains and two identical heavy (H) chains. IgM antibodies consist of 5 basic heterotetrameric units with an additional polypeptide called J chain and contain 10 antigen binding sites, while IgA antibodies include 2-5 basic 4-chain units that can be combined with J chains to form multivalent combinations. For IgG, the 4-chain unit is generally about 150,000 daltons. Each L chain is connected to the H chain by a covalent disulfide bond, and the two H chains are connected to each other by one or more disulfide bonds depending on the isotype of the H chain. Each H and L chain also has regularly spaced intrachain disulfide bridges. Each H chain has a variable domain (VH) at the N-terminus, followed by three constant domains (CH) for each of the α and γ chains, and four CH domains for the μ and ε isotypes. Each L chain has a variable domain (VL) at the N-terminus and a constant domain at its other end. VL corresponds to VH, and CL corresponds to the first constant domain (CH1) of the heavy chain. Specific amino acid residues are believed to form an interface between the light chain and heavy chain variable domains. VH and VL pair together to form a single antigen binding site. For the structure and properties of different classes of antibodies, see, for example, Basic and Clinical Immunology, 8th Edition, Daniel P. Sties, Abba I. Terr and Tristram G. Parsolw (eds), Appleton & Lange, Norwalk, Conn., 1994, page 71 and chapter 6. The L chain from any vertebrate species can be divided into one of two distinct types based on the amino acid sequence of its constant domain, called κ and λ. According to the amino acid sequence of the heavy chain (CH) constant domain, immunoglobulins can be divided into different classes or isotypes. There are currently five classes of immunoglobulins: IgA, IgD, IgE, IgG and IgM, with heavy chains named α, δ, ε, γ and μ, respectively. Based on the relatively small differences in CH sequence and function, γ and α classes are further divided into subclasses, for example, human expresses the following subclasses: IgG1, IgG2A, IgG2B, IgG3, IgG4, IgAQ1 and IgK1.

In this application, the term “CDR”, also known as “complementarity determining region”, generally refers to a region in an antibody variable domain whose sequence is highly variable and/or forms a structurally defined loop. Typically, an antibody includes six CDRs; three in VH (HCDR1, HCDR2, HCDR3), and three in VL (LCDR1, LCDR2, LCDR3). In certain embodiments, naturally occurring camelid antibodies consisting only of heavy chains can function normally and stably in the absence of light chains. See, e.g., Hamers-Casterman et al., Nature 363:446-448 (1993); Sheriff et al, Nature Struct. Biol. 3:733-736 (1996).

In the present application, the term “FR” generally refers to the more highly conserved portion of the variable domain of an antibody, which is called the framework region. Typically, the variable domains of natural heavy and light chains each contain four FR regions, namely four in VH (H-FR1, H-FR2, H-FR3, and H-FR4), and four in VL (L-FR1, L-FR2, L-FR3, and L-FR4). For example, the VL of the separated antigen-binding protein described in the present application may include the framework regions L-FR1, L-FR2, L-FR3, and L-FR4. The VH of the separated antigen-binding protein described in the present application may include the framework regions H-FR1, H-FR2, H-FR3, and H-FR4.

In the present application, the term “antigen-binding fragment” generally refers to one or more fragments having the ability to specifically bind to an antigen. In the present application, the antigen-binding fragment may include Fab, Fab′, F(ab)2, Fv fragment, F(ab′)2, scFv, di-scFv and/or dAb.

In this application, the term “Fab” generally refers to the antigen-binding fragment of an antibody. As described above, an intact antibody can be digested using papain. After papain digestion of an antibody, two identical antigen-binding fragments are produced, namely, a “Fab” fragment, and a residual “Fc” fragment (i.e., the Fc region, as above). The Fab fragment can be composed of a complete L chain with a variable region of a heavy chain and the first constant region (CH1) of the H chain (VH).

In this application, the term “Fab′ fragment” generally refers to a monovalent antigen-binding fragment of a human monoclonal antibody, which is slightly larger than the Fab fragment. For example, a Fab′ fragment can include all light chains, all heavy chain variable regions, and all or part of the first and second constant regions of the heavy chain. For example, a Fab′ fragment can also include part or all of the 220-330 amino acid residues of the heavy chain.

In this application, the term “F(ab′)2” generally refers to an antibody fragment produced by digestion of an intact antibody with pepsin. The F(ab′)2 fragment contains two Fab fragments held together by disulfide bonds and a portion of the hinge region. F(ab′)2 fragments have bivalent antigen binding activity and are capable of cross-linking antigens.

In the present application, the term “Fv fragment” generally refers to a monovalent antigen binding fragment of a human monoclonal antibody, including all or part of the heavy chain variable region and the light chain variable region, and lacking the heavy chain constant region and the light chain constant region. The heavy chain variable region and the light chain variable region include, for example, CDRs. For example, the Fv fragment includes all or part of the amino terminal variable region of about 110 amino acids of the heavy chain and the light chain.

In the present application, the term “scFv” generally refers to a fusion protein comprising at least one antibody fragment comprising a variable region of a light chain and at least one antibody fragment comprising a variable region of a heavy chain, wherein the light chain and heavy chain variable regions are adjacent (e.g., via a synthetic linker such as a short flexible polypeptide linker) and can be expressed in the form of a single chain polypeptide, and wherein the scFv retains the specificity of the intact antibody from which it is derived. Unless otherwise specified, as used in this application, an scFv may have the VL and VH variable regions in any order (e.g., relative to the N-terminus and C-terminus of the polypeptide), and the scFv may include VL-linker-VH or may include VH-linker-VL.

In the present application, the term “monoclonal antibody” generally refers to a population of substantially homologous antibodies, i.e., the individual antibodies comprising the population are identical except for possible naturally occurring mutations that may be present in minor amounts. Monoclonal antibodies are highly specific, being directed against a single antigenic site. For example, the monoclonal antibodies can be prepared by hybridoma techniques or can be produced in bacteria, eukaryotic animal or plant cells using recombinant DNA methods, or can be derived from phage antibody libraries using the techniques, for example, described in Clackson et al., Nature, 352:624-628 (1991) and Marks et al., Mol. Biol., 222:581-597 (1991).

In the present application, the term “chimeric antibody” generally refers to an antibody in which a portion of the amino acid sequence of each heavy or light chain is homologous to the corresponding amino acid sequence in an antibody from a particular species, or belongs to a particular class, while the remaining segments of the chain are homologous to the corresponding sequence in another species. For example, the variable regions of both the light and heavy chains are derived from the variable region of an antibody of one animal species (e.g., mouse, rat, etc.), while the constant portions are homologous to antibody sequences from another species (e.g., human). For example, to obtain chimeric antibodies, non-human-derived B cells or hybridoma cells can be used to generate variable regions, while which the constant regions in combination therewith are of human origin. The variable region has the advantage of being easy to prepare and its specificity is not influenced by the origin of the constant region with which it is combined. Also, since the constant region of the chimeric antibody can be derived from human, the chimeric antibodies are less likely to elicit an immune response upon injection than using antibodies whose constant regions are of non-human origin.

In the present application, the term “humanized antibody” generally refers to a chimeric antibody that contains fewer sequences from non-human immunoglobulins, thereby reducing the immunogenicity of a xenogenous antibody when introduced into humans, while maintaining the full antigen-binding affinity and specificity of the antibody. For example, non-human binding domains can be humanized using the technical means, such as CDR transplantation (Jones et al., Nature 321:522(1986)) and variants thereof, including “reshaping”, (Verhoeyen, et al., 1988 Science 239:1534-1536; Riechmann, et al., 1988 Nature 332:323-337; Tempest, et al., Bio/Technol 1991 9:266-271), “hyperimmunization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies.” In: Metcalf B W, Dalton B J, eds. Cellular adhesion: molecular definition to therapeutic potential. New York: Plenum Press, 1994: 291-312), and resurfacing (U.S. Pat. No. 5,639,641). Other regions, such as hinge and constant region domains, may also be humanized if they are also derived from non-human sources.

In the present application, the term “mouse antibody” generally refers to an antibody whose variable region framework and CDR region are derived from mouse germline immunoglobulin sequences. In addition, if the antibody comprises a constant region, it is also derived from mouse germline immunoglobulin sequences. The mouse antibody of the present application may comprise amino acid residues not encoded by mouse germline immunoglobulin sequences, for example, may comprise mutations introduced by in vitro random mutations or point mutations or by in vivo somatic mutations.

In the present application, the term “directly linked” is used in contrast to the term “indirect linked”, which usually means a direct connection. For example, said direct connection may be a direct connection between substances without spacers. Said spacer may be a linker. For example, said linker may be a peptide linker. The term “indirect linkage” generally refers to the case where there is no direct linkage between substances. For example, said indirect linkage may be a linkage through a spacer. For example, in the isolated antigen-binding proteins described in the present application, the C-terminal end of said L-FR1 and the N-terminal end of said LCDR1 may be directly or indirectly linked.

In this application, the term “TL1A” refers to TNF-like protein 1A. TL1A (TNFSF15) is a member of the TNF family that is expressed predominantly by endothelial cells, macrophages and dendritic cells (DCs). Its expression is induced by immune complexes (ICs) and cytokines. TL1A receptor DR3 is expressed mainly on T cells and NKT cells. In vitro, TL1A has been shown to enhance T cell proliferation and cytokine production in both humans and mice. In vivo, TL1A transgenic mice produce an IBD phenotype similar to human Crohn's disease.

In the present application, the p19 subunit of IL-23 (also referred to herein as “IL-23p19” and “p19 subunit”) is a 189 amino acid polypeptide containing a 21 amino acid leader sequence (Oppmann et al., Immunity 13:715 (2000), SEQ ID NO:181). The biological activity of this molecule is only detected when it is combined with the IL-12p40 subunit to form IL-23. IL-23 is mainly expressed by activated dendritic cells (DC) and phagocytes. The receptor for IL-23 was found to be composed of the IL-12Rβ1 subunit of the IL-12 receptor combined with a unique subunit called IL-23R (Parham et al., J. Immunol. 168:5699 (2002)). The receptor was detected to be expressed mainly on memory T cells and NK cells. Therefore, the expression of this cytokine: receptor pair seems to be restricted to specific immune cell populations. Although it was initially thought that IL-12 and IL-23 would share many functions, data show that this is not the case. Although IL-12 has a major role in the generation of Th1 cells, IL-23 has been found to be critically involved in the generation and maintenance of a recently identified Th cell subset called Th17 (Kikly et al., Curr. Opin. Immunol. 18: 670 (2006); Kastelein et al., Ann. Rev. Immunol. 25: 221 (2007)). These cells produce IL-17A, IL-17F, IL-22, and other proinflammatory cytokines, such as IL-6 and TNF-α. As described below, animal model studies on the role of these Th17 cells show their importance as a driving force in chronic inflammation and autoimmunity.

The present invention provides antibodies that bind to the p19 subunit of IL-23, in particular human IL-23p19. The present invention also relates to humanized antibodies that recognize the p19 subunit of IL-23. In specific embodiments, the sequences of these humanized antibodies have been identified based on the sequences of certain lead mouse antibodies.

The lead mouse antibodies of the present invention are produced by mouse fusion tumors. Mice are immunized using different techniques. For example, antibodies specific for human IL-23p19 protein or fragments thereof can be produced against immunogenic antigens such as isolated IL-23p19 protein, isolated IL-23 protein, isolated hybrid IL-23 protein and/or a portion of any of the above proteins (including synthetic peptides). For example, mice are immunized with a hybrid IL-23 protein comprising a mouse IL-23p40 subunit and a human IL-23p19 subunit. Immunogenic antigens can be prepared and monoclonal antibodies can be produced using any suitable technique known in the art.

The lead mouse antibodies are selected based on their high affinity for human IL-23. Thus, in one aspect, the present invention provides an antibody that binds to human IL-23 with high affinity. The selected mouse antibody is humanized to produce a humanized antibody. The humanized antibody of the present invention binds to human IL-23 with high affinity. Thus, in another aspect, the present invention provides a humanized antibody that binds to human IL-23 with high affinity.

In this application, the term “dAb” generally refers to an antigen-binding fragment having a VH domain, a VL domain, or having a VH domain or a VL domain, see, for example, Ward et al. (Nature, 1989 Oct. 12; 341(6242): 544-6), see Holt et al., Trends Biotechnol., 2003, 21(11): 484-490; and see, for example, WO 06/030220, WO 06/003388, and other published patent applications of Domantis Ltd.

In this application, the term “specific binding” or “specific” generally refers to a measurable and reproducible interaction, such as binding between a target and an antibody, which can determine the presence of a target in the presence of a heterogeneous population of molecules (including biomolecules). For example, an antibody that specifically binds to a target (which may be an epitope) may be an antibody that binds to the target with greater affinity, avidity, more readily, and/or for a greater duration than it binds to other targets. In certain embodiments, an antibody specifically binds to an epitope on a protein that is conserved among proteins of different species. In certain embodiments, specific binding may include but does not require exclusive binding.

In addition to the specific proteins and nucleotides mentioned herein, the present application may also include functional variants, derivatives, analogs, homologs, and fragments thereof.

The term “functional variant” refers to an amino acid sequence that is substantially identical to a naturally occurring sequence or a polypeptide encoded by a substantially identical nucleotide sequence and capable of having one or more activities of the naturally occurring sequence. In the context of the present application, a variant of any given sequence refers to a sequence in which the particular sequence of residues, whether amino acid or nucleotide residues, has been modified such that the polypeptide or polynucleotide substantially retains at least one endogenous function. Variant sequences may be obtained by the addition, deletion, substitution, modification, substitution and/or variation of at least one amino acid residue and/or nucleotide residue present in a naturally occurring protein and/or polynucleotide, so long as the original functional activity is retained.

In the present application, the term “derivative” generally refers to a polypeptide or polynucleotide of the present application comprising any substitution, variation, modification, replacement, deletion and/or addition of one (or more) amino acid residues from/on the sequence, so long as the resulting polypeptide or polynucleotide substantially retains at least one of its endogenous functions.

In the present application, the term “analog” generally refers to a polypeptide or polynucleotide including any mimetic of a polypeptide or polynucleotide, i.e., a chemical compound that possesses at least one endogenous function of the polypeptide or polynucleotide that the mimetic mimics.

Generally, amino acid substitutions, e.g., at least one (e.g., 1, 2, 3, 4, 5, 6, 7, 8, 9, 10, or 20 or more) amino acid substitution can be made, so long as the modified sequence substantially retains the desired activity or ability. Amino acid substitutions can include the use of non-naturally occurring analogs.

In the present application, the term “homologue” generally refers to an amino acid sequence or nucleotide sequence having certain homology to a naturally occurring sequence. The term “homology” can be equivalent to sequence “identity”. Homologous sequences may include amino acid sequences that may be at least 80%, 85%, 90%, 99.1%, 99.2%, 99.3%, 99.4%, 99.5%, 99.6%, 99.7%, 99.8%, or 99.9% identical to the subject sequence. Typically, the homologue will comprise the same active site or the like as the subject amino acid sequence. Homology may be considered in terms of similarity (i.e., amino acid residues having similar chemical properties/functions) or may be expressed in terms of sequence identity. In the present application, a sequence having percent identity in any one of the SEQ ID NO of a mentioned amino acid sequence or nucleotide sequence refers to a sequence having the percent identity over the entire length of the mentioned SEQ ID NO. To determine sequence identity, sequence alignments may be performed by a variety of ways known to those skilled in the art, e.g., using BLAST, BLAST-2, ALIGN, NEEDLE, or Megalign (DNASTAR) software, etc. Those skilled in the art can determine the appropriate parameters for the alignment, including any algorithm required to achieve the optimal alignment over the full length of the sequence being compared.

The proteins or polypeptides used in the present application may also have deletions, insertions, or substitutions of amino acid residues which produce silent changes and result in functionally equivalent proteins. Intentional amino acid substitutions may be made on the basis of similarity in polarity, charge, solubility, hydrophobicity, hydrophilicity, and/or amphipathic nature of the residues, so long as the endogenous function is retained. For example, negatively charged amino acids include aspartic acid and glutamic acid; positively charged amino acids include lysine and arginine; and amino acids containing uncharged polar head groups with similar hydrophilicity values include asparagine, glutamine, serine, threonine, and tyrosine.

In the present application, the term “immunoconjugate” generally refers to a conjugate formed by the conjugation of other agents (e.g., a chemotherapeutic agent, a radioactive element, a cytostatic agent, and a cytotoxic agent) to the isolated antigen-binding protein (e.g., via covalent attachment of a linking molecule), wherein the conjugate can deliver the other agents to a target cell (e.g., a tumor cell) via specific binding of the isolated antigen-binding protein to an antigen on the target cell. The immunoconjugate then undergoes such internalization and eventually enters the interior of the target cell (e.g., into vesicles such as a lysosome), at which point the linker molecule in the immunoconjugate can be cleaved to release the other agent, thereby exerting its cytotoxic effect. In addition, the antigen may also be secreted by the target cell and located in the space outside the target cell.

In the present application, the term “subject” generally refers to human or non-human animals, including, but not limited to, cats, dogs, horses, pigs, cows, caprid, rabbits, mice, rats, or monkeys.

In the present application, the term “nucleic acid molecule” generally refers to an isolated form of nucleotides, deoxyribonucleotides, or ribonucleotides or analogs thereof, of any length, isolated from their natural environment or artificially synthesized.

In the present application, the term “vector” generally refers to a nucleic acid molecule capable of self-replication in a suitable host. The vector can transfer the inserted nucleic acid molecule into and/or between cells. The vector may include a vector for primarily inserting DNA or RNA into a cell, a vector for primarily replicating DNA or RNA, and a vector for primarily expressing transcription and/or translation of DNA or RNA. The vector can be a polynucleotide capable of being transcribed and translated into a polypeptide when introduced into an appropriate cell. In general, the vector may produce the desired expression product by culturing an appropriate cell containing the vector. In the present application, the vector may include a lentiviral vector.

In the present application, the term “cell” generally refers to an individual cell, cell line or cell culture that may or may already contain a plasmid or vector comprising a nucleic acid molecule as described herein, or that is capable of expressing a polypeptide as described herein or an antigen-binding protein as described herein. The cell may include the progeny of a single cell. Due to natural, accidental, or deliberate mutations, the progeny cells may not necessarily be identical in morphology or in genome to the original parent cell, but are capable of expressing the polypeptide or antigen-binding protein as described herein. The cells can be obtained by transfecting cells in vitro with the vectors as described herein. The cells may be prokaryotic cells (e.g., E. coli) or eukaryotic cells (e.g., yeast cell, COS cells, Chinese Hamster Ovary (CHO) cells, HeLa cells, HEK293 cells, COS-1 cells, NSO cells, or myeloma cells). In some embodiments, the cells may be immune cells. For example, the immune cell may be selected from the group consisting of T cells, B cells, natural killer cells (NK cells), macrophages, NKT cells, monocytes, dendritic cells, granulocytes, lymphocytes, leukocytes and/or peripheral blood mononuclear cells.

In the present application, the term “treatment” generally refers to: (i) the prevention of the development of a disease, disorder, and/or condition in a patient who may be susceptible to, but has not yet been diagnosed with, that disease, disorder, or condition; (ii) the suppression of the disease, disorder, or condition, i.e., the curb of the development; and (iii) remission of the disease, disorder, or condition, i.e., causing regression of the disease, disorder, and/or condition and/or symptoms associated with the disease, disorder, and/or condition.

In the present application, the terms “polypeptide”, “peptide”, and “protein” are used interchangeably and generally refer to a polymer of amino acids of any length. The polymer may be linear or branched, and may comprise modified amino acids and it may be interrupted by non-amino acids. These terms also encompass amino acid polymers that have been modified. These modifications may comprise: disulfide bond formation, glycosylation, lipidation, acetylation, phosphorylation, or any other manipulation (e.g., binding to a labeling component). The term “amino acid” includes natural and/or non-natural or synthetic amino acids, including glycine as well as the D and L optical isomers, as well as amino acid analogs and peptidomimetics.

In the present application, the terms “polynucleotide”, “nucleotide”, “nucleotide sequence”, “nucleic acid”, and “oligonucleotide” are used interchangeably and refer generally to polymeric forms of nucleotides of any length, such as deoxyribonucleotides or ribonucleotides, or analogs thereof. A polynucleotide may have any three-dimensional structure and may perform any function that is known or unknown. Non-limiting examples of polynucleotides are as follows: a coding or noncoding region of a gene or gene fragment, a plurality of loci (one locus) as defined by ligation analysis, exons, introns, messenger RNA (mRNA), transport RNA, ribosomal RNA, short interfering RNA (siRNA), short hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modification of the nucleotide structure may be performed before or after assembly of the polymer. The sequence of nucleotides may be interrupted by non-nucleotide components. The polynucleotides may be further modified after polymerization, e.g., by conjugation to labeled components.

In the present application, the term “K_{D}” (likewise, “K_{D}” or “KD”) generally refers to an “affinity constant” or an “equilibrium dissociation constant” and refers to a value obtained at equilibrium in a titration measurement, or by dividing the dissociation rate constant (k_{d}) by the binding rate constant (kₐ). The binding affinity of a binding protein (e.g., an isolated antigen-binding protein as described herein) for an antigen is expressed using an association rate constant (kₐ), a dissociation rate constant (k_{d}), and an equilibrium dissociation constant (K_{D}). Methods for determining association and dissociation rate constants are well known in the art. The use of fluorescence-based techniques provides high sensitivity and the ability to examine samples at equilibrium in physiological buffers. For example, the K_{D} value can be determined by Octet assay, and other experimental approaches and instruments such as BIAcore (Biomolecular Interaction Analysis) can be used (e.g., instruments available from BIAcoreInternationalAB, aGEHealthcarecompany, Uppsala, Sweden). Alternatively, the K_{D} value can be determined using KinExA (dynamic exclusion assay (KineticExclusionAssay)) available from SapidyneInstruments (Boise, Idaho) or using a surface plasmon resonance (SPR) instrument.

In the present application, the term “IC50 value” or the term “half-maximal inhibitory concentration” (IC50) indicates the concentration of a particular compound that is required to achieve 50% inhibition of a biological process in vitro. The IC50 value can be logarithmically converted to a pIC50 value (−log IC50), where higher values indicate exponentially greater potency. The IC50 value is not an absolute value, but rather depends on experimental conditions, such as the concentration used. The IC50 value can be converted to an absolute inhibitory constant (Ki) using the Cheng-Prusoff equation (Biochem. Pharmacol. (1973) 22:3099).

The term “EC50”, in the context of in vitro or in vivo analyses using isolated antigen-binding fragments, refers to the concentration of the antibody or antigen-binding portion thereof when the induced response is 50% of the maximal response, i.e., halfway between the maximal response and the baseline.

The terms “TL1A inhibitor” and “TL1A antagonist” are used interchangeably. Each is a molecule that detectably inhibits at least one function of TL1A. Conversely, a “TL1A agonist” is a molecule that detectably enhances at least one function of TL1A. The inhibition caused by the TL1A inhibitor need not be complete, as long as it is detectable by, for example, using an assay. Any assay for α4β7 function may be employed, examples of which are provided herein. Examples of functions of TL1A that can be inhibited by TL1A inhibitors (or enhanced by TL1A agonists) include ligand binding (i.e., binding to MAdCAM-1), adhesion to cells expressing the ligand, transport to specific compartments (e.g., intestines), release of cytokines, chemokines, and other mediators, exacerbation or worsening of inflammatory responses and tissue damage, and the like. The TL1A inhibitors and the TL1A agonists of the Examples of types include, but are not limited to, TL1A-binding polypeptides, such as antigen-binding proteins (e.g., TL1A antigen-binding proteins), antibodies, antibody fragments, and antibody derivatives.

The terms “IL-23p19 inhibitor” and “IL-23p19 antagonist” are used interchangeably.

As used herein, the term “epitope” or “antigenic determinant cluster” generally refers to a site on an antigen to which an antibody binds. Epitopes may be formed from contiguous amino acids (linear epitopes) or discontinuous amino acids (conformational epitopes) adjacent to each other by tertiary folding of the protein. Epitopes formed from contiguous amino acids are typically retained upon exposure to denaturing solvents, whereas epitopes formed by tertiary folding are typically lost upon treatment with denaturing solvents. Epitopes typically include at least 3, and more typically at least 5 or 8-10 amino acids in a unique spatial conformation. Methods for determining the spatial conformation of epitopes include, for example, x-ray crystallography and two-dimensional nuclear magnetic resonance. See, e.g., Epitope Mapping Protocols in Methods in Molecular Biology, vol. 66, edited by Glenn E. Morris (1996).

As used herein, the term “conformational epitope” generally refers to non-contiguous amino acid residues of an antigen (such as the PD-L1 antigen) that are adjacent to the protein by tertiary folding. These non-contiguous amino acid residues may converge on the surface when the polypeptide chain folds to form a natural protein. Conformational epitopes include, but are not limited to, functional epitopes.

As used herein, the term “cross-linking” refers to the process by which an antigen-binding protein binds to more than one antigen at the same time, or an antigen binds to more than one antigen-binding protein at the same time. For example, RVT-3101 (Analogue) monoclonal antibody binds to huTL1A-his antigen, which can be seen as three monoclonal antibodies bound to a single TL1A trimer, i.e., a 1Ag (Ag represents the antigen)+3Ab (Ab represents the antibody) complex. tla0015-11 monoclonal antibody binds to the huTL1A-his antigen, which results in the formation of a multiple-antigen binding protein that binds to a number of antigen-binding proteins cross-linked complexes.

As used herein, the term “functional epitope” generally refers to the amino acid residues of an antigen that actively contribute to the binding of an antibody, i.e., form an “active epitope.” Mutation of any active promoting residue of the antigen to alanine will destroy the binding of the antibody, so that the relative KD ratio of the antibody (KD mutant/KD wild type) can be, for example, greater than 2 times, such as greater than 3 times, greater than 4 times, greater than 6 times, greater than 10 times, greater than 20 times, greater than 30 times, greater than 40 times, greater than 50 times, greater than 60 times, greater than 70 times, greater than 80 times, greater than 90 times, greater than 100 times, greater than 150 times, greater than 200 times or more.

In the present application, the term “and/or” should be understood as meaning any one of the alternatives or both of the alternatives.

In the present application, the term “comprising” or “containing” generally refers to the inclusion of explicitly specified features, but not excluding other elements. In certain instances, “comprising” or “contain” also encompasses the inclusion of only the specified components.

In the present application, the term “about” generally refers to a range from 0.5% to 10% above or below the specified value, for example, a range of 0.5%, 1%, 1.5%, 2%, 2.5%, 3%, 3.5%, 4%, 4.5%, 5, 55.5%, 6%, 6.5%, 7%, 7.5%, 8%, 8.5%, 9%, 9.5%, or 10% above or below the specified value.

In the present application, the term “including” generally means comprising, summing up, encompassing or covering. In certain instances, the meaning of “being”, “consisting of . . . ” is also indicated.

### DETAILED DESCRIPTION

### Frist Binding Domain

In one aspect, the present application provides an isolated antigen-binding protein, which includes a first binding domain that can specifically bind to TNF-like protein A (TL1A). It includes a heavy chain variable region (VH), wherein the VH includes at least one heavy chain complementary determining region (HCDR). The VH may include at least one, two or three of HCDR1, HCDR2 and HCDR3. In some embodiments, HCDR3, HCDR2 and HCDR1 are sequences as shown in SEQ ID NO:14, 5, and 1, respectively.

The HCDR3 of the antigen-binding protein may include the amino acid sequence shown in SEQ ID NO:14. For example, the HCDR3 sequence of the antigen-binding protein may be defined according to the Kabat coding system.

In some embodiments, the HCDR3 includes the amino acid sequence of any one of SEQ ID NO:15-18.

The HCDR2 of the antigen-binding protein may include the amino acid sequence of SEQ ID NO:5. For example, the HCDR3 sequence of the antigen-binding protein may be defined according to the Kabat coding system.

In some embodiments, the HCDR2 includes the amino acid sequence of any one of SEQ ID NO:6-13.

The HCDR1 of the antigen binding protein may comprise the amino acid sequence of SEQ ID NO:1. For example, the HCDR3 sequence of the antigen binding protein may be defined according to the Kabat coding system.

In some embodiments, the HCDR1 comprises the amino acid sequence of any one of SEQ ID NO:2-4.

In some embodiments, the VH comprises HFR1, and the C-terminus of the HFR1 is directly or indirectly connected to the N-terminus of the HCDR1. For example, the HFR1 sequence of the antigen binding protein may be defined according to the Kabat coding system. The HFR1 comprises the amino acid sequence shown in SEQ ID NO: 19.

In some embodiments, the VH comprises HFR1, and the C-terminus of the HFR1 is directly or indirectly connected to the N-terminus of the HCDR1. For example, the HFR1 sequence of the antigen binding protein may be defined according to the Kabat coding system. The HFR1 comprises the amino acid sequence shown in SEQ ID NO: 19.

In some embodiments, the HFR1 is selected from the amino acid sequence shown in any one of SEQ ID NO:20-28.

In some embodiments, the VH comprises HFR2, which is located between the HCDR1 and the HCDR2. For example, the HFR2 sequence of the antigen binding protein can be defined according to the Kabat coding system. The HFR2 comprises the amino acid sequence of SEQ ID NO:29.

In some embodiments, wherein the HFR2 is selected from the amino acid sequence shown in any one of SEQ ID NOs:30-32.

In some embodiments, the VH comprises HFR3, which is located between the HCDR2 and the HCDR3. For example, the HFR3 sequence of the antigen binding protein can be defined according to the Kabat coding system. The HFR3 comprises the amino acid sequence of SEQ ID NO:33.

In some embodiments, wherein the HFR3 is selected from the amino acid sequence shown in any one of SEQ ID NOs:34-40.

In some embodiments, the VH comprises HFR4, the N-terminus of the HFR4 is directly or indirectly connected to the C-terminus of the HCDR3. For example, the HFR4 sequence of the antigen binding protein can be defined according to the Kabat coding system. The HFR4 sequence comprises the amino acid sequence shown in SEQ ID NO:41.

In some embodiments, the isolated antigen-binding protein further comprises a light chain variable region (VL), the VL comprises at least one light chain complementary determining region (LCDR), the VL comprises LCDR1, LCDR2 and LCDR3 comprising an amino acid sequence selected from the amino acid sequences shown in SEQ ID NO:42, SEQ ID NO:43 and SEQ ID NO:44.

In some embodiments, the VL comprises the LCDR3 comprising the amino acid sequence of SEQ ID NO:44. For example, the LCDR3 sequence of the antigen-binding protein can be defined according to the Kabat coding system.

In some embodiments, the VL comprises the LCDR2 comprising the amino acid sequence of SEQ ID NO:43. For example, the LCDR2 sequence of the antigen-binding protein can be defined according to the Kabat coding system.

In some embodiments, the VL comprises the LCDR1 comprising the amino acid sequence of SEQ ID NO:42. For example, the LCDR1 sequence of the antigen-binding protein can be defined according to the Kabat coding system.

In some embodiments, the VL comprises LFR1, the C-terminus of the LFR1 is directly or indirectly connected to the N-terminus of the LCDR1. For example, the LFR1 sequence of the antigen binding protein can be defined according to the Kabat coding system. The LFR1 comprises the amino acid sequence of SEQ ID NO:45.

In some embodiments, the LFR1 comprises the amino acid sequence shown in any one of SEQ ID NOs:46-47.

In some embodiments, the VL comprises LFR2, and the LFR2 is located between the LCDR1 and the LCDR2. For example, the LFR2 sequence of the antigen binding protein can be defined according to the Kabat coding system. It comprises the amino acid sequence of SEQ ID NO:48.

In some embodiments, the LFR2 comprises the amino acid sequence shown in any one of SEQ ID NOs:49-52 In some embodiments, the VL comprises LFR3, and the LFR3 is located between the LCDR2 and the LCDR3, for example, the LFR3 sequence of the antigen binding protein can be defined according to the Kabat coding system. It comprises the amino acid sequence of SEQ ID NO:53.

In some embodiments, the LFR3 comprises an amino acid sequence as shown in any one of SEQ ID NOs: 54-57.

In some embodiments, the isolated antigen-binding protein comprises LFR4, the N-terminus of the LFR4 is directly or indirectly connected to the C-terminus of the LCDR3, for example, the LFR4 sequence of the antigen-binding protein can be defined according to the Kabat coding system. And the LFR4 comprises an amino acid sequence as shown in SEQ ID NO: 58.

In some embodiments, the LFR1 comprises an amino acid sequence as shown in any one of SEQ ID NOs: 59-61.

In some embodiments, the VH comprises a sequence as shown in any one of SEQ ID NOs: 62-99.

In some embodiments, the VL comprises a sequence as shown in any one of SEQ ID NOs: 102-106.

In some embodiments, the above-mentioned antigen-binding protein is as described in Tables A and B.

In some embodiments, the first binding domain comprises a VH as shown in SEQ ID NO: 70 and a VL as shown in SEQ ID NO: 106.

In some embodiments, the VH of the first binding domain comprises HCDR1 as shown in SEQ ID NO:2, HCDR2 as shown in SEQ ID NO:6, and HCDR3 as shown in SEQ ID NO:15, and further comprises a light chain variable region (VL), wherein the VL comprises at least one light chain complementary determining region (LCDR) comprising LCDR1 as shown in SEQ ID NO:42, LCDR2 as shown in SEQ ID NO:43, and LCDR3 as shown in SEQ TD NO: 44.

In some embodiments, the antibody distribution of the TLA-0015 series is shown in the following table, Table A is the heavy chain component distribution, and Table B is the light chain component distribution.

TABLE A

| | VH | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| Antibody | HFR1 | ID | HCDR1 | ID | HFR2 | ID | HCDR2 | ID | HFR3 | ID | HCDR3 | ID | HFR4 | ID |
| TLA0015 | | # | TLA0015- | 2 | TLA0015- | 32 | TLA0015- | 6 | | | TLA0015- | 15 | | # |
| (mouse) | | N/A | HCDR1-1 | | HFR2-3 | | HCDR2-1 | | | | HCDR3-1 | | | N/A |
| TLA0015-1 | TLA0015- | 20 | TLA0015- | 2 | TLA0015- | 30 | TLA0015- | 6 | TLA0015- | 34 | TLA0015- | 15 | TLA0015- | 41 |
| | HFR1-1 | | HCDR1-1 | | HFR2-1/ | | HCDR2-1 | | HFR3-1 | | HCDR3-1 | | HFR4-1/ | |
| | | | | | | | | | | | | | TLA-0238- | |
| | | | | | | | | | | | | | HFR4-2 | |
| TLA0015-2 | TLA0015- | 20 | TLA0015- | 2 | TLA0015- | 30 | TLA0015- | 6 | TLA0015- | 34 | TLA0015- | 15 | TLA0015- | 41 |
| | HFR1-1 | | HCDR1-1 | | HFR2-1/ | | HCDR2-1 | | HFR3-1 | | HCDR3-1 | | HFR4-1/ | |
| | | | | | | | | | | | | | TLA-0238- | |
| | | | | | | | | | | | | | HFR4-2 | |
| TLA0015-3 | TLA0015- | 21 | TLA0015- | 2 | TLA0015- | 31 | TLA0015- | 6 | TLA0015- | 35 | TLA0015- | 15 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-4 | TLA0015- | 21 | TLA0015- | 2 | TLA0015- | 31 | TLA0015- | 6 | TLA0015- | 35 | TLA0015- | 15 | TLA0015- | 41 |
| | HFR1-2 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-5 | TLA0015- | 22 | TLA0015- | 2 | TLA0015- | 32 | TLA0015- | 6 | TLA0015- | 35 | TLA0015- | 15 | TLA0015- | 41 |
| | HFR1-3 | | HCDR1-1 | | HFR2-3 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-6 | TLA0015- | 22 | TLA0015- | 2 | TLA0015- | 32 | TLA0015- | 6 | TLA0015- | 35 | TLA0015- | 15 | TLA0015- | 41 |
| | HFR1-3 | | HCDR1-1 | | HFR2-3 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-7 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLA0015-8 | TLA0015- | 23 | TLA0015- | 2 | TLA0015- | 31 | TLA0015- | 6 | TLA0015- | 37 | TLA0015- | 15 | TLA0015- | 41 |
| | HFR1-4 | | HCDR1-1 | | HFR2-2 | | HCDR2-1 | | HFR3-4 | | HCDR3-1 | | HFR4-1/ | |
| | | | | | | | | | | | | | TLA-0238- | |
| | | | | | | | | | | | | | HFR4-2 | |
| TLA0015-9 | TLA0015- | 23 | TLA0015- | 2 | TLA0015- | 31 | TLA0015- | 6 | TLA0015- | 35 | TLA0015- | 15 | TLA0015- | 41 |
| | HFR1-4 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-10 | TLA0015- | 24 | TLA0015- | 2 | TLA0015- | 31 | TLA0015- | 6 | TLA0015- | 38 | TLA0015- | 15 | TLA0015- | 41 |
| | HFR1-5 | | HCDR1-1 | | HFR2-2 | | HCDR2-1 | | HFR3-5 | | HCDR3-1 | | HFR4-1/ | |
| | | | | | | | | | | | | | TLA-0238- | |
| | | | | | | | | | | | | | HFR4-2 | |
| TLA0015-11 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-12 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-13 | TLA0015- | 25 | TLA0015- | 2 | TLA0015- | 31 | TLA0015- | 6 | TLA0015- | 35 | TLA0015- | 15 | TLA0015- | 41 |
| | HFR1-6 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA0015-14 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLA0015-15 | TLA0015- | 23 | TLA0015- | 2 | TLA0015- | 31 | TLA0015- | 6 | TLA0015- | 37 | TLA0015- | 15 | TLA0015- | 41 |
| | HFR1-4 | | HCDR1-1 | | HFR2-2 | | HCDR2-1 | | HFR3-4 | | HCDR3-1 | | HFR4-1/ | |
| | | | | | | | | | | | | | TLA-0238- | |
| | | | | | | | | | | | | | HFR4-2 | |
| TLA0015-16 | TLA0015- | 23 | TLA0015- | 2 | TLA0015- | 31 | TLA0015- | 6 | TLA0015- | 36 | TLA0015- | 15 | TLA0015- | 41 |
| | HFR1-4 | | HCDR1-1 | | HFR2-2 | | HCDR2-1 | | HFR3-3 | | HCDR3-1 | | HFR4-1/ | |
| | | | | | | | | | | | | | TLA-0238- | |
| | | | | | | | | | | | | | HFR4-2 | |
| TLa0015-17 | TLA0015- | 25 | TLA0015- | 2 | TLA0015- | 31 | TLA0015- | 6 | TLA0015- | 37 | TLA0015- | 15 | TLA0015- | 41 |
| | HFR1-6 | | HCDR1-1 | | HFR2-2 | | HCDR2-1 | | HFR3-4 | | HCDR3-1 | | HFR4-1 | |
| TLa0015-18 | TLA0015- | 25 | TLA0015- | 2 | TLA0015- | 31 | TLA0015- | 6 | TLA0015- | 39 | TLA0015- | 15 | TLA0015- | 41 |
| | HFR1-6 | | HCDR1-1 | | HFR2-2 | | HCDR2-1 | | HFR3-6 | | HCDR3-1 | | HFR4-1 | |
| TLa0015-19 | TLA0015- | 25 | TLA0015- | 2 | TLA0015- | 31 | TLA0015- | 7 | TLA0015- | 35 | TLA0015- | 15 | TLA0015- | 41 |
| | HFR1-6 | | HCDR1-1 | | HFR2-2 | | HCDR2-2 | | HFR3-2 | | HCDR3-1 | | HFR4-1 | |
| TLa0015-20 | TLA0015- | 25 | TLA0015- | 2 | TLA0015- | 31 | TLA0015- | 8 | TLA0015- | 35 | TLA0015- | 15 | TLA0015- | 41 |
| | HFR1-6 | | HCDR1-1 | | HFR2-2 | | HCDR2-3 | | HFR3-2 | | HCDR3-1 | | HFR4-1/ | |
| | | | | | | | | | | | | | TLA-0238- | |
| | | | | | | | | | | | | | HFR4-2 | |
| TLa0015-21 | TLA0015- | 25 | TLA0015- | 2 | TLA0015- | 31 | TLA0015- | 6 | TLA0015- | 40 | TLA0015- | 15 | TLA0015- | 41 |
| | HFR1-6 | | HCDR1-1 | | HFR2-2 | | HCDR2-1 | | HFR3-7 | | HCDR3-1 | | HFR4-1 | |
| TLA015-22 | TLA0015- | 25 | TLA0015- | 3 | TLA0015- | 31 | TLA0015- | 6 | TLA0015- | 35 | TLA0015- | 15 | TLA0015- | 41 |
| | HFR1-6 | | HCDR1-2 | | HFR2-2 | | HCDR2-1 | | HFR3-2 | | HCDR3-1 | | HFR4-1 | |
| TLA015-23 | TLA0015- | 25 | TLA0015- | 2 | TLA0015- | 31 | TLA0015- | 6 | TLA0015- | 35 | TLA0015- | 16 | TLA0015- | 41 |
| | HFR1-6 | | HCDR1-1 | | HFR2-2 | | HCDR2-1 | | HFR3-2 | | HCDR3-2 | | HFR4-1 | |
| TLA015-24 | TLA0015- | 25 | TLA0015- | 2 | TLA0015- | 31 | TLA0015- | 6 | TLA0015- | 35 | TLA0015- | 17 | TLA0015- | 41 |
| | HFR1-6 | | HCDR1-1 | | HFR2-2 | | HCDR2-1 | | HFR3-2 | | HCDR3-3 | | HFR4-1 | |
| TLA015-25 | TLA0015- | 25 | TLA0015- | 2 | TLA0015- | 31 | TLA0015- | 6 | TLA0015- | 35 | TLA0015- | 18 | TLA0015- | 41 |
| | HFR1-6 | | HCDR1-1 | | HFR2-2 | | HCDR2-1 | | HFR3-2 | | HCDR3-4 | | HFR4-1 | |
| TLA015-26 | TLA0015- | 25 | TLA0015- | 4 | TLA0015- | 31 | TLA0015- | 6 | TLA0015- | 35 | TLA0015- | 15 | TLA0015- | 41 |
| | HFR1-6 | | HCDR1-3 | | HFR2-2 | | HCDR2-1 | | HFR3-2 | | HCDR3-1 | | HFR4-1 | |
| TLA015-27 | TLA0015- | 26 | TLA0015- | 2 | TLA0015- | 31 | TLA0015- | 6 | TLA0015- | 35 | TLA0015- | 15 | TLA0015- | 41 |
| | HFR1-7 | | HCDR1-1 | | HFR2-2 | | HCDR2-1 | | HFR3-2 | | HCDR3-1 | | HFR4-1 | |
| TLA015-28 | TLA0015- | 25 | TLA0015- | 2 | TLA0015- | 31 | TLA0015- | 9 | TLA0015- | 35 | TLA0015- | 15 | TLA0015- | 41 |
| | HFR1-6 | | HCDR1-1 | | HFR2-2 | | HCDR2-4 | | HFR3-2 | | HCDR3-1 | | HFR4-1 | |
| TLA015-29 | TLA0015- | 27 | TLA0015- | 2 | TLA0015- | 31 | TLA0015- | 6 | TLA0015- | 35 | TLA0015- | 15 | TLA0015- | 41 |
| | HFR1-8 | | HCDR1-1 | | HFR2-2 | | HCDR2-1 | | HFR3-2 | | HCDR3-1 | | HFR4-1 | |
| TLA015-30 | TLA0015- | 25 | TLA0015- | 2 | TLA0015- | 31 | TLA0015- | 10 | TLA0015- | 35 | TLA0015- | 15 | TLA0015- | 41 |
| | HFR1-6 | | HCDR1-1 | | HFR2-2 | | HCDR2-5 | | HFR3-2 | | HCDR3-1 | | HFR4-1/ | |
| TLA015-31 | TLA0015- | 25 | TLA0015- | 2 | TLA0015- | 31 | TLA0015- | 11 | TLA0015- | 35 | TLA0015- | 15 | TLA0015- | 41 |
| | HFR1-6 | | HCDR1-1 | | HFR2-2 | | HCDR2-6 | | HFR3-2 | | HCDR3-1 | | HFR4-1/ | |
| | | | | | | | | | | | | | TLA-0238- | |
| | | | | | | | | | | | | | HFR4-2 | |
| TLA015-32 | TLA0015- | 28 | TLA0015- | 2 | TLA0015- | 31 | TLA0015- | 6 | TLA0015- | 35 | TLA0015- | 15 | TLA0015- | 41 |
| | HFR1-9 | | HCDR1-1 | | HFR2-2 | | HCDR2-1 | | HFR3-2 | | HCDR3-1 | | HFR4-1/ | |
| | | | | | | | | | | | | | TLA-0238- | |
| | | | | | | | | | | | | | HFR4-2 | |
| TLA015-33 | TLA0015- | 25 | TLA0015- | 2 | TLA0015- | 31 | TLA0015- | 12 | TLA0015- | 35 | TLA0015- | 15 | TLA0015- | 41 |
| | HFR1-6 | | HCDR1-1 | | HFR2-2 | | HCDR2-7 | | HFR3-2 | | HCDR3-1 | | HFR4-1/ | |
| | | | | | | | | | | | | | TLA-0238- | |
| | | | | | | | | | | | | | HFR4-2 | |
| TLA015-34 | TLA0015- | 25 | TLA0015- | 2 | TLA0015- | 31 | TLA0015- | 13 | TLA0015- | 35 | TLA0015- | 15 | TLA0015- | 41 |
| | HFR1-6 | | HCDR1-1 | | HFR2-2 | | HCDR2-8 | | HFR3-2 | | HCDR3-1 | | HFR4-1/ | |
| | | | | | | | | | | | | | TLA-0238- | |
| | | | | | | | | | | | | | HFR4-2 | |
| TLA015-35 | TLA0015- | 25 | TLA0015- | 3 | TLA0015- | 31 | TLA0015- | 6 | TLA0015- | 35 | TLA0015- | 16 | TLA0015- | 41 |
| | HFR1-6 | | HCDR1-2 | | HFR2-2 | | HCDR2-1 | | HFR3-2 | | HCDR3-2 | | HFR4-1/ | |
| TLA015-36 | TLA0015- | 25 | TLA0015- | 3 | TLA0015- | 31 | TLA0015- | 6 | TLA0015- | 35 | TLA0015- | 17 | TLA0015- | 41 |
| | HFR1-6 | | HCDR1-2 | | HFR2-2 | | HCDR2-1 | | HFR3-2 | | HCDR3-3 | | HFR4-1 | |
| TLA015-37 | TLA0015- | 25 | TLA0015- | 3 | TLA0015- | 31 | TLA0015- | 9 | TLA0015- | 35 | TLA0015- | 15 | TLA0015- | 41 |
| | HFR1-6 | | HCDR1-2 | | HFR2-2 | | HCDR2-4 | | HFR3-2 | | HCDR3-1 | | HFR4-1 | |
| TLA015-38 | TLA0015- | 25 | TLA0015- | 3 | TLA0015- | 31 | TLA0015- | 10 | TLA0015- | 35 | TLA0015- | 15 | TLA0015- | 41 |
| | HFR1-6 | | HCDR1-2 | | HFR2-2 | | HCDR2-5 | | HFR3-2 | | HCDR3-1 | | HFR4-1 | |
| TLA015-39 | TLA0015- | 25 | TLA0015- | 3 | TLA0015- | 31 | TLA0015- | 11 | TLA0015- | 35 | TLA0015- | 15 | TLA0015- | 41 |
| | HFR1-6 | | HCDR1-2 | | HFR2-2 | | HCDR2-6 | | HFR3-2 | | HCDR3-1 | | HFR4-1/ | |
| | | | | | | | | | | | | | TLA-0238- | |
| | | | | | | | | | | | | | HFR4-2 | |
| TLA015-40 | TLA0015- | 28 | TLA0015- | 3 | TLA0015- | 31 | TLA0015- | 6 | TLA0015- | 35 | TLA0015- | 15 | TLA0015- | 41 |
| | HFR1-9 | | HCDR1-2 | | HFR2-2 | | HCDR2-1 | | HFR3-2 | | HCDR3-1 | | HFR4-1 | |
| TLA015-41 | TLA0015- | 26 | TLA0015- | 2 | TLA0015- | 31 | TLA0015- | 6 | TLA0015- | 35 | TLA0015- | 16 | TLA0015- | 41 |
| | HFR1-7 | | HCDR1-1 | | HFR2-2 | | HCDR2-1 | | HFR3-2 | | HCDR3-2 | | HFR4-1 | |
| TLA015-42 | TLA0015- | 25 | TLA0015- | 2 | TLA0015- | 31 | TLA0015- | 9 | TLA0015- | 35 | TLA0015- | 16 | TLA0015- | 41 |
| | HFR1-6 | | HCDR1-1 | | HFR2-2 | | HCDR2-4 | | HFR3-2 | | HCDR3-2 | | HFR4-1 | |
| TLA015-43 | TLA0015- | 25 | TLA0015- | 2 | TLA0015- | 31 | TLA0015- | 10 | TLA0015- | 35 | TLA0015- | 16 | TLA0015- | 41 |
| | HFR1-6 | | HCDR1-1 | | HFR2-2 | | HCDR2-5 | | HFR3-2 | | HCDR3-2 | | HFR4-1 | |
| TLA015-44 | TLA0015- | 25 | TLA0015- | 2 | TLA0015- | 31 | TLA0015- | 11 | TLA0015- | 35 | TLA0015- | 16 | TLA0015- | 41 |
| | HFR1-6 | | HCDR1-1 | | HFR2-2 | | HCDR2-6 | | HFR3-2 | | HCDR3-2 | | HFR4-1 | |
| TLA015-45 | TLA0015- | 25 | TLA0015- | 2 | TLA0015- | 31 | TLA0015- | 12 | TLA0015- | 35 | TLA0015- | 16 | TLA0015- | 41 |
| | HFR1-6 | | HCDR1-1 | | HFR2-2 | | HCDR2-7 | | HFR3-2 | | HCDR3-2 | | HFR4-1 | |
| TLA015-46 | TLA0015- | 26 | TLA0015- | 2 | TLA0015- | 31 | TLA0015- | 6 | TLA0015- | 35 | TLA0015- | 17 | TLA0015- | 41 |
| | HFR1-7 | | HCDR1-1 | | HFR2-2 | | HCDR2-1 | | HFR3-2 | | HCDR3-3 | | HFR4-1 | |
| TLA015-47 | TLA0015- | 25 | TLA0015- | 2 | TLA0015- | 31 | TLA0015- | 10 | TLA0015- | 35 | TLA0015- | 17 | TLA0015- | 41 |
| | HFR1-6 | | HCDR1-1 | | HFR2-2 | | HCDR2-5 | | HFR3-2 | | HCDR3-3 | | HFR4-1/ | |
| | | | | | | | | | | | | | TLA-0238- | |
| | | | | | | | | | | | | | HFR4-2 | |

TABLE B

| | VL | | | | | | | | | | | | | |
|---|---|---|---|---|---|---|---|---|---|---|---|---|---|---|
| | | SEQ | | SEQ | | SEQ | | SEQ | | SEQ | | SEQ | | SEQ |
| antibody | LFR1 | ID | CDR1 | ID | LFR2 | ID | LCDR2 | ID | LFR3 | ID | LCDR3 | ID | LFR4 | ID |
| TLA0015 | | | TLA0015- | 42 | | | TL0015- | 43 | | | TL0015- | 44 | | |
| (mouse) | | | LCDR1-1 | | | | LCDR2-1 | | | | LCDR3-1 | | | |
| TLA0015-1 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-2 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-3 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-4 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-5 | TL0015- | 47 | TLA0015- | 42 | TL0015- | 50 | TL0015- | 43 | TL0015- | 55 | TL0015- | 44 | TL0015- | 60 |
| | LFR1-2/ | | LCDR1-1 | | LFR2-2 | | LCDR2-1 | | LFR3-2 | | LCDR3-1 | | LFR4-2 | |
| | TLA0241- | | | | | | | | | | | | | |
| | LFR1-1 | | | | | | | | | | | | | |
| TLA0015-6 | TL0015- | 47 | TLA0015- | 42 | TL0015- | 51 | TL0015- | 43 | TL0015- | 56 | TL0015- | 41 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-7 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-8 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-9 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-10 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-11 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA0015-12 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-13 | TL0015- | 47 | TLA0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLA0015-14 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-15 | TL0015- | 47 | TLA0015- | 42 | TL0015- | 52 | TL0015- | 43 | TL0015- | 57 | TL0015- | 44 | TL0015- | 61 |
| | LFR1-2/ | | LCDR1-1 | | LFR2-4/ | | LCDR2-1 | | LFR3-4/ | | LCDR3-1 | | LFR4-3/ | |
| | TLA0241- | | | | TLA0241- | | | | TLA0238- | | | | TLA0238- | |
| | LFR1-1 | | | | LFR2-1 | | | | LFR3-4/ | | | | LFR4-3 | |
| | | | | | | | | | TLA0241- | | | | | |
| | | | | | | | | | LFR3-2 | | | | | |
| TLA0015-16 | TL0015- | 47 | TLA0015- | 42 | TL0015- | 52 | TL0015- | 43 | TL0015- | 56 | TL0015- | 44 | TL0015- | 61 |
| | LFR1-2/ | | LCDR1-1 | | LFR2-4/ | | LCDR2-1 | | LFR3-3 | | LCDR3-1 | | LFR4-3/ | |
| | TLA0241- | | | | TLA0241- | | | | | | | | TLA0238- | |
| | LFR1-1 | | | | LFR2-1 | | | | | | | | LFR4-3 | |
| TLa0015-17 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-18 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-19 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-20 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLa0015-21 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-22 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-23 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-24 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-25 | TL0015- | 47 | TLA0015- | 42 | TL0015- | 51 | TL0015- | 43 | TL0015- | 56 | TL0015- | 41 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-26 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-27 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-28 | TL0015- | 47 | TLA0015- | 42 | TL0015- | 51 | TL0015- | 43 | TL0015- | 56 | TL0015- | 41 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-29 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-30 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-31 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-32 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-33 | TL0015- | 17 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-34 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-35 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-36 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-37 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-38 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-39 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-40 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-41 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-42 | TL0015- | 47 | TLA0015- | 42 | TL0015- | 51 | TL0015- | 43 | TL0015- | 56 | TL0015- | 44 | TL0015- | 60 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-43 | TL0015- | 17 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-44 | TL0015- | 47 | TLA0015- | 42 | TL0015- | 51 | TL0015- | 43 | TL0015- | 56 | TL0015- | 41 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-45 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-46 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |
| TLA015-47 | TL0015- | 47 | TLA0015- | 42 | TL0015- | 51 | TL0015- | 43 | TL0015- | 56 | TL0015- | 44 | TL0015- | 61 |
| | LFR1-2/ | | LCDR1-1 | | LFR2-3 | | LCDR2-1 | | LFR3-3 | | LCDR3-1 | | LFR4-3/ | |
| | TLA0241- | | | | | | | | | | | | TLA0238- | |
| | LFR1-1 | | | | | | | | | | | | LFR4-3 | |

### Second Binding Domain

In the present application, the isolated antigen binding protein may include a second binding domain.

The second binding domain does not specifically bind to TL1A.

The second binding domain specifically binds to IL-23. Further, the second binding domain specifically binds to IL-23P19.

In some embodiments, the VH of the second binding domain includes the HCDR1 as shown in SEQ ID NO: 113 (RYL[IM][HN], wherein [IM] represents I or M, and [HN] represents H or N), the HCDR2 as shown in SEQ ID NO: 117, and the HCDR3 as shown in SEQ ID NO: 124.

In some embodiments, the VH of the second binding domain includes the HCDR1 as shown in SEQ ID NO: 114, the HCDR2 as shown in SEQ ID NO: 118, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 119, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 120, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 116, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 115, the HCDR2 as shown in SEQ ID NO: 121, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 122, and the HCDR3 as shown in SEQ ID NO: 124.

In some embodiments, the VH of the second binding domain comprises the HCDR1 as shown in SEQ ID NO: 114, the HCDR2 as shown in SEQ ID NO: 123, and the HCDR3 as shown in SEQ ID NO: 124.

In some embodiments, the VL of the second binding domain comprises the LCDR1 as shown in SEQ ID NO: 125, the LCDR2 as shown in SEQ ID NO: 126, and the LCDR3 as shown in SEQ ID NO: 127.

In some embodiments, the VL of the second binding domain includes the LCDR1 as shown in SEQ ID NO: 125, the LCDR2 as shown in SEQ ID NO: 126, and the LCDR3 as shown in SEQ ID NO: 130.

In some embodiments, the VL of the second binding domain includes the LCDR1 as shown in SEQ ID NO: 125, the LCDR2 as shown in SEQ ID NO: 126, and the LCDR3 as shown in SEQ ID NO: 129.

In some embodiments, the VL of the second binding domain includes the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 119, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 120, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 116, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 115, the HCDR2 as shown in SEQ ID NO: 121, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the VH of the second binding domain comprises the HCDR1 as shown in SEQ ID NO: 114, the HCDR2 as shown in SEQ ID NO: 122, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 130.

In some embodiments, the VH of the second binding domain comprises the HCDR1 as shown in SEQ ID NO: 114, the HCDR2 as shown in SEQ ID NO: 118, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 129.

In some embodiments, the VH of the second binding domain comprises the HCDR1 as shown in SEQ ID NO: 114, the HCDR2 as shown in SEQ ID NO: 123, and the HCDR3 as shown in SEQ ID NO: 124, wherein the VL of the second binding domain comprises the LCDR1 as shown in SEQ ID NO: 125, the LCDR2 as shown in SEQ ID NO: 126, and the LCDR3 as shown in SEQ ID NO: 128.

In some embodiments, the second antigen binding domain VH comprises the HFR1 as shown in SEQ ID NO: 209, the HFR2 as shown in SEQ ID NO: 220, the FR3 as shown in SEQ ID NO: 229, the HFR4 as shown in SEQ ID NO: 272 and/or the second antigen binding domain VL comprises the LFR1 as shown in SEQ ID NO: 232, the LFR2 as shown in SEQ ID NO: 246, the LFR3 as shown in SEQ ID NO: 273, the LFR4 as shown in SEQ ID NO: 263.

In some embodiments, the distribution of VL and VH of the 18E5 system is shown in the following table, Table C is the distribution of heavy chain components, and Table D is the distribution of light chain components.

TABLE C

| | | | SEQ ID | | SEQ ID | | SEQ ID | | SEQ ID | | SEQ ID | | SEQ ID | | SEQ ID |
|---|---|---|---|---|---|---|---|---|---|---|---|---|---|---|---|
| antibody | VH | HFR1 | NO | HCDR1 | NO | HFR2 | NO | HCDR2 | NO | HFR3 | NO | HCDR3 | NO | HFR4 | NO |
| 16003-18E5 | EVQLQQSGPELVKPGAAVKMS | | | RYLIH | 114 | | | YINP | 118 | | | NWDLDY | 124 | WGQGT | |
| | CKASGYTFTRYLIHWVKQKPG | | | | | | | YNDG | | | | | | TLTVS | |
| | QGLEWIGYINPYNDGTKYNEK | | | | | | | TKYN | | | | | | S | |
| | FKGKATLTSDKSSSTAYMELS | | | | | | | EKFK | | | | | | | |
| | SLTSEDSAVYYCTSNWDLDYW | | | | | | | G | | | | | | | |
| | GQGTTLTVSS | | | | | | | | | | | | | | |
| 23A0144 | QVQLVQSGAEVKKPGSSVKVS | QVQLV | 209 | RYLIH | 114 | WVRQ | 217 | YINP | 119 | RVTITA | 222 | NWDLDY | 124 | WGQGT | 41 |
| | CKASGYTFTRYLIHWVRQAPG | QSGAE | | | | APGQ | | YNDG | | DESTST | | | | LVTVS | |
| | QGLEWIGYINPYNDGTKYAQK | VKKPG | | | | GLEW | | TKYA | | AYMELS | | | | S | |
| | FQGRVTITADESTSTAYMELS | SSVKV | | | | IG | | QKFQ | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0145 | EVQLVESGGGLVKPGGSLRLS | EVQLV | 210 | RYLIH | 114 | WVRQ | 218 | YINP | 120 | RFTISR | 223 | NWDLDY | 124 | WGQGT | 41 |
| | CAASGYTFTRYLIHWVRQAPG | ESGGG | | | | APGK | | YNDG | | DDSKNT | | | | LVTVS | |
| | KGLEWIGYINPYNDGTKYAAP | LVKPG | | | | GLEW | | TKYA | | LYLQMN | | | | S | |
| | VKGRFTISRDDSKNTLYLQMN | GSLRL | | | | IG | | APVK | | SLKTED | | | | | |
| | SLKTEDTAVYYCTSNWDLDYW | SCAAS | | | | | | G | | TAVYYC | | | | | |
| | GQGTLVTVSS | GYTFT | | | | | | | | TS | | | | | |
| 23A0146 | EVQLVESGGGLVKPGGSLRLS | EVQLV | 210 | RYLIH | 114 | WVRQ | 218 | YINP | 120 | RFTISR | 223 | NWDLDY | 124 | WGQGT | 41 |
| | CAASGYTFTRYLIHWVRQAPG | ESGGG | | | | APGK | | YNDG | | DDSKNT | | | | LVTVS | |
| | KGLEWIGYINPYNDGTKYAAP | LVKPG | | | | GLEW | | TKYA | | LYLQMN | | | | S | |
| | VKGRFTISRDDSKNTLYLQMN | GSLRL | | | | IG | | APVK | | SLKTED | | | | | |
| | SLKTEDTAVYYCTSNWDLDYW | SCAAS | | | | | | G | | TAVYYC | | | | | |
| | GQGTLVTVSS | GYTFT | | | | | | | | TS | | | | | |
| 23A0147 | EVQLVESGGGLVKPGGSLRLS | EVQLV | 210 | RYLIH | 114 | WVRQ | 218 | YINP | 120 | RFTISR | 223 | NWDLDY | 124 | WGQGT | 41 |
| | CAASGYTFTRYLIHWVRQAPG | ESGGG | | | | APGK | | YNDG | | DDSKNT | | | | LVTVS | |
| | KGLEWIGYINPYNDGTKYAAP | LVKPG | | | | GLEW | | TKYA | | LYLQMN | | | | S | |
| | VKGRFTISRDDSKNTLYLQMN | GSLRL | | | | IG | | APVK | | SLKTED | | | | | |
| | SLKTEDTAVYYCTSNWDLDYW | SCAAS | | | | | | G | | TAVYYC | | | | | |
| | GQGTLVTVSS | GYTFT | | | | | | | | TS | | | | | |
| 23A0148 | EVQLVESGGGLVKPGGSLRLS | EVQLV | 210 | RYLIH | 114 | WVRQ | 218 | YINP | 120 | RFTISR | 223 | NWDLDY | 124 | WGQGT | 41 |
| | CAASGYTFTRYLIHWVRQAPG | ESGGG | | | | APGK | | YNDG | | DDSKNT | | | | LVTVS | |
| | KGLEWIGYINPYNDGTKYAAP | LVKPG | | | | GLEW | | TKYA | | LYLQMN | | | | S | |
| | VKGRFTISRDDSKNTLYLQMN | GSLRL | | | | IG | | APVK | | SLKTED | | | | | |
| | SLKTEDTAVYYCTSNWDLDYW | SCAAS | | | | | | G | | TAVYYC | | | | | |
| | GQGTLVTVSS | GYTFT | | | | | | | | TS | | | | | |
| 23A0149 | EVQLQQSGPELVKPGASVKVS | EVQLQ | 211 | RYLMH | 115 | WVRQ | 217 | YINP | 121 | RVTITA | 224 | NWDLDY | 124 | WGQGT | 41 |
| | CKASGYTFTRYLMHWVRQAPG | QSGPE | | | | APGQ | | YNDG | | DKSTST | | | | LVTVS | |
| | QGLEWIGYINPYNDGTKYNQK | LVKPG | | | | GLEW | | TKYN | | AYMELS | | | | S | |
| | FQGRVTITADKSTSTAYMELS | ASVKV | | | | IG | | QKFQ | | SLTSED | | | | | |
| | SLTSEDTAVYYCTSNWDLDYW | SCKAS | | | | | | G | | TAVYYC | | | | | |
| | GQGTLVTVSS | GYTFT | | | | | | | | TS | | | | | |
| 23A0150 | EVQLQQSGPEVKKPGASVKVS | EVQLQ | 212 | RYLIH | 114 | WVRQ | 217 | YINP | 121 | RVTITA | 225 | NWDLDY | 124 | WGQGT | 230 |
| | CKASGYTFTRYLIHWVRQAPG | QSGPE | | | | APGQ | | YNDG | | DKSTST | | | | LVTVS | |
| | QGLEWIGYINPYNDGTKYNQK | VKKPG | | | | GLEW | | TKYN | | AYMELS | | | | S | |
| | FQGRVTITADKSTSTAYMELS | ASVKV | | | | IG | | QKFQ | | SLRSED | | | | | |
| | SLRSEDTAVYYCTSNWDLDYW | SCKAS | | | | | | G | | TAVYYC | | | | | |
| | GQGTTLTVSS | GYTFT | | | | | | | | TS | | | | | |
| 23A0151 | QVQLQQSGSELKKPGASVKVS | QVQLQ | 213 | RYLMH | 115 | WVRQ | 218 | YINP | 121 | RVTITA | 225 | NWDLDY | 124 | WGQGT | 41 |
| | CKASGYTFTRYLMHWVRQAPG | QSGSE | | | | APGK | | YNDG | | DKSTST | | | | LVTVS | |
| | KGLEWIGYINPYNDGTKYNQK | LKKPG | | | | GLEW | | TKYN | | AYMELS | | | | S | |
| | FQGRVTITADKSTSTAYMELS | ASVKV | | | | IG | | QKFQ | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0152 | EIQLQQSGSELKKPGASVKVS | EIQLQ | 214 | RYLMN | 116 | WVRQ | 218 | YINP | 121 | RVTITA | 225 | NWDLDY | 124 | WGQGT | 41 |
| | CKASGYTFTRYLMNWVRQAPG | QSGSE | | | | APGK | | YNDG | | DKSTST | | | | LVTVS | |
| | KGLEWIGYINPYNDGTKYNQK | LKKPG | | | | GLEW | | TKYN | | AYMELS | | | | S | |
| | FQGRVTITADKSTSTAYMELS | ASVKV | | | | IG | | QKFQ | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0153 | EVQLQQSGPEVKKPGASVKVS | EVQLQ | 212 | RYLMH | 115 | WVRQ | 219 | YINP | 121 | RVTITA | 225 | NWDLDY | 124 | WGQGT | 230 |
| | CKASGYTFTRYLMHWVRQPPG | QSGPE | | | | PPGQ | | YNDG | | DKSTST | | | | LVTVS | |
| | QGLEWIGYINPYNDGTKYNQK | VKKPG | | | | GLEW | | TKYN | | AYMELS | | | | S | |
| | FQGRVTITADKSTSTAYMELS | ASVKV | | | | IG | | QKFQ | | SLRSED | | | | | |
| | SLRSEDTAVYYCTSNWDLDYW | SCKAS | | | | | | G | | TAVYYC | | | | | |
| | GQGTTLTVSS | GYTFT | | | | | | | | TS | | | | | |
| 23A0175 | QVQLVQSGAEVKKPGASVKVS | QVQLV | 215 | RYLIH | 114 | WVRQ | 220 | YINP | 118 | RVTMTR | 226 | NWDLDY | 124 | WGQGT | 41 |
| | CKASGYTFTRYLIHWVRQAPG | QSGAE | | | | APGQ | | YNDG | | DTSTST | | | | LVTVS | |
| | QGLEWMGYINPYNDGTKYNEK | VKKPG | | | | GLEW | | TKYN | | VYMELS | | | | S | |
| | FKGRVTMTRDTSTSTVYMELS | ASVKV | | | | MG | | EKFK | | SLRSED | | | | | |
| | SLRSEDTAVYYCASNWDLDYW | SCKAS | | | | | | G | | TAVYYC | | | | | |
| | GQGTLVTVSS | GYTFT | | | | | | | | AS | | | | | |
| 23A0176 | QVQLVQSGAEVKKPGASVKVS | QVQLV | 215 | RYLIH | 114 | WVRQ | 220 | YINP | 118 | RVTMTR | 226 | NWDLDY | 124 | WGQGT | 41 |
| | CKASGYTFTRYLIHWVRQAPG | QSGAE | | | | APGQ | | YNDG | | DTSTST | | | | LVTVS | |
| | QGLEWMGYINPYNDGTKYNEK | VKKPG | | | | GLEW | | TKYN | | VYMELS | | | | S | |
| | FKGRVTMTRDTSTSTVYMELS | ASVKV | | | | MG | | EKFK | | SLRSED | | | | | |
| | SLRSEDTAVYYCASNWDLDYW | SCKAS | | | | | | G | | TAVYYC | | | | | |
| | GQGTLVTVSS | GYTFT | | | | | | | | AS | | | | | |
| 23A0177 | QVQLVQSGAEVKKPGASVKVS | QVQLV | 215 | RYLIH | 114 | WVRQ | 220 | YINP | 118 | RVTMTR | 226 | NWDLDY | 124 | WGQGT | 41 |
| | CKASGYTFTRYLIHWVRQAPG | QSGAE | | | | APGQ | | YNDG | | DTSTST | | | | LVTVS | |
| | QGLEWMGYINPYNDGTKYNEK | VKKPG | | | | GLEW | | TKYN | | VYMELS | | | | S | |
| | FKGRVTMTRDTSTSTVYMELS | ASVKV | | | | MG | | EKFK | | SLRSED | | | | | |
| | SLRSEDTAVYYCASNWDLDYW | SCKAS | | | | | | G | | TAVYYC | | | | | |
| | GQGTLVTVSS | GYTFT | | | | | | | | AS | | | | | |
| 23A0178 | QVQLVQSGAEVKKPGASVKVS | QVQLV | 215 | RYLIH | 114 | WVRQ | 220 | YINP | 118 | RVTMTR | 226 | NWDLDY | 124 | WGQGT | 41 |
| | CKASGYTFTRYLIHWVRQAPG | QSGAE | | | | APGQ | | YNDG | | DTSTST | | | | LVTVS | |
| | QGLEWMGYINPYNDGTKYNEK | VKKPG | | | | GLEW | | TKYN | | VYMELS | | | | S | |
| | FKGRVTMTRDTSTSTVYMELS | ASVKV | | | | MG | | EKFK | | SLRSED | | | | | |
| | SLRSEDTAVYYCASNWDLDYW | SCKAS | | | | | | G | | TAVYYC | | | | | |
| | GQGTLVTVSS | GYTFT | | | | | | | | AS | | | | | |
| 23A0179 | QVQLVQSGAEVKKPGASVKVS | QVQLV | 215 | RYLIH | 114 | WVRQ | 217 | YINP | 118 | RVTMTS | 227 | NWDLDY | 124 | WGQGT | 41 |
| | CKASGYTFTRYLIHWVRQAPG | QSGAE | | | | APGQ | | YNDG | | DKSTST | | | | LVTVS | |
| | QGLEWIGYINPYNDGTKYNEK | VKKPG | | | | GLEW | | TKYN | | AYMELS | | | | S | |
| | FKGRVTMTSDKSTSTAYMELS | ASVKV | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0180 | QVQLVQSGAEVKKPGASVKVS | QVQLV | 215 | RYLIH | 114 | WVRQ | 217 | YINP | 118 | RVTMTS | 227 | NWDLDY | 124 | WGQGT | 41 |
| | CKASGYTFTRYLIHWVRQAPG | QSGAE | | | | APGQ | | YNDG | | DKSTST | | | | LVTVS | |
| | QGLEWIGYINPYNDGTKYNEK | VKKPG | | | | GLEW | | TKYN | | AYMELS | | | | S | |
| | FKGRVTMTSDKSTSTAYMELS | ASVKV | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0181 | QVQLVQSGAEVKKPGASVKVS | QVQLV | 215 | RYLIH | 114 | WVRQ | 217 | YINP | 118 | RVTMTS | 227 | NWDLDY | 124 | WGQGT | 41 |
| | CKASGYTFTRYLIHWVRQAPG | QSGAE | | | | APGQ | | YNDG | | DKSTST | | | | LVTVS | |
| | QGLEWIGYINPYNDGTKYNEK | VKKPG | | | | GLEW | | TKYN | | AYMELS | | | | S | |
| | FKGRVTMTSDKSTSTAYMELS | ASVKV | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0182 | QVQLVQSGAEVKKPGASVKVS | QVQLV | 215 | RYLIH | 114 | WVRQ | 217 | YINP | 118 | RVTMTS | 227 | NWDLDY | 124 | WGQGT | 41 |
| | CKASGYTFTRYLIHWVRQAPG | QSGAE | | | | APGQ | | YNDG | | DKSTST | | | | LVTVS | |
| | QGLEWIGYINPYNDGTKYNEK | VKKPG | | | | GLEW | | TKYN | | AYMELS | | | | S | |
| | FKGRVTMTSDKSTSTAYMELS | ASVKV | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0183 | QVQLVQSGAEVVKPGASVKMS | QVQLV | 216 | RYLIH | 114 | WVKQ | 221 | YINP | 118 | RATLTS | 228 | NWDLDY | 124 | WGQGT | 41 |
| | CKASGYTFTRYLIHWVKQAPG | QSGAE | | | | APGQ | | YNDG | | DKSTST | | | | LVTVS | |
| | QGLEWIGYINPYNDGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0184 | QVQLVQSGAEVVKPGASVKMS | QVQLV | 216 | RYLIH | 114 | WVKQ | 221 | YINP | 118 | RATLTS | 228 | NWDLDY | 124 | WGQGT | 41 |
| | CKASGYTFTRYLIHWVKQAPG | QSGAE | | | | APGQ | | YNDG | | DKSTST | | | | LVTVS | |
| | QGLEWIGYINPYNDGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0185 | QVQLVQSGAEVVKPGASVKMS | QVQLV | 216 | RYLIH | 114 | WVKQ | 221 | YINP | 118 | RATLTS | 228 | NWDLDY | 124 | WGQGT | 41 |
| | CKASGYTFTRYLIHWVKQAPG | QSGAE | | | | APGQ | | YNDG | | DKSTST | | | | LVTVS | |
| | QGLEWIGYINPYNDGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186 | QVQLVQSGAEVVKPGASVKMS | QVQLV | 216 | RYLIH | 114 | WVKQ | 221 | YINP | 118 | RATLTS | 228 | NWDLDY | 124 | WGQGT | 41 |
| | CKASGYTFTRYLIHWVKQAPG | QSGAE | | | | APGQ | | YNDG | | DKSTST | | | | LVTVS | |
| | QGLEWIGYINPYNDGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 122 | RATLTS | 228 | NWDLDY | 124 | WGQGT | 41 |
| 1 | CKASGYTFTRYLIHWVKQAPG | QSGAE | | | | APGQ | | YNDA | | DKSTST | | | | LVTVS | |
| | QGLEWIGYINPYNDATKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 123 | RATLTS | 228 | NWDLDY | 124 | WGQGT | 41 |
| 2 | CKASGYTFTRYLIHWVKQAPG | QSGAE | | | | APGQ | | YNEG | | DKSTST | | | | LVTVS | |
| | QGLEWIGYINPYNEGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 122 | RATLTS | 228 | NWDLDY | 124 | WGQGT | 41 |
| 3 | CKASGYTFTRYLIHWVKQAPG | QSGAE | | | | APGQ | | YNDA | | DKSTST | | | | LVTVS | |
| | QGLEWIGYINPYNDATKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 123 | RATLTS | 228 | NWDLDY | 124 | WGQGT | 41 |
| 4 | CKASGYTFTRYLIHWVKQAPG | QSGAE | | | | APGQ | | YNEG | | DKSTST | | | | LVTVS | |
| | QGLEWIGYINPYNEGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 118 | RATLTS | 228 | NWDLDY | 124 | WGQGT | 41 |
| 5 | CKASGYTFTRYLIHWVKQAPG | QSGAE | | | | APGQ | | YNDG | | DKSTST | | | | LVTVS | |
| | QGLEWIGYINPYNDGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |
| 23A0186- | QVQLVQSGAEVVKPGASVKMS | QVQLV | 216 | RYLIH | 114 | WVKQ | 221 | YINP | 123 | RATLTS | 228 | NWDLDY | 124 | WGQGT | 41 |
| 6 | CKASGYTFTRYLIHWVKQAPG | QSGAE | | | | APGQ | | YNEG | | DKSTST | | | | LVTVS | |
| | QGLEWIGYINPYNEGTKYNEK | VVKPG | | | | GLEW | | TKYN | | AYMELS | | | | S | |
| | FKGRATLTSDKSTSTAYMELS | ASVKM | | | | IG | | EKFK | | SLRSED | | | | | |
| | SLRSEDTAVYYCTSNWDLDYW | SCKAS | | | | | | G | | TAVYYC | | | | | |
| | GQGTLVTVSS | GYTFT | | | | | | | | TS | | | | | |

TABLE D

| | | | SEQ ID | | SEQ ID | | SEQ ID | | SEQ ID | | SEQ ID | | SEQ ID | | SEQ ID |
|---|---|---|---|---|---|---|---|---|---|---|---|---|---|---|---|
| antibody | VL | LFR1 | NO | LCDR1 | NO | LFR2 | NO | LCDR2 | NO | LFR3 | NO | LCDR3 | NO | LFR4 | NO |
| 16003-18E5 | DIVMTQSPATLSVTPGDRVSLS | | | RASQ | 125 | | | FASQ | 126 | | | NGHS | 128 | | |
| | CRASQSISDYLYWYQQKSHESP | | | SISD | | | | SIS | | | | FPFT | | | |
| | RLLIKFASQSISGIPSRFTGSG | | | YLY | | | | | | | | | | | |
| | SGSDFTLSIDSVEPDDVGVFFC | | | | | | | | | | | | | | |
| | QNGHSFPFTFGGGTKLEIK | | | | | | | | | | | | | | |
| 23A0144 | AIQMTQSPSSLSASVGDRVTIT | AIQM | 231 | RASQ | 125 | WYQQ | 241 | FASQ | 126 | GVPSRFS | 248 | NGHS | 128 | FGPG | 259 |
| | CRASQSISDYLYWYQQKPGKAP | TQSP | | SISD | | KPGK | | SIS | | GSGSGTD | | FPFT | | TKVD | |
| | KLLIKFASQSISGVPSRFSGSG | SSLS | | YLY | | APKL | | | | FTLTISS | | | | IK | |
| | SGTDFTLTISSLQPEDFATYYC | ASVG | | | | LIK | | | | LQPEDFA | | | | | |
| | QNGHSFPFTFGPGTKVDIK | DRVT | | | | | | | | TYYCQ | | | | | |
| | | ITC | | | | | | | | | | | | | |
| 23A0145 | DIQMTQSPSSLSASVGDRVTIT | DIQM | 47 | RASQ | 125 | WFQQ | 242 | FASQ | 126 | GVPSRFS | 248 | NGHS | 128 | FGQG | 260 |
| | CRASQSISDYLYWFQQKPGKAP | TQSP | | SISD | | KPGK | | SIS | | GSGSGTD | | FPFT | | TRLE | |
| | KLLIKFASQSISGVPSRFSGSG | SSLS | | YLY | | APKL | | | | FTLTISS | | | | IK | |
| | SGTDFTLTISSLQPEDFATYYC | ASVG | | | | LIK | | | | LQPEDFA | | | | | |
| | QNGHSFPFTFGQGTRLEIK | DRVT | | | | | | | | TYYCQ | | | | | |
| | | ITC | | | | | | | | | | | | | |
| 23A0146 | NIQMTQSPSAMSASVGDRVTIT | NIQM | 233 | RASQ | 125 | WFQQ | 243 | FASQ | 126 | GVPSRFS | 249 | NGHS | 128 | FGPG | 259 |
| | CRASQSISDYLYWFQQKPGKVP | TQSP | | SISD | | KPGK | | SIS | | GSGSGTE | | FPFT | | TKVD | |
| | KLLIKFASQSISGVPSRFSGSG | SAMS | | YLY | | VPKL | | | | FTLTISS | | | | IK | |
| | SGTEFTLTISSLQPEDFATYYC | ASVG | | | | LIK | | | | LQPEDFA | | | | | |
| | QNGHSFPFTFGPGTKVDIK | DRVT | | | | | | | | TYYCQ | | | | | |
| | | ITC | | | | | | | | | | | | | |
| 23A0147 | DIQMTQSPSTLSASVGDRVTIT | DIQM | 234 | RASQ | 125 | WYQQ | 241 | FASQ | 126 | GVPSRFS | 250 | NGHS | 128 | FGPG | 259 |
| | CRASQSISDYLYWYQQKPGKAP | TQSP | | SISD | | KPGK | | SIS | | GSGSGTE | | FPFT | | TKVD | |
| | KLLIKFASQSISGVPSRFSGSG | STLS | | YLY | | APKL | | | | FTLTISS | | | | IK | |
| | SGTEFTLTISSLQPDDFATYYC | ASVG | | | | LIK | | | | LQPDDFA | | | | | |
| | QNGHSFPFTFGPGTKVDIK | DRVT | | | | | | | | TYYCQ | | | | | |
| | | ITC | | | | | | | | | | | | | |
| 23A0148 | AIQMTQSPSSLSASVGDRVTIT | AIQM | 231 | RASQ | 125 | WYQQ | 241 | FASQ | 126 | GVPSRFS | 248 | NGHS | 128 | FGQG | 261 |
| | CRASQSISDYLYWYQQKPGKAP | TQSP | | SISD | | KPGK | | SIS | | GSGSGTD | | FPFT | | TKVE | |
| | KLLIKFASQSISGVPSRFSGSG | SSLS | | YLY | | APKL | | | | FTLTISS | | | | IK | |
| | SGTDFTLTISSLQPEDFATYYC | ASVG | | | | LIK | | | | LQPEDFA | | | | | |
| | QNGHSFPFTFGQGTKVEIK | DRVT | | | | | | | | TYYCQ | | | | | |
| | | ITC | | | | | | | | | | | | | |
| 23A0149 | AIVMTQSPATLSVSPGERVTLS | AIVM | 235 | RASQ | 125 | WYQQ | 244 | FASQ | 126 | GVPSRFS | 251 | NGHS | 128 | FGGG | 262 |
| | CRASQSISDYLYWYQQKPDQSP | TQSP | | SISD | | KPDQ | | SIS | | GSGSGRD | | FPFT | | TKLE | |
| | KLLIKFASQSISGVPSRFSGSG | ATLS | | YLY | | SPKL | | | | FTLTISS | | | | IK | |
| | SGRDFTLTISSLQPEDFAVYYC | VSPG | | | | LIK | | | | LQPEDFA | | | | | |
| | QNGHSFPFTFGGGTKLEIK | ERVT | | | | | | | | VYYCQ | | | | | |
| | | LSC | | | | | | | | | | | | | |
| 23A0150 | NIVMTQSPATLSVSPGERATLS | NIVM | 236 | RASQ | 125 | WYQQ | 244 | FASQ | 126 | GVPSRFS | 252 | NGHS | 128 | FGGG | 262 |
| | CRASQSISDYLYWYQQKPDQSP | TQSP | | SISD | | KPDQ | | SIS | | GSGSGRD | | FPFT | | TKLE | |
| | KLLIKFASQSISGVPSRFSGSG | ATLS | | YLY | | SPKL | | | | FTLTISS | | | | IK | |
| | SGRDFTLTISSLEPEDFAVYYC | VSPG | | | | LIK | | | | LEPEDFA | | | | | |
| | QNGHSFPFTFGGGTKLEIK | ERAT | | | | | | | | VYYCQ | | | | | |
| | | LSC | | | | | | | | | | | | | |
| 23A0151 | EIVMTQSPATLSLSPGERATLS | EIVM | 237 | RASQ | 125 | WYQQ | 244 | FASQ | 126 | GVPSRFS | 253 | NGHS | 128 | FGGG | 263 |
| | CRASQSISDYLYWYQQKPDQSP | TQSP | | SISD | | KPDQ | | SIS | | GSGSGTE | | FPFT | | TKVE | |
| | KLLIKFASQSISGVPSRFSGSG | ATLS | | YLY | | SPKL | | | | FTLTISS | | | | IK | |
| | SGTEFTLTISSLQPEDFAVYYC | LSPG | | | | LIK | | | | LQPEDFA | | | | | |
| | QNGHSFPFTFGGGTKVEIK | ERAT | | | | | | | | VYYCQ | | | | | |
| | | LSC | | | | | | | | | | | | | |
| 23A0152 | DIVMTQSPATLSVSPGERVTLS | DIVM | 238 | RASQ | 125 | WYQQ | 244 | FASQ | 126 | GVPSRFS | 254 | NGHS | 128 | FGGG | 262 |
| | CRASQSISDYLYWYQQKPDQSP | TQSP | | SISD | | KPDQ | | SIS | | GSGSGSD | | FPFT | | TKLE | |
| | KLLIKFASQSISGVPSRFSGSG | ATLS | | YLY | | SPKL | | | | FTLTISS | | | | IK | |
| | SGSDFTLTISSLQPEDFATYYC | VSPG | | | | LIK | | | | LQPEDFA | | | | | |
| | QNGHSFPFTFGGGTKLEIK | ERVT | | | | | | | | TYYCQ | | | | | |
| | | LSC | | | | | | | | | | | | | |
| 23A0153 | DIVMTQSPATLSVSPGERVTLS | DIVM | 238 | RASQ | 125 | WYQQ | 245 | FASQ | 126 | GVPSRFS | 255 | NGHS | 128 | FGGG | 263 |
| | CRASQSISDYLYWYQQKPDQSP | TQSP | | SISD | | KPDQ | | SIS | | GSGSGSD | | FPFT | | TKVE | |
| | RLLIKFASQSISGVPSRFSGSG | ATLS | | YLY | | SPKL | | | | FTLKISS | | | | IK | |
| | SGSDFTLKISSVEAEDVGVYYC | VSPG | | | | LIK | | | | VEAEDVG | | | | | |
| | QNGHSFPFTFGGGTKVEIK | ERVT | | | | | | | | VYYCQ | | | | | |
| | | LSC | | | | | | | | | | | | | |
| 23A0175 | DIQMTQSPSSLSASVGDRVTIT | DIQM | 47 | RASQ | 125 | WYQQ | 52 | FASQ | 126 | GVPSRFS | 248 | NGHS | 128 | FGGG | 263 |
| | CRASQSISDYLYWYQQKPGKAP | TQSP | | SISD | | KPGK | | SIS | | GSGSGTD | | FPFT | | TKVE | |
| | KLLIYFASQSISGVPSRFSGSG | SSLS | | YLY | | APKL | | | | FTLTISS | | | | IK | |
| | SGTDFTLTISSLQPEDFATYYC | ASVG | | | | LIY | | | | LQPEDFA | | | | | |
| | QNGHSFPFTFGGGTKVEIK | DRVT | | | | | | | | TYYCQ | | | | | |
| | | ITC | | | | | | | | | | | | | |
| 23A0176 | DIQMTQSPSSLSASVGDRVTIT | DIQM | 47 | RASQ | 125 | WYQQ | 241 | FASQ | 126 | GIPSRFS | 256 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSLQPEDFATFYC | ASVG | | | | LIK | | | | LQPEDFA | | | | | |
| | QNGHSFPFTFGGGTKVEIK | DRVT | | | | | | | | TFYCQ | | | | | |
| | | ITC | | | | | | | | | | | | | |
| 23A0177 | DIVMTQSPSSLSASVGDRVTLT | DIVM | 239 | RASQ | 125 | WYQQ | 241 | FASQ | 126 | GIPSRFS | 257 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSVQPEDFATFFC | ASVG | | | | LIK | | | | VQPEDFA | | | | | |
| | QNGHSFPFTFGGGTKVEIK | DRVT | | | | | | | | TFFCQ | | | | | |
| | | LTC | | | | | | | | | | | | | |
| 23A0178 | EIVMTQSPATLSVSPGERVTLS | EIVM | 240 | RASQ | 125 | WYQQ | 247 | FASQ | 126 | GIPARFS | 258 | NGHS | 128 | FGGG | 263 |
| | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNGHSFPFTFGGGTKVEIK | ERVT | | | | | | | | VFFCQ | | | | | |
| | | LSC | | | | | | | | | | | | | |
| 23A0179 | DIQMTQSPSSLSASVGDRVTIT | DIQM | 47 | RASQ | 125 | WYQQ | 52 | FASQ | 126 | GVPSRFS | 248 | NGHS | 128 | FGGG | 263 |
| | CRASQSISDYLYWYQQKPGKAP | TQSP | | SISD | | KPGK | | SIS | | GSGSGTD | | FPFT | | TKVE | |
| | KLLIYFASQSISGVPSRFSGSG | SSLS | | YLY | | APKL | | | | FTLTISS | | | | IK | |
| | SGTDFTLTISSLQPEDFATYYC | ASVG | | | | LIY | | | | LQPEDFA | | | | | |
| | QNGHSFPFTFGGGTKVEIK | DRVT | | | | | | | | TYYCQ | | | | | |
| | | ITC | | | | | | | | | | | | | |
| 23A0180 | DIQMTQSPSSLSASVGDRVTIT | DIQM | 47 | RASQ | 125 | WYQQ | 241 | FASQ | 126 | GIPSRFS | 256 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSLQPEDFATFYC | ASVG | | | | LIK | | | | LQPEDFA | | | | | |
| | QNGHSFPFTFGGGTKVEIK | DRVT | | | | | | | | TFYCQ | | | | | |
| | | ITC | | | | | | | | | | | | | |
| 23A0181 | DIVMTQSPSSLSASVGDRVTLT | DIVM | 239 | RASQ | 125 | WYQQ | 241 | FASQ | 126 | GIPSRES | 257 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSVQPEDFATFFC | ASVG | | | | LIK | | | | VQPEDFA | | | | | |
| | QNGHSFPFTFGGGTKVEIK | DRVT | | | | | | | | TFFCQ | | | | | |
| | | LTC | | | | | | | | | | | | | |
| 23A0182 | EIVMTQSPATLSVSPGERVTLS | EIVM | 240 | RASQ | 125 | WYQQ | 247 | FASQ | 126 | GIPARFS | 258 | NGHS | 128 | FGGG | 263 |
| | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNGHSFPFTFGGGTKVEIK | ERVT | | | | | | | | VFFCQ | | | | | |
| | | LSC | | | | | | | | | | | | | |
| 23A0183 | DIQMTQSPSSLSASVGDRVTIT | DIQM | 47 | RASQ | 125 | WYQQ | 52 | FASQ | 126 | GVPSRFS | 248 | NGHS | 128 | FGGG | 263 |
| | CRASQSISDYLYWYQQKPGKAP | TQSP | | SISD | | KPGK | | SIS | | GSGSGTD | | FPFT | | TKVE | |
| | KLLIYFASQSISGVPSRFSGSG | SSLS | | YLY | | APKL | | | | FTLTISS | | | | IK | |
| | SGTDFTLTISSLQPEDFATYYC | ASVG | | | | LIY | | | | LQPEDFA | | | | | |
| | QNGHSFPFTFGGGTKVEIK | DRVT | | | | | | | | TYYCQ | | | | | |
| | | ITC | | | | | | | | | | | | | |
| 23A0184 | DIQMTQSPSSLSASVGDRVTIT | DIQM | 47 | RASQ | 125 | WYQQ | 241 | FASQ | 126 | GIPSRFS | 256 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSLQPEDFATFYC | ASVG | | | | LIK | | | | LQPEDFA | | | | | |
| | QNGHSFPFTFGGGTKVEIK | DRVT | | | | | | | | TFYCQ | | | | | |
| | | ITC | | | | | | | | | | | | | |
| 23A0185 | DIVMTQSPSSLSASVGDRVTLT | DIVM | 239 | RASQ | 125 | WYQQ | 241 | FASQ | 126 | GIPSRES | 257 | NGHS | 128 | FGGG | 263 |
| | CRASQSISDYLYWYQQKPGKAP | TQSP | | SISD | | KPGK | | SIS | | GSGSGSD | | FPFT | | TKVE | |
| | KLLIKFASQSISGIPSRFSGSG | SSLS | | YLY | | APKL | | | | FTLTISS | | | | IK | |
| | SGSDFTLTISSVQPEDFATFFC | ASVG | | | | LIK | | | | VQPEDFA | | | | | |
| | QNGHSFPFTFGGGTKVEIK | DRVT | | | | | | | | TFFCQ | | | | | |
| | | LTC | | | | | | | | | | | | | |
| 23A0186 | EIVMTQSPATLSVSPGERVTLS | EIVM | 240 | RASQ | 125 | WYQQ | 247 | FASQ | 126 | GIPARFS | 258 | NGHS | 128 | FGGG | 263 |
| | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNGHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | QGHS | 129 | FGGG | 263 |
| 1 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QQGHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | QGHS | 129 | FGGG | 263 |
| 2 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QQGHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | NAHS | 130 | FGGG | 263 |
| 3 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNAHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | NAHS | 130 | FGGG | 263 |
| 4 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNAHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | QGHS | 129 | FGGG | 263 |
| 5 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QQGHSFPFTFGGGTKVEIK | ERVT | | | | | | | | VFFCQ | | | | | |
| | | LSC | | | | | | | | | | | | | |
| 23A0186- | EIVMTQSPATLSVSPGERVTLS | EIVM | 240 | RASQ | 125 | WYQQ | 247 | FASQ | 126 | GIPARFS | 258 | NGHS | 128 | FGGG | 263 |
| 6 | CRASQSISDYLYWYQQKPGQAP | TQSP | | SISD | | KPGQ | | SIS | | GSGSGSE | | FPFT | | TKVE | |
| | RLLIKFASQSISGIPARFSGSG | ATLS | | YLY | | APRL | | | | FTLTISS | | | | IK | |
| | SGSEFTLTISSVQSEDFAVFFC | VSPG | | | | LIK | | | | VQSEDFA | | | | | |
| | QNGHSFPFTFGGGTKVEIK | ERVT | | | | | | | | VFFCQ | | | | | |
| | | LSC | | | | | | | | | | | | | |

In some embodiments, the VH of the second binding domain includes HCDR1 as shown in SEQ ID NO: 264, HCDR2 as shown in SEQ ID NO: 265, HCDR3 as shown in SEQ ID NO: 266, and the VL of the second binding domain includes LCDR1 as shown in SEQ ID NO: 267, LCDR2 as shown in SEQ ID NO: 268, LCDR3 as shown in SEQ ID NO: 269.

In some embodiments, the VH of the second binding domain is the sequence shown in SEQ ID NO: 148, and the VL of the second binding domain is the sequence shown in SEQ ID NO: 166.

### Antigen-Binding Protein

In one aspect, the present application provides an isolated antigen-binding protein that can be assayed in a Biacore assay with a KD value of about 5E-08M or less (e.g., the KD is no greater than about 5E-08M, no greater than about 4.5E-08M, no greater than about 4E-08M, no greater than about 3.5E-08M, no greater than about 3E-08M, no greater than about 2.5E-08M, no greater than about 2E-08M, no greater than about 1.5E-08M, no greater than about 1E-08M, no greater than about 5E-09M, no greater than about 4.5E-09M, not higher than about 4E-09M, not higher than about 3.5E-09M, not higher than about 3E-09M, not higher than about 2.5E-09M, not higher than about 2E-09M, not higher than about 1.5E-09M, not higher than about 1E-09M, not higher than about 9E-10M, not higher than about 5E-10M, not higher than about 1E-10M, not higher than about 5E-11M, not higher than about 1E-11M or not higher than 5E-12M or less) specifically binds to monomeric TL1A and/or trimeric TL1A. The monomeric TL1A and/or trimeric TL1A are human monomeric TL1A and/or human trimeric TL1A In the present application, the isolated antigen binding protein is an antagonist of TL1A receptors, including but not limited to DR3 and TR6/DcR3. In the present application, the isolated antigen binding protein blocks the interaction between TL1A and death receptor 3 (“DR3”). In the present application, the isolated antigen binding protein can block the binding of monomeric TL1A-protein and/or trimeric TL1A to DR3 (DR3: UniprotKB NO. Q93038) with an IC50 value of about 50 nM or less (e.g., the IC50 value is not higher than about 50 nM, not higher than about 45 nM, not higher than about 40 nM, not higher than about 35 nM, not higher than about 30 nM, not higher than about 25 nM or less) in a flow cytometry assay.

In the present application, the isolated antigen-binding proteins can inhibit the activity of the soluble TL1A-activated NF-κB signaling pathway. For example, the soluble TL1A-activated NF-κB signaling pathway can be tested for inhibition by a luciferase reporter gene system.

In the present application, said isolated antigen-binding protein can inhibit the activity of the membrane-bound TL1A-activated NF-κB signaling pathway. For example, whether the membrane-bound TL1A-activated NF-κB signaling pathway is inhibited can be detected by a luciferase reporter gene system.

In the present application, said isolated antigen-binding protein may function to inhibit Caspase3/7 activity. For example, the function of the Caspase3/7 activity can be tested for inhibition by a luciferase reporter gene system.

In the present application, said isolated antigen-binding protein blocks MAdCAM-1 mediated cell adhesion. The blocking ability can be determined, for example, by comparing curves of different antibody concentrations blocking MAdCAM-1 mediated cell adhesion to Hut 78.

In another aspect, the isolated antigen-binding protein of the present invention has all of the following properties.

It can specifically bind to IL-23 with a KD value of about 5E-08M or less (e.g., the KD is not higher than about 5E-08M, not higher than about 4.5E-08M, not higher than about 4E-08M, not higher than about 3.5E-08M, not higher than about 3E-08M, not higher than about 2.5E-08M, not higher than about 2E-08M, not higher than about 1.5E-08M, not higher than about 1E-08M, not higher than about 5E-09M, not higher than about 4.5E-09M, not higher than about 4E-09M, not higher than about 3.5E-09M, not higher than about 3E-09M, not higher than about 2.5E-09M, not higher than about 2E-09M, not higher than about 1.5E-09M, not higher than about 1E-09M, not higher than about 9E-10M, not higher than about 5E-10M, not higher than about 1E-10M, not higher than about 5E-11M, not higher than about 1E-11M or not higher than 5E-12M or less).

In the present application, the isolated antigen binding protein inhibits the production of IL-17 and IL-22 induced by IL-23 in mouse spleen cells.

In some aspects, the humanized antibody exhibits blocking activity, whereby it reduces the binding of IL-23 to the IL-23 receptor by at least 45%, at least 50%, at least 55%, at least 60%, at least 65%, at least 70%, at least 75%, at least 80%, at least 85%, at least 90%, or at least 95%. The ability of the antibody to block the binding of IL-23 to the IL-23 receptor can be measured using competitive binding assays known in the art. Alternatively, the blocking activity of the antibody can be measured by assessing the biological effects of IL-23 (e.g., the production of IL-17 and IL-22) to determine whether signaling mediated by the IL-23 receptor is inhibited.

### The Structure of Bispecific Antibody

In the present application, the antigen binding protein includes Fab, Fab′, Fv fragment, F(ab)′2, scFv, di-scFv and/or dAb, Fab, Fab′, F(ab′)2, Fd and Fv fragment, disulfide bond stabilized Fv fragment (dsFv), (dsFv)2, bispecific dsFv (dsFv-dsFv′), disulfide bond stabilized double chain antibody (diabody) (ds double chain antibody), single chain antibody molecule (scFv), single domain antibody (sdab) scFv dimer (bivalent double chain antibody), multispecific antibody formed from a part of an antibody comprising one or more CDRs, camelized single domain antibody, nanobody, domain antibody, domain antibody consisting of one VH domain or one VL domain (domain antibody, dAb), shark variable IgNAR domain, camelized VH domain, VHH domain, minimal recognition unit composed of CDRs mimicking antibodies (such as FR3-CDR3-FR4 portion, HCDR1, HCDR2, and/or HCDR3, and LCDR1, LCDR2, and/or LCDR3), alternative scaffolds for binding to antigens, bivalent domain antibodies, multispecific proteins (including antigen binding fragments or any other antibody fragments that bind to antigens, but not including complete antibody structures).

In the present application, the antigen binding protein includes scFv of the first antigen binding domain or scFv of the second binding domain.

In the present application, the scFv of the first antigen binding domain includes VL and VH of the first antigen binding domain.

In the present application, the C-terminus of the VL of the first antigen binding domain is directly or through a linker connected to the N-terminus of the VH of the first antigen binding domain, or the N-terminus of the VL of the first antigen binding domain is directly or through a linker connected to the C-terminus of the VH of the first antigen binding domain.

In the present application, the scFv of the first antigen binding domain includes a sequence as described in any one of SEQ ID NOs: 131-134.

In the present application, the scFv of the second binding domain includes VL and VH of the second binding domain.

In the present application, in the second binding domain, the C-terminus of the VL of the second binding domain is directly or through a linker connected to the N-terminus of the VH of the second binding domain, or the N-terminus of the VL of the second binding domain is directly or through a linker connected to the C-terminus of the VH of the second binding domain.

In some embodiments, the first binding domain comprises an scFv comprising a sequence as described in any one of SEQ ID NOs: 131-134 (e.g., the scFv of the first binding domain is SEQ ID NO: 131, SEQ ID NO: 132, SEQ ID NO: 133, or SEQ ID NO: 134), and the second antigen binding domain is a Fab fragment, wherein the VH and VL of the second antigen binding domain are selected from the following combination:
VH as set forth in SEQ ID NO:135 and VL as set forth in SEQ ID NO: 149;
VH as set forth in SEQ ID NO:136 and VL as set forth in SEQ ID NO:150;
VH as set forth in SEQ ID NO:137 and VL as set forth in SEQ ID NO:151;
VH as set forth in SEQ ID NO:137 and VL as set forth in SEQ ID NO:152;
VH as set forth in SEQ ID NO:137 and VL as set forth in SEQ ID NO:153;
VH as set forth in SEQ ID NO:137 and VL as set forth in SEQ ID NO:154;
VH as set forth in SEQ ID NO:138 and VL as set forth in SEQ ID NO:155;
VH as set forth in SEQ ID NO: 139 and VL as set forth in SEQ ID NO: 156;
VH as set forth in SEQ ID NO:140 and VL as set forth in SEQ ID NO:157;
VH as set forth in SEQ ID NO:141 and VL as set forth in SEQ ID NO:158;
VH as set forth in SEQ ID NO:142 and VL as set forth in SEQ ID NO:159;
VH as set forth in SEQ ID NO:143 and VL as set forth in SEQ ID NO:160;
VH as set forth in SEQ ID NO:143 and VL as set forth in SEQ ID NO:161;
VH as set forth in SEQ ID NO:143 and VL as set forth in SEQ ID NO:162;
VH as set forth in SEQ ID NO:143 and VL as set forth in SEQ ID NO:165;
VH as set forth in SEQ ID NO:144 and VL as set forth in SEQ ID NO:160;
VH as set forth in SEQ ID NO:144 and VL as set forth in SEQ ID NO:161;
VH as set forth in SEQ ID NO:144 and VL as set forth in SEQ ID NO:162;
VH as set forth in SEQ ID NO:144 and VL as set forth in SEQ ID NO:165;
VH as set forth in SEQ ID NO:146 and VL as set forth in SEQ ID NO:160;
VH as set forth in SEQ ID NO:146 and VL as set forth in SEQ ID NO:161;
VH as set forth in SEQ ID NO:146 and VL as set forth in SEQ ID NO:162;
VH as set forth in SEQ ID NO:146 and VL as set forth in SEQ ID NO:165;
VH as set forth in SEQ ID NO:145 and VL as set forth in SEQ ID NO:164;
VH as set forth in SEQ ID NO:147 and VL as set forth in SEQ ID NO:164;
VH as set forth in SEQ ID NO:145 and VL as set forth in SEQ ID NO:163;
VH as set forth in SEQ ID NO:147 and VL as set forth in SEQ ID NO:163;
VH as set forth in SEQ ID NO:146 and VL as set forth in SEQ ID NO:164;
VH as set forth in SEQ ID NO:147 and VL as set forth in SEQ ID NO:165;
VH as set forth in SEQ ID NO:148 and VL as set forth in SEQ ID NO:166.

In the present application, the isolated antigen-binding protein further comprises the heavy chain constant region of IgG and/or the light chain constant region of human antibody.

In the present application, the isolated antigen-binding protein comprises the heavy chain constant region of IgG1, IgG2, IgG3 or IgG4.

In the present application, the separated antigen-binding protein comprises a Kappa or Lambda light chain constant region.

In the present application, the separated antigen-binding protein is IgG, IgM, IgA, IgD or IgE., In the present application, the antigen-binding protein comprises one or more first heavy chains and one or more first light chains.

The first heavy chain comprises the VH of the first antigen-binding domain and/or the VH of the second binding domain and/or the scFv of the first antigen-binding domain and/or the scFv of the second binding domain; The first light chain comprises the VL of the first antigen-binding domain or the VL of the second binding domain.

In the present application, wherein, the first heavy chain comprises the VH of the first antigen-binding domain and the scFv of the second binding domain, or the first heavy chain comprises the VH of the second binding domain and the scFv of the first antigen-binding domain.

In the present application, the N-terminus of the scFv of the first antigen binding domain or the second binding domain is directly or through a linker connected to the C-terminus of the first heavy chain, or the C-terminus of the scFv of the first antigen binding domain or the second binding domain is directly or through a linker connected to the N-terminus of the first heavy chain.

In some embodiments, the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and the second antigen-binding domain comprises HCDR1 as set forth in SEQ ID NO: 264, HCDR2 as set forth in SEQ ID NO: 265, HCDR3 as set forth in SEQ ID NO: 266, LCDR1 as set forth in SEQ ID NO: 267, LCDR2 as set forth in SEQ ID NO: 268, and LCDR3 as set forth in SEQ ID NO: 269.

In some embodiments, the first antigen-binding domain comprises a VH as set forth in SEQ ID NO: 70 and a VL as set forth in SEQ ID NO: 106; and the second antigen-binding domain comprises a VH as set forth in SEQ ID NO: 148 and a VL as set forth in SEQ ID NO: 166.

In some embodiments, the first antigen-binding domain comprises an scFv as set forth in SEQ ID NO: 133; and the second antigen-binding domain comprises the VH as set forth in SEQ ID NO: 148 and the VL as set forth in SEQ ID NO: 166.

In some embodiments, the isolated antigen-binding protein includes a light chain as shown in SEQ ID NO: 182 and heavy chain as shown in SEQ ID NO: 207, or includes a light chain as shown in SEQ ID NO: 182 and heavy chain as shown in SEQ ID NO: 208.

In the present application, the isolated antigen-binding protein is selected from one of the following situations:
(1) the C-terminus of the first antigen-binding domain VH is directly or through a linker connected to the N-terminus of the first heavy chain, and the N-terminus of the scFv of the second binding domain is directly or through a linker connected to the C-terminus of the first heavy chain; the C-terminus of the VL of the first antigen-binding domain is connected to the N-terminus of the constant region of the light chain;
(2) the C-terminus of the VH of the second binding domain is directly or through a linker connected to the N-terminus of the first heavy chain, and the N-terminus of the scFv of the first antigen-binding domain is directly or through a linker connected to the C-terminus of the first heavy chain; the C-terminus of the VL of the second binding domain is connected to the N-terminus of the constant region of the light chain;
(3) the C-terminus of the scFv of the first antigen-binding domain is directly or through a linker connected to the N-terminus of the VH of the second binding domain, and the C-terminus of the VL of the second binding domain is connected to the N-terminus of the constant region of the light chain;
(4) the C-terminus of the scFv of the second binding domain is directly or through a linker connected to the N-terminus of the first antigen binding domain VH, and the C-terminus of the VL of the first antigen binding domain is connected to the N-terminus of the light chain constant region.

The first heavy chain is selected from the sequence described in any one of SEQ ID NO: 199-208.

The antigen binding protein is selected from the following combination:
1) a light chain as shown in SEQ ID NO: 179 and a heavy chain as shown in SEQ ID NO: 199;
2) a light chain as shown in SEQ ID NO: 179 and a heavy chain as shown in SEQ ID NO: 200;
3) a light chain as shown in SEQ ID NO: 179 and a heavy chain as shown in SEQ ID NO: 201;
4) a light chain as shown in SEQ ID NO: 179 and a heavy chain as shown in SEQ ID NO: 202;
5) a light chain as shown in SEQ ID NO: 180 and a heavy chain as shown in SEQ ID NO: 203;
6) a light chain as shown in SEQ ID NO: 181 and a heavy chain as shown in SEQ ID NO: 204;
7) a light chain as shown in SEQ ID NO: 182 and a heavy chain as shown in SEQ ID NO: 205;
8) a light chain as shown in SEQ ID NO: 182 and a heavy chain as shown in SEQ ID NO: 206;
9) a light chain as shown in SEQ ID NO: 182 and a heavy chain as shown in SEQ ID NO: 207;
10) a light chain shown in SEQ ID NO: 182 and the heavy chain shown in SEQ ID NO: 208.

In some embodiments, the isolated antigen-binding protein includes a light chain as shown in SEQ ID NO: 182 and heavy chain as shown in SEQ ID NO: 207, or includes a light chain as shown in SEQ ID NO: 182 and heavy chain as shown in SEQ ID NO: 208.

In some embodiments, the antigen binding protein described in the present application has one, two, three or four groups of the above light chains and heavy chains.

In the present application, the antigen binding protein comprises one or more first light chains, one or more third heavy chains, and one or more fourth heavy chains, wherein:
1) the third heavy chain comprises the VH of the first antigen binding domain or the second binding domain, and the fourth heavy chain comprises the scFv of the first or second binding domain, or;
2) the third heavy chain comprises the scFv of the first antigen binding domain or the second binding domain, and the fourth heavy chain comprises the VH of the first or second binding domain.

In the present application, the N-terminus of the VH of the first antigen binding domain or the second binding domain is directly or through a linker connected to the C-terminus of the third heavy chain, or the N-terminus of the scFv of the first or second binding domain is directly or through a linker connected to the C-terminus of the fourth heavy chain.

In the present application, wherein
1) the third heavy chain includes the VH of the first antigen binding domain, and the fourth heavy chain includes the scFv of the second binding domain, or;
2) the third heavy chain includes the scFv of the first antigen binding domain, and the fourth heavy chain includes the VH of the second binding domain, or;
3) the third heavy chain includes the VH of the second binding domain, and the fourth heavy chain includes the scFv of the first antigen binding domain, or 4) the third heavy chain includes the scFv of the second binding domain, and the fourth heavy chain includes the VH of the first antigen binding domain.

In the present application, the antigen binding protein is selected from the following:
1) a light chain as shown in SEQ ID NO: 175, a third heavy chain as shown in SEQ ID NO: 183, and a fourth heavy chain as shown in SEQ ID NO: 191;
2) a light chain as shown in SEQ ID NO: 176, a third heavy chain as shown in SEQ ID NO: 184, and a fourth heavy chain as shown in SEQ ID NO: 192;
3) a light chain as shown in SEQ ID NO: 177, a third heavy chain as shown in SEQ ID NO: 185, and a fourth heavy chain as shown in SEQ ID NO: 193;
4) a light chain as shown in SEQ ID NO: 178, a third heavy chain as shown in SEQ ID NO: 186, and a fourth heavy chain as shown in SEQ ID NO: 194;
5) a light chain as shown in SEQ ID NO: 179, a third heavy chain as shown in SEQ ID NO: 187, and a fourth heavy chain as shown in SEQ ID NO: 195;
6) a light chain as shown in SEQ ID NO: 179, a third heavy chain as shown in SEQ ID NO: 188 and a fourth heavy chain as shown in SEQ ID NO: 196;
7) the light chain as shown in SEQ ID NO: 182, the third heavy chain as shown in SEQ ID NO: 189 and the fourth heavy chain as shown in SEQ ID NO: 197;
8) the light chain as shown in SEQ ID NO: 182, the third heavy chain as shown in SEQ ID NO: 190 and the fourth heavy chain as shown in SEQ ID NO: 198.

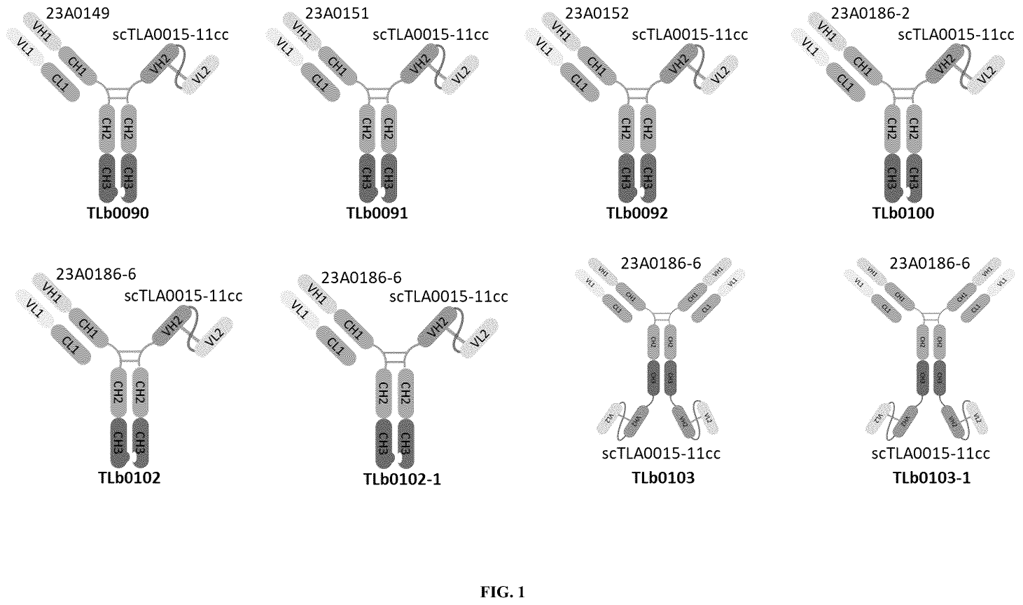

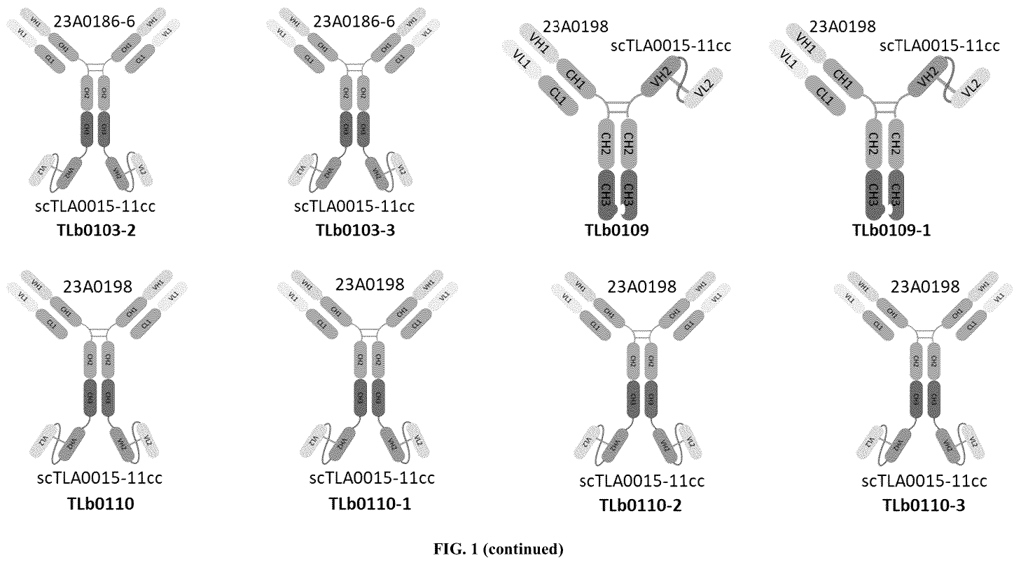

Specifically, as shown in FIG. 1, the antigen binding protein of the present application may have the following structure (excluding the linker). The specific types of the first antigen binding domain and/or the second binding domain are shown in the figure:
Heavy chain from N-terminus to C-terminus: VH, CH1, CH2, CH3 of the second domain, VH of the first domain scFV, VL of the first domain scFV; light chain from N-terminus to C-terminus: VH, CH1, CH2, CH3 of the second domain, VL of the first domain scFV, VH of the first domain scFV; light chain from N-terminus to C-terminus: VH, CH1, CH2, CH3 of the second domain, VL of the first domain scFV; light chain from N-terminus to C-terminus: VL of the second domain, light chain constant region;
Heavy chain from N-terminus to C-terminus: VL of the first domain scFv, VH of the first domain scFv, VH, CH1, CH2, CH3 of the second domain; light chain from N-terminus to C-terminus: VL of the second domain, light chain constant region.

Heavy chain from N-terminus to C-terminus: VH, CH1, CH2, CH3 of the first domain, VH of the second domain scFV, VL of the second domain scFV; light chain from N-terminus to C-terminus: VL of the first domain, light chain constant region;
Heavy chain from N-terminus to C-terminus: VH, CH1, CH2, CH3 of the first domain, VL of the second domain scFV, VH of the second domain scFV; light chain from N-terminus to C-terminus: VL of the first domain, light chain constant region;
Heavy chain from N-terminus to C-terminus: VL of the second domain scFv, VH of the second domain scFv, VH of the first domain, CH1, CH2, CH3; light chain from N-terminus to C-terminus: VL of the first domain, light chain constant region.

### Recombinant Antibodies

In the present application, the antigen-binding protein may include a heavy chain constant region, and the heavy chain constant region may include a constant region derived from IgG or a constant region derived from IgY.

For example, the antigen-binding protein may include a constant region derived from IgG. For example, the antigen-binding protein may include a heavy chain. Wherein the heavy chain may include a heavy chain variable region and a heavy chain constant region. The heavy chain variable region and the heavy chain constant region may be directly or indirectly connected. For example, the heavy chain variable region may be directly connected to the heavy chain constant region. For example, the heavy chain constant region may include a constant region derived from a protein selected from the group consisting of IgG1, IgG2, IgG3 and IgG4. In some embodiments, the heavy chain constant region may include a constant region derived from a protein selected from the group consisting of IgG1. The Fc constant region of the antigen-binding protein may include an amino acid sequence as shown in SEQ ID NO: 169 or SEQ ID NO: 170 or SEQ ID NO: 171-174, and CH1 may be an amino acid sequence as shown in SEQ ID NO: 168.

In the present application, the antigen-binding protein may include a light chain constant region, and the light chain constant region may include a constant region derived from Igκ or a constant region derived from Igλ. For example, the light chain constant region may include an Igκ light chain constant region, such as SEQ ID NO: 167.

For example, the light chain constant region of the antigen-binding protein includes the amino acid sequence shown in SEQ ID NO: 167.

### Peptides and Immunoconjugates

In another aspect, the present application provides one or more polypeptides, which may comprise an isolated antigen-binding protein of the present application.

In another aspect, the present application provides one or more immunoconjugates, which may comprise an isolated antigen-binding protein of the present application. In some embodiments, the immunoconjugate also comprises a pharmaceutically acceptable therapeutic agent.

### Nucleic Acid, Vector, and Cell

In another aspect, the present application also provides one or more isolated nucleic acid molecules that may encode the isolated antigen-binding proteins described herein. For example, each of the one or more nucleic acid molecules may encode the entire antigen-binding protein or a portion thereof (e.g., one or more of the HCDR1-3 and the heavy chain variable regions).

The nucleic acid molecules described herein may be isolated. For example, it may be produced or synthesized by the following methods: (i) in vitro amplification, e.g., by polymerase chain reaction (PCR) amplification, (ii) clonal recombination, (iii) purification, e.g., by digestion and gel electrophoresis fractionation, or (iv) synthesis, e.g., by chemical synthesis. For example, the isolated nucleic acid may be a nucleic acid molecule prepared by recombinant DNA technology.

In the present application, the nucleic acids encoding the isolated antigen-binding proteins may be prepared by a variety of methods known in the art including, but not limited to, using reverse transcription PCR and PCR to obtain nucleic acid molecules of the isolated antigen-binding proteins described herein.

In another aspect, the present application provides one or more vectors comprising one or more nucleic acid molecules described herein. Each vector may comprise one or more of the nucleic acid molecules. In addition, the vector may also comprise other genes, such as marker genes that allow for selection of the vector in an appropriate host cell and under appropriate conditions. In addition, the vector may also comprise expression control elements that allow for the proper expression of the coding region in an appropriate host. Such control elements are well known to those skilled in the art and may include, for example, promoters, ribosome binding sites, enhancers, and other control elements that regulate gene transcription or mRNA translation, etc. In some embodiments, the expression control sequence is a regulatable element. The specific structure of the expression control sequences may vary depending on the function of the species or cell type, but typically comprise 5′ non-transcribed sequences and 5′ and 3′ non-translated sequences involved in transcription and translation initiation, respectively, such as TATA cassettes, capping sequences, CAAT sequences, etc. For example, the 5′ non-transcribed expression control sequence may comprise a promoter region, which may comprise a promoter sequence for transcription control of a functionally linked nucleic acid. The expression control sequences may also include enhancer sequences or upstream activator sequences. In the present application, suitable promoters may include, for example, promoters for SP6, T3, and T7 polymerases, human U6RNA promoters, CMV promoters, and artificial hybrid promoters thereof (e.g., CMV), wherein some portion of the promoter may be fused to some portion of the promoter of a gene for another cellular proteins (e.g., human GAPDH, glyceraldehyde-3-phosphate dehydrogenase), which may or may not comprise an additional intron. One or more nucleic acid molecules described herein can be operably linked to the expression control elements.

Such vectors may include, for example, plasmids, cosmids, viruses, phages, or other vectors commonly used in, for example, genetic engineering. For example, the vector may be an expression vector. For example, the vector may be a viral vector. The patient may be administered directly (in vivo) with the viral vector or may be administered indirectly, e.g., the patient may be administered with the cell treated with the virus in vitro (ex vivo). Viral vector technology is well known in the art and is described, for example, in Sambrook et al., (2001, Molecular Cloning: A Laboratory Manual, Cold Spring Harbor Laboratory, New York) and other virology and molecular biology manuals. Conventional virus-based systems may include retroviral vectors, lentiviral vectors, adenoviral vectors, adeno-associated viral vectors, and herpes simplex viral vectors for gene transfer. In some cases, retroviral, lentiviral, and adeno-associated viral methods can be used to transfer and integrate gene into the host genome for long term expression of the inserted gene. Lentiviral vectors are retroviral vectors capable of transducing or infecting non-dividing cells and typically producing higher viral titers. Lentiviral vectors may comprise a long terminal repeat 5′ LTR and a truncated 3′ LTR, a RRE, a rev response element (cPPT), a central termination sequence (CTS), and/or a post-translational regulatory element (WPRE). The vectors described herein can be introduced into cells.

According to another aspect, the present application provides a cell. The cell may comprise an isolated antigen-binding protein as described herein, a polypeptide as described herein, an immunoconjugate as described herein, one or more nucleic acid molecules and/or one or more vectors as described herein. For example, each or every cell may comprise a nucleic acid molecule or vector described herein. For example, each or every cell may comprise many (e.g., 2 or more) or multiple (e.g., 2 or more) kinds of nucleic acid molecules or vectors described herein. For example, the vectors described herein can be introduced into said host cells, such as prokaryotic cells (e.g., bacterial cells), CHO cells, NS/0 cells, HEK293T cells, 293F cells, or HEK293A cells, or other eukaryotic cells, such as cells from plants, fungal or yeast cells, etc. The vectors described herein can be introduced into the host cells by methods known in the art, such as electroporation, lipofectine transfection, lipofectamin transfection, etc. For example, the cells may include yeast cells. For example, the cells may include E. coli cells. For example, the cells may include mammalian cells. For example, the cells may include immune cells.

The cells may include immune cells. In some cases, the cells may include immune cells. For example, the cells may include T cells, B cells, natural killer cells (NK cells), macrophages, NKT cells, monocytes, dendritic cells, granulocytes, lymphocytes, leukocytes and/or peripheral blood mononuclear cells.

### Pharmaceutical Composition and Pharmaceutical Combination

In another aspect, the present application provides a pharmaceutical composition. The pharmaceutical composition may comprise the isolated antigen-binding protein, the polypeptide, the immunoconjugate, the isolated nucleic acid molecule, the vector, the cell, and/or the pharmaceutically acceptable adjuvant and/or excipient as described herein. In the present application, the pharmaceutically acceptable adjuvants may include buffers, antioxidants, preservatives, low molecular weight polypeptides, proteins, hydrophilic polymers, amino acids, sugars, chelating agents, counter ions, metal complexes and/or nonionic surfactants. Any conventional media or agent is contemplated for the pharmaceutical compositions of the present application, unless they are incompatible with the cells described herein. In the present application, the pharmaceutically acceptable excipients may include an additive other than the main drug in the pharmaceutical preparation, which may also be referred to as pharmaceutical necessities. For example, the excipients may include binders, fillers, disintegrants, and lubricants in tablets. For example, the excipients may include alcohol, vinegar, medicinal juice, etc. in traditional Chinese medicine pills. For example, the excipients may include the base portion of semi-solid formulation ointments and creams. For example, the excipients may include preservatives, antioxidants, flavoring agents, perfuming agents, solubilizing assistant, emulsifiers, solubilizers, osmotic pressure regulators, and colorants in liquid formulations.

In another aspect, the present application provides an isolated antigen binding protein, the polypeptide, the immunoconjugate, the isolated nucleic acid molecule, the vector, and the pharmaceutical composition for preventing, alleviating and/or treating a disease or condition.

In another aspect, in the present application, the kit and/or the drug combination are used to prevent, alleviate and/or treat a disease or condition.

In another aspect, the present application provides a use of a drug combination in the preparation of a drug, which is used to prevent, alleviate and/or treat a disease or condition.

In another aspect, the present application provides a method for preventing and/or treating a disease or condition, which comprises administering the isolated antigen binding protein, the isolated nucleic acid molecule, the vector, the cell, and the pharmaceutical composition to a subject in need thereof.

The disease or condition disclosed herein may be an inflammatory disease, a fibrostenotic disease, or a fibrotic disease. In some instances, the disease or the condition is a TL1A or IL-23P19 mediated disease or condition. The term, “TL1A or IL-23P19 mediated disease or condition” refers to a disease or a condition pathology or pathogenesis that is driven, at least in part, by TL1A or IL-23P19 signaling. In some instances, the disease or the condition is immune-mediated disease or condition, such as those mediated by TL1A or IL-23P19.

In some embodiments, the disease or the condition is an inflammatory disease or disorder that is mediated, at least in part, by TL1A or IL-23P19 signaling. Non-limiting examples of inflammatory disease include, allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

In some embodiments, the disease or the condition is an autoimmune disease that is mediated, at least in part, by TL1A or IL-23P19 signaling. Non-limiting examples of autoimmune disease or disorder include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal MotorNeuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the disease or the condition is a cancer that is mediated, at least in part, by TL1A or IL-23P19 signaling. Non-limiting examples of cancers include Adenoid Cystic Carcinoma, Adrenal Gland Cancer, Amyloidosis, Anal Cancer, Ataxia-Telangiectasia, Atypical Mole Syndrome, Basal Cell Carcinoma, Bile Duct Cancer, Birt Hogg Dube Syndrome, Bladder Cancer, Bone Cancer, Brain Tumor, Breast Cancer, Breast Cancer in Men, Carcinoid Tumor, Cervical Cancer, Colorectal Cancer, Ductal Carcinoma, Endometrial Cancer, Esophageal Cancer, Gastric Cancer, Gastrointestinal Stromal Tumor (GIST), HER2-Positive Breast Cancer, Islet Cell Tumor, Juvenile Polyposis Syndrome, Kidney Cancer, Laryngeal Cancer, Leukemia—Acute Lymphoblastic Leukemia, Leukemia—Acute Lymphocytic (ALL), Leukemia—Acute Myeloid AML, Leukemia—Adult, Leukemia—Childhood, Leukemia Chronic Lymphocytic (CLL), Leukemia—Chronic Myeloid (CIVIL), Liver Cancer, Lobular Carcinoma, Lung Cancer, Lung Cancer-Small Cell (SCLC), Lung Cancer—Non-small Cell (NSCLC), Lymphoma—Hodgkin's, Lymphoma—Non-Hodgkin's, Malignant Glioma, Melanoma, Meningioma, Multiple Myeloma, Myelodysplastic Syndrome (MDS), Nasopharyngeal Cancer, Neuroendocrine Tumor, Oral Cancer, Osteosarcoma, Ovarian Cancer, Pancreatic Cancer, Pancreatic Neuroendocrine Tumors, Parathyroid Cancer, Penile Cancer, Peritoneal Cancer, Peutz-Jeghers Syndrome, Pituitary Gland Tumor, Polycythemia Vera, Prostate Cancer, Renal Cell Carcinoma, Retinoblastoma, Salivary Gland Cancer, Sarcoma, Sarcoma-Kaposi, Skin Cancer, Small Intestine Cancer, Stomach Cancer, Testicular Cancer, Thymoma, Thyroid Cancer, Uterine (Endometrial) Cancer, Vaginal Cancer, and Wilms' Tumor.

In some embodiments, the disease or the condition is an inflammatory bowel disease, such as Crohn's disease (CD) or ulcerative colitis (UC). A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a standard treatment (e.g., anti-TNFα therapy). Non-limiting examples of standard treatment include glucocorticosteriods, anti-TNF therapy, anti-a4-b7 therapy (vedolizumab), anti-IL12p40 therapy (ustekinumab), Thalidomide, and Cytoxin.

In the present application, the subject may include a human or non-human animal. For example, the non-human animal may be selected from the group consisting of: a monkey, a chicken, a goose, a cat, a dog, a mouse, and a rat. In addition, a non-human animal may also include any animal species other than human, such as livestock animals, or rodents, or primates, or domestic animals, or poultry animals. The human can be Caucasian, African, Asian, Sumerian, or other ethnicity, or a hybrid of various ethnicities. As another example, the human may be the elderly, adults, adolescents, children or infants.

Effective amounts in human can be extrapolated from the effective amounts in experimental animals. For example, Freireich et al. described the interrelationship of dosages for animals and humans (based on milligrams per meter squared of body surface) (Freireich et al., Cancer Chemother. Rep. 50, 219 (1966)). Body surface area can be approximately determined from the height and weight of the patient. See, e.g., Scientific Tables, Geigy Pharmaceuticals, Ardsley, N.Y, 537 (1970).

Without intending to be bound by any theory, the following examples are intended merely to illustrate the fusion proteins, methods of preparation, uses, etc., of the present application and are not intended to limit the scope of the present application.

### Example

### Example 1 Anti-TL1A and Antibody IL-23p19 Monoclonal Antibody Sequences

Anti-TL1A monoclonal antibody: The anti-TL1A monoclonal antibody uses the TLA0015-11 antibody screened internally. Its VH and VL amino acid sequences and the sequence after adding disulfide bonds to its scFv configuration (scTLA0015-11cc) are shown in Table 1. Anti-IL-23p19 monoclonal antibody: The 18E5 mouse monoclonal antibody sequence was used and humanized, and the PTM was removed. The VH and VL amino acid sequences of the humanized and modified antibodies are shown in Table 1; in addition, the VH and VL amino acid sequences of the IL-23p19 antibody A5M21 are shown in Table 1. The VH and VL amino acid sequences of the anti-TL1A control antibody and the anti-IL-23p19 control antibody are shown in Table 2. The sequences of the human IgG1 antibody CK, CH1 and Fc, and Fc mutants are shown in Table 3.

TABLE 1
Amino acid sequences of anti-TL1A Ab and anti-IL-23p19 Ab

| antibody | VH | VL |
|---|---|---|
| TLA0015-11 | EIQLVESGGGLIQPGGSLRISCAVSGFSLSSYGVDWVRQAPGKGLEW | DIQMTQSPSSLSASVGDRVTITCRAS |
| | LGVIWGFGGTNYNSALKSRLTISKDNSKNTVYLQMNSLRAEDTAVY | QDISNYLNWYQQKPGKAVKLLIYYT |
| | YCASGNFDAMDYWGQGTLVTVSS | SRLHSGVPSRFSGSGSGTDYTLTISSL |
| | | QPEDFATYFCQQGNTLPFTFGQGTK |
| | | LEIK |
| scTLA0015-11cc | EIQLVESGGGLIQPGGSLRISCAVSGFSLSSYGVDWVRQAPGKCLEW | / |
| | LGVIWGFGGTNYNSALKSRLTISKDNSKNTVYLQMNSLRAEDTAVY | |
| | YCASGNFDAMDYWGQGTLVTVSSGGGGSGGGGSGGGGSGGGGSDI | |
| | QMTQSPSSLSASVGDRVTITCRASQDISNYLNWYQQKPGKAVKLLIY | |
| | YTSRLHSGVPSRFSGSGSGTDYTLTISSLQPEDFATYFCQQGNTLPFTF | |
| | GCGTKLEIK | |
| 16003-18E5 | EVQLQQSGPELVKPGAAVKMSCKASGYTFTRYLIHWVKQKPGQGL | DIVMTQSPATLSVTPGDRVSLSCRAS |
| | EWIGYINPYNDGTKYNEKFKGKATLTSDKSSSTAYMELSSLTSEDSA | QSISDYLYWYQQKSHESPRLLIKFAS |
| | VYYCTSNWDLDYWGQGTTLTVSS | QSISGIPSRFTGSGSGSDFTLSIDSVEP |
| | | DDVGVFFCQNGHSFPFTFGGGTKLEI |
| | | K |
| 23A0144 | QVQLVQSGAEVKKPGSSVKVSCKASGYTFTRYLIHWVRQAPGQGLE | AIQMTQSPSSLSASVGDRVTITCRAS |
| | WIGYINPYNDGTKYAQKFQGRVTITADESTSTAYMELSSLRSEDTAV | QSISDYLYWYQQKPGKAPKLLIKFAS |
| | YYCTSNWDLDYWGQGTLVTVSS | QSISGVPSRFSGSGSGTDFTLTISSLQP |
| | | EDFATYYCQNGHSFPFTFGPGTKVDI |
| | | K |
| 23A0145 | EVQLVESGGGLVKPGGSLRLSCAASGYTFTRYLIHWVRQAPGKGLE | DIQMTQSPSSLSASVGDRVTITCRAS |
| | WIGYINPYNDGTKYAAPVKGRFTISRDDSKNTLYLQMNSLKTEDTA | QSISDYLYWFQQKPGKAPKLLIKFAS |
| | VYYCTSNWDLDYWGQGTLVTVSS | QSISGVPSRFSGSGSGTDFTLTISSLQP |
| | | EDFATYYCQNGHSFPFTFGQGTRLEI |
| | | K |
| 23A0146 | EVQLVESGGGLVKPGGSLRLSCAASGYTFTRYLIHWVRQAPGKGLE | NIQMTQSPSAMSASVGDRVTITCRAS |
| | WIGYINPYNDGTKYAAPVKGRFTISRDDSKNTLYLQMNSLKTEDTA | QSISDYLYWFQQKPGKVPKLLIKFAS |
| | VYYCTSNWDLDYWGQGTLVTVSS | QSISGVPSRFSGSGSGTEFTLTISSLQP |
| | | EDFATYYCQNGHSFPFTFGPGTKVDI |
| | | K |
| 23A0147 | EVQLVESGGGLVKPGGSLRLSCAASGYTFTRYLIHWVRQAPGKGLE | DIQMTQSPSTLSASVGDRVTITCRAS |
| | WIGYINPYNDGTKYAAPVKGRFTISRDDSKNTLYLQMNSLKTEDTA | QSISDYLYWYQQKPGKAPKLLIKFAS |
| | VYYCTSNWDLDYWGQGTLVTVSS | QSISGVPSRFSGSGSGTEFTLTISSLQP |
| | | DDFATYYCQNGHSFPFTFGPGTKVDI |
| | | K |
| 23A0148 | EVQLVESGGGLVKPGGSLRLSCAASGYTFTRYLIHWVRQAPGKGLE | AIQMTQSPSSLSASVGDRVTITCRAS |
| | WIGYINPYNDGTKYAAPVKGRFTISRDDSKNTLYLQMNSLKTEDTA | QSISDYLYWYQQKPGKAPKLLIKFAS |
| | VYYCTSNWDLDYWGQGTLVTVSS | QSISGVPSRFSGSGSGTDFTLTISSLQP |
| | | EDFATYYCQNGHSFPFTFGQGTKVEI |
| | | K |
| 23A0149 | EVQLQQSGPELVKPGASVKVSCKASGYTFTRYLMHWVRQAPGQGL | AIVMTQSPATLSVSPGERVTLSCRAS |
| | EWIGYINPYNDGTKYNQKFQGRVTITADKSTSTAYMELSSLTSEDTA | QSISDYLYWYQQKPDQSPKLLIKFAS |
| | VYYCTSNWDLDYWGQGTLVTVSS | QSISGVPSRFSGSGSGRDFTLTISSLQP |
| | | EDFAVYYCQNGHSFPFTFGGGTKLEI |
| | | K |
| 23A0150 | EVQLQQSGPEVKKPGASVKVSCKASGYTFTRYLIHWVRQAPGQGLE | NIVMTQSPATLSVSPGERATLSCRAS |
| | WIGYINPYNDGTKYNQKFQGRVTITADKSTSTAYMELSSLRSEDTAV | QSISDYLYWYQQKPDQSPKLLIKFAS |
| | YYCTSNWDLDYWGQGTTLTVSS | QSISGVPSRFSGSGSGRDFTLTISSLEP |
| | | EDFAVYYCQNGHSFPFTFGGGTKLEI |
| | | K |
| 23A0151 | QVQLQQSGSELKKPGASVKVSCKASGYTFTRYLMHWVRQAPGKGL | EIVMTQSPATLSLSPGERATLSCRAS |
| | EWIGYINPYNDGTKYNQKFQGRVTITADKSTSTAYMELSSLRSEDTA | QSISDYLYWYQQKPDQSPKLLIKFAS |
| | VYYCTSNWDLDYWGQGTLVTVSS | QSISGVPSRFSGSGSGTEFTLTISSLQP |
| | | EDFAVYYCQNGHSFPFTFGGGTKVEI |
| | | K |
| 23A0152 | EIQLQQSGSELKKPGASVKVSCKASGYTFTRYLMNWVRQAPGKGLE | DIVMTQSPATLSVSPGERVTLSCRAS |
| | WIGYINPYNDGTKYNQKFQGRVTITADKSTSTAYMELSSLRSEDTAV | QSISDYLYWYQQKPDQSPKLLIKFAS |
| | YYCTSNWDLDYWGQGTLVTVSS | QSISGVPSRFSGSGSGSDFTLTISSLQP |
| | | EDFATYYCQNGHSFPFTFGGGTKLEI |
| | | K |
| 23A0153 | EVQLQQSGPEVKKPGASVKVSCKASGYTFTRYLMHWVRQPPGQGL | DIVMTQSPATLSVSPGERVTLSCRAS |
| | EWIGYINPYNDGTKYNQKFQGRVTITADKSTSTAYMELSSLRSEDTA | QSISDYLYWYQQKPDQSPRLLIKFAS |
| | VYYCTSNWDLDYWGQGTTLTVSS | QSISGVPSRFSGSGSGSDFTLKISSVE |
| | | AEDVGVYYCQNGHSFPFTFGGGTKV |
| | | EIK |
| 23A0175 | QVQLVQSGAEVKKPGASVKVSCKASGYTFTRYLIHWVRQAPGQGL | DIQMTQSPSSLSASVGDRVTITCRAS |
| | EWMGYINPYNDGTKYNEKFKGRVTMTRDTSTSTVYMELSSLRSEDT | QSISDYLYWYQQKPGKAPKLLIYFAS |
| | AVYYCASNWDLDYWGQGTLVTVSS | QSISGVPSRFSGSGSGTDFTLTISSLQP |
| | | EDFATYYCQNGHSFPFTFGGGTKVEI |
| | | K |
| 23A0176 | QVQLVQSGAEVKKPGASVKVSCKASGYTFTRYLIHWVRQAPGQGL | DIQMTQSPSSLSASVGDRVTITCRAS |
| | EWMGYINPYNDGTKYNEKFKGRVTMTRDTSTSTVYMELSSLRSEDT | QSISDYLYWYQQKPGKAPKLLIKFAS |
| | AVYYCASNWDLDYWGQGTLVTVSS | QSISGIPSRFSGSGSGSDFTLTISSLQP |
| | | EDFATFYCQNGHSFPFTFGGGTKVEI |
| | | K |
| 23A0177 | QVQLVQSGAEVKKPGASVKVSCKASGYTFTRYLIHWVRQAPGQGL | DIVMTQSPSSLSASVGDRVTLTCRAS |
| | EWMGYINPYNDGTKYNEKFKGRVTMTRDTSTSTVYMELSSLRSEDT | QSISDYLYWYQQKPGKAPKLLIKFAS |
| | AVYYCASNWDLDYWGQGTLVTVSS | QSISGIPSRFSGSGSGSDFTLTISSVQP |
| | | EDFATFFCQNGHSFPFTFGGGTKVEI |
| | | K |
| 23A0178 | QVQLVQSGAEVKKPGASVKVSCKASGYTFTRYLIHWVRQAPGQGL | EIVMTQSPATLSVSPGERVTLSCRAS |
| | EWMGYINPYNDGTKYNEKFKGRVTMTRDTSTSTVYMELSSLRSEDT | QSISDYLYWYQQKPGQAPRLLIKFAS |
| | AVYYCASNWDLDYWGQGTLVTVSS | QSISGIPARFSGSGSGSEFTLTISSVQS |
| | | EDFAVFFCQNGHSFPFTFGGGTKVEI |
| | | K |
| 23A0179 | QVQLVQSGAEVKKPGASVKVSCKASGYTFTRYLIHWVRQAPGQGL | DIQMTQSPSSLSASVGDRVTITCRAS |
| | EWIGYINPYNDGTKYNEKFKGRVTMTSDKSTSTAYMELSSLRSEDT | QSISDYLYWYQQKPGKAPKLLIYFAS |
| | AVYYCTSNWDLDYWGQGTLVTVSS | QSISGVPSRFSGSGSGTDFTLTISSLQP |
| | | EDFATYYCQNGHSFPFTFGGGTKVEI |
| | | K |
| 23A0180 | QVQLVQSGAEVKKPGASVKVSCKASGYTFTRYLIHWVRQAPGQGL | DIQMTQSPSSLSASVGDRVTITCRAS |
| | EWIGYINPYNDGTKYNEKFKGRVTMTSDKSTSTAYMELSSLRSEDT | QSISDYLYWYQQKPGKAPKLLIKFAS |
| | AVYYCTSNWDLDYWGQGTLVTVSS | QSISGIPSRFSGSGSGSDFTLTISSLQP |
| | | EDFATFYCQNGHSFPFTFGGGTKVEI |
| | | K |
| 23A0181 | QVQLVQSGAEVKKPGASVKVSCKASGYTFTRYLIHWVRQAPGQGL | DIVMTQSPSSLSASVGDRVTLTCRAS |
| | EWIGYINPYNDGTKYNEKFKGRVTMTSDKSTSTAYMELSSLRSEDT | QSISDYLYWYQQKPGKAPKLLIKFAS |
| | AVYYCTSNWDLDYWGQGTLVTVSS | QSISGIPSRFSGSGSGSDFTLTISSVQP |
| | | EDFATFFCQNGHSFPFTFGGGTKVEI |
| | | K |
| 23A0182 | QVQLVQSGAEVKKPGASVKVSCKASGYTFTRYLIHWVRQAPGQGL | EIVMTQSPATLSVSPGERVTLSCRAS |
| | EWIGYINPYNDGTKYNEKFKGRVTMTSDKSTSTAYMELSSLRSEDT | QSISDYLYWYQQKPGQAPRLLIKFAS |
| | AVYYCTSNWDLDYWGQGTLVTVSS | QSISGIPARFSGSGSGSEFTLTISSVQS |
| | | EDFAVFFCQNGHSFPFTFGGGTKVEI |
| | | K |
| 23A0183 | QVQLVQSGAEVVKPGASVKMSCKASGYTFTRYLIHWVKQAPGQGL | DIQMTQSPSSLSASVGDRVTITCRAS |
| | EWIGYINPYNDGTKYNEKFKGRATLTSDKSTSTAYMELSSLRSEDTA | QSISDYLYWYQQKPGKAPKLLIYFAS |
| | VYYCTSNWDLDYWGQGTLVTVSS | QSISGVPSRFSGSGSGTDFTLTISSLQP |
| | | EDFATYYCQNGHSFPFTFGGGTKVEI |
| | | K |
| 23A0184 | QVQLVQSGAEVVKPGASVKMSCKASGYTFTRYLIHWVKQAPGQGL | DIQMTQSPSSLSASVGDRVTITCRAS |
| | EWIGYINPYNDGTKYNEKFKGRATLTSDKSTSTAYMELSSLRSEDTA | QSISDYLYWYQQKPGKAPKLLIKFAS |
| | VYYCTSNWDLDYWGQGTLVTVSS | QSISGIPSRFSGSGSGSDFTLTISSLQP |
| | | EDFATFYCQNGHSFPFTFGGGTKVEI |
| | | K |
| 23A0185 | QVQLVQSGAEVVKPGASVKMSCKASGYTFTRYLIHWVKQAPGQGL | DIVMTQSPSSLSASVGDRVTLTCRAS |
| | EWIGYINPYNDGTKYNEKFKGRATLTSDKSTSTAYMELSSLRSEDTA | QSISDYLYWYQQKPGKAPKLLIKFAS |
| | VYYCTSNWDLDYWGQGTLVTVSS | QSISGIPSRFSGSGSGSDFTLTISSVQP |
| | | EDFATFFCQNGHSFPFTFGGGTKVEI |
| | | K |
| 23A0186 | QVQLVQSGAEVVKPGASVKMSCKASGYTFTRYLIHWVKQAPGQGL | EIVMTQSPATLSVSPGERVTLSCRAS |
| | EWIGYINPYNDGTKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQNGHSFPFTFGGGTKVEI |
| | | K |
| 23A0186-1 | QVQLVQSGAEVVKPGASVKMSCKASGYTFTRYLIHWVKQAPGQGL | EIVMTQSPATLSVSPGERVTLSCRAS |
| | EWIGYINPYNDATKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQQGHSFPFTFGGGTKVEI |
| | | K |
| 23A0186-2 | QVQLVQSGAEVVKPGASVKMSCKASGYTFTRYLIHWVKQAPGQGL | EIVMTQSPATLSVSPGERVTLSCRAS |
| | EWIGYINPYNEGTKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQQGHSFPFTFGGGTKVEI |
| | | K |
| 23A0186-3 | QVQLVQSGAEVVKPGASVKMSCKASGYTFTRYLIHWVKQAPGQGL | EIVMTQSPATLSVSPGERVTLSCRAS |
| | EWIGYINPYNDATKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQNAHSFPFTFGGGTKVEI |
| | | K |
| 23A0186-4 | QVQLVQSGAEVVKPGASVKMSCKASGYTFTRYLIHWVKQAPGQGL | EIVMTQSPATLSVSPGERVTLSCRAS |
| | EWIGYINPYNEGTKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQNAHSFPFTFGGGTKVEI |
| | | K |
| 23A0186-5 | QVQLVQSGAEVVKPGASVKMSCKASGYTFTRYLIHWVKQAPGQGL | EIVMTQSPATLSVSPGERVTLSCRAS |
| | EWIGYINPYNDGTKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQQGHSFPFTFGGGTKVEI |
| | | K |
| 23A0186-6 | QVQLVQSGAEVVKPGASVKMSCKASGYTFTRYLIHWVKQAPGQGL | EIVMTQSPATLSVSPGERVTLSCRAS |
| | EWIGYINPYNEGTKYNEKFKGRATLTSDKSTSTAYMELSSLRSEDTA | QSISDYLYWYQQKPGQAPRLLIKFAS |
| | VYYCTSNWDLDYWGQGTLVTVSS | QSISGIPARFSGSGSGSEFTLTISSVQS |
| | | EDFAVFFCQNGHSFPFTFGGGTKVEI |
| | | K |
| 23A0198 (A5M21) | QVQLVQSGAEVKKPGSSVKVSCKASGYTFTSYGITWVRQAPGQGLE | DIVMTQSPDSLAVSLGERATINCKAS |
| | WMGENYPRSGNTYYNEKFKGRVTITADKSTSTAYMELSSLRSEDTA | KKVTIYGSTSALHWYQQKPGQPPKL |
| | VYYCARAEFISTVVAPYYYALDYWGQGTLVTVSS | IYNLAKPESGVPDRFSGSGSGTDFTL |
| | | TISSLQAEDVAVYYCLQNKEVPYTF |
| | | GGGTKVEIK |

TABLE 2
anti-TL1A control antibody and anti-IL-23 control antibody VH and VL amino
acid sequences

| Reference | | |
|---|---|---|
| antibody | VH | VL |
| RVT-3101 | QVQLVQSGAEVKKPGASVKVSCKASGYDFTYYGISWVRQA | EIVLTQSPATLSLSPGERATLSCRASQSVSSYLAW |
| | PGQGLEWMGWISTYNGNTHYARMLQGRVTMTTDTSTRTA | YQQKPGQAPRLLIYDASNRATGIPARFSGSGSGT |
| | YMELRSLRSDDTAVYYCARENYYGSGAYRGGMDVWGQGT | DFTLTISSLEPEDFAVYYCQQRSNWPWTFGQGT |
| | TVTVSS | KVEIK |
| MK-7240 | QVQLVQSGAEVKKPGASVKVSCKASGFDIQDTYMHWVKQR | EIVLTQSPGTLSLSPGERATLSCRASSSVSYMYW |
| | PGQGLEWMGRIDPASGHTKYDPKFQVRVTITRDTSTSTVYLE | YQQKPGQAPRPLIYATSNLASGIPDRFSGSGSGT |
| | LSSLRSEDTAVYYCARSGGLPDVWGQGTTVTVSS | DFTLTISRLEPEDFAVYYCQQWEGNPRTFGGGTK |
| | | LEIK |
| Risankizumab | QVQLVQSGAEVKKPGSSVKVSCKASGYTFTDQTIHWMRQA | DIQMTQSPSSLSASVGDRVTITCKASRDVAIAVA |
| | PGQGLEWIGYIYPRDDSPKYNENFKGKVTITADKSTSTAYME | WYQQKPGKVPKLLIYWASTRHTGVPSRFSGSGS |
| | LSSLRSEDTAVYYCAIPDRSGYAWFIYWGQGTLVTVSS | RTDFTLTISSLQPEDVADYFCHQYSSYPFTFGSGT |
| | | KLEIK |
| Mirikizumab | QVQLVQSGAEVKKPGSSVKVSCKASGYKFTRYVMHWVRQ | DIQMTQSPSSLSASVGDRVTITCKASDHILKFLT |
| | APGQGLEWMGYINPYNDGTNYNEKFKGRVTITADKSTSTAY | WYQQKPGKAPKLLIYGATSLETGVPSRFSGSGSG |
| | MELSSLRSEDTAVYYCARNWDTGLWGQGTTVTVSS | TDFTLTISSLQPEDFATYYCQMYWSTPFTFGGGT |
| | | KVEIK |
| Ustekinumab | EVQLVQSGAEVKKPGESLKISCKGSGYSFTTYWLGWVRQMP | DIQMTQSPSSLSASVGDRVTITCRASQGISSWLA |
| | GKGLDWIGIMSPVDSDIRYSPSFQGQVTMSVDKSITTAYLQW | WYQQKPEKAPKSLIYAASSLQSGVPSRFSGSGSG |
| | NSLKASDTAMYYCARRRPGQGYFDFWGQGTLVTVSS | TDFTLTISSLQPEDFATYYCQQYNIYPYTFGQGT |
| | | KLEIK |

TABLE 3
Human IgG1

| name | sequence |
|---|---|
| Cκ | RTVAAPSVFIFPPSDEQLKSGTASVVCLLNNFYPREAKVQWKVDNALQSGNSQESVTEQDSKDSTYSLSSTLTLSKAD |
| | YEKHKVYACEVTHQGLSSPVTKSFNRGEC |
| CH1 | ASTKGPSVFPLAPSSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSC |
| Fc | DKTHTCPPCPAPEAAGGPSVFLFPPKPKDTLYITREPEVTCVVVDVSHEDPEVKFNWYVDGVEVHNAKTKPREEQYNS |
| (LALA + | TYRVVSVLTVLHQDWLNGKEYKCKVSNKALPAPIEKTISKAKGQPREPQVYTLPPSRDELTKNQVSLTCLVKGFYPSD |
| YTE) | LAVEWESNGQPENNYKTTPPVLDSDGSFFLYSKLTVDKSRWQQGNVFSCSVMHEALHNHYTQKSLSLSPGK |
| Fc | DKTHTCPPCPAPEAAGGPSVFLFPPKPKDTLMISRTPEVTCVVVDVSHEDPEVKFNWYVDGVEVHNAKTKPREEQYN |
| (LALA + | STYRVVSVLTVLHQDWLNGKEYKCKVSNKALPAPIEKTISKAKGQPREPQVYTLPPSRDELTKNQVSLTCLVKGFYPS |
| LS) | DIAVEWESNGQPENNYKTTPPVLDSDGSFFLYSKLTVDKSRWQQGNVFSCSVLHEALHSHYTQKSLSLSPGK |
| Fc- | DKTHTCPPCPAPEAAGGPSVFLFPPKPKDTLYITREPEVTCVVVDVSHEDPEVKFNWYVDGVEVHNAKTKPREEQYNS |
| Knob | TYRVVSVLTVLHQDWLNGKEYKCKVSNKALPAPIEKTISKAKGQPREPQVYTLPPSRDELTKNQVSLWCLVKGFYPS |
| (LALA + | DIAVEWESNGQPENNYKTTPPVLDSDGSFFLYSKLTVDKSRWQQGNVFSCSVMHEALHNRFTQKSLSLSPGK |
| YTE) | |
| Fc- | DKTHTCPPCPAPEAAGGPSVFLFPPKPKDTLYITREPEVTCVVVDVSHEDPEVKFNWYVDGVEVHNAKTKPREEQYNS |
| Hole | TYRVVSVLTVLHQDWLNGKEYKCKVSNKALPAPIEKTISKAKGQPREPQVCTLPPSREEMTKNQVSLSCAVKGFYPS |
| (LALA + | DIAVEWESNGQPENNYKTTPPVLDSDGSFFLVSKLTVDKSRWQQGNVFSCSVMHEALHNRFTQKSLSLSPGK |
| YTE) | |
| Fc- | DKTHTCPPCPAPEAAGGPSVFLFPPKPKDTLMISRTPEVTCVVVDVSHEDPEVKFNWYVDGVEVHNAKTKPREEQYN |
| Knob | STYRVVSVLTVLHQDWLNGKEYKCKVSNKALPAPIEKTISKAKGQPREPQVYTLPPCREEMTKNQVSLWCLVKGFYP |
| (LALA + | SDIAVEWESNGQPENNYKTTPPVLDSDGSFFLYSKLTVDKSRWQQGNVFSCSVLHEALHSHYTQKSLSLSPGK |
| LS) | |
| Fc- | DKTHTCPPCPAPEAAGGPSVFLFPPKPKDTLMISRTPEVTCVVVDVSHEDPEVKFNWYVDGVEVHNAKTKPREEQYN |
| Hole | STYRVVSVLTVLHQDWLNGKEYKCKVSNKALPAPIEKTISKAKGQPREPQVCTLPPSREEMTKNQVSLSCAVKGFYPS |
| (LALA + | DIAVEWESNGQPENNYKTTPPVLDSDGSFFLVSKLTVDKSRWQQGNVFSCSVLHEALHSRFTQKSLSLSPGK |
| LS) | |

### Example 2 Design of Anti-TL1A+IL-23p19 Bispecific Antibody

The anti-TL1A antibody TLA0015-11 was assembled with the anti-IL-23p19 monoclonal antibody into bispecific antibody molecules of different configurations, wherein the amino acid sequence of the 2+2 configuration bispecific antibody is shown in Table 4, the amino acid sequence of the 1+1 configuration bispecific antibody is shown in Table 5, and the schematic diagrams of the configurations of each bispecific antibody are shown in FIG. 1.

TABLE 4
sequence of 2 + 2 bispecific antibodies

| antibody | VL | VH |
|---|---|---|
| TLb0103 | EIVMTQSPATLSVSPGERVTLSCRASQSISDYLY | QVQLVQSGAEVVKPGASVKMSCKASGYTFTRYLIHWVKQAPGQG |
| | WYQQKPGQAPRLLIKFASQSISGIPARFSGSGSG | LEWIGYINPYNEGTKYNEKFKGRATLTSDKSTSTAYMELSSLRSED |
| | SEFTLTISSVQSEDFAVFFCQNGHSFPFTFGGGT | TAVYYCTSNWDLDYWGQGTLVTVSSASTKGPSVFPLAPSSKSTSG |
| | KVEIKRTVAAPSVFIFPPSDEQLKSGTASVVCLL | GTAALGCLVKDYFPEPVTVSWNSGALTSGVHTFPAVLQSSGLYSL |
| | NNFYPREAKVQWKVDNALQSGNSQESVTEQD | SSVVTVPSSSLGTQTYICNVNHKPSNTKVDKKVEPKSCDKTHTCPP |
| | SKDSTYSLSSTLTLSKADYEKHKVYACEVTHQ | CPAPEAAGGPSVFLFPPKPKDTLYITREPEVTCVVVDVSHEDPEVK |
| | GLSSPVTKSFNRGEC | FNWYVDGVEVHNAKTKPREEQYNSTYRVVSVLTVLHQDWLNGK |
| | | EYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVMHEALHNHYTQKSLSLSPGKGGGGSG |
| | | GGGSGGGGSGGGGSEIQLVESGGGLIQPGGSLRISCAVSGFSLSSYG |
| | | VDWVRQAPGKCLEWLGVIWGFGGTNYNSALKSRLTISKDNSKNT |
| | | VYLQMNSLRAEDTAVYYCASGNFDAMDYWGQGTLVTVSSGGGG |
| | | SGGGGSGGGGSGGGGSDIQMTQSPSSLSASVGDRVTITCRASQDIS |
| | | NYLNWYQQKPGKAVKLLIYYTSRLHSGVPSRFSGSGSGTDYTLTIS |
| | | SLQPEDFATYFCQQGNTLPFTFGCGTKLEIK |
| TLb0103-1 | EIVMTQSPATLSVSPGERVTLSCRASQSISDYLY | QVQLVQSGAEVVKPGASVKMSCKASGYTFTRYLIHWVKQAPGQG |
| | WYQQKPGQAPRLLIKFASQSISGIPARFSGSGSG | LEWIGYINPYNEGTKYNEKFKGRATLTSDKSTSTAYMELSSLRSED |
| | SEFTLTISSVQSEDFAVFFCQNGHSFPFTFGGGT | TAVYYCTSNWDLDYWGQGTLVTVSSASTKGPSVFPLAPSSKSTSG |
| | KVEIKRTVAAPSVFIFPPSDEQLKSGTASVVCLL | GTAALGCLVKDYFPEPVTVSWNSGALTSGVHTFPAVLQSSGLYSL |
| | NNFYPREAKVQWKVDNALQSGNSQESVTEQD | SSVVTVPSSSLGTQTYICNVNHKPSNTKVDKKVEPKSCDKTHTCPP |
| | SKDSTYSLSSTLTLSKADYEKHKVYACEVTHQ | CPAPEAAGGPSVFLFPPKPKDTLMISRTPEVTCVVVDVSHEDPEVK |
| | GLSSPVTKSFNRGEC | FNWYVDGVEVHNAKTKPREEQYNSTYRVVSVLTVLHQDWLNGK |
| | | EYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVLHEALHSHYTQKSLSLSPGKGGGGSG |
| | | GGGSGGGGSGGGGSEIQLVESGGGLIQPGGSLRISCAVSGFSLSSYG |
| | | VDWVRQAPGKCLEWLGVIWGFGGTNYNSALKSRLTISKDNSKNT |
| | | VYLQMNSLRAEDTAVYYCASGNFDAMDYWGQGTLVTVSSGGGG |
| | | SGGGGSGGGGSGGGGSDIQMTQSPSSLSASVGDRVTITCRASQDIS |
| | | NYLNWYQQKPGKAVKLLIYYTSRLHSGVPSRFSGSGSGTDYTLTIS |
| | | SLQPEDFATYFCQQGNTLPFTFGCGTKLEIK |
| TLb0103-2 | EIVMTQSPATLSVSPGERVTLSCRASQSISDYLY | QVQLVQSGAEVVKPGASVKMSCKASGYTFTRYLIHWVKQAPGQG |
| | WYQQKPGQAPRLLIKFASQSISGIPARFSGSGSG | LEWIGYINPYNEGTKYNEKFKGRATLTSDKSTSTAYMELSSLRSED |
| | SEFTLTISSVQSEDFAVFFCQNGHSFPFTFGGGT | TAVYYCTSNWDLDYWGQGTLVTVSSASTKGPSVFPLAPSSKSTSG |
| | KVEIKRTVAAPSVFIFPPSDEQLKSGTASVVCLL | GTAALGCLVKDYFPEPVTVSWNSGALTSGVHTFPAVLQSSGLYSL |
| | NNFYPREAKVQWKVDNALQSGNSQESVTEQD | SSVVTVPSSSLGTQTYICNVNHKPSNTKVDKKVEPKSCDKTHTCPP |
| | SKDSTYSLSSTLTLSKADYEKHKVYACEVTHQ | CPAPEAAGGPSVFLFPPKPKDTLMISRTPEVTCVVVDVSHEDPEVK |
| | GLSSPVTKSFNRGEC | FNWYVDGVEVHNAKTKPREEQYNSTYRVVSVLTVLHQDWLNGK |
| | | EYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVLHEALHSHYTQKSLSLSPGKGGGGSG |
| | | GGGSEIQLVESGGGLIQPGGSLRISCAVSGFSLSSYGVDWVRQAPG |
| | | KCLEWLGVIWGFGGTNYNSALKSRLTISKDNSKNTVYLQMNSLR |
| | | AEDTAVYYCASGNFDAMDYWGQGTLVTVSSGGGGSGGGGSGGG |
| | | GSGGGGSDIQMTQSPSSLSASVGDRVTITCRASQDISNYLNWYQQ |
| | | KPGKAVKLLIYYTSRLHSGVPSRFSGSGSGTDYTLTISSLQPEDFAT |
| | | YFCQQGNTLPFTFGCGTKLEIK |
| TLb0103-3 | EIVMTQSPATLSVSPGERVTLSCRASQSISDYLY | QVQLVQSGAEVVKPGASVKMSCKASGYTFTRYLIHWVKQAPGQG |
| | WYQQKPGQAPRLLIKFASQSISGIPARFSGSGSG | LEWIGYINPYNEGTKYNEKFKGRATLTSDKSTSTAYMELSSLRSED |
| | SEFTLTISSVQSEDFAVFFCQNGHSFPFTFGGGT | TAVYYCTSNWDLDYWGQGTLVTVSS |
| | KVEIKRTVAAPSVFIFPPSDEQLKSGTASVVCLL | ASTKGPSVFPLAPSSKSTSGGTAALGCLVKDYFPEPVTVSWNSGAL |
| | NNFYPREAKVQWKVDNALQSGNSQESVTEQD | TSGVHTFPAVLQSSGLYSLSSVVTVPSSSLGTQTYICNVNHKPSNT |
| | SKDSTYSLSSTLTLSKADYEKHKVYACEVTHQ | KVDKKVEPKSCDKTHTCPPCPAPEAAGGPSVFLFPPKPKDTLMISR |
| | GLSSPVTKSFNRGEC | TPEVTCVVVDVSHEDPEVKFNWYVDGVEVHNAKTKPREEQYNST |
| | | YRVVSVLTVLHQDWLNGKEYKCKVSNKALPAPIEKTISKAKGQPR |
| | | EPQVYTLPPSRDELTKNQVSLTCLVKGFYPSDIAVEWESNGQPENN |
| | | YKTTPPVLDSDGSFFLYSKLTVDKSRWQQGNVFSCSVLHEALHSH |
| | | YTQKSLSLSPGKGGGGSGGGGSGGGGSEIQLVESGGGLIQPGGSLR |
| | | ISCAVSGFSLSSYGVDWVRQAPGKCLEWLGVIWGFGGTNYNSAL |
| | | KSRLTISKDNSKNTVYLQMNSLRAEDTAVYYCASGNFDAMDYWG |
| | | QGTLVTVSSGGGGSGGGGSGGGGSGGGGSDIQMTQSPSSLSASVG |
| | | DRVTITCRASQDISNYLNWYQQKPGKAVKLLIYYTSRLHSGVPSRF |
| | | SGSGSGTDYTLTISSLQPEDFATYFCQQGNTLPFTFGCGTKLEIK |
| TLb0107 | EIVLTQSPATLSLSPGERATLSCRASQSVSSNYL | QVQLVQSGAEVKKPGSSVKVSCKASGGTFSSNYISWVRQAPGQGL |
| | AWYQQKPGQAPRLLIYYASRRATGVPARFSGS | EWMGAYAQKFQGRVTITADESTSTAYMELSSLRSEDTAVYYCAR |
| | GSGTDFTLTISSLEPEDFAYYCQQTSNTPFTFGQ | SKKGMYGGWTYPLMMFDLWGQGTLVTVSSASTKGPSVFPLAPSS |
| | GTKVEIKRTVAAPSVFIFPPSDEQLKSGTASVVC | KSTSGGTAALGCLVKDYFPEPVTVSWNSGALTSGVHTFPAVLQSS |
| | LLNNFYPREAKVQWKVDNALQSGNSQESVTEQ | GLYSLSSVVTVPSSSLGTQTYICNVNHKPSNTKVDKKVEPKSCDKT |
| | DSKDSTYSLSSTLTLSKADYEKHKVYACEVTH | HTCPPCPAPEAAGGPSVFLFPPKPKDTLMISRTPEVTCVVVDVSHE |
| | QGLSSPVTKSFNRGEC | DPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVLHEALHSHYTQKSLSLSPGKG |
| | | GGGSGGGGSGGGGSGGGGSEIQLVESGGGLIQPGGSLRISCAVSGF |
| | | SLSSYGVDWVRQAPGKCLEWLGVIWGFGGTNYNSALKSRLTISKD |
| | | NSKNTVYLQMNSLRAEDTAVYYCASGNFDAMDYWGQGTLVTVS |
| | | SGGGGSGGGGSGGGGSGGGGSDIQMTQSPSSLSASVGDRVTITCR |
| | | ASQDISNYLNWYQQKPGKAVKLLIYYTSRLHSGVPSRFSGSGSGT |
| | | DYTLTISSLQPEDFATYFCQQGNTLPFTFGCGTKLEIK |
| TLb0108 | EIVLTQSPATLSLSPGERATLSCRASKSVRESDY | QVQLVQSGAEVVKPGSSVKVSCKASGYTFTDYYMNWVRQAPGQ |
| | FYMHWYQQKPGQAPRLLIYLASNLESGIPARFS | GLEWIGDFNHNNDVITYNPKFKGRVTLTSDKSTSTAYMELSSLRSE |
| | GSGSGTDFTLTISSLEPEDFAVYYCQNARELPYT | DTAVYYCARGLRGYYAMDYWGQGTLVTVSSASTKGPSVFPLAPS |
| | FGQGTKLEIKRTVAAPSVFIFPPSDEQLKSGTAS | SKSTSGGTAALGCLVKDYFPEPVTVSWNSGALTSGVHTFPAVLQS |
| | VVCLLNNFYPREAKVQWKVDNALQSGNSQES | SGLYSLSSVVTVPSSSLGTQTYICNVNHKPSNTKVDKKVEPKSCDK |
| | VTEQDSKDSTYSLSSTLTLSKADYEKHKVYACE | THTCPPCPAPEAAGGPSVFLFPPKPKDTLMISRTPEVTCVVVDVSH |
| | VTHQGLSSPVTKSFNRGEC | EDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGS |
| | | FFLYSKLTVDKSRWQQGNVFSCSVLHEALHSHYTQKSLSLSPGKG |
| | | GGGSGGGGSGGGGSGGGGSEIQLVESGGGLIQPGGSLRISCAVSGF |
| | | SLSSYGVDWVRQAPGKCLEWLGVIWGFGGTNYNSALKSRLTISKD |
| | | NSKNTVYLQMNSLRAEDTAVYYCASGNFDAMDYWGQGTLVTVS |
| | | SGGGGSGGGGSGGGGSGGGGSDIQMTQSPSSLSASVGDRVTITCR |
| | | ASQDISNYLNWYQQKPGKAVKLLIYYTSRLHSGVPSRFSGSGSGT |
| | | DYTLTISSLQPEDFATYFCQQGNTLPFTFGCGTKLEIK |
| TLb0110 | DIVMTQSPDSLAVSLGERATINCKASKKVTIYG | QVQLVQSGAEVKKPGSSVKVSCKASGYTFTSYGITWVRQAPGQG |
| | STSALHWYQQKPGQPPKLIYNLAKPESGVPDRF | LEWMGENYPRSGNTYYNEKFKGRVTITADKSTSTAYMELSSLRSE |
| | SGSGSGTDFTLTISSLQAEDVAVYYCLQNKEVP | DTAVYYCARAEFISTVVAPYYYALDYWGQGTLVTVSSASTKGPSV |
| | YTFGGGTKVEIKRTVAAPSVFIFPPSDEQLKSGT | FPLAPSSKSTSGGTAALGCLVKDYFPEPVTVSWNSGALTSGVHTFP |
| | ASVVCLLNNFYPREAKVQWKVDNALQSGNSQ | AVLQSSGLYSLSSVVTVPSSSLGTQTYICNVNHKPSNTKVDKKVEP |
| | ESVTEQDSKDSTYSLSSTLTLSKADYEKHKVYA | KSCDKTHTCPPCPAPEAAGGPSVFLFPPKPKDTLYITREPEVTCVVV |
| | CEVTHQGLSSPVTKSFNRGEC | DVSHEDPEVKFNWYVDGVEVHNAKTKPREEQYNSTYRVVSVLTV |
| | | LHQDWLNGKEYKCKVSNKALPAPIEKTISKAKGQPREPQVYTLPP |
| | | SRDELTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGKGGGGSGGGGSGGGGSGGGGSEIQLVESGGGLIQPGGSLRIS |
| | | CAVSGFSLSSYGVDWVRQAPGKCLEWLGVIWGFGGTNYNSALKS |
| | | RLTISKDNSKNTVYLQMNSLRAEDTAVYYCASGNFDAMDYWGQ |
| | | GTLVTVSSGGGGSGGGGSGGGGSGGGGSDIQMTQSPSSLSASVGD |
| | | RVTITCRASQDISNYLNWYQQKPGKAVKLLIYYTSRLHSGVPSRFS |
| | | GSGSGTDYTLTISSLQPEDFATYFCQQGNTLPFTFGCGTKLEIK |
| TLb0110-1 | DIVMTQSPDSLAVSLGERATINCKASKKVTIYG | QVQLVQSGAEVKKPGSSVKVSCKASGYTFTSYGITWVRQAPGQG |
| | STSALHWYQQKPGQPPKLIYNLAKPESGVPDRF | LEWMGENYPRSGNTYYNEKFKGRVTITADKSTSTAYMELSSLRSE |
| | SGSGSGTDFTLTISSLQAEDVAVYYCLQNKEVP | DTAVYYCARAEFISTVVAPYYYALDYWGQGTLVTVSSASTKGPSV |
| | YTFGGGTKVEIKRTVAAPSVFIFPPSDEQLKSGT | FPLAPSSKSTSGGTAALGCLVKDYFPEPVTVSWNSGALTSGVHTFP |
| | ASVVCLLNNFYPREAKVQWKVDNALQSGNSQ | AVLQSSGLYSLSSVVTVPSSSLGTQTYICNVNHKPSNTKVDKKVEP |
| | ESVTEQDSKDSTYSLSSTLTLSKADYEKHKVYA | KSCDKTHTCPPCPAPEAAGGPSVFLFPPKPKDTLMISRTPEVTCVV |
| | CEVTHQGLSSPVTKSFNRGEC | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSRDELTKNQVSLTCLVKGFYPSDIAVEWESNGQPENNYKTTPPVL |
| | | DSDGSFFLYSKLTVDKSRWQQGNVFSCSVLHEALHSHYTQKSLSL |
| | | SPGKGGGGSGGGGSGGGGSGGGGSEIQLVESGGGLIQPGGSLRISC |
| | | AVSGFSLSSYGVDWVRQAPGKCLEWLGVIWGFGGTNYNSALKSR |
| | | LTISKDNSKNTVYLQMNSLRAEDTAVYYCASGNFDAMDYWGQG |
| | | TLVTVSSGGGGSGGGGSGGGGSGGGGSDIQMTQSPSSLSASVGDR |
| | | VTITCRASQDISNYLNWYQQKPGKAVKLLIYYTSRLHSGVPSRFSG |
| | | SGSGTDYTLTISSLQPEDFATYFCQQGNTLPFTFGCGTKLEIK |
| TLb0110-2 | DIVMTQSPDSLAVSLGERATINCKASKKVTIYG | QVQLVQSGAEVKKPGSSVKVSCKASGYTFTSYGITWVRQAPGQG |
| | STSALHWYQQKPGQPPKLIYNLAKPESGVPDRF | LEWMGENYPRSGNTYYNEKFKGRVTITADKSTSTAYMELSSLRSE |
| | SGSGSGTDFTLTISSLQAEDVAVYYCLQNKEVP | DTAVYYCARAEFISTVVAPYYYALDYWGQGTLVTVSSASTKGPSV |
| | YTFGGGTKVEIKRTVAAPSVFIFPPSDEQLKSGT | FPLAPSSKSTSGGTAALGCLVKDYFPEPVTVSWNSGALTSGVHTFP |
| | ASVVCLLNNFYPREAKVQWKVDNALQSGNSQ | AVLQSSGLYSLSSVVTVPSSSLGTQTYICNVNHKPSNTKVDKKVEP |
| | ESVTEQDSKDSTYSLSSTLTLSKADYEKHKVYA | KSCDKTHTCPPCPAPEAAGGPSVFLFPPKPKDTLMISRTPEVTCVV |
| | CEVTHQGLSSPVTKSFNRGEC | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSRDELTKNQVSLTCLVKGFYPSDIAVEWESNGQPENNYKTTPPVL |
| | | DSDGSFFLYSKLTVDKSRWQQGNVFSCSVLHEALHSHYTQKSLSL |
| | | SPGKGGGGSGGGGSEIQLVESGGGLIQPGGSLRISCAVSGFSLSSYG |
| | | VDWVRQAPGKCLEWLGVIWGFGGTNYNSALKSRLTISKDNSKNT |
| | | VYLQMNSLRAEDTAVYYCASGNFDAMDYWGQGTLVTVSSGGGG |
| | | SGGGGSGGGGSGGGGSDIQMTQSPSSLSASVGDRVTITCRASQDIS |
| | | NYLNWYQQKPGKAVKLLIYYTSRLHSGVPSRFSGSGSGTDYTLTIS |
| | | SLQPEDFATYFCQQGNTLPFTFGCGTKLEIK |
| TLb0110-3 | DIVMTQSPDSLAVSLGERATINCKASKKVTIYG | QVQLVQSGAEVKKPGSSVKVSCKASGYTFTSYGITWVRQAPGQG |
| | STSALHWYQQKPGQPPKLIYNLAKPESGVPDRF | LEWMGENYPRSGNTYYNEKFKGRVTITADKSTSTAYMELSSLRSE |
| | SGSGSGTDFTLTISSLQAEDVAVYYCLQNKEVP | DTAVYYCARAEFISTVVAPYYYALDYWGQGTLVTVSSASTKGPSV |
| | YTFGGGTKVEIKRTVAAPSVFIFPPSDEQLKSGT | FPLAPSSKSTSGGTAALGCLVKDYFPEPVTVSWNSGALTSGVHTFP |
| | ASVVCLLNNFYPREAKVQWKVDNALQSGNSQ | AVLQSSGLYSLSSVVTVPSSSLGTQTYICNVNHKPSNTKVDKKVEP |
| | ESVTEQDSKDSTYSLSSTLTLSKADYEKHKVYA | KSCDKTHTCPPCPAPEAAGGPSVFLFPPKPKDTLMISRTPEVTCVV |
| | CEVTHQGLSSPVTKSFNRGEC | VDVSHEDPEVKFNWYVDGVEVHNAKTKPREEQYNSTYRVVSVLT |
| | | VLHQDWLNGKEYKCKVSNKALPAPIEKTISKAKGQPREPQVYTLP |
| | | PSRDELTKNQVSLTCLVKGFYPSDIAVEWESNGQPENNYKTTPPVL |
| | | DSDGSFFLYSKLTVDKSRWQQGNVFSCSVLHEALHSHYTQKSLSL |
| | | SPGKGGGGSGGGGSGGGGSEIQLVESGGGLIQPGGSLRISCAVSGF |
| | | SLSSYGVDWVRQAPGKCLEWLGVIWGFGGTNYNSALKSRLTISKD |
| | | NSKNTVYLQMNSLRAEDTAVYYCASGNFDAMDYWGQGTLVTVS |
| | | SGGGGSGGGGSGGGGSGGGGSDIQMTQSPSSLSASVGDRVTITCR |
| | | ASQDISNYLNWYQQKPGKAVKLLIYYTSRLHSGVPSRFSGSGSGT |
| | | DYTLTISSLQPEDFATYFCQQGNTLPFTFGCGTKLEIK |

TABLE 5
sequence of 1 + 1 bispecific antibodies

| | light chain amino | Heavy chain-1 amino | Heavy chain-2 amino |
|---|---|---|---|
| name | acid sequence | acid sequence | acid sequence |
| TLb0090 | AIVMTQSPATLSVSPGERVTL | EVQLQQSGPELVKPGASVKVSCKASGY | DIQMTQSPSSLSASVGDRVTITCRASQDI |
| | SCRASQSISDYLYWYQQKPD | TFTRYLMHWVRQAPGQGLEWIGYINPY | SNYLNWYQQKPGKAVKLLIYYTSRLHS |
| | QSPKLLIKFASQSISGVPSRFS | NDGTKYNQKFQGRVTITADKSTSTAYM | GVPSRFSGSGSGTDYTLTISSLQPEDFAT |
| | GSGSGRDFTLTISSLQPEDFA | ELSSLTSEDTAVYYCTSNWDLDYWGQG | YFCQQGNTLPFTFGCGTKLEIKGGGGSG |
| | VYYCQNGHSFPFTFGGGTKL | TLVTVSSASTKGPSVFPLAPSSKSTSGGT | GGGSGGGGSGGGGSEIQLVESGGGLIQP |
| | EIKRTVAAPSVFIFPPSDEQL | AALGCLVKDYFPEPVTVSWNSGALTSG | GGSLRISCAVSGFSLSSYGVDWVRQAPG |
| | KSGTASVVCLLNNFYPREAK | VHTFPAVLQSSGLYSLSSVVTVPSSSLGT | KCLEWLGVIWGFGGTNYNSALKSRLTIS |
| | VQWKVDNALQSGNSQESVT | QTYICNVNHKPSNTKVDKKVEPKSCDK | KDNSKNTVYLQMNSLRAEDTAVYYCAS |
| | EQDSKDSTYSLSSTLTLSKAD | THTCPPCPAPEAAGGPSVFLFPPKPKDTL | GNFDAMDYWGQGTLVTVSSEPKSADKT |
| | YEKHKVYACEVTHQGLSSP | YITREPEVTCVVVDVSHEDPEVKFNWY | HTCPPCPAPEAAGGPSVFLFPPKPKDTLY |
| | VTKSFNRGEC | VDGVEVHNAKTKPREEQYNSTYRVVSV | ITREPEVTCVVVDVSHEDPEVKFNWYV |
| | | LTVLHQDWLNGKEYKCKVSNKALPAPI | DGVEVHNAKTKPREEQYNSTYRVVSVL |
| | | EKTISKAKGQPREPQVYTLPPCREEMTK | TVLHQDWLNGKEYKCKVSNKALPAPIE |
| | | NQVSLWCLVKGFYPSDIAVEWESNGQP | KTISKAKGQPREPQVCTLPPSREEMTKN |
| | | ENNYKTTPPVLDSDGSFFLYSKLTVDKS | QVSLSCAVKGFYPSDIAVEWESNGQPEN |
| | | RWQQGNVFSCSVMHEALHNHYTQKSLS | NYKTTPPVLDSDGSFFLVSKLTVDKSRW |
| | | LSPGK | QQGNVFSCSVMHEALHNRFTQKSLSLSP |
| | | | GK |
| TLb0091 | EIVMTQSPATLSLSPGERATL | QVQLQQSGSELKKPGASVKVSCKASGY | DIQMTQSPSSLSASVGDRVTITCRASQDI |
| | SCRASQSISDYLYWYQQKPD | TFTRYLMHWVRQAPGKGLEWIGYINPY | SNYLNWYQQKPGKAVKLLIYYTSRLHS |
| | QSPKLLIKFASQSISGVPSRFS | NDGTKYNQKFQGRVTITADKSTSTAYM | GVPSRFSGSGSGTDYTLTISSLQPEDFAT |
| | GSGSGTEFTLTISSLQPEDFA | ELSSLRSEDTAVYYCTSNWDLDYWGQG | YFCQQGNTLPFTFGCGTKLEIKGGGGSG |
| | VYYCQNGHSFPFTFGGGTKV | TLVTVSSASTKGPSVFPLAPSSKSTSGGT | GGGSGGGGSGGGGSEIQLVESGGGLIQP |
| | EIKRTVAAPSVFIFPPSDEQL | AALGCLVKDYFPEPVTVSWNSGALTSG | GGSLRISCAVSGFSLSSYGVDWVRQAPG |
| | KSGTASVVCLLNNFYPREAK | VHTFPAVLQSSGLYSLSSVVTVPSSSLGT | KCLEWLGVIWGFGGTNYNSALKSRLTIS |
| | VQWKVDNALQSGNSQESVT | QTYICNVNHKPSNTKVDKKVEPKSCDK | KDNSKNTVYLQMNSLRAEDTAVYYCAS |
| | EQDSKDSTYSLSSTLTLSKAD | THTCPPCPAPEAAGGPSVFLFPPKPKDTL | GNFDAMDYWGQGTLVTVSSEPKSADKT |
| | YEKHKVYACEVTHQGLSSP | YITREPEVTCVVVDVSHEDPEVKFNWY | HTCPPCPAPEAAGGPSVFLFPPKPKDTLY |
| | VTKSFNRGEC | VDGVEVHNAKTKPREEQYNSTYRVVSV | ITREPEVTCVVVDVSHEDPEVKFNWYV |
| | | LTVLHQDWLNGKEYKCKVSNKALPAPI | DGVEVHNAKTKPREEQYNSTYRVVSVL |
| | | EKTISKAKGQPREPQVYTLPPCREEMTK | TVLHQDWLNGKEYKCKVSNKALPAPIE |
| | | NQVSLWCLVKGFYPSDIAVEWESNGQP | KTISKAKGQPREPQVCTLPPSREEMTKN |
| | | ENNYKTTPPVLDSDGSFFLYSKLTVDKS | QVSLSCAVKGFYPSDIAVEWESNGQPEN |
| | | RWQQGNVFSCSVMHEALHNHYTQKSLS | NYKTTPPVLDSDGSFFLVSKLTVDKSRW |
| | | LSPGK | QQGNVFSCSVMHEALHNRFTQKSLSLSP |
| | | | GK |
| TLb0092 | DIVMTQSPATLSVSPGERVTL | EIQLQQSGSELKKPGASVKVSCKASGYT | DIQMTQSPSSLSASVGDRVTITCRASQDI |
| | SCRASQSISDYLYWYQQKPD | FTRYLMNWVRQAPGKGLEWIGYINPYN | SNYLNWYQQKPGKAVKLLIYYTSRLHS |
| | QSPKLLIKFASQSISGVPSRFS | DGTKYNQKFQGRVTITADKSTSTAYME | GVPSRFSGSGSGTDYTLTISSLQPEDFAT |
| | GSGSGSDFTLTISSLQPEDFA | LSSLRSEDTAVYYCTSNWDLDYWGQGT | YFCQQGNTLPFTFGCGTKLEIKGGGGSG |
| | TYYCQNGHSFPFTFGGGTKL | LVTVSSASTKGPSVFPLAPSSKSTSGGTA | GGGSGGGGSGGGGSEIQLVESGGGLIQP |
| | EIKRTVAAPSVFIFPPSDEQL | ALGCLVKDYFPEPVTVSWNSGALTSGV | GGSLRISCAVSGFSLSSYGVDWVRQAPG |
| | KSGTASVVCLLNNFYPREAK | HTFPAVLQSSGLYSLSSVVTVPSSSLGTQ | KCLEWLGVIWGFGGTNYNSALKSRLTIS |
| | VQWKVDNALQSGNSQESVT | TYICNVNHKPSNTKVDKKVEPKSCDKT | KDNSKNTVYLQMNSLRAEDTAVYYCAS |
| | EQDSKDSTYSLSSTLTLSKAD | HTCPPCPAPEAAGGPSVFLFPPKPKDTLY | GNFDAMDYWGQGTLVTVSSEPKSADKT |
| | YEKHKVYACEVTHQGLSSP | ITREPEVTCVVVDVSHEDPEVKFNWYV | HTCPPCPAPEAAGGPSVFLFPPKPKDTLY |
| | VTKSFNRGEC | DGVEVHNAKTKPREEQYNSTYRVVSVL | ITREPEVTCVVVDVSHEDPEVKFNWYV |
| | | TVLHQDWLNGKEYKCKVSNKALPAPIE | DGVEVHNAKTKPREEQYNSTYRVVSVL |
| | | KTISKAKGQPREPQVYTLPPCREEMTKN | TVLHQDWLNGKEYKCKVSNKALPAPIE |
| | | QVSLWCLVKGFYPSDIAVEWESNGQPE | KTISKAKGQPREPQVCTLPPSREEMTKN |
| | | NNYKTTPPVLDSDGSFFLYSKLTVDKSR | QVSLSCAVKGFYPSDIAVEWESNGQPEN |
| | | WQQGNVFSCSVMHEALHNHYTQKSLSL | NYKTTPPVLDSDGSFFLVSKLTVDKSRW |
| | | SPGK | QQGNVFSCSVMHEALHNRFTQKSLSLSP |
| | | | GK |
| TLb0100 | EIVMTQSPATLSVSPGERVTL | QVQLVQSGAEVVKPGASVKMSCKASGY | DIQMTQSPSSLSASVGDRVTITCRASQDI |
| | SCRASQSISDYLYWYQQKPG | TFTRYLIHWVKQAPGQGLEWIGYINPYN | SNYLNWYQQKPGKAVKLLIYYTSRLHS |
| | QAPRLLIKFASQSISGIPARFS | EGTKYNEKFKGRATLTSDKSTSTAYMEL | GVPSRFSGSGSGTDYTLTISSLQPEDFAT |
| | GSGSGSEFTLTISSVQSEDFA | SSLRSEDTAVYYCTSNWDLDYWGQGTL | YFCQQGNTLPFTFGCGTKLEIKGGGGSG |
| | VFFCQQGHSFPFTFGGGTKV | VTVSSASTKGPSVFPLAPSSKSTSGGTAA | GGGSGGGGSGGGGSEIQLVESGGGLIQP |
| | EIKRTVAAPSVFIFPPSDEQL | LGCLVKDYFPEPVTVSWNSGALTSGVH | GGSLRISCAVSGFSLSSYGVDWVRQAPG |
| | KSGTASVVCLLNNFYPREAK | TFPAVLQSSGLYSLSSVVTVPSSSLGTQT | KCLEWLGVIWGFGGTNYNSALKSRLTIS |
| | VQWKVDNALQSGNSQESVT | YICNVNHKPSNTKVDKKVEPKSCDKTH | KDNSKNTVYLQMNSLRAEDTAVYYCAS |
| | EQDSKDSTYSLSSTLTLSKAD | TCPPCPAPEAAGGPSVFLFPPKPKDTLYI | GNFDAMDYWGQGTLVTVSSEPKSADKT |
| | YEKHKVYACEVTHQGLSSP | TREPEVTCVVVDVSHEDPEVKFNWYVD | HTCPPCPAPEAAGGPSVFLFPPKPKDTLY |
| | VTKSFNRGEC | GVEVHNAKTKPREEQYNSTYRVVSVLT | ITREPEVTCVVVDVSHEDPEVKFNWYV |
| | | VLHQDWLNGKEYKCKVSNKALPAPIEK | DGVEVHNAKTKPREEQYNSTYRVVSVL |
| | | TISKAKGQPREPQVYTLPPCREEMTKNQ | TVLHQDWLNGKEYKCKVSNKALPAPIE |
| | | VSLWCLVKGFYPSDIAVEWESNGQPEN | KTISKAKGQPREPQVCTLPPSREEMTKN |
| | | NYKTTPPVLDSDGSFFLYSKLTVDKSRW | QVSLSCAVKGFYPSDIAVEWESNGQPEN |
| | | QQGNVFSCSVMHEALHNHYTQKSLSLS | NYKTTPPVLDSDGSFFLVSKLTVDKSRW |
| | | PGK | QQGNVFSCSVMHEALHNRFTQKSLSLSP |
| | | | GK |
| TLb0102 | EIVMTQSPATLSVSPGERVTL | QVQLVQSGAEVVKPGASVKMSCKASGY | DIQMTQSPSSLSASVGDRVTITCRASQDI |
| | SCRASQSISDYLYWYQQKPG | TFTRYLIHWVKQAPGQGLEWIGYINPYN | SNYLNWYQQKPGKAVKLLIYYTSRLHS |
| | QAPRLLIKFASQSISGIPARFS | EGTKYNEKFKGRATLTSDKSTSTAYMEL | GVPSRFSGSGSGTDYTLTISSLQPEDFAT |
| | GSGSGSEFTLTISSVQSEDFA | SSLRSEDTAVYYCTSNWDLDYWGQGTL | YFCQQGNTLPFTFGCGTKLEIKGGGGSG |
| | VFFCQNGHSFPFTFGGGTKV | VTVSSASTKGPSVFPLAPSSKSTSGGTAA | GGGSGGGGSGGGGSEIQLVESGGGLIQP |
| | EIKRTVAAPSVFIFPPSDEQL | LGCLVKDYFPEPVTVSWNSGALTSGVH | GGSLRISCAVSGFSLSSYGVDWVRQAPG |
| | KSGTASVVCLLNNFYPREAK | TFPAVLQSSGLYSLSSVVTVPSSSLGTQT | KCLEWLGVIWGFGGTNYNSALKSRLTIS |
| | VQWKVDNALQSGNSQESVT | YICNVNHKPSNTKVDKKVEPKSCDKTH | KDNSKNTVYLQMNSLRAEDTAVYYCAS |
| | EQDSKDSTYSLSSTLTLSKAD | TCPPCPAPEAAGGPSVFLFPPKPKDTLYI | GNFDAMDYWGQGTLVTVSSEPKSADKT |
| | YEKHKVYACEVTHQGLSSP | TREPEVTCVVVDVSHEDPEVKFNWYVD | HTCPPCPAPEAAGGPSVFLFPPKPKDTLY |
| | VTKSFNRGEC | GVEVHNAKTKPREEQYNSTYRVVSVLT | ITREPEVTCVVVDVSHEDPEVKFNWYV |
| | | VLHQDWLNGKEYKCKVSNKALPAPIEK | DGVEVHNAKTKPREEQYNSTYRVVSVL |
| | | TISKAKGQPREPQVYTLPPCREEMTKNQ | TVLHQDWLNGKEYKCKVSNKALPAPIE |
| | | VSLWCLVKGFYPSDIAVEWESNGQPEN | KTISKAKGQPREPQVCTLPPSREEMTKN |
| | | NYKTTPPVLDSDGSFFLYSKLTVDKSRW | QVSLSCAVKGFYPSDIAVEWESNGQPEN |
| | | QQGNVFSCSVMHEALHNHYTQKSLSLS | NYKTTPPVLDSDGSFFLVSKLTVDKSRW |
| | | PGK | QQGNVFSCSVMHEALHNRFTQKSLSLSP |
| | | | GK |
| TLb0102-1 | EIVMTQSPATLSVSPGERVTL | QVQLVQSGAEVVKPGASVKMSCKASGY | DIQMTQSPSSLSASVGDRVTITCRASQDI |
| | SCRASQSISDYLYWYQQKPG | TFTRYLIHWVKQAPGQGLEWIGYINPYN | SNYLNWYQQKPGKAVKLLIYYTSRLHS |
| | QAPRLLIKFASQSISGIPARFS | EGTKYNEKFKGRATLTSDKSTSTAYMEL | GVPSRFSGSGSGTDYTLTISSLQPEDFAT |
| | GSGSGSEFTLTISSVQSEDFA | SSLRSEDTAVYYCTSNWDLDYWGQGTL | YFCQQGNTLPFTFGCGTKLEIKGGGGSG |
| | VFFCQNGHSFPFTFGGGTKV | VTVSSASTKGPSVFPLAPSSKSTSGGTAA | GGGSGGGGSGGGGSEIQLVESGGGLIQP |
| | EIKRTVAAPSVFIFPPSDEQL | LGCLVKDYFPEPVTVSWNSGALTSGVH | GGSLRISCAVSGFSLSSYGVDWVRQAPG |
| | KSGTASVVCLLNNFYPREAK | TFPAVLQSSGLYSLSSVVTVPSSSLGTQT | KCLEWLGVIWGFGGTNYNSALKSRLTIS |
| | VQWKVDNALQSGNSQESVT | YICNVNHKPSNTKVDKKVEPKSCDKTH | KDNSKNTVYLQMNSLRAEDTAVYYCAS |
| | EQDSKDSTYSLSSTLTLSKAD | TCPPCPAPEAAGGPSVFLFPPKPKDTLMI | GNFDAMDYWGQGTLVTVSSEPKSADKT |
| | YEKHKVYACEVTHQGLSSP | SRTPEVTCVVVDVSHEDPEVKFNWYVD | HTCPPCPAPEAAGGPSVFLFPPKPKDTL |
| | VTKSFNRGEC | GVEVHNAKTKPREEQYNSTYRVVSVLT | MISRTPEVTCVVVDVSHEDPEVKFNWY |
| | | VLHQDWLNGKEYKCKVSNKALPAPIEK | VDGVEVHNAKTKPREEQYNSTYRVVSV |
| | | TISKAKGQPREPQVYTLPPCREEMTKNQ | LTVLHQDWLNGKEYKCKVSNKALPAPI |
| | | VSLWCLVKGFYPSDIAVEWESNGQPEN | EKTISKAKGQPREPQVCTLPPSREEMTK |
| | | NYKTTPPVLDSDGSFFLYSKLTVDKSRW | NQVSLSCAVKGFYPSDIAVEWESNGQPE |
| | | QQGNVFSCSVLHEALHSHYTQKSLSLSP | NNYKTTPPVLDSDGSFFLVSKLTVDKSR |
| | | GK | WQQGNVFSCSVLHEALHSRFTQKSLSLS |
| | | | PGK |
| TLb0109 | DIVMTQSPDSLAVSLGERATI | QVQLVQSGAEVKKPGSSVKVSCKASGY | DIQMTQSPSSLSASVGDRVTITCRASQDI |
| | NCKASKKVTIYGSTSALHWY | TFTSYGITWVRQAPGQGLEWMGENYPR | SNYLNWYQQKPGKAVKLLIYYTSRLHS |
| | QQKPGQPPKLIYNLAKPESG | SGNTYYNEKFKGRVTITADKSTSTAYME | GVPSRFSGSGSGTDYTLTISSLQPEDFAT |
| | VPDRFSGSGSGTDFTLTISSL | LSSLRSEDTAVYYCARAEFISTVVAPYY | YFCQQGNTLPFTFGCGTKLEIKGGGGSG |
| | QAEDVAVYYCLQNKEVPYT | YALDYWGQGTLVTVSSASTKGPSVFPL | GGGSGGGGSGGGGSEIQLVESGGGLIQP |
| | FGGGTKVEIKRTVAAPSVFIF | APSSKSTSGGTAALGCLVKDYFPEPVTV | GGSLRISCAVSGFSLSSYGVDWVRQAPG |
| | PPSDEQLKSGTASVVCLLNN | SWNSGALTSGVHTFPAVLQSSGLYSLSS | KCLEWLGVIWGFGGTNYNSALKSRLTIS |
| | FYPREAKVQWKVDNALQSG | VVTVPSSSLGTQTYICNVNHKPSNTKVD | KDNSKNTVYLQMNSLRAEDTAVYYCAS |
| | NSQESVTEQDSKDSTYSLSST | KKVEPKSCDKTHTCPPCPAPEAAGGPSV | GNFDAMDYWGQGTLVTVSSEPKSADKT |
| | LTLSKADYEKHKVYACEVT | FLFPPKPKDTLYITREPEVTCVVVDVSHE | HTCPPCPAPEAAGGPSVFLFPPKPKDTLY |
| | HQGLSSPVTKSFNRGEC | DPEVKFNWYVDGVEVHNAKTKPREEQ | ITREPEVTCVVVDVSHEDPEVKFNWYV |
| | | YNSTYRVVSVLTVLHQDWLNGKEYKC | DGVEVHNAKTKPREEQYNSTYRVVSVL |
| | | KVSNKALPAPIEKTISKAKGQPREPQVYT | TVLHQDWLNGKEYKCKVSNKALPAPIE |
| | | LPPSRDELTKNQVSLWCLVKGFYPSDIA | KTISKAKGQPREPQVCTLPPSREEMTKN |
| | | VEWESNGQPENNYKTTPPVLDSDGSFFL | QVSLSCAVKGFYPSDIAVEWESNGQPEN |
| | | YSKLTVDKSRWQQGNVFSCSVMHEALH | NYKTTPPVLDSDGSFFLVSKLTVDKSRW |
| | | NRFTQKSLSLSPGK | QQGNVFSCSVMHEALHNRFTQKSLSLSP |
| | | | GK |
| TLb0109-1 | DIVMTQSPDSLAVSLGERATI | QVQLVQSGAEVKKPGSSVKVSCKASGY | DIQMTQSPSSLSASVGDRVTITCRASQDI |
| | NCKASKKVTIYGSTSALHWY | TFTSYGITWVRQAPGQGLEWMGENYPR | SNYLNWYQQKPGKAVKLLIYYTSRLHS |
| | QQKPGQPPKLIYNLAKPESG | SGNTYYNEKFKGRVTITADKSTSTAYME | GVPSRFSGSGSGTDYTLTISSLQPEDFAT |
| | VPDRFSGSGSGTDFTLTISSL | LSSLRSEDTAVYYCARAEFISTVVAPYY | YFCQQGNTLPFTFGCGTKLEIKGGGGSG |
| | QAEDVAVYYCLQNKEVPYT | YALDYWGQGTLVTVSSASTKGPSVFPL | GGGSGGGGSGGGGSEIQLVESGGGLIQP |
| | FGGGTKVEIKRTVAAPSVFIF | APSSKSTSGGTAALGCLVKDYFPEPVTV | GGSLRISCAVSGFSLSSYGVDWVRQAPG |
| | PPSDEQLKSGTASVVCLLNN | SWNSGALTSGVHTFPAVLQSSGLYSLSS | KCLEWLGVIWGFGGTNYNSALKSRLTIS |
| | FYPREAKVQWKVDNALQSG | VVTVPSSSLGTQTYICNVNHKPSNTKVD | KDNSKNTVYLQMNSLRAEDTAVYYCAS |
| | NSQESVTEQDSKDSTYSLSST | KKVEPKSCDKTHTCPPCPAPEAAGGPSV | GNFDAMDYWGQGTLVTVSSEPKSADKT |
| | LTLSKADYEKHKVYACEVT | FLFPPKPKDTLMISRTPEVTCVVVDVSHE | HTCPPCPAPEAAGGPSVFLFPPKPKDTL |
| | HQGLSSPVTKSFNRGEC | DPEVKFNWYVDGVEVHNAKTKPREEQ | MISRTPEVTCVVVDVSHEDPEVKFNWY |
| | | YNSTYRVVSVLTVLHQDWLNGKEYKC | VDGVEVHNAKTKPREEQYNSTYRVVSV |
| | | KVSNKALPAPIEKTISKAKGQPREPQVYT | LTVLHQDWLNGKEYKCKVSNKALPAPI |
| | | LPPCREEMTKNQVSLWCLVKGFYPSDIA | EKTISKAKGQPREPQVCTLPPSREEMTK |
| | | VEWESNGQPENNYKTTPPVLDSDGSFFL | NQVSLSCAVKGFYPSDIAVEWESNGQPE |
| | | YSKLTVDKSRWQQGNVFSCSVLHEALH | NNYKTTPPVLDSDGSFFLVSKLTVDKSR |
| | | SHYTQKSLSLSPGK | WQQGNVFSCSVLHEALHSRFTQKSLSLS |
| | | | PGK |

### Example 3 Expression of Anti-TL1A+IL-23p19 Bispecific Antibody

The designed bispecific antibodies were expressed, and the bispecific antibodies with a purity lower than 9000 after the first step of purification were purified in the second step. The statistical information of the expression yield and purity is shown in Table 6. Most bispecific antibodies were expressed in a volume of 100 ml, and bispecific antibodies with purity meeting the requirements could be obtained after the second step of purification.

TABLE 6
Anti-TL1A + IL-23p19 bispecific antibody expression details

| | | | Purity | Purity | Expres- | |
|---|---|---|---|---|---|---|
| | Conc. | Amount | (SDS- | (SEC- | sion | Host |
| Abs | (mg/ml) | (mg) | PAGE) | HPLC) | Volume | cell |
| TLb0090 | 0.45 | 2.25 | >95% | 100% | 100 ml | CHO—S |
| TLb0091 | 0.62 | 4.40 | >95% | 100% | | |
| TLb0092 | 0.64 | 3.46 | >95% | 100% | | |
| TLb0100 | 1.57 | 12.56 | >95% | 97% | | |
| TLb0102 | 0.50 | 12.00 | >95% | 99% | | |
| TLb0102-1 | 1.01 | 22.22 | >95% | 97% | | |
| TLb0103 | 0.59 | 10.97 | >95% | 99% | | |
| TLb0103-1 | 0.17 | 2.72 | >95% | 98% | | |
| TLb0103-2 | 0.63 | 11.34 | >95% | 99% | | |
| TLb0103-3 | 0.52 | 8.32 | >95% | 99% | | |
| TLb0107 | 0.19 | 0.95 | >90% | 89% | | |
| TLb0108 | 0.32 | 1.60 | >75% | 85% | | |
| TLb0109 | 1.67 | 30.06 | >85% | 94% | | |
| TLb0109-1 | 2.08 | 41.60 | >90% | 97% | | |
| TLb0110 | 1.06 | 16.96 | >95% | 99% | | |
| TLb0110-1 | 0.60 | 8.10 | >95% | 99% | | |
| TLb0110-2 | 0.90 | 16.65 | >95% | 98% | | |
| TLb0110-3 | 0.80 | 6.00 | >95% | 99% | | |

### Example 4 Biacore Detection of the Affinity of IL-23p19 Monoclonal Antibody to IL-23 Protein, and the Affinity of the Bispecific Antibody to IL-23 Protein and TL1A-his Protein

Based on the Biacore biomolecular interaction instrument, the affinity of the multiple IL-23p19 monoclonal antibodies prepared by Examples 1-3 of the present invention to human IL-23 (Acrobiosystems, ILB-H52W5) and monkey IL-23 (Acrobiosystems, ILB-CM52W8) proteins, as well as the affinity of the TL1A+IL-23p19 bispecific antibodies to human IL-23 protein and monkey IL-23 protein, and human TL1A (Acrobiosystems, TLA-H5243) protein and monkey TL1A (Acrobiosystems, TLA-C5241) protein were detected.

A certain amount of the antibody to be tested was captured by affinity using the biosensor chip Protein A (Cytiva, 29127556), and then a series of different concentrations of antigen (50 nM, 25 nM, 12.5 nM, 6.25 nM, 3.125 nM, 1.5625 nM) flowed over the chip surface. The reaction signal was detected in real time using Biacore 1K (Cytiva) to obtain the binding and dissociation curves. After each cycle of dissociation was completed, the biochip was washed and regenerated with 10 mM glycine-hydrochloric acid solution pH 1.5 (Cytiva, BR-1003-54). The experimental data was fitted with a 1:1 model using Biacore Insight Evaluation Software 5.0 to calculate the affinity value.

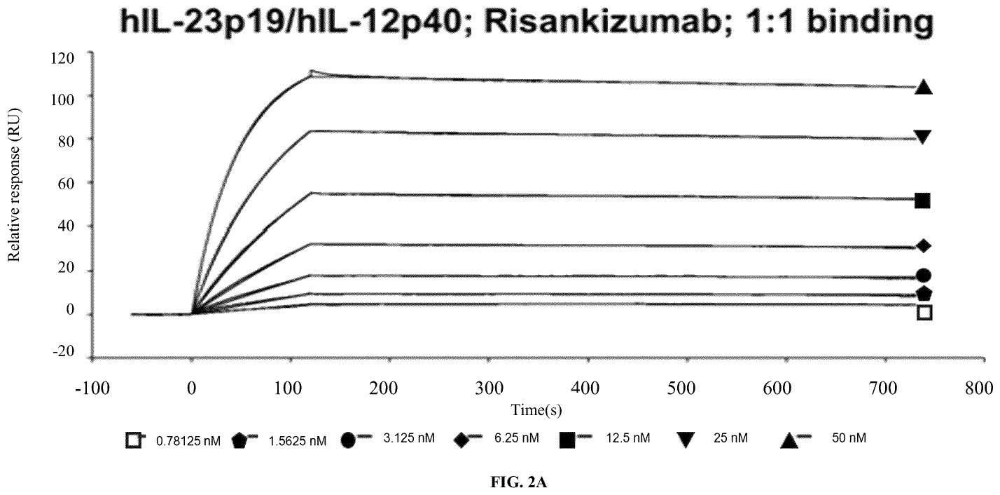

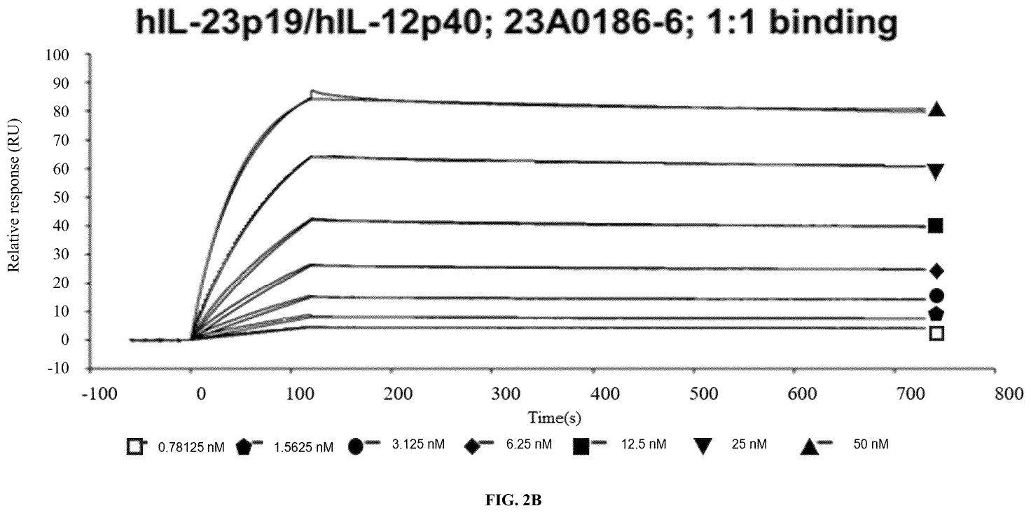

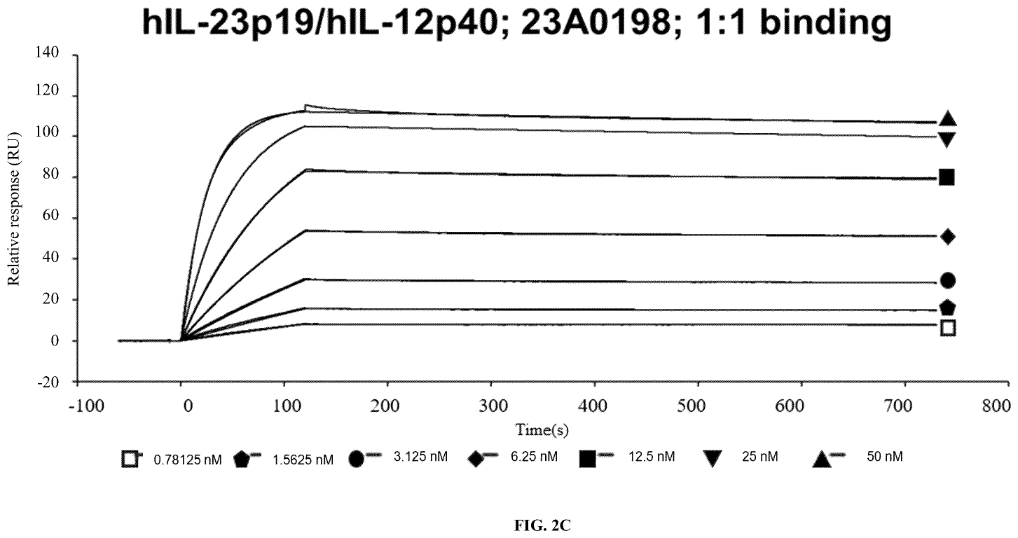

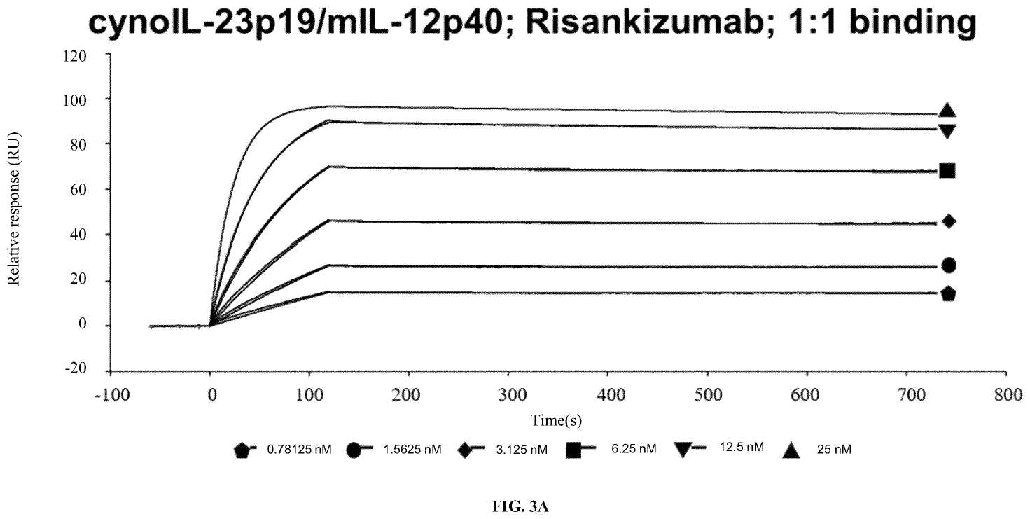

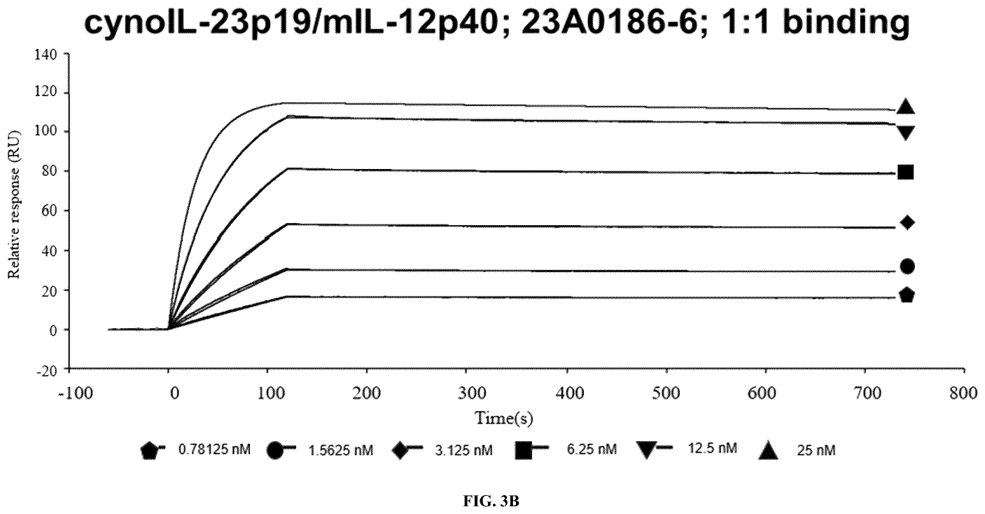

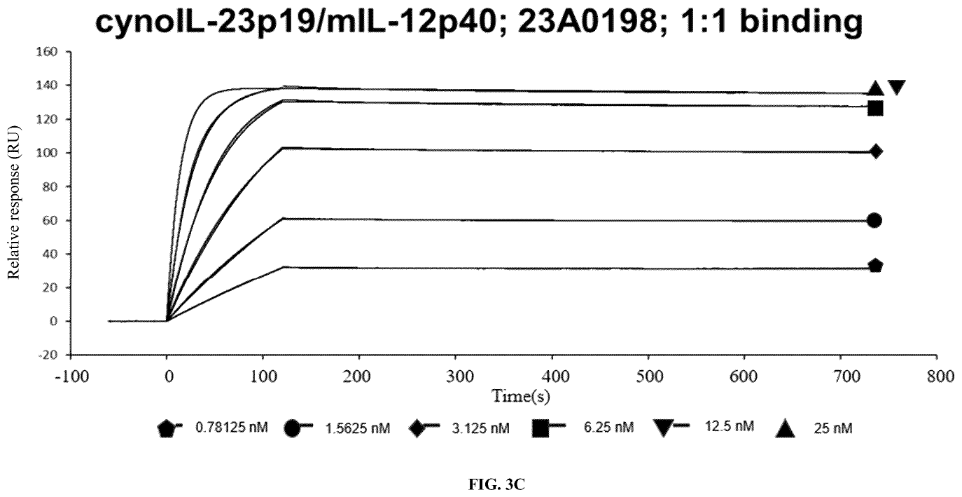

The affinity results of anti-IL-23p19 monoclonal antibody to human IL-23 protein are shown in FIG. 2, and the affinity results to monkey IL-23 protein are shown in FIG. 3. The statistical affinity values are shown in Table 7. The results show that anti-IL-23 monoclonal antibody has a high affinity to both human and monkey IL-23 proteins, and is comparable to the control antibody Risankizumab (Analogue).

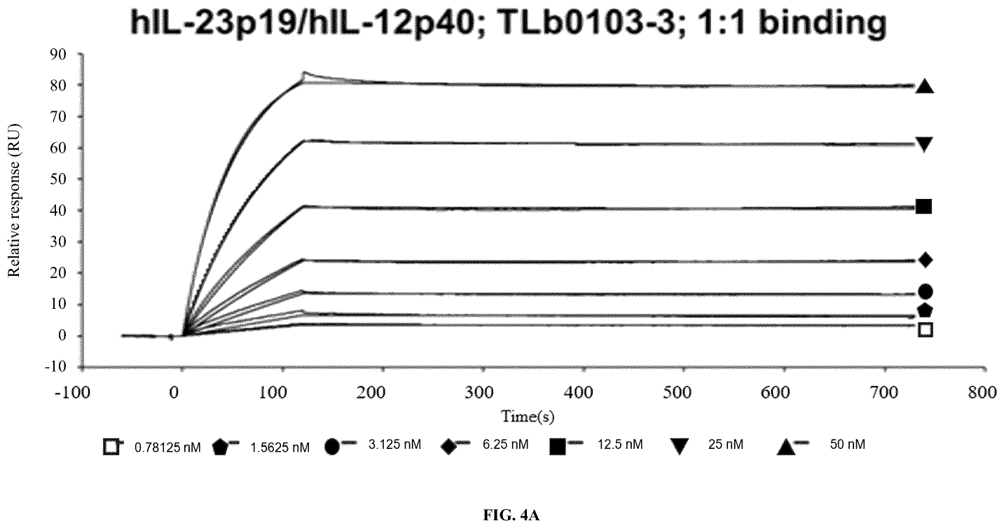

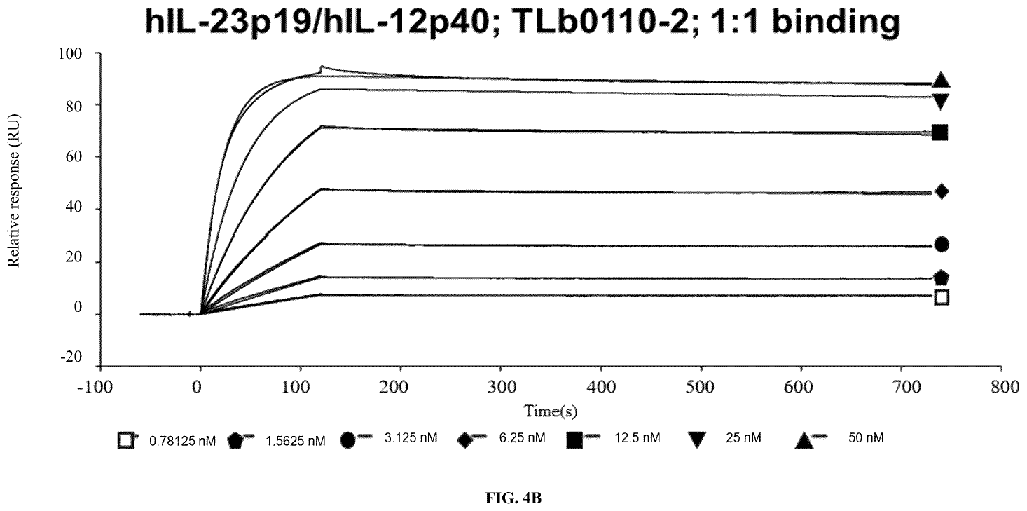

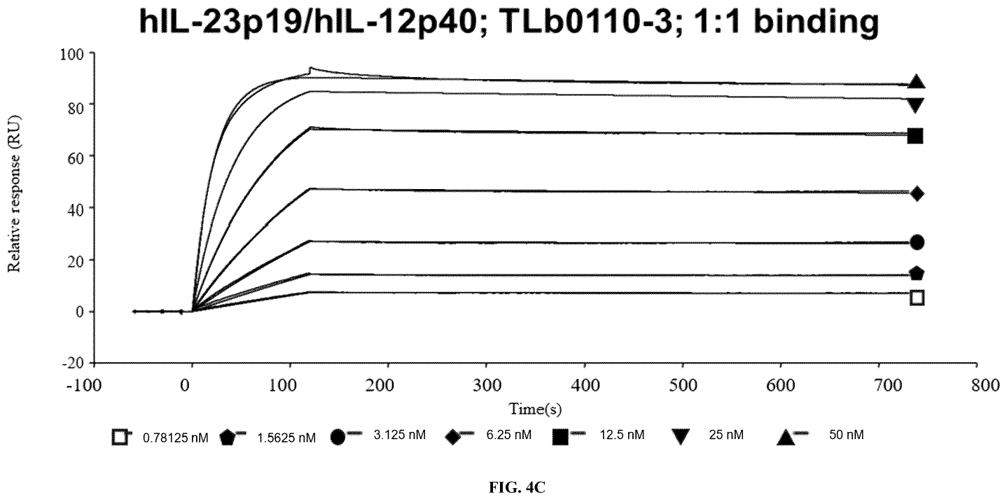

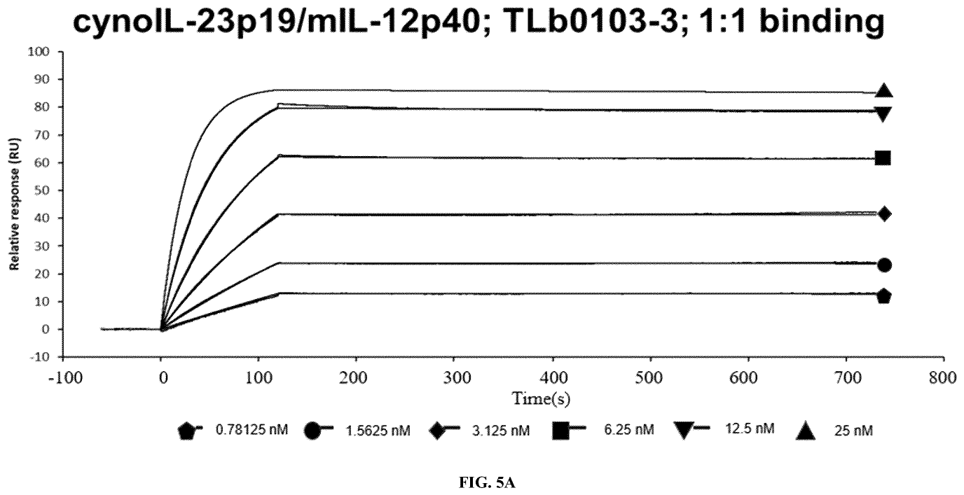

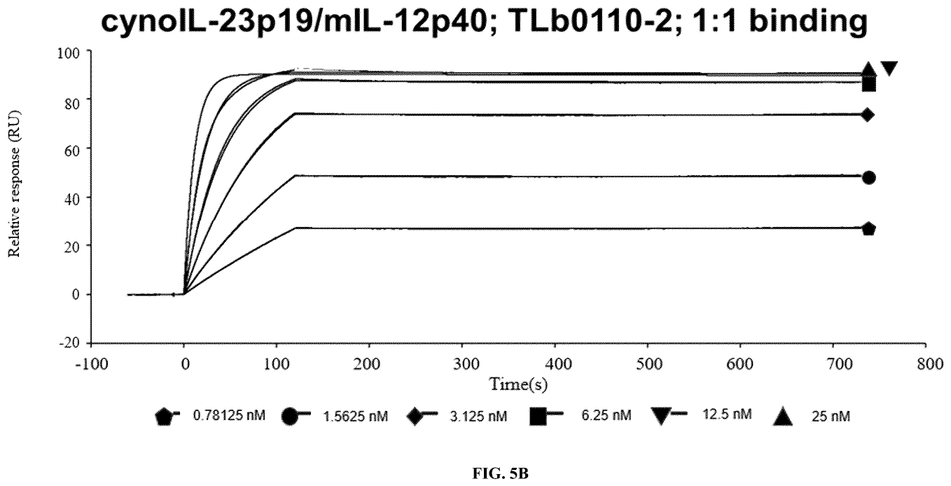

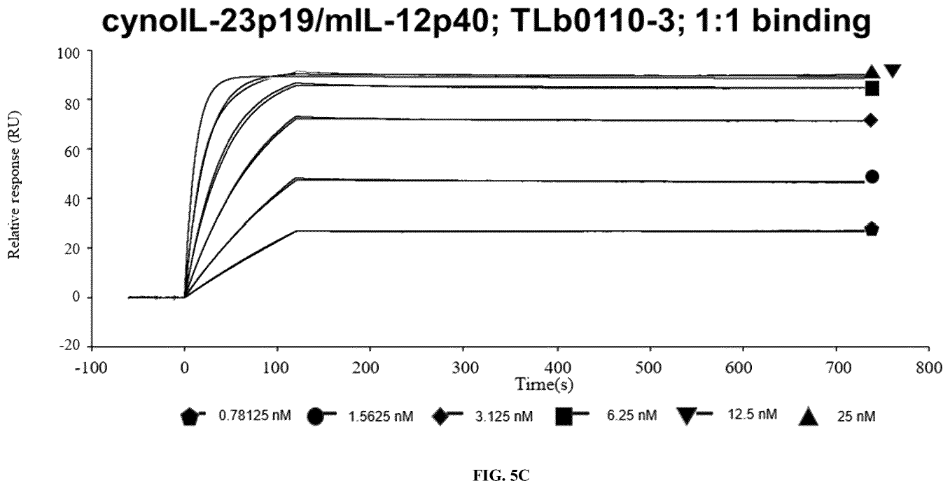

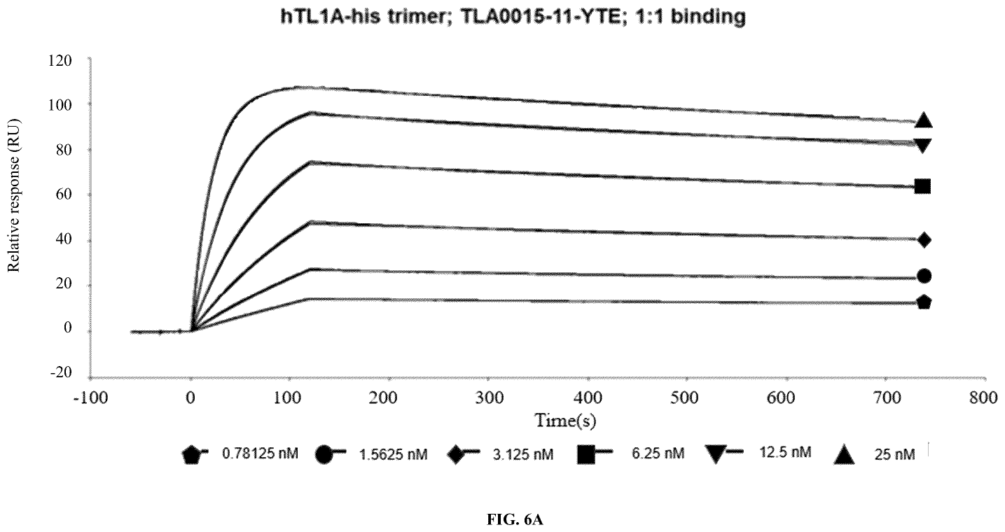

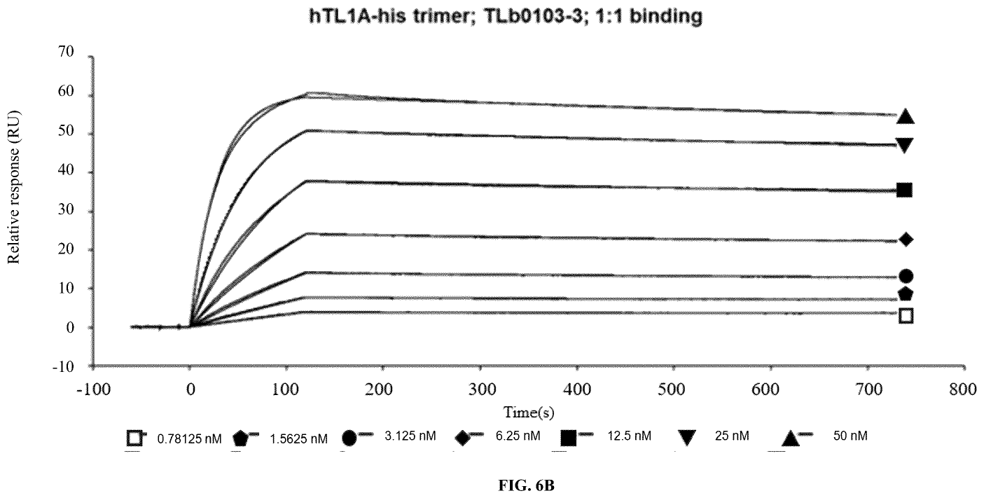

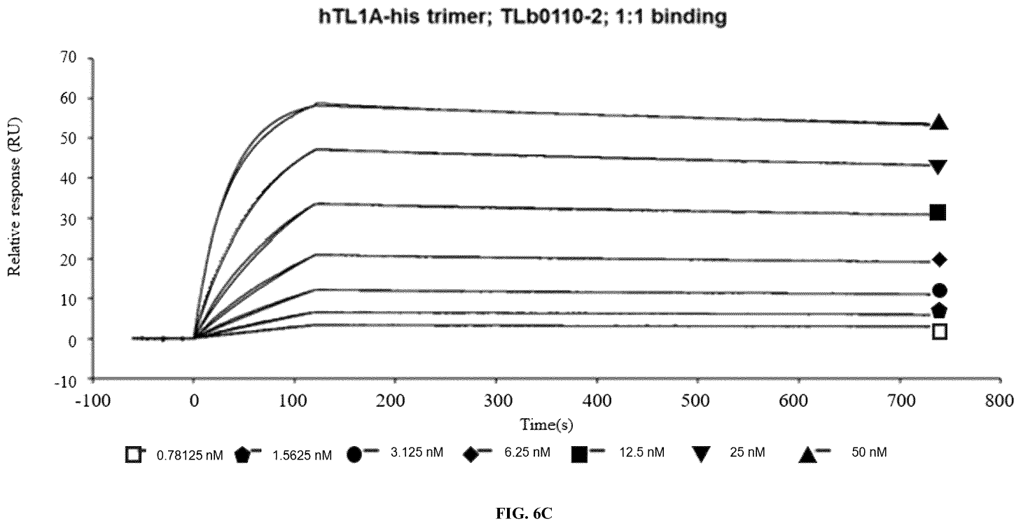

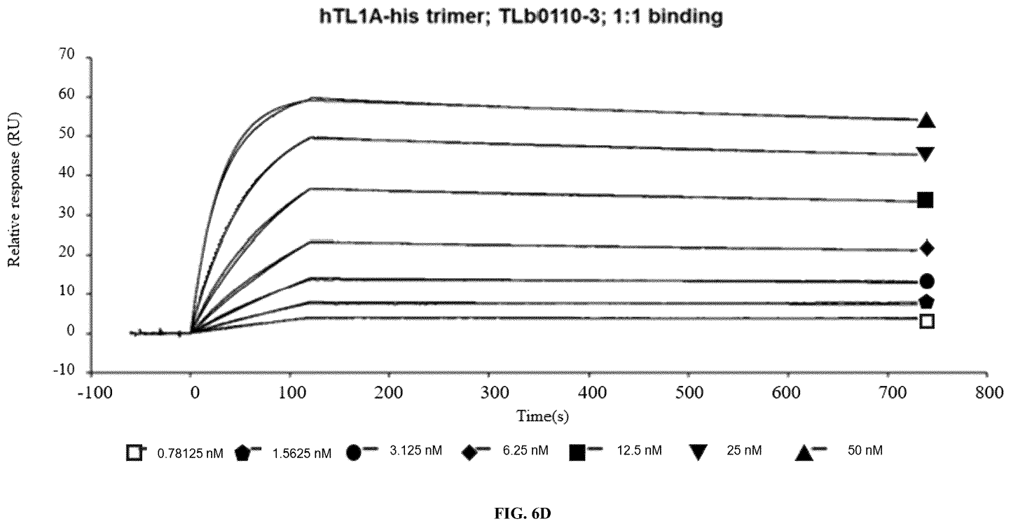

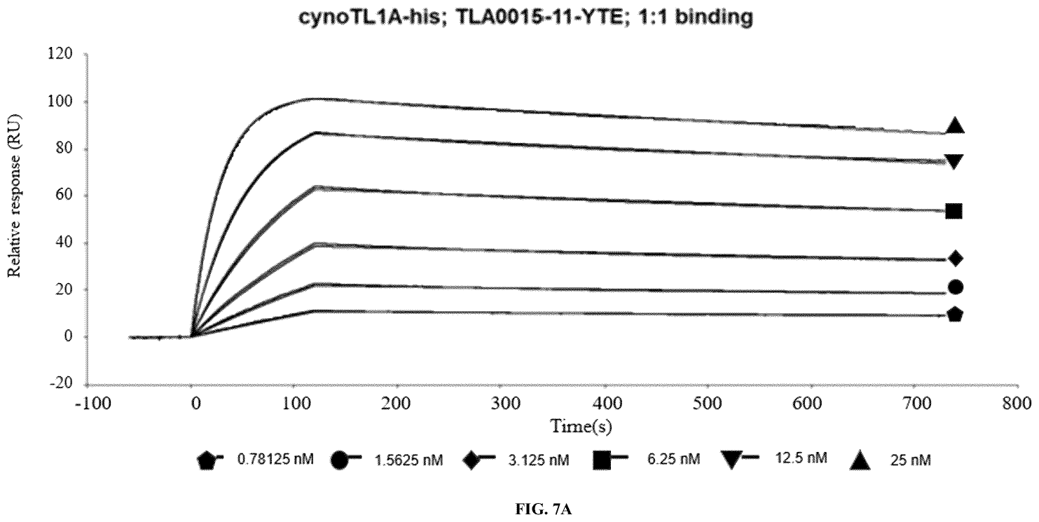

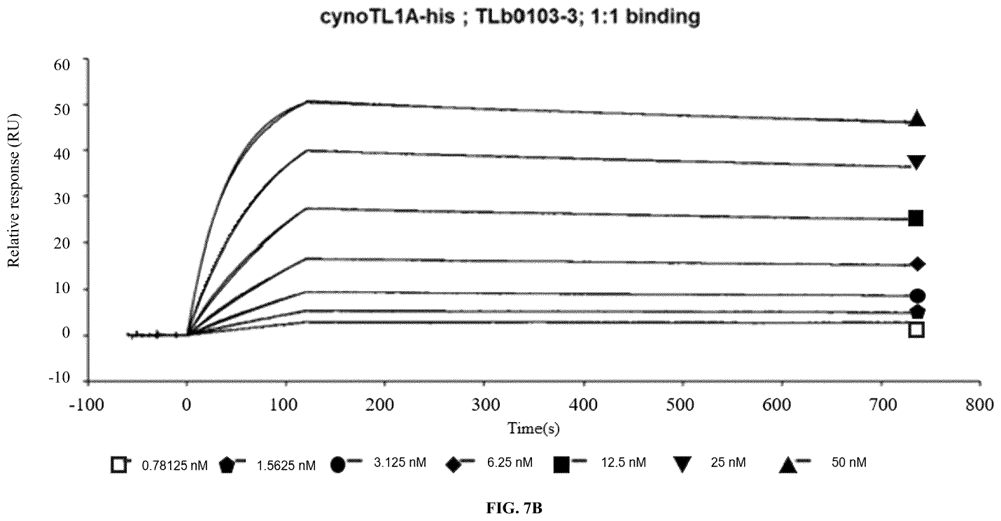

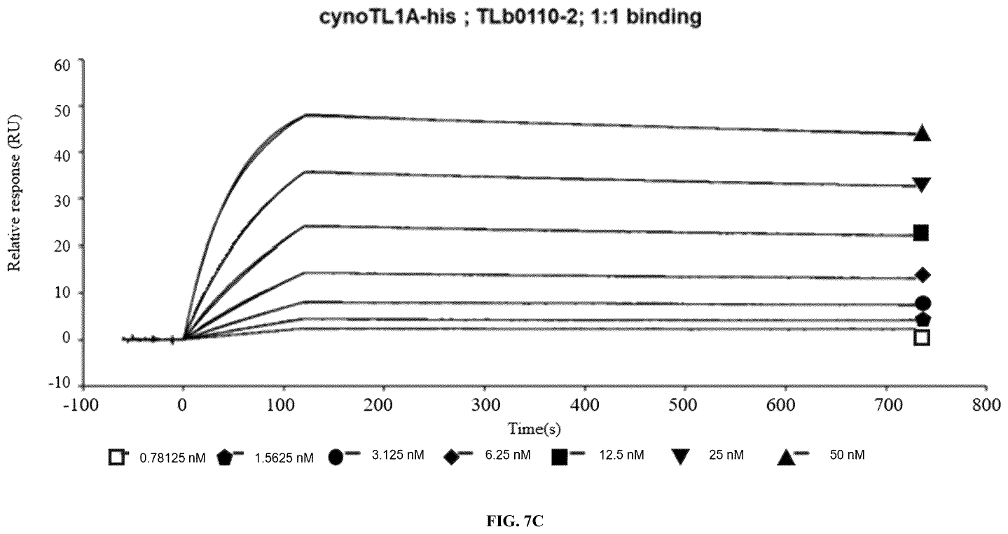

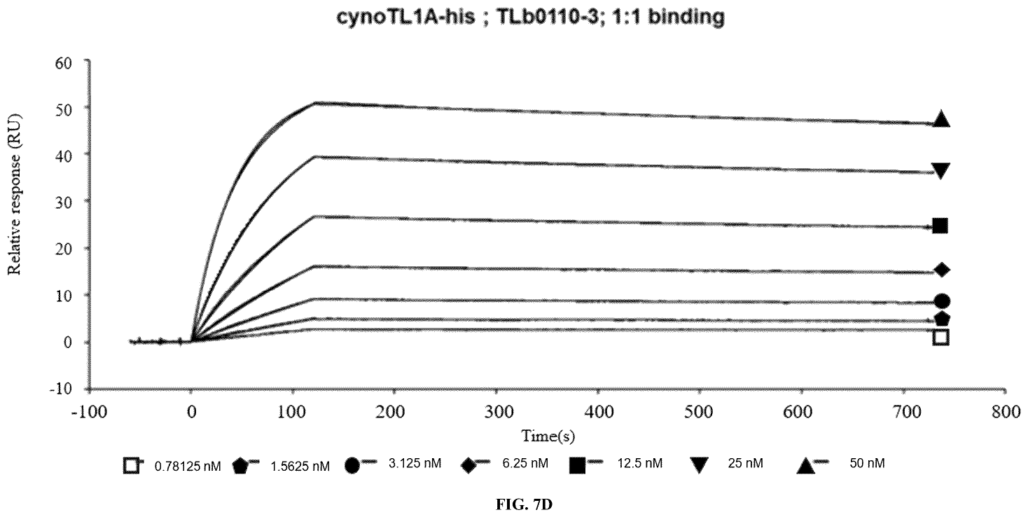

The affinity results of TL1A+IL-23p19 bispecific antibody to human IL-23 protein are shown in FIG. 4, and the affinity results to monkey IL-23 protein are shown in FIG. 5. The statistical affinity values are shown in Table 8. TL1A+IL-23p19 bispecific antibody has a high affinity to both human and monkey IL-23 proteins. The affinity results of TL1A monoclonal antibody and TL1A+IL-23p19 bispecific antibody to human TL1A protein are shown in FIG. 6, and the affinity results to monkey TL1A protein are shown in FIG. 7. The statistical affinity values are shown in Table 9. The results show that TL1A monoclonal antibody and TL1A+IL-23p19 bispecific antibody have high affinity to both human and monkey TL1A proteins.

TABLE 7
Affinity of anti-IL-23 mAbs to IL-23
proteins from different species

| | | ka | kd | KD |
|---|---|---|---|---|
| Abs | Antigen | (1/Ms) | (1/s) | (M) |
| Risankizumab | hIL- | 4.23E+05 | 7.41E−05 | 1.75E−10 |
| (Analogue) | 23p19/hIL- | | | |
| 23A0186-6 | 12p40 | 4.23E+05 | 9.06E−05 | 2.14E−10 |
| 23A0198 | | 8.18E+05 | 8.26E−05 | 1.01E−10 |
| Risankizumab | cynoIL- | 1.73E+06 | 5.93E−05 | 3.43E−11 |
| (Analogue) | 23p19/mIL- | | | |
| 23A0186-6 | 12p40 | 1.49E+06 | 5.16E−05 | 3.46E−11 |
| 23A0198 | | 3.05E+06 | 3.60E−05 | 1.18E−11 |

TABLE 8
Affinity of the bispecific antibodies
to IL-23 proteins of different species

| | | ka | kd | KD |
|---|---|---|---|---|
| Abs | Antigen | (1/Ms) | (1/s) | (M) |
| TLb0103-3 | hIL- | 4.21E+05 | 2.92E−05 | 6.93E−11 |
| TLb0110-2 | 23p19/ | 9.19E+05 | 6.03E−05 | 6.56E−11 |
| TLb0110-3 | hIL-12p40 | 9.26E+05 | 5.45E−05 | 5.88E−11 |
| TLb0103-3 | cynoIL- | 1.53E+06 | 1.76E−05 | 1.16E−11 |
| TLb0110-2 | 23p19/ | 3.66E+06 | 1.05E−05 | 2.87E−12 |
| TLb0110-3 | mIL-12p40 | 3.66E+06 | 1.68E−05 | 4.60E−12 |

TABLE 9
Affinity of antibodies to TL1A proteins from different species

| | | ka | kd | KD |
|---|---|---|---|---|
| Abs | Antigen | (1/Ms) | (1/s) | (M) |
| TLA0015-11 | human | 1.88e+06 | 2.51e−04 | 1.33e−10 |
| TLb0103-3 | TL1A | 7.02e+05 | 1.31e−04 | 1.86e−10 |
| TLb0110-2 | | 5.75e+05 | 1.42e−04 | 2.48e−10 |
| TLb0110-3 | | 6.72e+05 | 1.47e−04 | 2.19e−10 |
| TLA0015-11 | Cyno | 1.45e+06 | 2.58e−04 | 1.78e−10 |
| TLb0103-3 | TL1A | 4.75e+05 | 1.50e−04 | 3.16e−10 |
| TLb0110-2 | | 3.92e+05 | 1.44e−04 | 3.69e−10 |
| TLb0110-3 | | 4.54e+05 | 1.45e−04 | 3.18e−10 |

### Example 5 FACS Detection of the Ability of Bispecific Antibodies to Block the Binding of TL1A-his Protein to 293-DR3 Cells

Based on the flow cytometry method, the ability of the multiple TL1A+IL-23p19 bispecific antibodies prepared by Examples 1-3 of the present invention to block the binding of huTL1A-his protein to human DR3 (DR3: UniprotKB NO. Q93038) expressed on the surface of Expi293 cells was detected. The blocking ability was determined by comparing the curves of different concentrations of antibodies blocking the binding of huTL1A-his protein to 293-huDR3 cells.

The DNA sequence encoding the extracellular region protein of the receptor DR3 of TL1A was cloned into the mDeZ-TM (with transmembrane sequence and Zeocin resistance gene) vector, transiently transfected into Expi293 cells, and 100 μg/mL Zeocin (final concentration) was added 24 hours later for 7 days of screening to obtain the Expi293 cell line (293-huDR3) expressing human DR3 on the cell surface. The huTL1A-his protein was conjugated with Biotin using a biotin labeling kit (ThermoFisher, A35389) to obtain huTL1A-his-Biotin protein.

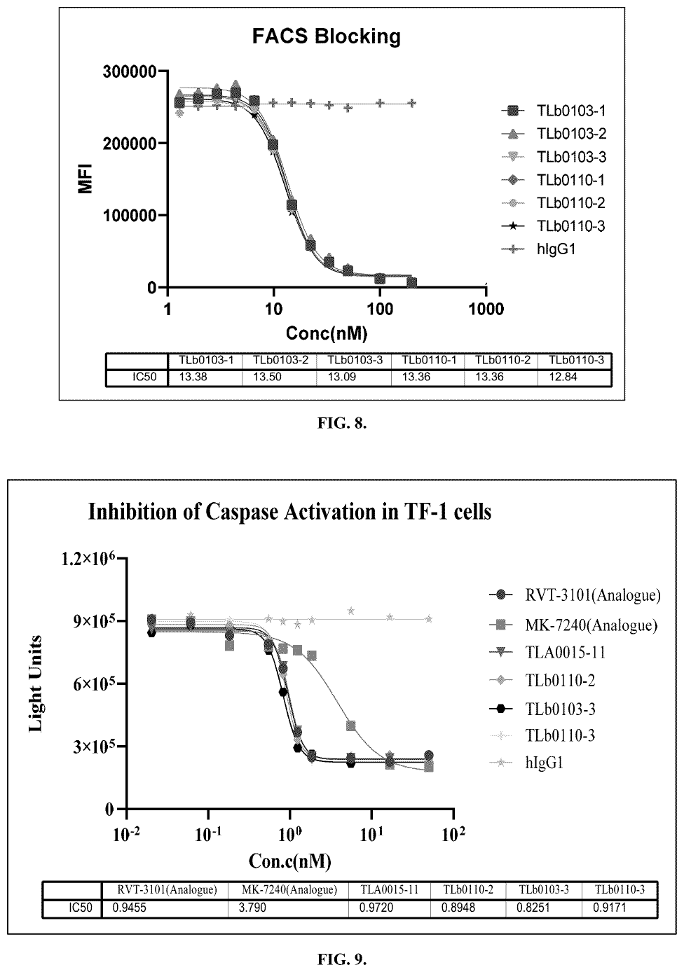

The binding curve between huTL1A-his-Biotin protein and 293-huDR3 cells was detected by FACS Binding, and the EC80 concentration of protein binding was determined (data not shown). 293-huDR3 cells were washed three times with staining buffer (PBS+2% FBS (Gibco, Cat #10091148)+5 mM EDTA (Gibco, Cat #15575020)), centrifuged at 300 g for 5 minutes each time, and the supernatant was discarded. The cells were resuspended in PBS, the cell density was adjusted to 5×105 cells/mL, 100 μl/well, added to a 96-well plate, centrifuged to remove the supernatant, and 2×EC80 concentration of huTL1A-his-Biotin protein was added, 50 μl/well. The double antibody and positive control antibody were diluted to 400 nM, 2-fold stepwise dilution for 12 gradients (the highest final concentration was 200 nM), 50 μl/well, added to a 96-well plate, mixed evenly with huTL1A-his-Biotin protein and 293-huDR3 cells, and incubated at 4° C. for 30 min. The cells were washed twice with PBS to remove unbound antibodies to be tested. Then 100 μl of Streptavidin-PE secondary antibody was added to each well and incubated at 4° C. for 30 minutes. Centrifuged at 300 g for 5 minutes, and the cells were washed twice with PBS to remove unbound secondary antibodies. Finally, the cells were resuspended in 200 μl PBS, and the ability of the polyclonal antibody to block TL1A protein binding to 293-huDR3 cells was determined by Beckman Coulter CytoFLEX flow cytometer. The obtained data were fitted and analyzed by GraphPad Prism software. The experimental results are shown in FIG. 8. All the bispecific antibodies have the ability to block the binding of TL1A-his protein to 293-DR3 cells, and the blocking abilities of the bispecific antibodies are comparable.

### Example 6 Detection of the Ability of Bispecific Antibodies to Inhibit Caspase 3/7 Activated by TL1A-his Protein

The ability of multiple TL1A+IL-23p19 bispecific antibodies prepared by Examples 1-3 of the present invention to inhibit TL1A-induced TF-1 (endogenously expressing DR3) cell apoptosis signaling pathway was detected by using a method based on the Caspase-Glo® 3/7 Assay System activity detection kit (Promega, Cat #G8092). The inhibitory ability was determined by comparing the inhibitory curves of different concentrations of antibodies against Caspase3/7 activity in TF-1 cells.

The huTL1A-his protein was configured to a 4× working concentration of 16 nM; the Cycloheximide small molecule drug was configured to a 4× working concentration of 10 g/ml. 25 μl of the mixture was added to each well of a 96-well plate with a white bottom. The antibody starting concentration was then configured to 200 nM (final concentration of 50 nM), and after 4-fold dilution at four concentration points, it was further diluted 2-fold, for a total of 10 concentrations, and 25 μl was added to each well of a 96-well plate with a white bottom. The above mixture was pre-incubated in a 37° C. incubator for 1 h. At the same time, the TF-1 cells were centrifuged (300 g for 5 minutes), resuspended in buffer (1640 medium+10% FBS) and counted, and the cell density was adjusted to 1×106/ml. After the antigen-antibody incubation time was over, the white 96-well flat-bottom plate was taken out, and the TF-1 cells with the adjusted density were inoculated at 50 μl/well, and the plate was placed in a carbon dioxide incubator for further incubation for 6 hours. After the incubation was completed, the plate was removed from the incubator and equilibrated at room temperature for about 30 minutes. At the same time, the Caspase-Glo® 3/7 Assay System reagent was taken out in advance and equilibrated at room temperature. Caspase-Glo® 3/7 Assay System reagent was added, 50 μl was added to each well, and the mixture was shaken at 300 rpm for 30 seconds. The mixture was then allowed to stand for 15-30 minutes to allow the cells to fully lyse. The RLU value was then read on a multifunctional microplate reader. The logarithm of the sample concentration was taken using Graphpad and a four-parameter fitting plot was performed with the measured RLU value. The results are shown in FIG. 9. TLb0103-3, TLb0110-2 and TLb0110-3 bispecific antibodies all have good inhibitory functions for Caspase3/7 activity, and their inhibitory functions are comparable to those of the TLA0015-11 monoclonal antibody and the control antibody RVT-3101 (Analogue), and are superior to the control antibody MK-7240 (Analogue).

### Example 7 Fluorescence Reporter Gene Method to Detect the Ability of Bispecific Antibodies to Inhibit TL1A-Activated NF-κB Signaling Pathway

The ability of multiple TL1A+IL-23p19 bispecific antibodies prepared by Examples 1-3 of the present invention to inhibit the NF-κB signaling pathway of TF-1 (endogenously expressing DR3) cells activated by TL1A-his protein was detected by a luciferase reporter gene method. The TF-1-NFκB-Luc cell line was purchased from Jiman Biotechnology Co., Ltd. (GM-C30289, H_TNFSF15(TL1A) Reporter Cell Line) and was screened by infecting TF-1 cells with NF-κB-Luciferase lentivirus.

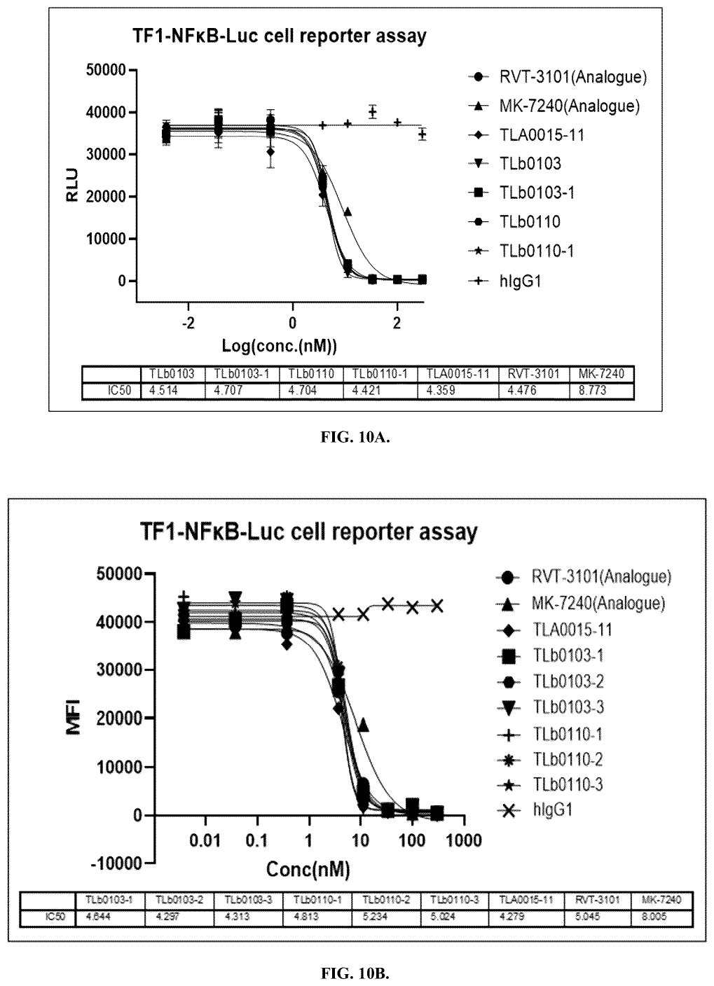

This experiment used TF1-NFκB-luc cells to detect the biological activity of bispecific antibodies in inhibiting the DR3-NFκB signaling pathway activated by TL1A-his protein. The experiment used an assay buffer (RPMI 1640 medium+1% FBS) to prepare a 3× assay concentration of huTL1A-his antigen solution of 0.9 g/ml (final concentration of 0.3 g/ml), which was added to a white 96-well flat-bottom plate at 40 μl/well. Prepare 3× analytical concentration of double antibody, the highest concentration point is 300 nM (final concentration is 100 nM), 5-fold gradient dilution, a total of 8 concentration points. After the antibody is diluted, add it to the above-mentioned white 96-well flat-bottom plate at 40 μl/well, and then place the white 96-well flat-bottom plate in a carbon dioxide incubator to pre-incubate the double antibody and antigen for 1 h. During the pre-incubation of double antibody and antigen, centrifuge TF1-NFκB-luc cells (300 g, 5 min), resuspend them in analysis buffer and count them, and adjust the cell density to 5×105/ml. After the antigen-antibody incubation time is over, remove the white 96-well flat-bottom plate, inoculate the TF1-NFκB-luc cells with the adjusted density at 40 μl/well, and continue incubating in a carbon dioxide incubator for 6 h. After the incubation, the white 96-well flat-bottom plate was taken out from the incubator, and 60 μl of Bio-Lite Luciferase Assay reagent (Novozyme, DD1201-02) was added to each well. After slight shaking for 5 minutes, the RLU value was read on a multifunctional microplate reader. The logarithm of the sample concentration was taken using Graphpad and the measured signal value was subjected to a four-parameter fitting plot. The results are shown in FIGS. 10A and 10B: TLb0103, TLb0103-1, TLb0103-2, TLb0103-3, TLb0110, TLb0110-1, TLb0110-2, TLb0110-3 bispecific antibodies all have good inhibitory activity on the NF-κB signaling pathway activated by TL1A protein, and the inhibitory function is comparable to that of the TLA0015-11 monoclonal antibody and the control antibody RVT-3101 (Analogue), and is superior to the control antibody MK-7240 (Analogue).

### Example 8 Fluorescence Reporter Gene Method to Detect the Ability of Bispecific Antibodies to Inhibit IL-23-Activated STAT3 Signaling Pathway

The ability of the multiple TL1A+IL-23p19 bispecific antibodies prepared by Examples 1-3 of the present invention to inhibit the STAT3 signaling pathway activated by IL-23 protein was detected based on the luciferase reporter gene method. The inhibitory ability was determined by comparing the inhibition curves of the fluorescence signal of the bispecific antibodies at different concentrations.

This study used H_IL-23 Reporter 293 cells (purchased from Jiman Bio, product number GM-C06722) to detect the biological activity of anti-IL-23 antibodies in inhibiting the IL-23/IL-23R-JAK-STAT3 signaling pathway. H_IL-23 Reporter 293 cells overexpress IL-23R and IL-12Rβ1 on the surface of the HEK293 tool cell membrane, and STAT3-luc can be stably expressed in the cells. The inhibitory activity of the anti-IL-23 antibody was evaluated by measuring the Luciferase signal value of the cell. In this experiment, the IL-23 antigen solution with 3× analytical concentration was prepared with Assay buffer (DMEM medium+1% FBS) to be 1.5 ng/ml (final concentration was 0.5 ng/ml), and was inoculated in a white 96-well flat-bottom plate at 50 μl/well. The antibody with 3× analytical concentration was prepared, with a top dose of 8 nM (final was 2 nM), 4-fold gradient dilution, and a total of 10 concentration points. After the antibody was diluted, it was inoculated in the above-mentioned white 96-well flat-bottom plate at 50 μl/well, and then the white 96-well flat-bottom plate was placed in a carbon dioxide incubator to pre-incubate the anti-IL-23 antibody and Acro IL-23 antigen for 1h. During the pre-incubation of the antibody and antigen, the H_IL-23 Reporter 293 cells were digested, centrifuged (300 g, 5 min), resuspended in Assay buffer and counted, and the cell density was adjusted to 4×105/ml. After the pre-incubation time is over, take out the white 96-well flat-bottom plate and inoculate H_IL-23 Reporter 293 cells with adjusted density at 50 μl/well. Then place the white 96-well flat-bottom plate in a carbon dioxide incubator for further incubation for 18 h. After the incubation is over, take out the white 96-well flat-bottom plate from the incubator, equilibrate at room temperature for 10 min, add 60 μl of Bio-Lite Luciferase Assay reagent (Vazyme, Cat #DD1208) to each well, shake for 5 min, and read the RLU value on a multifunctional microplate reader (Tecan, Spark model). Use Graphpad to take the logarithm of the sample concentration and perform a four-parameter fitting plot with the measured RLU value.

The results of antibody inhibition of STAT3 activated by IL-23 are shown in FIGS. 11A and 11B, where the inhibitory activity of anti-IL-23p19 monoclonal antibody 23A0198 is slightly better than that of the control antibody Risankizumab (Analogue), and the inhibitory activity of monoclonal antibody 23A0186-6 is slightly weaker than that of the control antibody Risankizumab (Analogue). The bispecific antibodies TLb0103, TLb0103-1, TLb0103-3, TLb0110, TLb0110-1, TLb0110-2, and TLb0110-3 also have good inhibitory activity, and the bispecific antibody inhibitory activity of TLb0110, TLb0110-1, TLb0110-2, and TLb0110-3 is slightly better than that of the control antibody Risankizumab (Analogue).

### Example 9 Detection of the Inhibitory Activity of Bispecific Antibodies on the Secretion of IL-17 Cytokines by Mouse Spleen Cells

In this study, single splenocytes were isolated from the spleen of C57BL/6 mice (purchased from Jicui Yaokang Biotechnology Co., Ltd.), and hIL-23 was used to bind to mIL-23R on mouse spleen cells to induce the differentiation of spleen cells into Th17 cells. The biological activity of anti-IL-23p19 monoclonal antibody and TL1A+IL-23p19 bispecific antibody in blocking the binding of hIL-23 to its receptor was evaluated by detecting the changes in the secretion of the cytokine mouse IL-17A. In this experiment, an IL-23 antigen solution with a 4× analytical concentration of 20 ng/ml (final concentration of 5 ng/ml) was prepared with Assay buffer (RPMI 1640 medium+10% FBS+50 ng/ml IL-2), and was inoculated in a 96-well round-bottom plate at 50 μl/well. Prepare 4× analytical concentration of antibody, top dose is 800 nM (final is 200 nM), 4-fold gradient dilution for 3 concentration points, 8-fold gradient dilution for 7 concentration points. After the antibody is diluted, inoculate it in the above 96-well round-bottom plate at 50 μl/well, and then place the 96-well round-bottom plate in a carbon dioxide incubator to pre-incubate the antibody and IL-23 antigen for 1 h. During the pre-incubation of the antibody and antigen, grind the mouse spleen, pass it through the cell sieve, add PBS to resuspend it, transfer it to a 50 ml centrifuge tube, and centrifuge it at 400 g for 10 min. Discard the supernatant, add 10 ml of red blood cell lysis buffer to the cell pellet and lyse it at room temperature for 5 min, and centrifuge it at 400 g for 10 min. Discard the supernatant again, add 40 ml of PBS to resuspend the cell pellet, and wash it once by centrifugation at 400 g for 10 min. Resuspend the cells with assay buffer, count and adjust the cell density to 5×106/ml for use. After the pre-incubation of the antibody and antigen is completed, the 96-well round-bottom plate is taken out and the spleen cells prepared above are inoculated at 100 μl/well. The 96-well round-bottom plate is placed in a carbon dioxide incubator for further incubation for 72 h. After the incubation is completed, the 96-well round-bottom plate is taken out, centrifuged at 300 g for 5 min, and the culture supernatant is aspirated. According to the operating instructions of the Mouse IL-17A ELISA Kit (Lianke Bio, Cat #EK217/2-96), the mIL-17A is measured, and the OD450 nm-OD570 nm value is read on the microplate reader (Tecan, Spark model). The level of Mouse IL-17A in the test sample is converted according to the standard curve, and then the logarithm of the antibody concentration is taken using Graphpad to perform a four-parameter fitting plot with the Mouse IL-17A level.

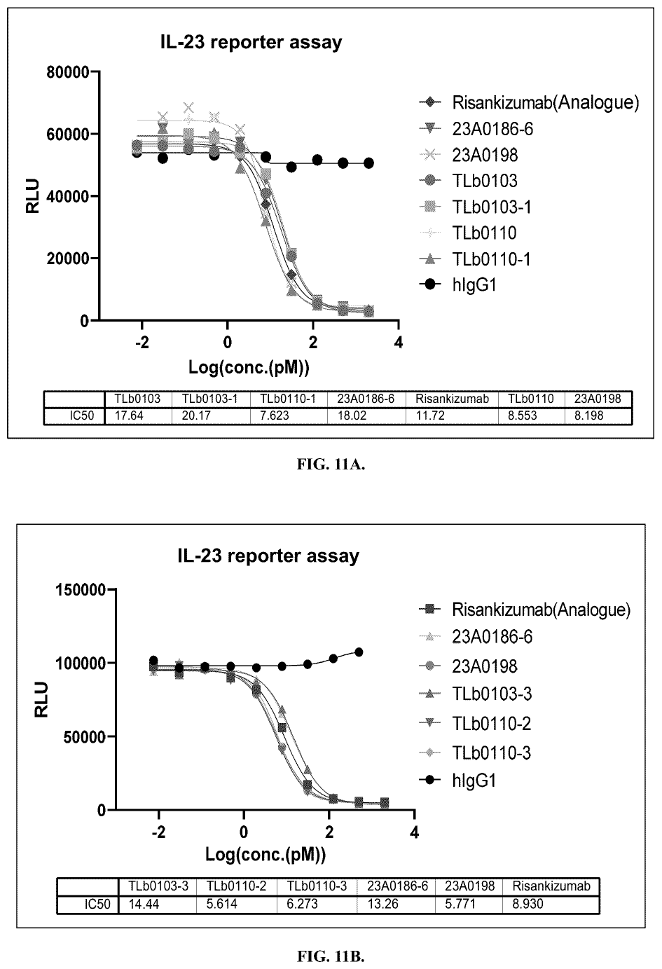

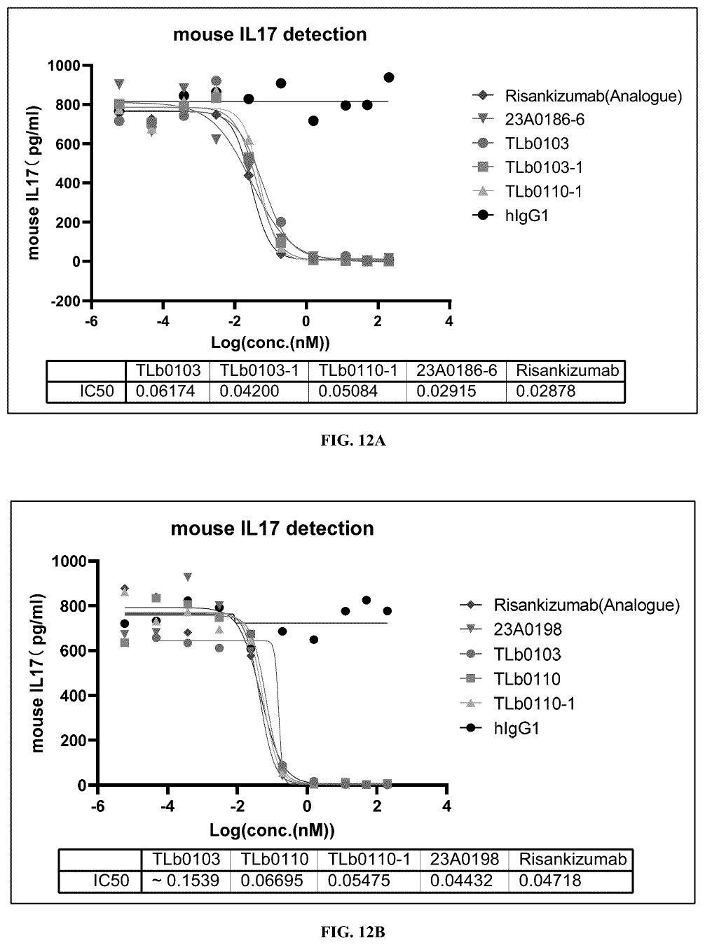

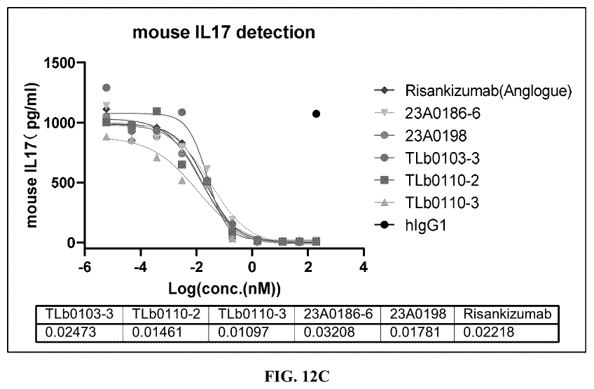

The results of antibody inhibition on mouse IL-17 cytokine are shown in FIGS. 12A, 12B and 12C, where the inhibitory activity of anti-IL-23p19 monoclonal antibodies 23A0186-6 and 23A0198 on mIL-17 is comparable to that of the control antibody Risankizumab (Analogue). The bispecific antibodies TLb0103, TLb0103-1, TLb0103-3, TLb0110, TLb0110-1, TLb0110-2 and TLb0110-3 also have good inhibitory activity, which is comparable to that of the control antibody Risankizumab (Analogue).

### Example 10 Detection of the Inhibitory Activity of the Bispecific Antibody on the Secretion of IL-22 Cytokine by Mouse Spleen Cells

In this study, single splenocytes were isolated from the spleen of C57BL/6 mice. hTL1A and hIL-23 were used to stimulate mouse splenocytes to secrete cytokines. The changes in the secretion of the cytokine mouse IL-22 were detected to evaluate the inhibitory activity of anti-TL1A monoclonal antibody, anti-IL-23p19 monoclonal antibody, and TL1A+IL-23p19 bispecific antibody on the secretion of IL-22 cytokines by mouse splenocytes. In this experiment, assay buffer (RPMI 1640 medium+10% FBS+50 ng/mL IL-2) was used to prepare 4× analytical concentrations of hTL1A solution 0.4 g/mL (final concentration 0.1 g/mL) and hIL-23 solution 2 ng/mL (final concentration 0.5 ng/mL). The antibody was prepared at 4× analytical concentration, with the highest concentration of 80 nM (final concentration 200 nM), and 8 concentration points were diluted 5-fold downward. Add 50 μl/well of 4×hTL1A solution, 50 μl/well of 4×hIL-23 solution and 50 μl/well of antibody to a 96-well round-bottom plate and pre-incubate for 1 h. During the pre-incubation process, grind the mouse spleen, pass through the cell sieve, resuspend in PBS, transfer to a 50 ml centrifuge tube, and centrifuge at 400 g for 10 min. Discard the supernatant, add 10 ml of red blood cell lysis buffer to the cell pellet and lyse at room temperature for 5 min, and centrifuge at 400 g for 10 min. Discard the supernatant again, resuspend the cell pellet with 40 ml of PBS, and wash once by centrifugation at 400 g for 10 min. Resuspend the cells with Assay buffer, count and adjust the cell density to 1×107/mL for later use. After the pre-incubation of the antibody and antigen is completed, remove the 96-well round-bottom plate and inoculate the spleen cells prepared above at 50 μl/well. Place the 96-well round-bottom plate in a carbon dioxide incubator and continue incubating for 72 h. After the incubation, the 96-well round-bottom plate was removed, centrifuged at 300 g for 5 minutes, and the culture supernatant was carefully aspirated. The mIL-22 was determined according to the operating instructions of the Mouse IL-22 ELISA Kit (Lianke Bio, Cat #EK222/2-96), and the values of OD450 nm-OD570 nm were read on a microplate reader (Tecan, Spark model). The level of Mouse IL-22 in the test sample was converted according to the standard curve fitting formula, and then the antibody concentration was logarithmically fitted with the Mouse IL-22 level using Graphpad to perform a four-parameter fitting diagram

The results of mouse IL-22 detection are shown in FIGS. 13A and 13B. The simple TL1A protein stimulation had no stimulating effect on the secretion of IL-22 in mouse spleen cells; the simple IL-23 protein stimulation had a certain stimulating effect on the secretion of IL-22 in mouse spleen cells; and the addition of TL1A and IL-23 at the same time could well stimulate the secretion of IL-22 in mouse spleen cells. After TL1A+IL-23 co-stimulation, anti-TL1A monoclonal antibody TLA0015-11, anti-IL-23p19 monoclonal antibodies 23A0186-6 and 23A0198, TLA0015-11+23A0186-6, TLA0015-11+23A0198, TLb0103-3 bispecific antibody, TLb0110-2 bispecific antibody and TLb0110-3 bispecific antibody were added. The inhibitory activity was roughly ranked as follows: bispecific antibody>monoclonal antibody combination>anti-IL-23p19 monoclonal antibody>anti-TL1A monoclonal antibody. Among them, the inhibitory activity of TLb0110-2 and TLb0110-3 bispecific antibodies against mIL-22 was better than that of the control antibody Risankizumab (Analogue).

### Example 11 Detection of the Tm Value of the Bispecific Antibody

This experiment is based on the Panta instrument (NanoTemper Technologies GmbH) to detect the Tm values of multiple bispecific antibodies prepared by Examples 1-3 of the present invention. The Panta instrument includes micro-differential scanning fluorescence technology (nanoDSF) technology, which tracks the folding state of the protein by detecting the endogenous fluorescence of the protein. The ratio of the fluorescence signal will change with the increase of temperature or the increase of the concentration of the chemical denaturant, thereby determining the protein stability parameter Tm value. The specific Tm values of each antibody are shown in Table 10.

TABLE 10
Antibody Tm

| antibody | Tm1(° C.) | Tm2(° C.) | Ton-set(° C.) |
|---|---|---|---|
| TLb0103 | 65.84 | 83.29 | 57.84 |
| TLb0103-1 | 66.37 | 78.71 | 59.62 |
| TLb0103-2 | 66.37 | / | 58.07 |
| TLb0103-3 | 66.62 | 79.06 | 59.80 |
| TLb0110 | 66.75 | 76.51 | 59.15 |
| TLb0110-1 | 68.06 | 76.38 | 61.10 |
| TLb0110-2 | 68.04 | 76.67 | 61.00 |
| TLb0110-3 | 67.99 | 76.33 | 61.27 |

### Example 12 Detection of the Pharmacodynamic Activity of the Bispecific Antibody in the DSS-Induced hTL1A/hIL-23 Mouse Enteritis Model

In this study, hTL1A/hIL-23 transgenic mice were used to study the in vivo pharmacodynamic activity of the bispecific antibody in the DSS-induced enteritis model. hTL1A/hIL-23 transgenic mice were purchased from Biocytogen (Beijing) Pharmaceutical Technology Co., Ltd. The humanized transgenic mice are C57BL/6 strain mice, which are hybridized by B-hTL1A transgenic mice and B-hIL-23A/hIL-12B transgenic mice. The transgenic mice can express human TL1A and human IL-23 proteins simultaneously. The dextran sulfate sodium salt (DSS)-induced mouse enteritis model is the most widely used chemically induced mouse IBD model. Acute ulcerative enteritis or chronic colitis is induced by dissolving DSS in drinking water, which destroys the intestinal epithelial cells of mice, and nonspecific immune cells release cytokines, which ultimately leads to the destruction of the integrity of the mucosal barrier. The animals show obvious weight loss, loose stools, blood in the stool, and granulocyte infiltration. The clinical symptoms and pathological characteristics are extremely similar to human ulcerative colitis. Therefore, the DSS-induced enteritis model can be used to evaluate the in vivo pharmacodynamic activity of the diabody. 26 male hTL1A/hTL-23 transgenic mice were randomly divided into five groups, including two mice in the G1 negative control group, six mice in the G2 modeling group, six mice in the G3 TLb0103-3 drug group, six mice in the G4 TLb0110-2 drug group, and six mice in the G5 TLb0110-3 drug group. The detailed grouping of mice is shown in Table 11. The drug was injected intraperitoneally on days 0, 3, and 6, respectively, at a dose of 10 mg/kg; the mice were given drinking water containing 3% DSS from day 0 to day 7, and normal drinking water was used from day 8 to day 9, and the experiment was ended on day 9. During the experiment, the weight of the experimental animals was measured and recorded every two days, and the daily activities of the animals were observed; the fecal characteristics of the experimental animals were scored every day (0=normal, 1=moist/sticky, 2=soft, 3=liquid). After the experiment ended on the 9th day, all experimental animals were euthanized by asphyxiation with excessive carbon dioxide, and then the abdominal cavity was cut open, the colon was removed, and the length of the colon was recorded.

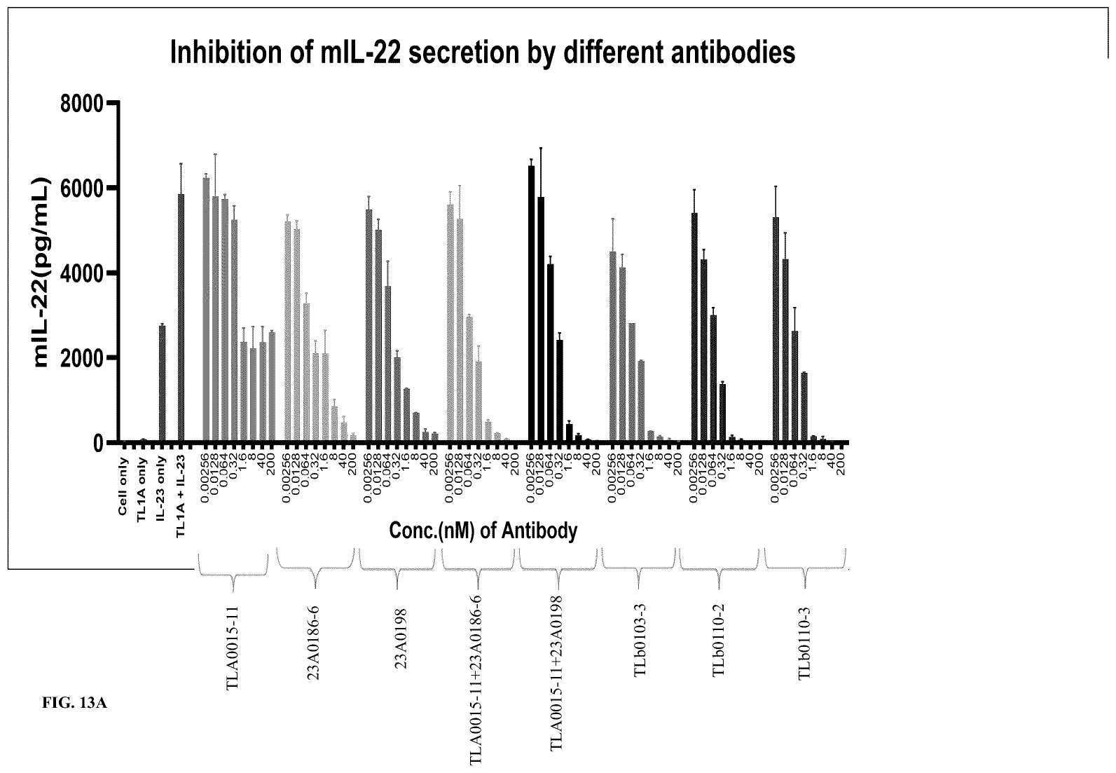

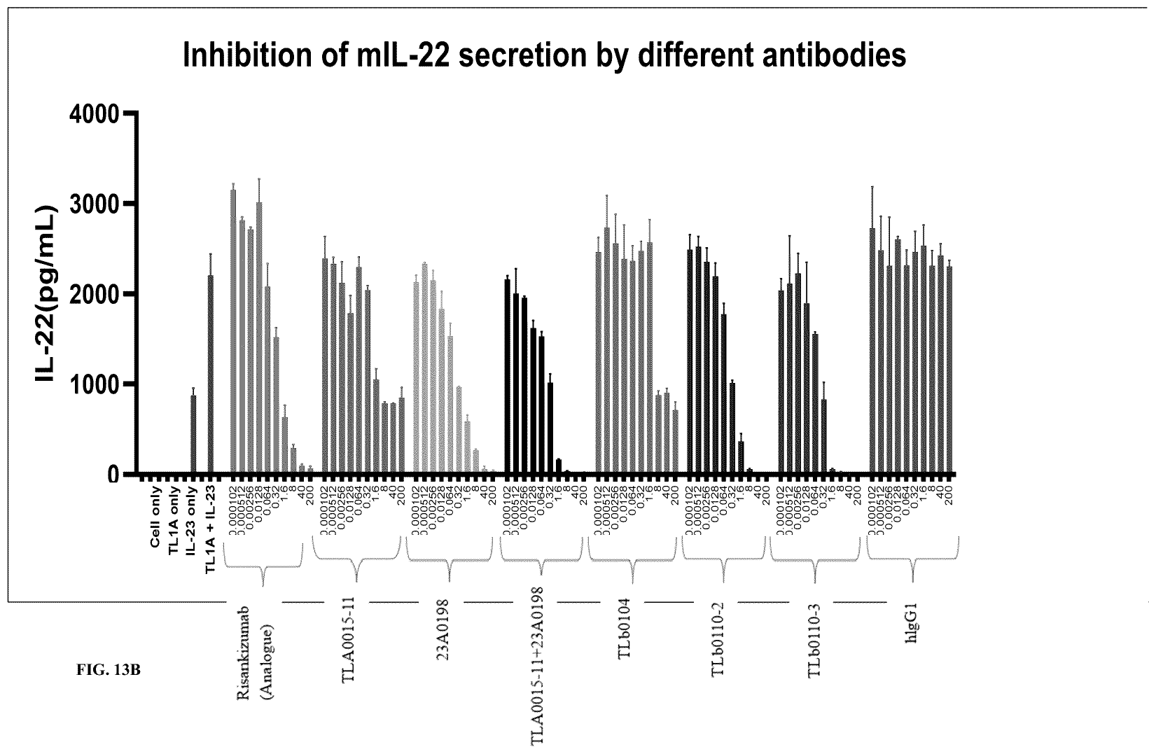

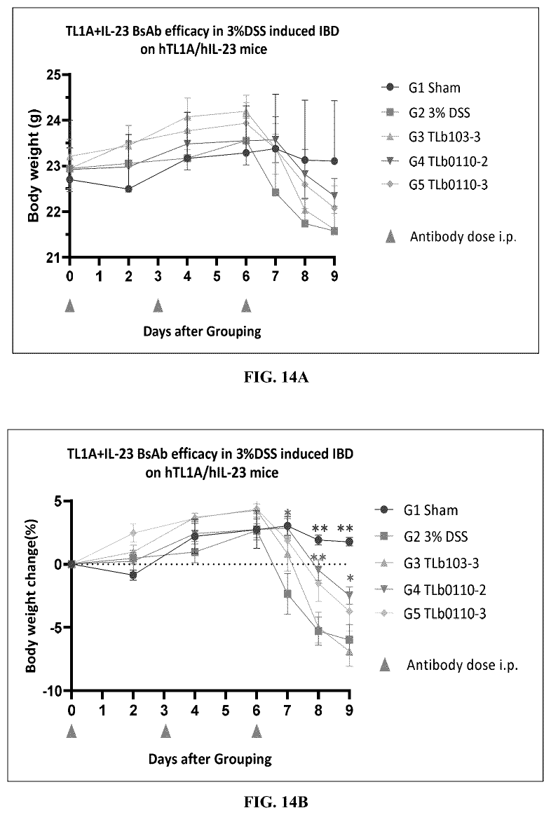

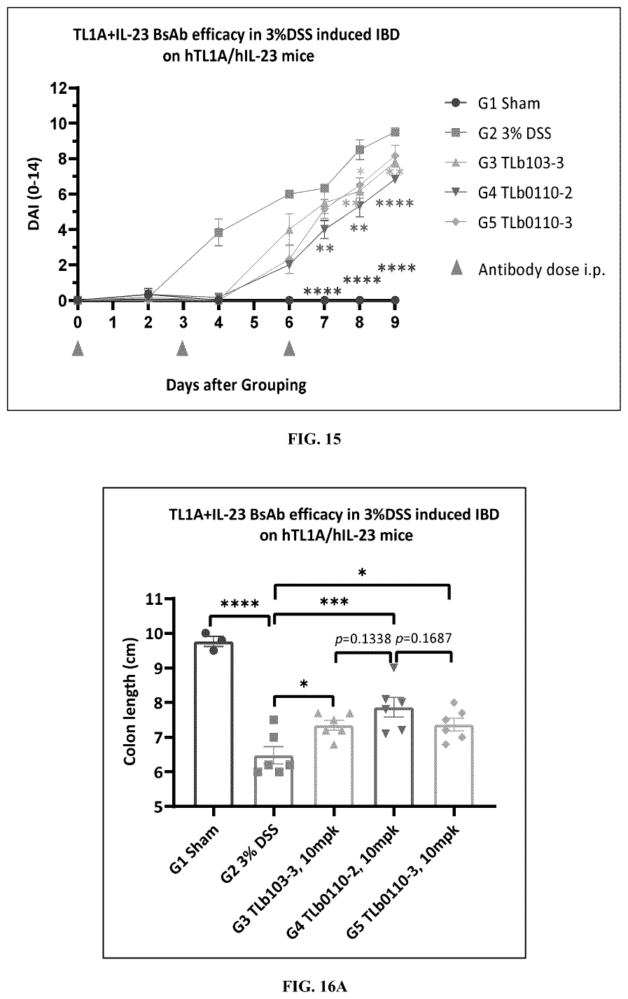

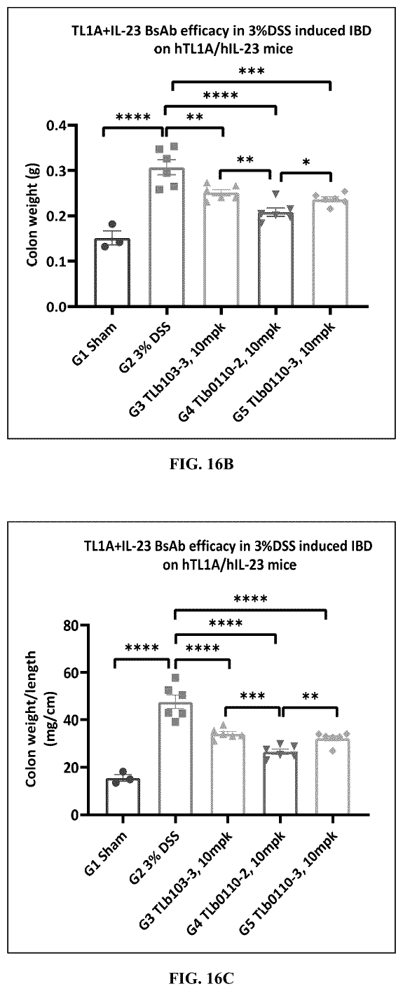

After the experiment, the results were statistically analyzed, and the results of the weight change of mice are shown in FIGS. 14A and 14B. Compared with the G2 modeling group, the TLb0110-2 and TLb0110-3 groups showed that the weight loss of mice was alleviated. Therefore, from the perspective of weight, TLb0110-2 and TLb0110-3 bispecific antibodies have certain efficacy. From the daily activity score results (FIG. 15), the score of the TLb0110-2 group was lower than that of the TLb0103-3 and TLb0110-3 groups, and significantly lower than that of the G2 modeling group. At the end of the 9th day of the experiment, all mice were alive, and the length and weight of the colorectum were measured after dissection. The results of the colorectal length of mice are shown in FIG. 16A, the colorectal weight results are shown in FIG. 16B, and the colorectal weight to length ratio results are shown in FIG. 16C. The G2 modeling group can see that the colorectal length is significantly shortened and the weight is significantly increased. The TLb0103-3, TLb0110-2 and TLb0110-3 bispecific antibody administration groups can see the efficacy of the colorectal length and weight of mice, among which the efficacy of TLb0110-2 bispecific antibody is slightly better than that of TLb0103-3 and TLb0110-3 bispecific antibodies.

In summary, TLb0110-2 and TLb0110-3 bispecific antibodies can see obvious efficacy in the DSS-induced enteritis model.

TABLE 11
Mouse grouping and dosing details

| Group | Animal | Modeling | Test articles | Number | Dosing |
|---|---|---|---|---|---|
| G1 | hTL1A/ | Sham | PBS | 2 | / |
| G2 | hIL-23 | 3% DSS | PBS | 6 | |
| G3 | mice | | TLb0103-3 | 6 | 10 mg/kg, |
| G4 | | | TLb0110-2 | 6 | 3 times |
| G5 | | | TLb0110-3 | 6 | a week |

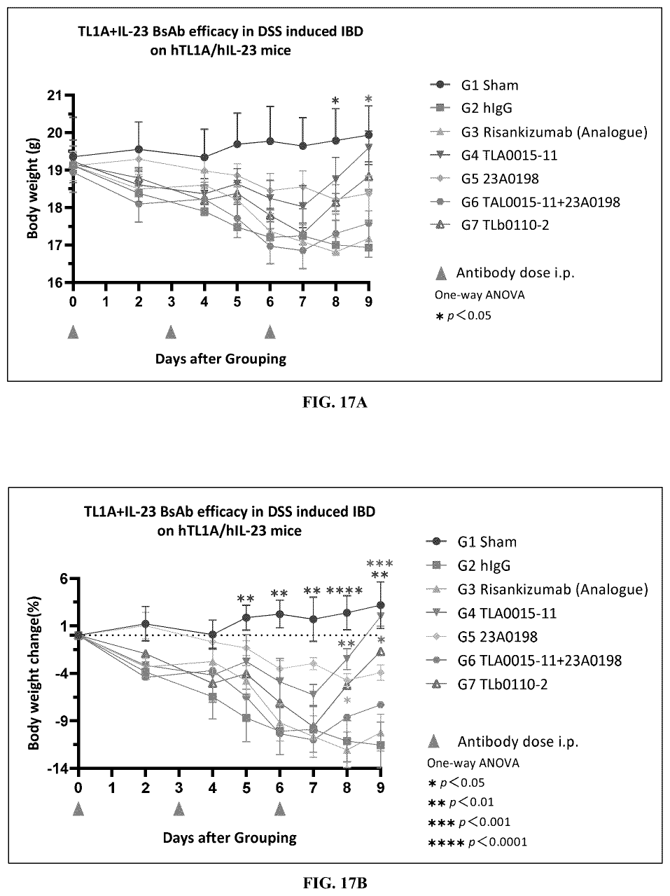

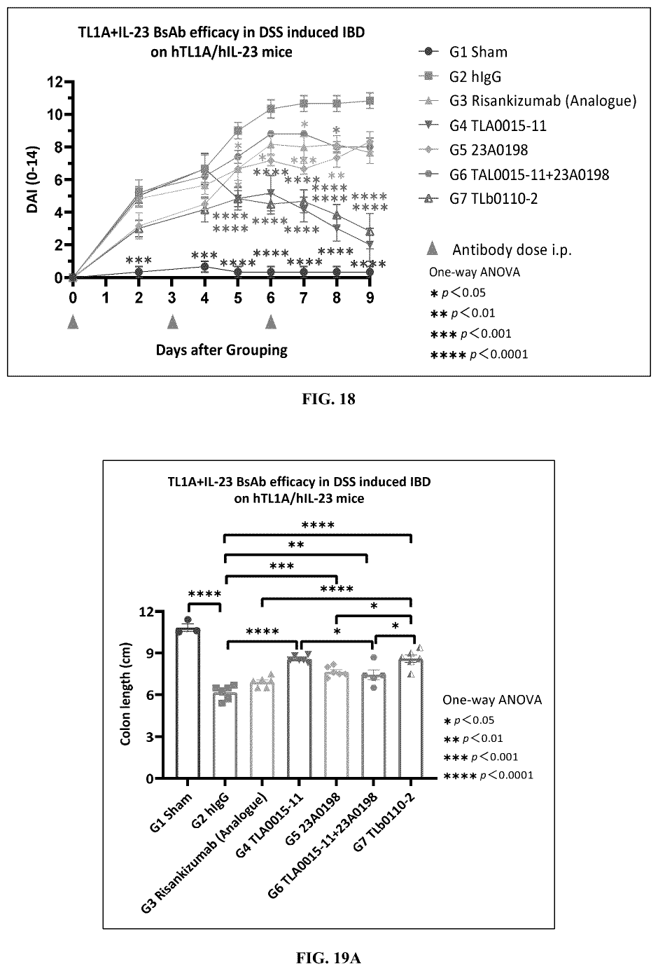

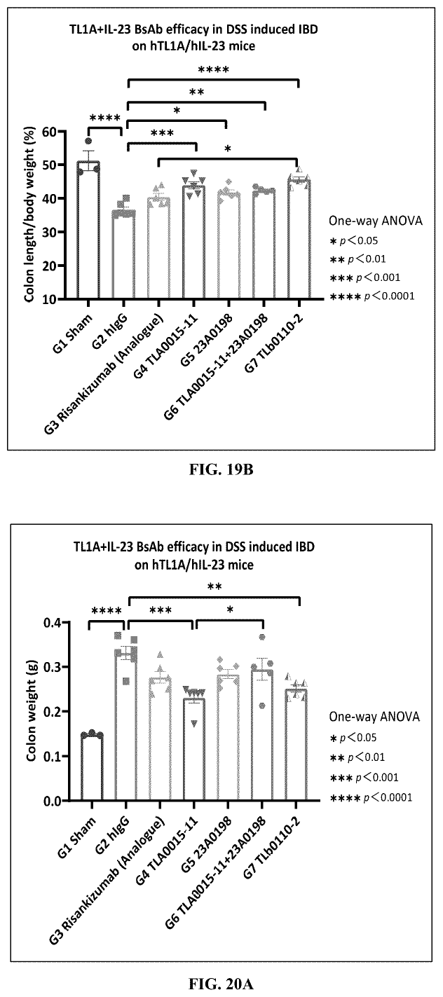

In addition, the efficacy of anti-TL1A monoclonal antibody, anti-IL-23 monoclonal antibody and bispecific antibody in the DSS-induced enteritis model was verified. The specific experimental design scheme is shown in Table 12. The results of mouse weight change are shown in FIGS. 17A and 17B, where TLA0015-11, 23A0198 and bispecific antibody TLb0110-2 can see obvious efficacy results in the weight index. The results of the daily activity score of mice are shown in FIG. 18, where TLA0015-11 and TLb0110-2 can see obvious efficacy in the daily scoring indicators. At the end of the experiment, all mice survived, and there was no difference in survival rate between the groups. After the experiment, the length and weight of the colorectum were measured by dissection. The results of the colorectal length of mice are shown in FIGS. 19A and 19B, and the weight results of the colorectum are shown in FIGS. 20A and 20B, where TLA0015-11 and TLb0110-2 can see obvious efficacy in the colorectal length and weight indicators.

TABLE 12
Mouse grouping and dosing details

| | | | | Number of | Dosing |
|---|---|---|---|---|---|
| Group | Animal | Modeling | Treatment | animals | (Q2W) |
| G1 | hTL1A/ | / | / | 3 | |
| G2 | hIL-23 | DSS | IgG | 6 | 10 mg/kg |
| G3 | mice | | Risankizumab | 6 | 10 mg/kg |
| | | | (Analogue) | | |
| G4 | | | TLA0015-11 | 6 | 10 mg/kg |
| G5 | | | 23A0198 | 6 | 10 mg/kg |
| G6 | | | TLA0015-11 + | 6 | 10 mg/kg + |
| | | | 23A0198 | | 10 mg/kg |
| G7 | | | TLb0110-2 | 6 | 10 mg/kg |

### Example 13 Detection of the Pharmacodynamic Activity of the Bispecific Antibody in the IMQ-Induced hTL1A/hIL-23 Transgenic Mouse Psoriasis Model

This experiment used hTL1A/hIL-23 transgenic mice to study the in vivo pharmacodynamic activity of the bispecific antibody in the imiquimod (IMQ)-induced psoriasis model. IMQ (Aldara, Ensign Laboratories Pty Ltd) can cause skin damage in mice, increased rash and desquamation, thickening of the skin epidermis, and histopathological findings show that the dermis is mainly infiltrated with parakeratosis and inflammatory leukocytes. The phenotype of the imiquimod-induced skin damage and skin inflammation model is similar to that of human psoriasis. Therefore, the IMQ-induced psoriasis model can be used to evaluate the in vivo pharmacodynamic activity of the bispecific antibody.

46 female hTL1A/hIL-23 transgenic mice were randomly divided into eight groups, including four mice in the G1 negative control group (no modeling), and 42 mice in the modeling group, including seven groups, namely G2 modeling group, G3 TLA0015-11 administration group, G4 Risankizumab (Analogue) administration group, G5 23A0198 administration group, G6 TLA0015-11 and 23A0198 monoclonal antibody combination group, G7 TLb0110-2 bispecific antibody administration group and G8 TLb0110-3 bispecific antibody administration group, with six mice in each group. The detailed grouping of mice is shown in Table 13. On the first day after the mice were grouped, the hair in the central area of the back of the mice was carefully shaved to form an exposed area of 2 cm×3 cm. The modeling mice were smeared with 5% imiquimod (62.5 mg per application of 2 cm×3 cm skin) on the back skin of the mice, once a day for five consecutive days. The drug administration group was intraperitoneally injected with drugs on the 1st, 3rd and 5th days, with a dose of 10 mg/kg, and the experiment was terminated on the 6th day. During the experiment, the weight of the experimental animals and the thickness of the skin at the modeling site were measured and recorded every day; the Psoriasis area and severity index (PASI) of the mice was scored every day, and the scales, erythema, skin lesions and infiltration of the skin in the modeling area of the mice were scored separately. After the experiment ended on the 6th day, all experimental animals were euthanized by asphyxiation with excessive carbon dioxide, and the abdominal cavity was cut open, the spleen was removed and weighed. At the same time, the skin at the modeling site on the back of the mouse was taken for H&E staining, and the thickness of the epidermis was counted and the lesion score was performed.

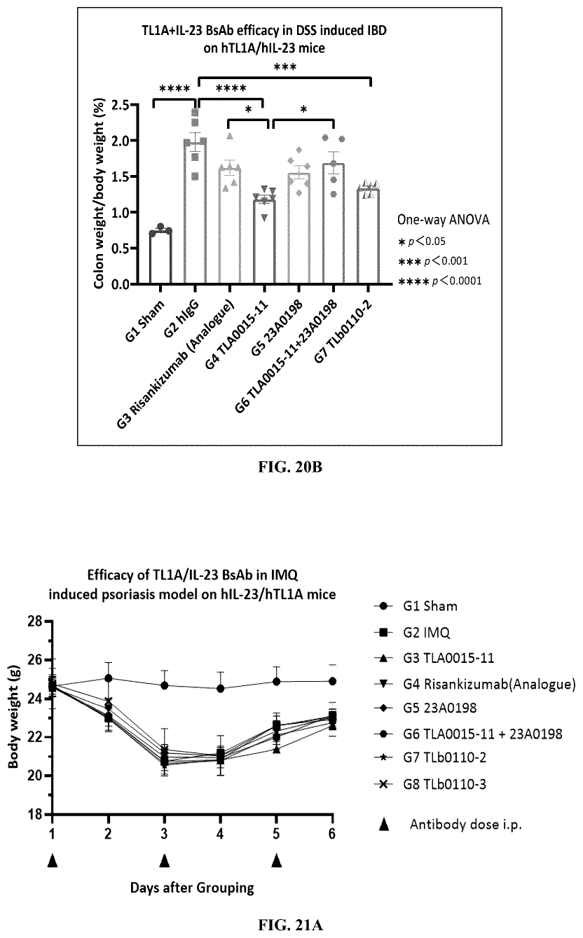

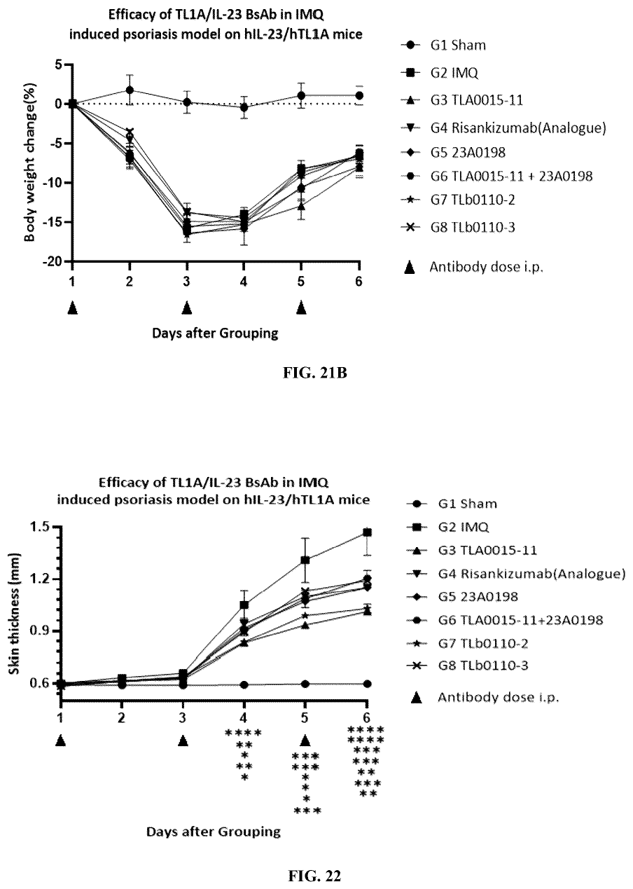

After the experiment, the results were statistically analyzed, and the results of the weight changes of the mice are shown in FIGS. 21A and 21B. Except for the negative control group, the weight of the mice basically did not change, and the weight of the mice in each administration group after modeling was not much different.

The results of the changes in the skin thickness of the mouse modeling site are shown in FIG. 22. It can be seen that in the G2 modeling group, the skin thickness of the mouse modeling site increased significantly, and obvious drug effects were observed in all six drug administration groups. Among them, the improvement in the mouse skin thickness in the G3 TLA0015-11 monoclonal antibody administration group and the G7 TLb0110-2 bispecific antibody administration group was the most obvious; the four groups of G4 Risankizumab (Analogue) control antibody administration group, G5 23A0198 monoclonal antibody administration group, G6 monoclonal antibody combination group and G8 TLb0110-3 bispecific antibody administration group were not much different, and compared with the G2 modeling group, obvious drug effects were also observed.

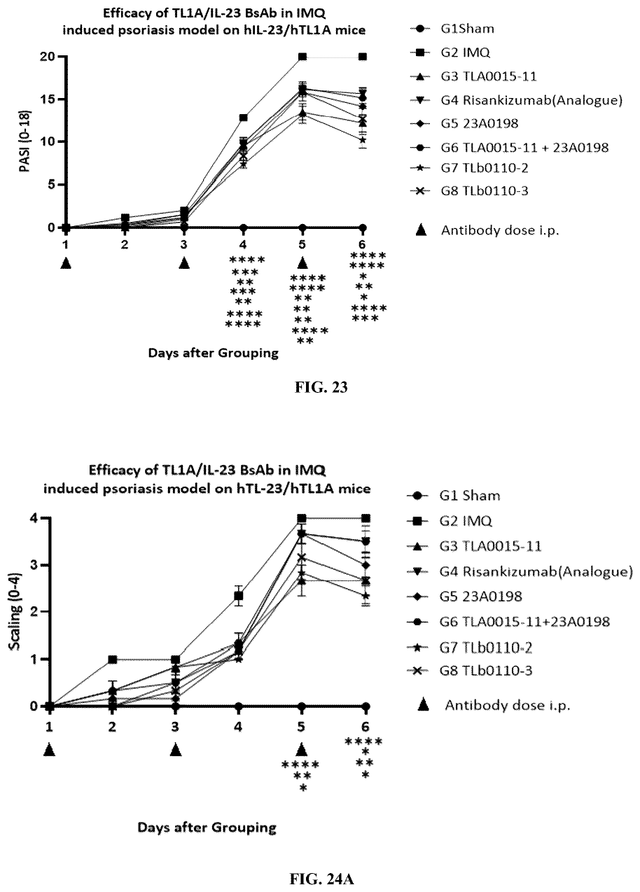

The results of the mouse PASI score are shown in FIG. 23. It can be seen that in the G2 modeling group, the PSAI score increased significantly, and obvious drug effects were observed in all six drug administration groups. Among them, the G7 TLb0110-2 bispecific antibody administration group had the lowest score, the G3 TLA0015-11 monoclonal antibody administration group and the G8 TLb0110-3 bispecific antibody administration group had comparable effects, and the other three groups had comparable effects.

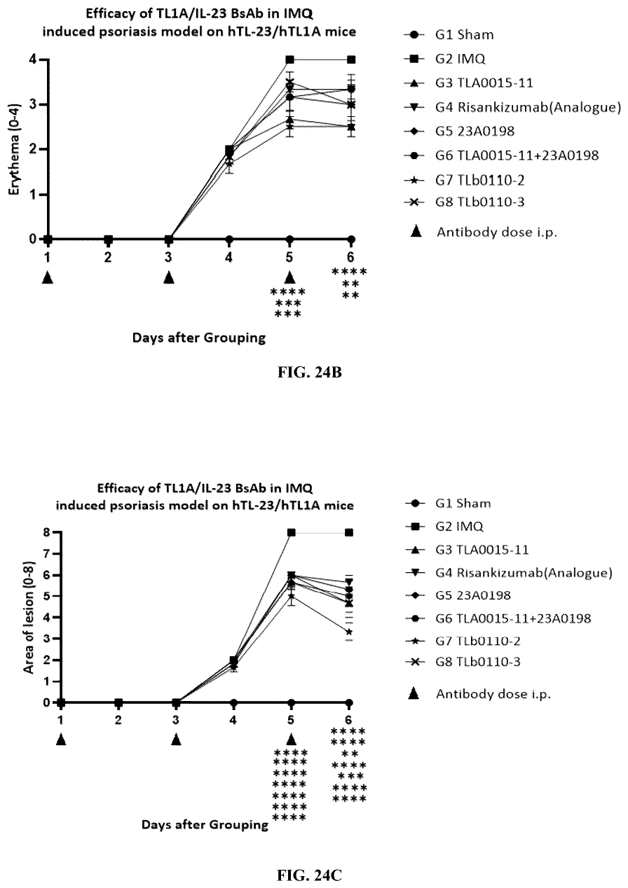

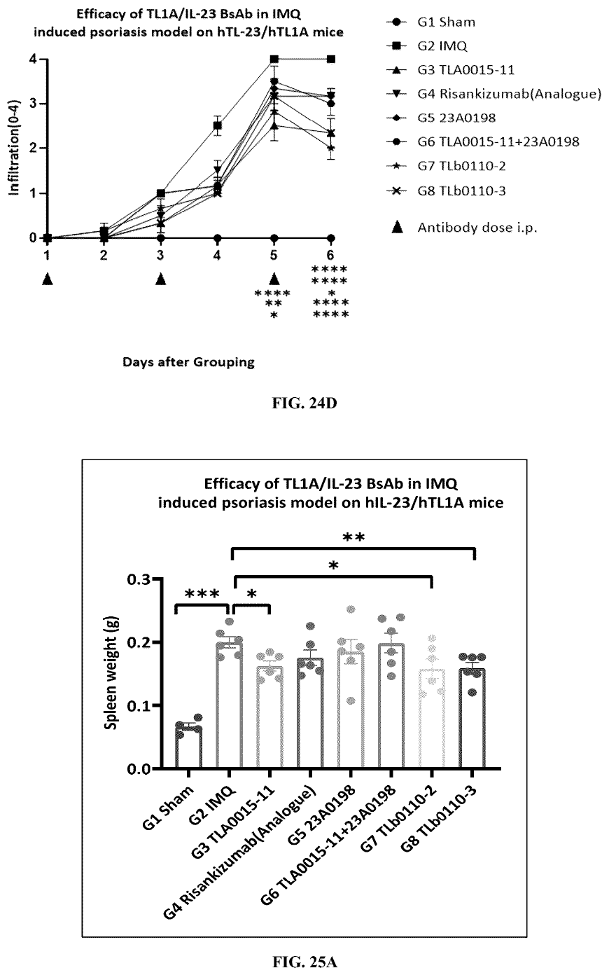

The results of daily scale scores, erythema scores, skin damage scores, and skin infiltration scores at the modeling site of mice are shown in FIGS. 24A, 24B, 24C, and 24D, respectively. Compared with the G2 modeling group, each treatment group can see obvious drug effects, among which the G3 TLA0015-11 monoclonal antibody administration group and the G7 TLb0110-2 bispecific antibody administration group have the best drug effects, which are better than the G4 Risankizumab (Analogue) control antibody administration group.

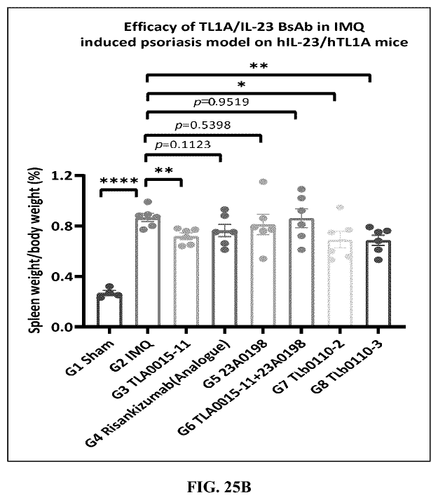

The results of weighing the mouse spleen weight after the experiment are shown in FIG. 25A. The spleen weight of the mice in the G2 modeling group increased significantly, and the spleen weight of the mice in the G3 TLA0015-11 monoclonal antibody administration group, the G7 TLb0110-2 bispecific antibody administration group, and the G8 TLb0110-3 bispecific antibody administration group was improved compared with the G2 modeling group. The results of the ratio of mouse spleen weight to body weight are shown in FIG. 25B. The results are consistent with the results of mouse spleen weight. The spleen weight of the mice in the G3 TLA0015-11 monoclonal antibody administration group, the G7 TLb0110-2 bispecific antibody administration group, and the G8 TLb0110-3 bispecific antibody administration group was improved compared with the G2 modeling group.

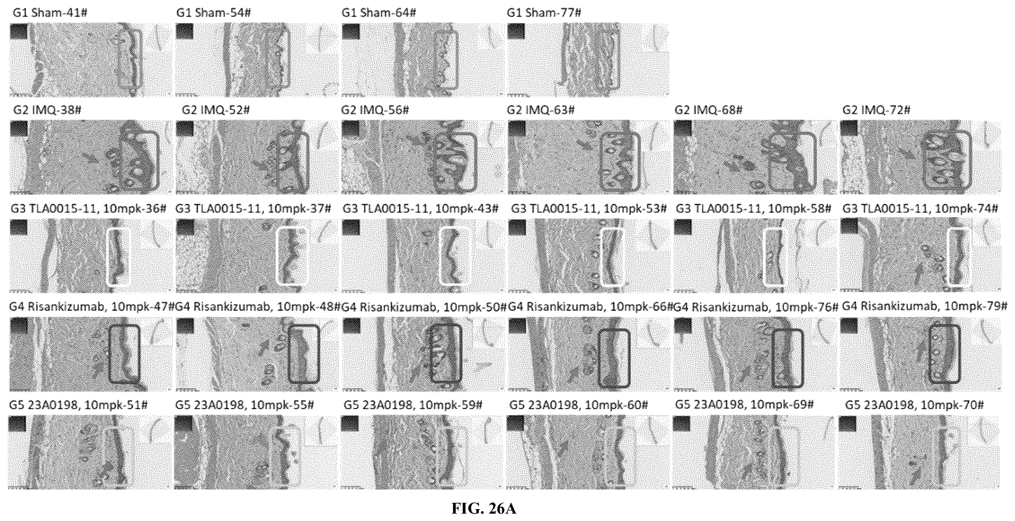

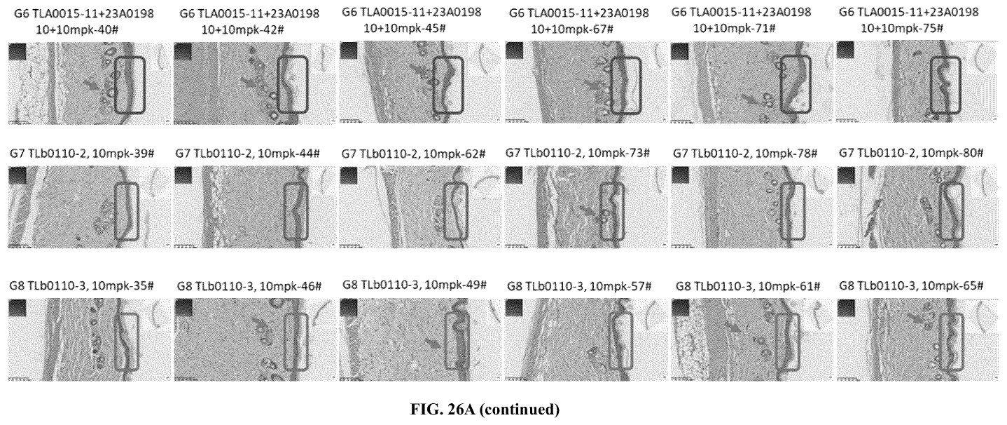

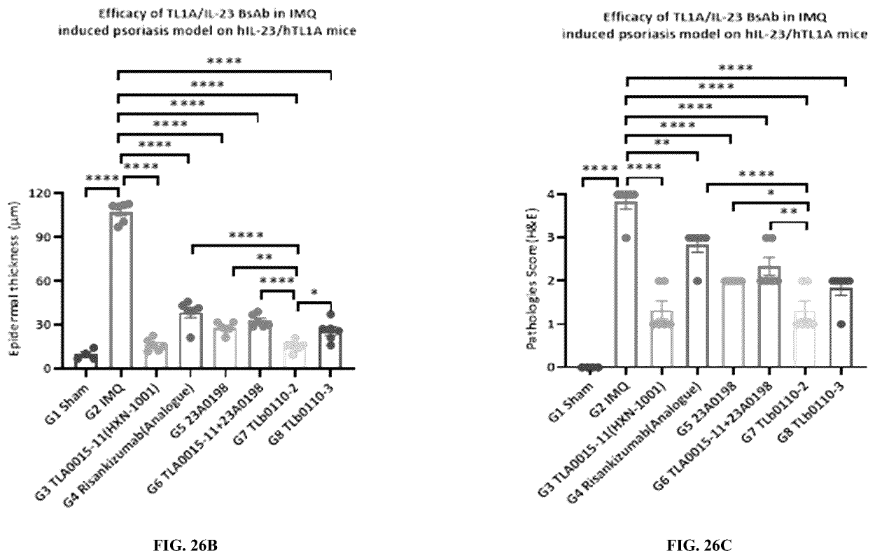

The results of H&E staining of the modeling site are shown in FIG. 26A. The thickness of the epidermis layer of the skin was statistically analyzed, and the results are shown in FIG. 26B. The epidermis of the mice in the G2 modeling group was significantly thickened, among which the epidermis thickness of the G3 TLA0015-11 monoclonal antibody administration group and the G7 TLb0110-2 bispecific antibody administration group improved most significantly, and the drug efficacy was the best. In addition, the pathological scoring of the H&E results of the sections is shown in FIG. 26C. The epidermis thickness of the G3 TLA0015-11 monoclonal antibody administration group and the G7 TLb0110-2 bispecific antibody administration group improved most significantly, and the drug efficacy was the best.

In summary, the bispecific antibody can see obvious drug efficacy in the IMQ-induced psoriasis model, and is better than the control antibody Risankizumab (Analogue).

TABLE 13
Mouse grouping and dosing details

| Group | Animal | Modeling | Treatment | Number | Dosing |
|---|---|---|---|---|---|
| G1 | hTL1A/ | / | / | 4 | / |
| G2 | hIL-23 | IMQ | PBS | 6 | |
| G3 | mice | | TLA0015-11 | 6 | 10 mg/kg |
| G4 | | | Risankizumab | 6 | 10 mg/kg |
| | | | (Analogue) | | |
| G5 | | | 23A0198 | 6 | 10 mg/kg |
| G6 | | | TLA0015-11 + | 6 | 10 mg/kg + |
| | | | 23A0198 | | 10 mg/kg |
| G7 | | | TLb0110-2 | 6 | 10 mg/kg |
| G8 | | | TLb0110-3 | 6 | 10 mg/kg |

### Example 14 Detection of the Pharmacodynamic Activity of the Bispecific Antibody in the IL-23+TL1A-Induced Rat Psoriasis Model

This experiment uses the IL-23+TL1A protein-induced rat psoriasis model to evaluate the in vivo pharmacodynamic activity of the bispecific antibody. Direct injection of IL-23 into the rat ear skin can stimulate and promote Th17 cell differentiation and induce the secretion of cytokines such as IL-17 and IL-22, thereby producing pathological characteristics similar to psoriasis. On the basis of IL-23, the simultaneous injection of TL1A protein can aggravate the symptoms of psoriasis. Therefore, the psoriasis model induced by IL-23+TL1A can be used to evaluate the in vivo pharmacodynamic activity of the bispecific antibody.

Thirty-seven female SD rats (purchased from Vital River) were randomly divided into eight groups, including two rats in the G1 negative control group (no modeling), and 35 rats in the modeling group, including seven groups, namely G2 modeling group, G3 TLA0015-11 administration group, G4 Risankizumab (Analogue) administration group, G5 23A0198 administration group, G6 TLA0015-11 and 23A0198 monoclonal antibody combination group, G7 TLb0110-2 bispecific antibody administration group and G8 TLb0110-3 bispecific antibody administration group, with five rats in each group. The detailed grouping of rats is shown in Table 14. After the rats were randomly divided into groups on day 0, the modeling was started on day 1. The G1 negative control group was injected with PBS in the ear skin, and the G2 to G8 groups were injected with a mixture of 500 ng IL-23 and 500 ng TL1A protein in the ear skin, and then injected every other day until the 13th day. The drug administration group was injected with drugs through the tail vein on the 0th, 4th, 8th and 12th days, with a dose of 5 mg/kg. During the experiment, the weight of the experimental animals and the thickness of the skin at the modeling site were measured and recorded every other day; the mouse skin lesion area and severity index (PASI) was scored every other day, and the scales, erythema and infiltration of the mouse ear skin were scored. The experiment ended on the 17th day, and all experimental animals were euthanized by excessive carbon dioxide asphyxiation, the ear tissue was retained, and the rat carcasses were harmlessly disposed of.

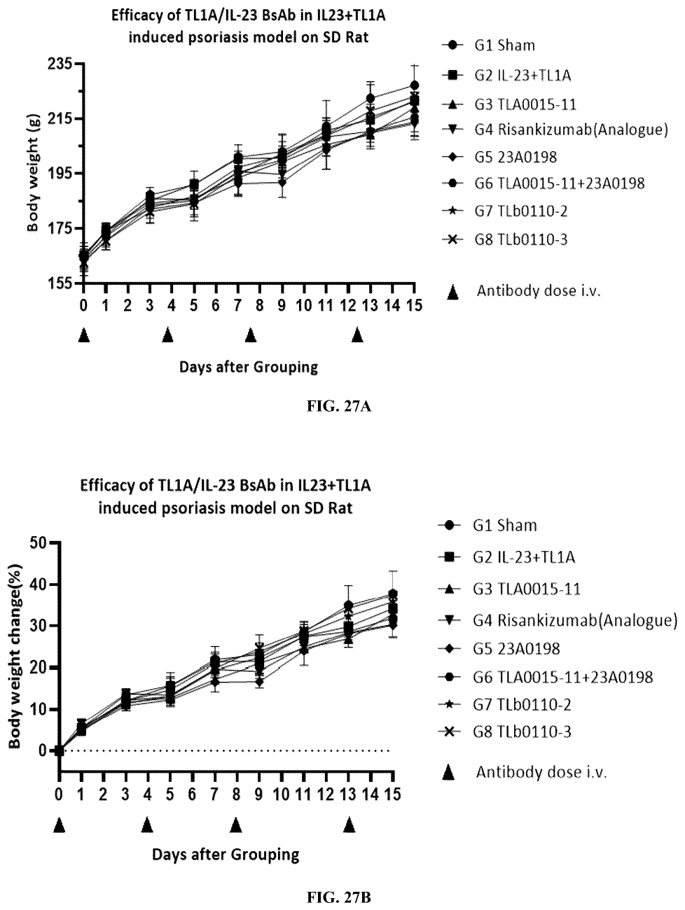

After the experiment, the results were statistically analyzed, and the results of the rat weight change are shown in FIGS. 27A and 27B. The weight of rats in each group is steadily increasing, and the difference in weight between rats in each group is very small.

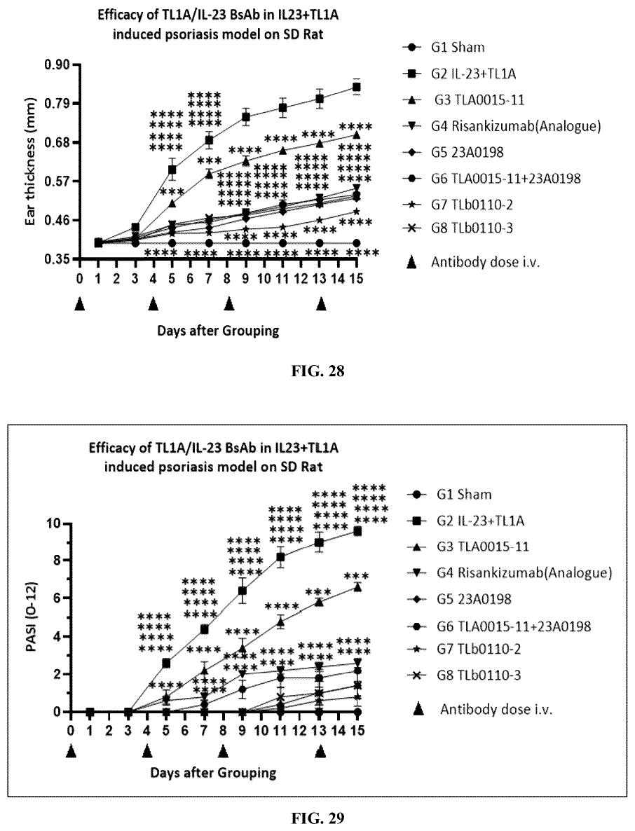

The results of the change in skin thickness at the modeling site of rats are shown in FIG. 28. It can be seen that in the G2 modeling group, the skin thickness of the rat modeling site increased significantly, and the six drug administration groups G3-G8 can see obvious drug effects. Among them, the skin thickness of rats in the G7 TLb0110-2 bispecific antibody administration group improved most significantly; the four groups of G4 Risankizumab (Analogue) control antibody administration group, G5 23A0198 monoclonal antibody administration group, G6 monoclonal antibody combination group and G8 TLb0110-3 bispecific antibody administration group had little difference, and compared with the G2 modeling group, they could also see obvious drug effects; the G3 TLA0015-11 monoclonal antibody group had the weakest drug effect.

The PASI score results of rats are shown in FIG. 29. It can be seen that the PSAI score of the G2 modeling group increased significantly, and all six administration groups showed obvious drug effects. Among them, the scores of G5 23A0198 monoclonal antibody administration group, G7 TLb0110-2 bispecific antibody administration group and G8 TLb0110-3 bispecific antibody administration group were the lowest, the scores of G4 Risankizumab (Analogue) control antibody administration group and G6 monoclonal antibody combination group were close, and the scores of G3 TLA0015-11 monoclonal antibody administration group were higher than other administration groups.

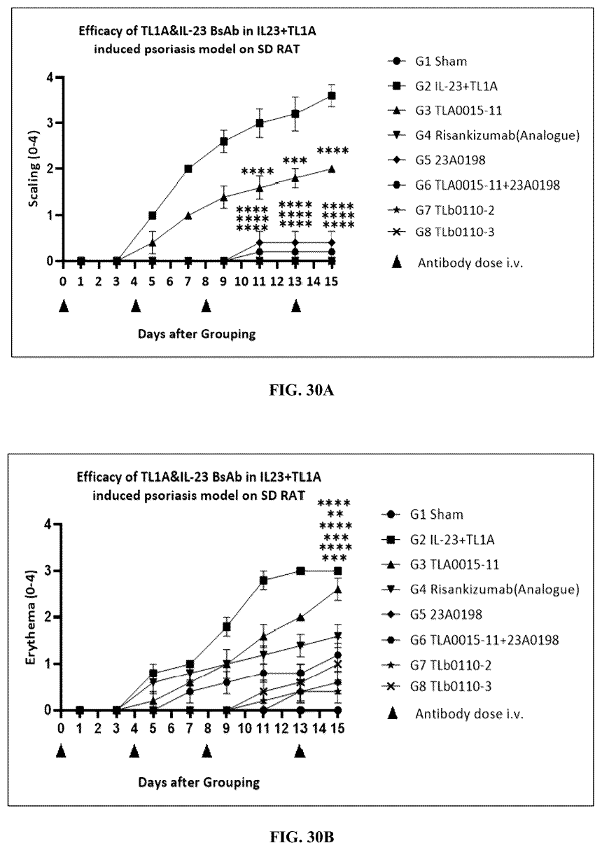

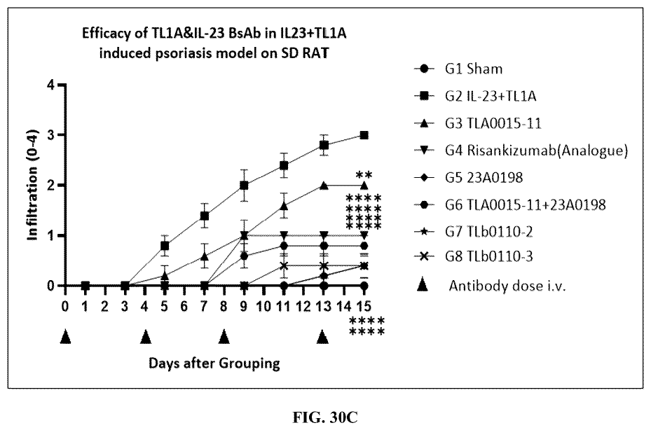

The results of daily scale score, erythema score and skin infiltration degree score of rat ear skin are shown in FIGS. 30A, 30B and 30C respectively. Compared with the G2 modeling group, each treatment group can see obvious drug efficacy. In the scale score, the G4 Risankizumab (Analogue) control antibody administration group, the G7 TLb0110-2 bispecific antibody administration group, and the G8 TLb0110-3 bispecific antibody administration group had the most obvious efficacy (FIG. 30A); in the erythema score, the G7 TLb0110-2 bispecific antibody administration group and the G5 23A0198 monoclonal antibody administration group had the most obvious efficacy (FIG. 30B); in the infiltration score, the G7 TLb0110-2 bispecific antibody administration group and the G5 23A0198 monoclonal antibody administration group had the most obvious efficacy (FIG. 30C).

In summary, the bispecific antibody can be seen in the IL-23+TL1A-induced psoriasis model, and is superior to the control antibody Risankizumab (Analogue).

TABLE 14
Details of rat grouping and dosing

| Group | Animal | Modeling | Treatment | Number | Dosing |
|---|---|---|---|---|---|
| G1 | SD Rat | / | / | 2 | / |
| G2 | | 500 ng | PBS | 5 | |
| G3 | | IL-23 + | TLA0015-11 | 5 | 5 mg/kg |
| G4 | | 500 ng | Risankizumab | 5 | 5 mg/kg |
| | | TL1A | (Analogue) | | |
| G5 | | | 23A0198 | 5 | 5 mg/kg |
| G6 | | | TLA0015-11 + | 5 | 5 mg/kg + |
| | | | 23A0198 | | 5 mg/kg |
| G7 | | | TLb0110-2 | 5 | 5 mg/kg |
| G8 | | | TLb0110-3 | 5 | 5 mg/kg |

In summary, the sequences used in the present application are shown in the table below, wherein [ ] represents that the position can be arbitrarily replaced by the amino acids shown in [ ].

| ID | LABEL | SEQ |
|---|---|---|
| 1 | TLA0015-HCDR1 | [STY]YGVD |
| 2 | TLA0015-HCDR1-1 | SYGVD |
| 3 | TLA0015-HCDR1-2 | YYGVD |
| 4 | TLA0015-HCDR1-3 | TYGVD |
| 5 | TLA0015-HCDR2 | [RV]IWGFG[GK]T[DHN]Y[NQ] |
| | | [APS]ALKS |
| 6 | TLA0015-HCDR2-1 | VIWGFGGTNYNSALKS |
| 7 | TLA0015-HCDR2-2 | VIWGFGGTNYQSALKS |
| 8 | TLA0015-HCDR2-3 | VIWGFGGTNYNAALKS |
| 9 | TLA0015-HCDR2-4 | VIWGFGGTNYNPALKS |
| 10 | TLA0015-HCDR2-5 | VIWGFGGTHYNSALKS |
| 11 | TLA0015-HCDR2-6 | RIWGFGGTNYNSALKS |
| 12 | TLA0015-HCDR2-7 | VIWGFGKTNYNSALKS |
| 13 | TLA0015-HCDR2-8 | VIWGFGGTDYNSALKS |
| 14 | TLA0015-HCDR3 | G[DLN][FY]DAMDY |
| 15 | TLA0015-HCDR3-1 | GNFDAMDY |
| 16 | TLA0015-HCDR3-2 | GNYDAMDY |
| 17 | TLA0015-HCDR3-3 | GDFDAMDY |
| 18 | TLA0015-HCDR3-4 | GLEDAMDY |
| 19 | TLA0015-HFR1 | E[IV]QL[LVJESGGGL[IV]QPGGSLR |
| | | [IL]SCAVSGF[DS]L[IKS] |
| 20 | TLA0015-HFR1-1 | EIQLLESGGGLVQPGGSLRLSCAVSGFSLS |
| 21 | TLA0015-HFR1-2 | EIQLLESGGGLVQPGGSLRISCAVSGFSLS |
| 22 | TLA0015-HFR1-3 | EIQLVESGGGLVQPGGSLRISCAVSGFSLS |
| 23 | TLA0015-HFR1-4 | EVQLLESGGGLVQPGGSLRLSCAVSGFSLS |
| 24 | TLA0015-HFR1-5 | EIQLVESGGGLIQPGGSLRLSCAVSGFSLS |
| 25 | TLA0015-HFR1-6 | EIQLVESGGGLIQPGGSLRISCAVSGFSLS |
| 26 | TLA0015-HFR1-7 | EIQLVESGGGLIQPGGSLRISCAVSGFSLI |
| 27 | TLA0015-HFR1-8 | EIQLVESGGGLIQPGGSLRISCAVSGFSLK |
| 28 | TLA0015-HFR1-9 | EIQLVESGGGLIQPGGSLRISCAVSGFDLS |
| 29 | TLA0015-HFR2 | WVRQ[AS]PGKGLEW[LV]G |
| 30 | TLA0015-HFR2-1/ | WVRQAPGKGLEWVG |
| | TLA-0238-HFR2-3 | |
| 31 | TLA0015-HFR2-2 | WVRQAPGKGLEWLG |
| 32 | TLA0015-HFR2-3 | WVRQSPGKGLEWLG |
| 33 | TLA0015-HFR3 | R[FL]TIS[AKRV]DNSKNT[LV]YLQMNS |
| | | LRAEDTAVYYCA[RS] |
| 34 | TLA0015-HFR3-1 | RLTISRDNSKNTLYLQMNSLRAEDTAVYYC |
| | | AS |
| 35 | TLA0015-HFR3-2 | RLTISKDNSKNTVYLQMNSLRAEDTAVYYC |
| | | AS |
| 36 | TLA0015-HFR3-3 | RFTISRDNSKNTVYLQMNSLRAEDTAVYYC |
| | | AS |
| 37 | TLA0015-HFR3-4 | RLTISRDNSKNTVYLQMNSLRAEDTAVYYC |
| | | AS |
| 38 | TLA0015-HFR3-5 | RLTISRDNSKNTVYLQMNSLRAEDTAVYYC |
| | | AR |
| 39 | TLA0015-HFR3-6 | RLTISVDNSKNTVYLQMNSLRAEDTAVYYC |
| | | AS |
| 40 | TLA0015-HFR3-7 | RLTISADNSKNTVYLQMNSLRAEDTAVYYC |
| | | AS |
| 41 | TLA0015-HFR4-1/ | WGQGTLVTVSS |
| | TLA-0238-HFR4-2 | |
| 42 | TLA0015-LCDR1-1 | RASQDISNYLN |
| 43 | TL0015-LCDR2-1 | YTSRLHS |
| 44 | TL0015-LCDR3-1 | QQGNTLPFT |
| 45 | TL0015-LFR1 | DIQMTQ[ST][PT]SSLSASVGDRVTI |
| | | [TS]C |
| 46 | TL0015-LFR1-1 | DIQMTQTTSSLSASLGDRVTISC |
| 47 | TL0015-LFR1-2/ | DIQMTQSPSSLSASVGDRVTITC |
| | TLA0241-LFR1-1 | |
| 48 | TL0015-LFR2 | WYQQKPGK[AV][PV]KLLIY |
| 49 | TL0015-LFR2-1 | WYQQKPDGTVKLLIY |
| 50 | TL0015-LFR2-2 | WYQQKPGKVVKLLIY |
| 51 | TL0015-LFR2-3 | WYQQKPGKAVKLLIY |
| 52 | TL0015-LFR2-4/ | WYQQKPGKAPKLLIY |
| | TLA0241-LFR2-1 | |
| 53 | TL0015-LFR3 | GVPSRFSGSGSGTD[FY]TLTISSLQPED |
| | | [FV]ATY[FY]C |
| 54 | TL0015-LFR3-1 | GVPSRFSGSGSGTDYSLTISNLEQEDIATY |
| | | FC |
| 55 | TL0015-LFR3-2 | GVPSRFSGSGSGTDYTLTISSLQPEDVATY |
| | | FC |
| 56 | TL0015-LFR3-3 | GVPSRFSGSGSGTDYTLTISSLQPEDFATY |
| | | FC |
| 57 | TL0015-LFR3-4/ | GVPSRFSGSGSGTDFTLTISSLQPEDFATY |
| | TLA0238-LFR3-4/ | YC |
| | TLA0241-LFR3-2 | |
| 58 | TL0015-LFR4 | FG[PQ]GTKLEIK |
| 59 | TL0015-LFR4-1 | FGSGTKLEIK |
| 60 | TL0015-LFR4-2 | FGPGTKLEIK |
| 61 | TL0015-LFR4-3/ | FGQGTKLEIK |
| | TLA0238-LFR4-3 | |
| 62 | TLA0015-VH-1 | QIQLKESGPGLVAPSQSLSITCTVSGFSLS |
| | | SYGVDWVRQSPGKGLEWLGVIWGFGGTNYN |
| | | SALKSRLSISKDNSKSQVFLKMNSLQSDDS |
| | | AIYYCASGNFDAMDYWGQGTSVTVSS |
| 63 | TLA0015-VH-2 | EIQLLESGGGLVQPGGSLRLSCAVSGFSLS |
| | | SYGVDWVRQAPGKGLEWVGVIWGFGGTNYN |
| | | SALKSRLTISRDNSKNTLYLQMNSLRAEDT |
| | | AVYYCASGNFDAMDYWGQGTLVTVSS |
| 64 | TLA0015-VH-3 | EIQLLESGGGLVQPGGSLRISCAVSGESLS |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 65 | TLA0015-VH-4 | EIQLVESGGGLVQPGGSLRISCAVSGFSLS |
| | | SYGVDWVRQSPGKGLEWLGVIWGFGGTNYN |
| | | SALKSRLTISKDNSKNTVYLQMNSLRAEDT |
| | | AVYYCASGNFDAMDYWGQGTLVTVSS |
| 66 | TLA0015-VH-5 | EVQLLESGGGLVQPGGSLRLSCAVSGFSLS |
| | | SYGVDWVRQAPGKGLEWLGVIWGFGGTNYN |
| | | SALKSRFTISRDNSKNTVYLQMNSLRAED |
| | | TAVYYCASGNFDAMDYWGQGTLVTVSS |
| 67 | TLA0015-VH-6 | EVQLLESGGGLVQPGGSLRLSCAVSGFSLS |
| | | SYGVDWVRQAPGKGLEWLGVIWGFGGTNYN |
| | | SALKSRLTISRDNSKNTVYLQMNSLRAED |
| | | TAVYYCASGNFDAMDYWGQGTLVTVSS |
| 68 | TLA0015-VH-7 | EVQLLESGGGLVQPGGSLRLSCAVSGFSLS |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 69 | TLA0015-VH-8 | EIQLVESGGGLIQPGGSLRLSCAVSGFSLS |
| | | SYGVDWVRQAPGKGLEWLGVIWGFGGTNYN |
| | | SALKSRLTISRDNSKNTVYLQMNSLRAEDT |
| | | AVYYCARGNFDAMDYWGQGTLVTVSS |
| 70 | TLA0015-VH-9 | EIQLVESGGGLIQPGGSLRISCAVSGFSLS |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 71 | TLA0015-VH-10 | EIQLVESGGGLIQPGGSLRISCAVSGFSLS |
| | | SYGVDWVRQAPGKGLEWLGVIWGFGGTNYN |
| | | SALKSRLTISRDNSKNTVYLQMNSLRAEDT |
| | | AVYYCASGNFDAMDYWGQGTLVTVSS |
| 72 | TLA0015-VH-11 | EIQLVESGGGLIQPGGSLRISCAVSGFSLS |
| | | SYGVDWVRQAPGKGLEWLGVIWGFGGTNYN |
| | | SALKSRLTISVDNSKNTVYLQMNSLRAEDT |
| | | AVYYCASGNFDAMDYWGQGTLVTVSS |
| 73 | TLA0015-VH-12 | EIQLVESGGGLIQPGGSLRISCAVSGFSLS |
| | | SYGVDWVRQAPGKGLEWLGVIWGFGGTNYQ |
| | | SALKSRLTISKDNSKNTVYLQMNSLRAEDT |
| | | AVYYCASGNFDAMDYWGQGTLVTVSS |
| 74 | TLA0015-VH-13 | EIQLVESGGGLIQPGGSLRISCAVSGFSLS |
| | | SYGVDWVRQAPGKGLEWLGVIWGFGGTNYN |
| | | AALKSRLTISKDNSKNTVYLQMNSLRAEDT |
| | | AVYYCASGNFDAMDYWGQGTLVTVSS |
| 75 | TLA0015-VH-14 | EIQLVESGGGLIQPGGSLRISCAVSGFSLS |
| | | SYGVDWVRQAPGKGLEWLGVIWGFGGTNYN |
| | | SALKSRLTISADNSKNTVYLQMNSLRAEDT |
| | | AVYYCASGNFDAMDYWGQGTLVTVSS |
| 76 | TLA0015-VH-15 | EIQLVESGGGLIQPGGSLRISCAVSGFSLS |
| | | YYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 77 | TLA0015-VH-16 | EIQLVESGGGLIQPGGSLRISCAVSGFSLS |
| | | SYGVDWVRQAPGKGLEWLGVIWGFGGTNYN |
| | | SALKSRLTISKDNSKNTVYLQMNSLRAEDT |
| | | AVYYCASGNYDAMDYWGQGTLVTVSS |
| 78 | TLA0015-VH-17 | EIQLVESGGGLIQPGGSLRISCAVSGFSLS |
| | | SYGVDWVRQAPGKGLEWLGVIWGFGGTNYN |
| | | SALKSRLTISKDNSKNTVYLQMNSLRAEDT |
| | | AVYYCASGDFDAMDYWGQGTLVTVSS |
| 79 | TLA0015-VH-18 | EIQLVESGGGLIQPGGSLRISCAVSGESLS |
| | | SYGVDWVRQAPGKGLEWLGVIWGFGGTNYN |
| | | SALKSRLTISKDNSKNTVYLQMNSLRAEDT |
| | | AVYYCASGLEDAMDYWGQGTLVTVSS |
| 80 | TLA0015-VH-19 | EIQLVESGGGLIQPGGSLRISCAVSGFSLS |
| | | TYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 81 | TLA0015-VH-20 | EIQLVESGGGLIQPGGSLRISCAVSGFSLI |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 82 | TLA0015-VH-21 | EIQLVESGGGLIQPGGSLRISCAVSGFSLS |
| | | SYGVDWVRQAPGKGLEWLGVIWGFGGTNYN |
| | | PALKSRLTISKDNSKNTVYLQMNSLRAEDT |
| | | AVYYCASGNFDAMDYWGQGTLVTVSS |
| 83 | TLA0015-VH-22 | EIQLVESGGGLIQPGGSLRISCAVSGFSLK |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 84 | TLA0015-VH-23 | EIQLVESGGGLIQPGGSLRISCAVSGFSLS |
| | | SYGVDWVRQAPGKGLEWLGVIWGFGGTHYN |
| | | SALKSRLTISKDNSKNTVYLQMNSLRAEDT |
| | | AVYYCASGNFDAMDYWGQGTLVTVSS |
| 85 | TLA0015-VH-24 | EIQLVESGGGLIQPGGSLRISCAVSGFSLS |
| | | SYGVDWVRQAPGKGLEWLGRIWGFGGTNYN |
| | | SALKSRLTISKDNSKNTVYLQMNSLRAEDT |
| | | AVYYCASGNFDAMDYWGQGTLVTVSS |
| 86 | TLA0015-VH-25 | EIQLVESGGGLIQPGGSLRISCAVSGFDLS |
| | | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 87 | TLA0015-VH-26 | EIQLVESGGGLIQPGGSLRISCAVSGFSLS |
| | | SYGVDWVRQAPGKGLEWLGVIWGFGKTNYN |
| | | SALKSRLTISKDNSKNTVYLQMNSLRAEDT |
| | | AVYYCASGNFDAMDYWGQGTLVTVSS |
| 88 | TLA0015-VH-27 | EIQLVESGGGLIQPGGSLRISCAVSGFSLS |
| | | SYGVDWVRQAPGKGLEWLGVIWGFGGTDYN |
| | | SALKSRLTISKDNSKNTVYLQMNSLRAEDT |
| | | AVYYCASGNFDAMDYWGQGTLVTVSS |
| 89 | TLA0015-VH-28 | EIQLVESGGGLIQPGGSLRISCAVSGFSLS |
| | | YYGVDWVRQAPGKGLEWLGVIWGFGGTNYN |
| | | SALKSRLTISKDNSKNTVYLQMNSLRAEDT |
| | | AVYYCASGNYDAMDYWGQGTLVTVSS |
| 90 | TLA0015-VH-29 | EIQLVESGGGLIQPGGSLRISCAVSGFSLS |
| | | YYGVDWVRQAPGKGLEWLGVIWGFGGTNYN |
| | | SALKSRLTISKDNSKNTVYLQMNSLRAEDT |
| | | AVYYCASGDFDAMDYWGQGTLVTVSS |
| 91 | TLA0015-VH-30 | EIQLVESGGGLIQPGGSLRISCAVSGESLS |
| | | YYGVDWVRQAPGKGLEWLGVIWGFGGTNYN |
| | | PALKSRLTISKDNSKNTVYLQMNSLRAEDT |
| | | AVYYCASGNFDAMDYWGQGTLVTVSS |
| 92 | TLA0015-VH-31 | EIQLVESGGGLIQPGGSLRISCAVSGFSLS |
| | | YYGVDWVRQAPGKGLEWLGVIWGFGGTHYN |
| | | SALKSRLTISKDNSKNTVYLQMNSLRAEDT |
| | | AVYYCASGNFDAMDYWGQGTLVTVSS |
| 93 | TLA0015-VH-32 | EIQLVESGGGLIQPGGSLRISCAVSGFSLS |
| | | YYGVDWVRQAPGKGLEWLGRIWGFGGTNYN |
| | | SALKSRLTISKDNSKNTVYLQMNSLRAEDT |
| | | AVYYCASGNFDAMDYWGQGTLVTVSS |
| 94 | TLA0015-VH-33 | EIQLVESGGGLIQPGGSLRISCAVSGFDLS |
| | | YYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSS |
| 95 | TLA0015-VH-34 | EIQLVESGGGLIQPGGSLRISCAVSGFSLI |
| | | SYGVDWVRQAPGKGLEWLGVIWGFGGTNYN |
| | | SALKSRLTISKDNSKNTVYLQMNSLRAEDT |
| | | AVYYCASGNYDAMDYWGQGTLVTVSS |
| 96 | TLA0015-VH-35 | EIQLVESGGGLIQPGGSLRISCAVSGFSLS |
| | | SYGVDWVRQAPGKGLEWLGVIWGFGGTNYN |
| | | PALKSRLTISKDNSKNTVYLQMNSLRAEDT |
| | | AVYYCASGNYDAMDYWGQGTLVTVSS |
| 97 | TLA0015-VH-36 | EIQLVESGGGLIQPGGSLRISCAVSGFSLS |
| | | SYGVDWVRQAPGKGLEWLGVIWGFGGTHYN |
| | | SALKSRLTISKDNSKNTVYLQMNSLRAEDT |
| | | AVYYCASGNYDAMDYWGQGTLVTVSS |
| 98 | TLA0015-VH-37 | EIQLVESGGGLIQPGGSLRISCAVSGFSLS |
| | | SYGVDWVRQAPGKGLEWLGRIWGFGGTNYN |
| | | SALKSRLTISKDNSKNTVYLQMNSLRAEDT |
| | | AVYYCASGNYDAMDYWGQGTLVTVSS |
| 99 | TLA0015-VH-38 | EIQLVESGGGLIQPGGSLRISCAVSGFSLS |
| | | SYGVDWVRQAPGKGLEWLGVIWGFGKTNYN |
| | | SALKSRLTISKDNSKNTVYLQMNSLRAEDT |
| | | AVYYCASGNYDAMDYWGQGTLVTVSS |
| 100 | TLA0015-VH-39 | EIQLVESGGGLIQPGGSLRISCAVSGFSLI |
| | | SYGVDWVRQAPGKGLEWLGVIWGFGGTNYN |
| | | SALKSRLTISKDNSKNTVYLQMNSLRAEDT |
| | | AVYYCASGDFDAMDYWGQGTLVTVSS |
| 101 | TLA0015-VH-40 | EIQLVESGGGLIQPGGSLRISCAVSGFSLS |
| | | SYGVDWVRQAPGKGLEWLGVIWGFGGTHYN |
| | | SALKSRLTISKDNSKNTVYLQMNSLRAEDT |
| | | AVYYCASGDFDAMDYWGQGTLVTVSS |
| 102 | TLA0015-VL-1 | DIQMTQTTSSLSASLGDRVTISCRASQDIS |
| | | NYLNWYQQKPDGTVKLLIYYTSRLHSGVPS |
| | | RFSGSGSGTDYSLTISNLEQEDIATYFCQQ |
| | | GNTLPFTFGSGTKLEIK |
| 103 | TLA0015-VL-2 | DIQMTQSPSSLSASVGDRVTITCRASQDIS |
| | | NYLNWYQQKPGKVVKLLIYYTSRLHSGVPS |
| | | RFSGSGSGTDYTLTISSLQPEDVATYFCQQ |
| | | GNTLPFTFGPGTKLEIK |
| 104 | TLA0015-VL-3 | DIQMTQSPSSLSASVGDRVTITCRASQDIS |
| | | NYLNWYQQKPGKAPKLLIYYTSRLHSGVPS |
| | | RFSGSGSGTDFTLTISSLQPEDFATYYCQQ |
| | | GNTLPFTFGQGTKLEIK |
| 105 | TLA0015-VL-4 | DIQMTQSPSSLSASVGDRVTITCRASQDIS |
| | | NYLNWYQQKPGKAPKLLIYYTSRLHSGVPS |
| | | RFSGSGSGTDYTLTISSLQPEDFATYFCQQ |
| | | GNTLPFTFGQGTKLEIK |
| 106 | TLA0015-VL-5 | DIQMTQSPSSLSASVGDRVTITCRASQDIS |
| | | NYLNWYQQKPGKAVKLLIYYTSRLHSGVPS |
| | | RFSGSGSGTDYTLTISSLQPEDFATYFCQQ |
| | | GNTLPFTFGQGTKLEIK |
| 107 | Human TL1A | MAEDLGLSFGETASVEMLPEHGSCRPKARS |
| | (M1-L251) | SSARWALTCCLVLLPFLAGLTTYLLVSQLR |
| | | AQGEACVQFQALKGQEFAPSHQQVYAPLRA |
| | | DGDKPRAHLTVVRQTPTQHFKNQFPALHWE |
| | | HELGLAFTKNRMNYTNKFLLIPESGDYFIY |
| | | SQVTFRGMTSECSEIRQAGRPNKPDSITVV |
| | | ITKVTDSYPEPTQLLMGTKSVCEVGSNWFQ |
| | | PIYLGAMFSLQEGDKLMVNVSDISLVDYTK |
| | | EDKTFFGAFLL |
| 108 | Human sTL1A | LKGQEFAPSHQQVYAPLRADGDKPRAHLTV |
| | (L72-L251) | VRQTPTQHFKNQFPALHWEHELGLAFTKNR |
| | | MNYTNKFLLIPESGDYFIYSQVTFRGMTSE |
| | | CSEIRQAGRPNKPDSITVVITKVTDSYPEP |
| | | TQLLMGTKSVCEVGSNWFQPIYLGAMESLQ |
| | | EGDKLMVNVSDISLVDYTKEDKTFFGAFLL |
| 109 | Cyno sTL1A | LKGQEFAPSHQQVYAPLRADGDKPRAHLTV |
| | (L72-L251) | VRQTPTQHLKNQFPALHWEHELGLAFTKNR |
| | | MNYTNKFLLIPESGDYFVYSQVTFRGMTSE |
| | | CSEIRQAGRPNKPDSITVVITKVTDSYPEP |
| | | TQLLMGTKSVCEVGSNWFQPIYLGAMFSLQ |
| | | EGDKLMVNVSDISLVDYTKEDKTFFGAFLL |
| 110 | Mouse sTL1A | AGQLRVPGKDCMLRAITEERSEPSPQQVYS |
| | (A61-L252) | PPRGKPRAHLTIKKQTPAPHLKNQLSALHW |
| | | EHDLGMAFTKNGMKYINKSLVIPESGDYFI |
| | | YSQITFRGTTSVCGDISRGRRPNKPDSITV |
| | | VITKVADSYPEPARLLTGSKSVCEISNNWF |
| | | QSLYLGAMESLEEGDRLMVNVSDISLVDYT |
| | | KEDKTFFGAFLL |
| 111 | Rat sTL1A | TGQLRIPGKDCMFPTVTEERSAPSAQPVYT |
| | (T61-1252) | PSRDKPKAHLTIMRQTPVPHLKNELAALHW |
| | | ENNLGMAFTKNRMNYTNKFLVIPESGDYFI |
| | | YSQITFRGTTSECGDISRVRRPKKPDSITV |
| | | VITKVADSYPEPAHLLTGTKSVCEISSNWF |
| | | QPIYLGAMESLEEGDRLMVNVSDISLVDYT |
| | | KEDKTFFGAFLI |
| 112 | Human DR3-ECD | QGGTRSPRCDCAGDFHKKIGLFCCRGCPAG |
| | (Q25-Q199) | HYLKAPCTEPCGNSTCLVCPQDTFLAWENH |
| | | HNSECARCQACDEQASQVALENCSAVADTR |
| | | CGCKPGWFVECQVSQCVSSSPFYCQPCLDC |
| | | GALHRHTRLLCSRRDTDCGTCLPGFYEHGD |
| | | GCVSCPTSTLGSCPERCAAVCGWRQ |
| 113 | 18E5-HCDR-1 | RYL[IM][HN] |
| 114 | 18E5-HCDR-1-1 | RYLIH |
| 115 | 18E5-HCDR-1-2 | RYLMH |
| 116 | 18E5-HCDR-1-3 | RYLMN |
| 117 | 18E5-HCDR-2 | YINPYN[DE][AG]TKY[AN][AEQ][KP] |
| | | [FV][KQIG |
| 118 | 18E5-HCDR-2-1 | YINPYNDGTKYNEKFKG |
| 119 | 18E5-HCDR-2-2 | YINPYNDGTKYAQKFQG |
| 120 | 18E5-HCDR-2-3 | YINPYNDGTKYAAPVKG |
| 121 | 18E5-HCDR-2-4 | YINPYNDGTKYNQKFQG |
| 122 | 18E5-HCDR-2-5 | YINPYNDATKYNEKFKG |
| 123 | 18E5-HCDR-2-6 | YINPYNEGTKYNEKFKG |
| 124 | 18E5-HCDR-3 | NWDLDY |
| 125 | 18E5-LCDR-1 | RASQSISDYLY |
| 126 | 18E5-LCDR-2 | FASQSIS |
| 127 | 18E5-LCDR-3 | [NQ][AG]HSFPFT |
| 128 | 18E5-LCDR-3-1 | NGHSFPFT |
| 129 | 18E5-LCDR-3-2 | QGHSFPFT |
| 130 | 18E5-LCDR-3-3 | NAHSFPFT |
| 131 | scTLA0015-11 | EIQLVESGGGLIQPGGSLRISCAVSGFSLS |
| | VHVL | SYGVDWVRQAPGKGLEWLGVIWGFGGTNYN |
| | | SALKSRLTISKDNSKNTVYLQMNSLRAEDT |
| | | AVYYCASGNFDAMDYWGQGTLVTVSSGGGG |
| | | SGGGGSGGGGSGGGGSDIQMTQSPSSLSAS |
| | | VGDRVTITCRASQDISNYLNWYQQKPGKAV |
| | | KLLIYYTSRLHSGVPSRFSGSGSGTDYTLT |
| | | ISSLQPEDFATYFCQQGNTLPFTFGQGTKL |
| | | EIK |
| 132 | scTLA0015-11 | DIQMTQSPSSLSASVGDRVTITCRASQDIS |
| | VLVH | NYLNWYQQKPGKAVKLLIYYTSRLHSGVPS |
| | | RFSGSGSGTDYTLTISSLQPEDFATYFCQQ |
| | | GNTLPFTFGQGTKLEIKGGGGSGGGGSGGG |
| | | GSGGGGSEIQLVESGGGLIQPGGSLRISCA |
| | | VSGFSLSSYGVDWVRQAPGKGLEWLGVIWG |
| | | FGGTNYNSALKSRLTISKDNSKNTVYLQMN |
| | | SLRAEDTAVYYCASGNFDAMDYWGQGTLVT |
| | | VSS |
| 133 | scTLA0015-11cc | EIQLVESGGGLIQPGGSLRISCAVSGFSLS |
| | VHVL | SYGVDWVRQAPGKCLEWLGVIWGFGGTNYN |
| | | SALKSRLTISKDNSKNTVYLQMNSLRAEDT |
| | | AVYYCASGNFDAMDYWGQGTLVTVSSGGGG |
| | | SGGGGSGGGGSGGGGSDIQMTQSPSSLSAS |
| | | VGDRVTITCRASQDISNYLNWYQQKPGKAV |
| | | KLLIYYTSRLHSGVPSRFSGSGSGTDYTLT |
| | | ISSLQPEDFATYFCQQGNTLPFTFGCGTKL |
| | | EIK |
| 134 | scTLA0015-11cc | DIQMTQSPSSLSASVGDRVTITCRASQDIS |
| | VLVH | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSS |
| 135 | 16003-18E5VH | EVQLQQSGPELVKPGAAVKMSCKASGYTFT |
| | | RYLIHWVKQKPGQGLEWIGYINPYNDGTKY |
| | | NEKFKGKATLTSDKSSSTAYMELSSLTSED |
| | | SAVYYCTSNWDLDYWGQGTTLTVSS |
| 136 | 23A0144VH | QVQLVQSGAEVKKPGSSVKVSCKASGYTFT |
| | | RYLIHWVRQAPGQGLEWIGYINPYNDGTKY |
| | | AQKFQGRVTITADESTSTAYMELSSLRSED |
| | | TAVYYCTSNWDLDYWGQGTLVTVSS |
| 137 | 23A0148VH | EVQLVESGGGLVKPGGSLRLSCAASGYTFT |
| | | RYLIHWVRQAPGKGLEWIGYINPYNDGTKY |
| | | AAPVKGRFTISRDDSKNTLYLQMNSLKTED |
| | | TAVYYCTSNWDLDYWGQGTLVTVSS |
| 138 | 23A0149VH | EVQLQQSGPELVKPGASVKVSCKASGYTFT |
| | | RYLMHWVRQAPGQGLEWIGYINPYNDGTKY |
| | | NQKFQGRVTITADKSTSTAYMELSSLTSED |
| | | TAVYYCTSNWDLDYWGQGTLVTVSS |
| 139 | 23A0150VH | EVQLQQSGPEVKKPGASVKVSCKASGYTFT |
| | | RYLIHWVRQAPGQGLEWIGYINPYNDGTKY |
| | | NOKFQGRVTITADKSTSTAYMELSSLRSED |
| | | TAVYYCTSNWDLDYWGQGTTLTVSS |
| 140 | 23A0151VH | QVQLQQSGSELKKPGASVKVSCKASGYTFT |
| | | RYLMHWVRQAPGKGLEWIGYINPYNDGTKY |
| | | NQKFQGRVTITADKSTSTAYMELSSLRSED |
| | | TAVYYCTSNWDLDYWGQGTLVTVSS |
| 141 | 23A0152VH | EIQLQQSGSELKKPGASVKVSCKASGYTFT |
| | | RYLMNWVRQAPGKGLEWIGYINPYNDGTKY |
| | | NQKFQGRVTITADKSTSTAYMELSSLRSED |
| | | TAVYYCTSNWDLDYWGQGTLVTVSS |
| 142 | 23A0153VH | EVQLQQSGPEVKKPGASVKVSCKASGYTFT |
| | | RYLMHWVRQPPGQGLEWIGYINPYNDGTKY |
| | | NQKFQGRVTITADKSTSTAYMELSSLRSED |
| | | TAVYYCTSNWDLDYWGQGTTLTVSS |
| 143 | 23A0178VH | QVQLVQSGAEVKKPGASVKVSCKASGYTFT |
| | | RYLIHWVRQAPGQGLEWMGYINPYNDGTK |
| | | YNEKFKGRVTMTRDTSTSTVYMELSSLRSE |
| | | DTAVYYCASNWDLDYWGQGTLVTVSS |
| 144 | 23A0182VH | QVQLVQSGAEVKKPGASVKVSCKASGYTFT |
| | | RYLIHWVRQAPGQGLEWIGYINPYNDGTKY |
| | | NEKFKGRVTMTSDKSTSTAYMELSSLRSED |
| | | TAVYYCTSNWDLDYWGQGTLVTVSS |
| 145 | 23A0186-3VH | QVQLVQSGAEVVKPGASVKMSCKASGYTFT |
| | | RYLIHWVKQAPGQGLEWIGYINPYNDATKY |
| | | NEKFKGRATLTSDKSTSTAYMELSSLRSED |
| | | TAVYYCTSNWDLDYWGQGTLVTVSS |
| 146 | 23A0186-5VH | QVQLVQSGAEVVKPGASVKMSCKASGYTFT |
| | | RYLIHWVKQAPGQGLEWIGYINPYNDGTKY |
| | | NEKFKGRATLTSDKSTSTAYMELSSLRSED |
| | | TAVYYCTSNWDLDYWGQGTLVTVSS |
| 147 | 23A0186-6VH | QVQLVQSGAEVVKPGASVKMSCKASGYTFT |
| | | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSS |
| 148 | 23A0198 | QVQLVQSGAEVKKPGSSVKVSCKASGYTFT |
| | (A5M21)VH | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSS |
| 149 | 16003-18E5VL | DIVMTQSPATLSVTPGDRVSLSCRASQSIS |
| | | DYLYWYQQKSHESPRLLIKFASQSISGIPS |
| | | RFTGSGSGSDFTLSIDSVEPDDVGVFFCQN |
| | | GHSFPFTFGGGTKLEIK |
| 150 | 23A0144VL | AIQMTQSPSSLSASVGDRVTITCRASQSIS |
| | | DYLYWYQQKPGKAPKLLIKFASQSISGVPS |
| | | RFSGSGSGTDFTLTISSLQPEDFATYYCQN |
| | | GHSFPFTFGPGTKVDIK |
| 151 | 23A0145VL | DIQMTQSPSSLSASVGDRVTITCRASQSIS |
| | | DYLYWFQQKPGKAPKLLIKFASQSISGVPS |
| | | RFSGSGSGTDFTLTISSLQPEDFATYYCQN |
| | | GHSFPFTFGQGTRLEIK |
| 152 | 23A0146VL | NIQMTQSPSAMSASVGDRVTITCRASQSIS |
| | | DYLYWFQQKPGKVPKLLIKFASQSISGVPS |
| | | RFSGSGSGTEFTLTISSLQPEDFATYYCQN |
| | | GHSFPFTFGPGTKVDIK |
| 153 | 23A0147VL | DIQMTQSPSTLSASVGDRVTITCRASQSIS |
| | | DYLYWYQQKPGKAPKLLIKFASQSISGVPS |
| | | RFSGSGSGTEFTLTISSLQPDDFATYYCQN |
| | | GHSFPFTFGPGTKVDIK |
| 154 | 23A0148VL | AIQMTQSPSSLSASVGDRVTITCRASQSIS |
| | | DYLYWYQQKPGKAPKLLIKFASQSISGVPS |
| | | RFSGSGSGTDFTLTISSLQPEDFATYYCQN |
| | | GHSFPFTFGQGTKVEIK |
| 155 | 23A0149VL | AIVMTQSPATLSVSPGERVTLSCRASQSIS |
| | | DYLYWYQQKPDQSPKLLIKFASQSISGVPS |
| | | RFSGSGSGRDFTLTISSLQPEDFAVYYCQN |
| | | GHSFPFTFGGGTKLEIK |
| 156 | 23A0150VL | NIVMTQSPATLSVSPGERATLSCRASQSIS |
| | | DYLYWYQQKPDQSPKLLIKFASQSISGVPS |
| | | RFSGSGSGRDFTLTISSLEPEDFAVYYCQN |
| | | GHSFPFTFGGGTKLEIK |
| 157 | 23A0151VL | EIVMTQSPATLSLSPGERATLSCRASQSIS |
| | | DYLYWYQQKPDQSPKLLIKFASQSISGVPS |
| | | RFSGSGSGTEFTLTISSLQPEDFAVYYCQN |
| | | GHSFPFTFGGGTKVEIK |
| 158 | 23A0152VL | DIVMTQSPATLSVSPGERVTLSCRASQSIS |
| | | DYLYWYQQKPDQSPKLLIKFASQSISGVPS |
| | | RFSGSGSGSDFTLTISSLQPEDFATYYCQN |
| | | GHSFPFTFGGGTKLEIK |
| 159 | 23A0153VL | DIVMTQSPATLSVSPGERVTLSCRASQSIS |
| | | DYLYWYQQKPDQSPRLLIKFASQSISGVPS |
| | | RFSGSGSGSDFTLKISSVEAEDVGVYYCQN |
| | | GHSFPFTFGGGTKVEIK |
| 160 | 23A0183VL | DIQMTQSPSSLSASVGDRVTITCRASQSIS |
| | | DYLYWYQQKPGKAPKLLIYFASQSISGVPS |
| | | RFSGSGSGTDFTLTISSLQPEDFATYYCQN |
| | | GHSFPFTFGGGTKVEIK |
| 161 | 23A0184VL | DIQMTQSPSSLSASVGDRVTITCRASQSIS |
| | | DYLYWYQQKPGKAPKLLIKFASQSISGIPS |
| | | RFSGSGSGSDFTLTISSLQPEDFATFYCQN |
| | | GHSFPFTFGGGTKVEIK |
| 162 | 23A0185VL | DIVMTQSPSSLSASVGDRVTLTCRASQSIS |
| | | DYLYWYQQKPGKAPKLLIKFASQSISGIPS |
| | | RESGSGSGSDFTLTISSVQPEDFATFFCQN |
| | | GHSFPFTFGGGTKVEIK |
| 163 | 23A0186-4VL | EIVMTQSPATLSVSPGERVTLSCRASQSIS |
| | | DYLYWYQQKPGQAPRLLIKFASQSISGIPA |
| | | RFSGSGSGSEFTLTISSVQSEDFAVFFCQN |
| | | AHSFPFTFGGGTKVEIK |
| 164 | 23A0186-5VL | EIVMTQSPATLSVSPGERVTLSCRASQSIS |
| | | DYLYWYQQKPGQAPRLLIKFASQSISGIPA |
| | | RFSGSGSGSEFTLTISSVQSEDFAVFFCQQ |
| | | GHSFPFTFGGGTKVEIK |
| 165 | 23A0186-6VL | EIVMTQSPATLSVSPGERVTLSCRASQSIS |
| | | DYLYWYQQKPGQAPRLLIKFASQSISGIPA |
| | | RFSGSGSGSEFTLTISSVQSEDFAVFFCQN |
| | | GHSFPFTFGGGTKVEIK |
| 166 | 23A0198 | DIVMTQSPDSLAVSLGERATINCKASKKVT |
| | (A5M21)VL | IYGSTSALHWYQQKPGQPPKLIYNLAKPES |
| | | GVPDRFSGSGSGTDFTLTISSLQAEDVAVY |
| | | YCLQNKEVPYTFGGGTKVEIK |
| 167 | Cκ | RTVAAPSVFIFPPSDEQLKSGTASVVCLLN |
| | | NFYPREAKVQWKVDNALQSGNSQESVTEQD |
| | | SKDSTYSLSSTLTLSKADYEKHKVYACEVT |
| | | HQGLSSPVTKSFNRGEC |
| 168 | CH1 | ASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPS |
| | | NTKVDKKVEPKSC |
| 169 | Fc | DKTHTCPPCPAPEAAGGPSVFLFPPKPKDT |
| | (LALA + YTE) | LYITREPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPSRDELTKNQVSLTCLVK |
| | | GFYPSDIAVEWESNGQPENNYKTTPPVLDS |
| | | DGSFFLYSKLTVDKSRWQQGNVFSCSVMHE |
| | | ALHNHYTQKSLSLSPGK |
| 170 | Fc | DKTHTCPPCPAPEAAGGPSVFLFPPKPKDT |
| | (LALA + LS) | LMISRTPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPSRDELTKNQVSLTCLVK |
| | | GFYPSDIAVEWESNGQPENNYKTTPPVLDS |
| | | DGSFFLYSKLTVDKSRWQQGNVFSCSVLHE |
| | | ALHSHYTQKSLSLSPGK |
| 171 | Fc-Knob | DKTHTCPPCPAPEAAGGPSVFLFPPKPKDT |
| | (LALA + YTE) | LYITREPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPSRDELTKNQVSLWCLVK |
| | | GFYPSDIAVEWESNGQPENNYKTTPPVLDS |
| | | DGSFFLYSKLTVDKSRWQQGNVFSCSVMHE |
| | | ALHNRFTQKSLSLSPGK |
| 172 | Fc-Hole | DKTHTCPPCPAPEAAGGPSVFLFPPKPKDT |
| | (LALA + YTE) | LYITREPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVCTLPPSREEMTKNQVSLSCAVK |
| | | GFYPSDIAVEWESNGQPENNYKTTPPVLDS |
| | | DGSFFLVSKLTVDKSRWQQGNVFSCSVMHE |
| | | ALHNRFTQKSLSLSPGK |
| 173 | Fc-Knob | DKTHTCPPCPAPEAAGGPSVFLFPPKPKDT |
| | (LALA + LS) | LMISRTPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVYTLPPCREEMTKNQVSLWCLVK |
| | | GFYPSDIAVEWESNGQPENNYKTTPPVLDS |
| | | DGSFFLYSKLTVDKSRWQQGNVFSCSVLHE |
| | | ALHSHYTQKSLSLSPGK |
| 174 | Fc-Hole | DKTHTCPPCPAPEAAGGPSVFLFPPKPKDT |
| | (LALA + LS) | LMISRTPEVTCVVVDVSHEDPEVKFNWYVD |
| | | GVEVHNAKTKPREEQYNSTYRVVSVLTVLH |
| | | QDWLNGKEYKCKVSNKALPAPIEKTISKAK |
| | | GQPREPQVCTLPPSREEMTKNQVSLSCAVK |
| | | GFYPSDIAVEWESNGQPENNYKTTPPVLDS |
| | | DGSFFLVSKLTVDKSRWQQGNVFSCSVLHE |
| | | ALHSRFTQKSLSLSPGK |
| 175 | light chain 1 | AIVMTQSPATLSVSPGERVTLSCRASQSIS |
| | | DYLYWYQQKPDQSPKLLIKFASQSISGVPS |
| | | RFSGSGSGRDFTLTISSLQPEDFAVYYCQN |
| | | GHSFPFTFGGGTKLEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 176 | light chain 2 | EIVMTQSPATLSLSPGERATLSCRASQSIS |
| | | DYLYWYQQKPDQSPKLLIKFASQSISGVPS |
| | | RFSGSGSGTEFTLTISSLQPEDFAVYYCQN |
| | | GHSFPFTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 177 | light chain 3 | DIVMTQSPATLSVSPGERVTLSCRASQSIS |
| | | DYLYWYQQKPDQSPKLLIKFASQSISGVPS |
| | | RFSGSGSGSDFTLTISSLQPEDFATYYCQN |
| | | GHSFPFTFGGGTKLEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 178 | light chain 4 | EIVMTQSPATLSVSPGERVTLSCRASQSIS |
| | | DYLYWYQQKPGQAPRLLIKFASQSISGIPA |
| | | RFSGSGSGSEFTLTISSVQSEDFAVFFCQQ |
| | | GHSFPFTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 179 | light chain 5 | EIVMTQSPATLSVSPGERVTLSCRASQSIS |
| | | DYLYWYQQKPGQAPRLLIKFASQSISGIPA |
| | | RFSGSGSGSEFTLTISSVQSEDFAVFFCQN |
| | | GHSFPFTFGGGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 180 | light chain 6 | EIVLTQSPATLSLSPGERATLSCRASQSVS |
| | | SNYLAWYQQKPGQAPRLLIYYASRRATGVP |
| | | ARFSGSGSGTDFTLTISSLEPEDFAYYCQQ |
| | | TSNTPFTFGQGTKVEIKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 181 | light chain 7 | EIVLTQSPATLSLSPGERATLSCRASKSVR |
| | | ESDYFYMHWYQQKPGQAPRLLIYLASNLES |
| | | GIPARFSGSGSGTDFTLTISSLEPEDFAVY |
| | | YCQNARELPYTFGQGTKLEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 182 | light chain 8 | DIVMTQSPDSLAVSLGERATINCKASKKVT |
| | | IYGSTSALHWYQQKPGQPPKLIYNLAKPES |
| | | GVPDRFSGSGSGTDFTLTISSLQAEDVAVY |
| | | YCLQNKEVPYTFGGGTKVEIKRTVAAPSVF |
| | | IFPPSDEQLKSGTASVVCLLNNFYPREAKV |
| | | QWKVDNALQSGNSQESVTEQDSKDSTYSLS |
| | | STLTLSKADYEKHKVYACEVTHQGLSSPVT |
| | | KSFNRGEC |
| 183 | TLb0090 heavy | EVQLQQSGPELVKPGASVKVSCKASGYTFT |
| | chain 1 | RYLMHWVRQAPGQGLEWIGYINPYNDGTKY |
| | | NQKFQGRVTITADKSTSTAYMELSSLTSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPGK |
| 184 | TLb0091 heavy | QVQLQQSGSELKKPGASVKVSCKASGYTFT |
| | chain 1 | RYLMHWVRQAPGKGLEWIGYINPYNDGTKY |
| | | NQKFQGRVTITADKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPGK |
| 185 | TLb0092 heavy | EIQLQQSGSELKKPGASVKVSCKASGYTFT |
| | chain 1 | RYLMNWVRQAPGKGLEWIGYINPYNDGTKY |
| | | NQKFQGRVTITADKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPGK |
| 186 | TLb0100 heavy | QVQLVQSGAEVVKPGASVKMSCKASGYTFT |
| | chain 1 | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPGK |
| 187 | TLb0102 heavy | QVQLVQSGAEVVKPGASVKMSCKASGYTFT |
| | chain 1 | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVMHEALHNHYTQKSLSLSPGK |
| 188 | TLb0102-1 heavy | QVQLVQSGAEVVKPGASVKMSCKASGYTFT |
| | chain 1 | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPCREEMTKNQ |
| | | VSLWCLVKGFYPSDIAVEWESNGQPENNYK |
| | | TTPPVLDSDGSFFLYSKLTVDKSRWQQGNV |
| | | FSCSVLHEALHSHYTQKSLSLSPGK |
| 189 | TLb0109 heavy | QVQLVQSGAEVKKPGSSVKVSCKASGYTFT |
| | chain 1 | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLYITREPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPSRDELTKNQVSLWCLVKGFYPSDIAVEW |
| | | ESNGQPENNYKTTPPVLDSDGSFFLYSKLT |
| | | VDKSRWQQGNVFSCSVMHEALHNRFTQKSL |
| | | SLSPGK |
| 190 | TLb0109-1 heavy | QVQLVQSGAEVKKPGSSVKVSCKASGYTFT |
| | chain 1 | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLMISRTPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPCREEMTKNQVSLWCLVKGFYPSDIAVEW |
| | | ESNGQPENNYKTTPPVLDSDGSFFLYSKLT |
| | | VDKSRWQQGNVFSCSVLHEALHSHYTQKSL |
| | | SLSPGK |
| 191 | TLb0090 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 192 | TLb0091 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 193 | TLb0092 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 194 | TLb0100 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 195 | TLb0102 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 196 | TLb0102-1 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVLHEALHSRFTQKSLSLSPGK |
| 197 | TLb0109 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLYITREPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVMHEALHNRFTQKSLSLSPGK |
| 198 | TLb0109-1 heavy | DIQMTQSPSSLSASVGDRVTITCRASQDIS |
| | chain 2 | NYLNWYQQKPGKAVKLLIYYTSRLHSGVPS |
| | | RFSGSGSGTDYTLTISSLQPEDFATYFCQQ |
| | | GNTLPFTFGCGTKLEIKGGGGSGGGGSGGG |
| | | GSGGGGSEIQLVESGGGLIQPGGSLRISCA |
| | | VSGFSLSSYGVDWVRQAPGKCLEWLGVIWG |
| | | FGGTNYNSALKSRLTISKDNSKNTVYLQMN |
| | | SLRAEDTAVYYCASGNFDAMDYWGQGTLVT |
| | | VSSEPKSADKTHTCPPCPAPEAAGGPSVFL |
| | | FPPKPKDTLMISRTPEVTCVVVDVSHEDPE |
| | | VKENWYVDGVEVHNAKTKPREEQYNSTYRV |
| | | VSVLTVLHQDWLNGKEYKCKVSNKALPAPI |
| | | EKTISKAKGQPREPQVCTLPPSREEMTKNQ |
| | | VSLSCAVKGFYPSDIAVEWESNGQPENNYK |
| | | TTPPVLDSDGSFFLVSKLTVDKSRWQQGNV |
| | | FSCSVLHEALHSRFTQKSLSLSPGK |
| 199 | TLb0103 heavy | QVQLVQSGAEVVKPGASVKMSCKASGYTFT |
| | chain | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLYITREPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPSRDELTKNQ |
| | | VSLTCLVKGFYPSDIAVEWESNGQPENNYK |
| | | TTPPVLDSDGSFFLYSKLTVDKSRWQQGNV |
| | | FSCSVMHEALHNHYTQKSLSLSPGKGGGGS |
| | | GGGGSGGGGSGGGGSEIQLVESGGGLIQPG |
| | | GSLRISCAVSGFSLSSYGVDWVRQAPGKCL |
| | | EWLGVIWGFGGTNYNSALKSRLTISKDNSK |
| | | NTVYLQMNSLRAEDTAVYYCASGNFDAMDY |
| | | WGQGTLVTVSSGGGGSGGGGSGGGGSGGGG |
| | | SDIQMTQSPSSLSASVGDRVTITCRASQDI |
| | | SNYLNWYQQKPGKAVKLLIYYTSRLHSGVP |
| | | SRFSGSGSGTDYTLTISSLQPEDFATYFCQ |
| | | QGNTLPFTFGCGTKLEIK |
| 200 | TLb0103-1 heavy | QVQLVQSGAEVVKPGASVKMSCKASGYTFT |
| | chain | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPSRDELTKNQ |
| | | VSLTCLVKGFYPSDIAVEWESNGQPENNYK |
| | | TTPPVLDSDGSFFLYSKLTVDKSRWQQGNV |
| | | FSCSVLHEALHSHYTQKSLSLSPGKGGGGS |
| | | GGGGSGGGGSGGGGSEIQLVESGGGLIQPG |
| | | GSLRISCAVSGFSLSSYGVDWVRQAPGKCL |
| | | EWLGVIWGFGGTNYNSALKSRLTISKDNSK |
| | | NTVYLQMNSLRAEDTAVYYCASGNFDAMDY |
| | | WGQGTLVTVSSGGGGSGGGGSGGGGSGGGG |
| | | SDIQMTQSPSSLSASVGDRVTITCRASQDI |
| | | SNYLNWYQQKPGKAVKLLIYYTSRLHSGVP |
| | | SRFSGSGSGTDYTLTISSLQPEDFATYFCQ |
| | | QGNTLPFTFGCGTKLEIK |
| 201 | TLb0103-2 heavy | QVQLVQSGAEVVKPGASVKMSCKASGYTFT |
| | chain | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPSRDELTKNQ |
| | | VSLTCLVKGFYPSDIAVEWESNGQPENNYK |
| | | TTPPVLDSDGSFFLYSKLTVDKSRWQQGNV |
| | | FSCSVLHEALHSHYTQKSLSLSPGKGGGGS |
| | | GGGGSEIQLVESGGGLIQPGGSLRISCAVS |
| | | GFSLSSYGVDWVRQAPGKCLEWLGVIWGFG |
| | | GTNYNSALKSRLTISKDNSKNTVYLQMNSL |
| | | RAEDTAVYYCASGNFDAMDYWGQGTLVTVS |
| | | SGGGGSGGGGSGGGGSGGGGSDIQMTQSPS |
| | | SLSASVGDRVTITCRASQDISNYLNWYQQK |
| | | PGKAVKLLIYYTSRLHSGVPSRFSGSGSGT |
| | | DYTLTISSLQPEDFATYFCQQGNTLPFTFG |
| | | CGTKLEIK |
| 202 | TLb0103-3 heavy | QVQLVQSGAEVVKPGASVKMSCKASGYTFT |
| | chain | RYLIHWVKQAPGQGLEWIGYINPYNEGTKY |
| | | NEKFKGRATLTSDKSTSTAYMELSSLRSED |
| | | TAVYYCTSNWDLDYWGQGTLVTVSSASTKG |
| | | PSVFPLAPSSKSTSGGTAALGCLVKDYFPE |
| | | PVTVSWNSGALTSGVHTFPAVLQSSGLYSL |
| | | SSVVTVPSSSLGTQTYICNVNHKPSNTKVD |
| | | KKVEPKSCDKTHTCPPCPAPEAAGGPSVFL |
| | | FPPKPKDTLMISRTPEVTCVVVDVSHEDPE |
| | | VKFNWYVDGVEVHNAKTKPREEQYNSTYRV |
| | | VSVLTVLHQDWLNGKEYKCKVSNKALPAPI |
| | | EKTISKAKGQPREPQVYTLPPSRDELTKNQ |
| | | VSLTCLVKGFYPSDIAVEWESNGQPENNYK |
| | | TTPPVLDSDGSFFLYSKLTVDKSRWQQGNV |
| | | FSCSVLHEALHSHYTQKSLSLSPGKGGGGS |
| | | GGGGSGGGGSEIQLVESGGGLIQPGGSLRI |
| | | SCAVSGFSLSSYGVDWVRQAPGKCLEWLGV |
| | | IWGFGGTNYNSALKSRLTISKDNSKNTVYL |
| | | QMNSLRAEDTAVYYCASGNFDAMDYWGQGT |
| | | LVTVSSGGGGSGGGGSGGGGSGGGGSDIQM |
| | | TQSPSSLSASVGDRVTITCRASQDISNYLN |
| | | WYQQKPGKAVKLLIYYTSRLHSGVPSRFSG |
| | | SGSGTDYTLTISSLQPEDFATYFCQQGNTL |
| | | PFTFGCGTKLEIK |
| 203 | TLb0107 heavy | QVQLVQSGAEVKKPGSSVKVSCKASGGTFS |
| | chain | SNYISWVRQAPGQGLEWMGAYAQKFQGRVT |
| | | ITADESTSTAYMELSSLRSEDTAVYYCARS |
| | | KKGMYGGWTYPLMMFDLWGQGTLVTVSSAS |
| | | TKGPSVFPLAPSSKSTSGGTAALGCLVKDY |
| | | FPEPVTVSWNSGALTSGVHTFPAVLQSSGL |
| | | YSLSSVVTVPSSSLGTQTYICNVNHKPSNT |
| | | KVDKKVEPKSCDKTHTCPPCPAPEAAGGPS |
| | | VFLFPPKPKDTLMISRTPEVTCVVVDVSHE |
| | | DPEVKFNWYVDGVEVHNAKTKPREEQYNST |
| | | YRVVSVLTVLHQDWLNGKEYKCKVSNKALP |
| | | APIEKTISKAKGQPREPQVYTLPPSRDELT |
| | | KNQVSLTCLVKGFYPSDIAVEWESNGQPEN |
| | | NYKTTPPVLDSDGSFFLYSKLTVDKSRWQQ |
| | | GNVFSCSVLHEALHSHYTQKSLSLSPGKGG |
| | | GGSGGGGSGGGGSGGGGSEIQLVESGGGLI |
| | | QPGGSLRISCAVSGFSLSSYGVDWVRQAPG |
| | | KCLEWLGVIWGFGGTNYNSALKSRLTISKD |
| | | NSKNTVYLQMNSLRAEDTAVYYCASGNFDA |
| | | MDYWGQGTLVTVSSGGGGSGGGGSGGGGSG |
| | | GGGSDIQMTQSPSSLSASVGDRVTITCRAS |
| | | QDISNYLNWYQQKPGKAVKLLIYYTSRLHS |
| | | GVPSRFSGSGSGTDYTLTISSLQPEDFATY |
| | | FCQQGNTLPFTFGCGTKLEIK |
| 204 | TLb0108 heavy | QVQLVQSGAEVVKPGSSVKVSCKASGYTFT |
| | chain | DYYMNWVRQAPGQGLEWIGDFNHNNDVITY |
| | | NPKFKGRVTLTSDKSTSTAYMELSSLRSED |
| | | TAVYYCARGLRGYYAMDYWGQGTLVTVSSA |
| | | STKGPSVFPLAPSSKSTSGGTAALGCLVKD |
| | | YFPEPVTVSWNSGALTSGVHTFPAVLQSSG |
| | | LYSLSSVVTVPSSSLGTQTYICNVNHKPSN |
| | | TKVDKKVEPKSCDKTHTCPPCPAPEAAGGP |
| | | SVFLFPPKPKDTLMISRTPEVTCVVVDVSH |
| | | EDPEVKFNWYVDGVEVHNAKTKPREEQYNS |
| | | TYRVVSVLTVLHQDWLNGKEYKCKVSNKAL |
| | | PAPIEKTISKAKGQPREPQVYTLPPSRDEL |
| | | TKNQVSLTCLVKGFYPSDIAVEWESNGQPE |
| | | NNYKTTPPVLDSDGSFFLYSKLTVDKSRWQ |
| | | QGNVFSCSVLHEALHSHYTQKSLSLSPGKG |
| | | GGGSGGGGSGGGGSGGGGSEIQLVESGGGL |
| | | IQPGGSLRISCAVSGFSLSSYGVDWVRQAP |
| | | GKCLEWLGVIWGFGGTNYNSALKSRLTISK |
| | | DNSKNTVYLQMNSLRAEDTAVYYCASGNFD |
| | | AMDYWGQGTLVTVSSGGGGSGGGGSGGGGS |
| | | GGGGSDIQMTQSPSSLSASVGDRVTITCRA |
| | | SQDISNYLNWYQQKPGKAVKLLIYYTSRLH |
| | | SGVPSRFSGSGSGTDYTLTISSLQPEDFAT |
| | | YFCQQGNTLPFTFGCGTKLEIK |
| 205 | TLb0110 heavy | QVQLVQSGAEVKKPGSSVKVSCKASGYTFT |
| | chain | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLYITREPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPSRDELTKNQVSLTCLVKGFYPSDIAVEW |
| | | ESNGQPENNYKTTPPVLDSDGSFFLYSKLT |
| | | VDKSRWQQGNVFSCSVMHEALHNHYTQKSL |
| | | SLSPGKGGGGSGGGGSGGGGSGGGGSEIQL |
| | | VESGGGLIQPGGSLRISCAVSGFSLSSYGV |
| | | DWVRQAPGKCLEWLGVIWGFGGTNYNSALK |
| | | SRLTISKDNSKNTVYLQMNSLRAEDTAVYY |
| | | CASGNFDAMDYWGQGTLVTVSSGGGGSGGG |
| | | GSGGGGSGGGGSDIQMTQSPSSLSASVGDR |
| | | VTITCRASQDISNYLNWYQQKPGKAVKLLI |
| | | YYTSRLHSGVPSRFSGSGSGTDYTLTISSL |
| | | QPEDFATYFCQQGNTLPFTFGCGTKLEIK |
| 206 | TLb0110-1 heavy | QVQLVQSGAEVKKPGSSVKVSCKASGYTFT |
| | chain | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLMISRTPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPSRDELTKNQVSLTCLVKGFYPSDIAVEW |
| | | ESNGQPENNYKTTPPVLDSDGSFFLYSKLT |
| | | VDKSRWQQGNVFSCSVLHEALHSHYTQKSL |
| | | SLSPGKGGGGSGGGGSGGGGSGGGGSEIQL |
| | | VESGGGLIQPGGSLRISCAVSGFSLSSYGV |
| | | DWVRQAPGKCLEWLGVIWGFGGTNYNSALK |
| | | SRLTISKDNSKNTVYLQMNSLRAEDTAVYY |
| | | SCAGNFDAMDYWGQGTLVTVSSGGGGSGGG |
| | | GSGGGGSGGGGSDIQMTQSPSSLSASVGDR |
| | | VTITCRASQDISNYLNWYQQKPGKAVKLLI |
| | | YYTSRLHSGVPSRFSGSGSGTDYTLTISSL |
| | | QPEDFATYFCQQGNTLPFTFGCGTKLEIK |
| 207 | TLb0110-2 heavy | QVQLVQSGAEVKKPGSSVKVSCKASGYTFT |
| | chain | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLMISRTPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPSRDELTKNQVSLTCLVKGFYPSDIAVEW |
| | | ESNGQPENNYKTTPPVLDSDGSFFLYSKLT |
| | | VDKSRWQQGNVFSCSVLHEALHSHYTQKSL |
| | | SLSPGKGGGGSGGGGSEIQLVESGGGLIQP |
| | | GGSLRISCAVSGFSLSSYGVDWVRQAPGKC |
| | | LEWLGVIWGFGGTNYNSALKSRLTISKDNS |
| | | KNTVYLQMNSLRAEDTAVYYCASGNFDAMD |
| | | YWGQGTLVTVSSGGGGSGGGGSGGGGSGGG |
| | | GSDIQMTQSPSSLSASVGDRVTITCRASQD |
| | | ISNYLNWYQQKPGKAVKLLIYYTSRLHSGV |
| | | PSRFSGSGSGTDYTLTISSLQPEDFATYFC |
| | | QQGNTLPFTFGCGTKLEIK |
| 208 | TLb0110-3 heavy | QVQLVQSGAEVKKPGSSVKVSCKASGYTFT |
| | chain | SYGITWVRQAPGQGLEWMGENYPRSGNTYY |
| | | NEKFKGRVTITADKSTSTAYMELSSLRSED |
| | | TAVYYCARAEFISTVVAPYYYALDYWGQGT |
| | | LVTVSSASTKGPSVFPLAPSSKSTSGGTAA |
| | | LGCLVKDYFPEPVTVSWNSGALTSGVHTFP |
| | | AVLQSSGLYSLSSVVTVPSSSLGTQTYICN |
| | | VNHKPSNTKVDKKVEPKSCDKTHTCPPCPA |
| | | PEAAGGPSVFLFPPKPKDTLMISRTPEVTC |
| | | VVVDVSHEDPEVKFNWYVDGVEVHNAKTKP |
| | | REEQYNSTYRVVSVLTVLHQDWLNGKEYKC |
| | | KVSNKALPAPIEKTISKAKGQPREPQVYTL |
| | | PPSRDELTKNQVSLTCLVKGFYPSDIAVEW |
| | | ESNGQPENNYKTTPPVLDSDGSFFLYSKLT |
| | | VDKSRWQQGNVFSCSVLHEALHSHYTQKSL |
| | | SLSPGKGGGGSGGGGSGGGGSEIQLVESGG |
| | | GLIQPGGSLRISCAVSGFSLSSYGVDWVRQ |
| | | APGKCLEWLGVIWGFGGTNYNSALKSRLTI |
| | | SKDNSKNTVYLQMNSLRAEDTAVYYCASGN |
| | | FDAMDYWGQGTLVTVSSGGGGSGGGGSGGG |
| | | GSGGGGSDIQMTQSPSSLSASVGDRVTITC |
| | | RASQDISNYLNWYQQKPGKAVKLLIYYTSR |
| | | LHSGVPSRFSGSGSGTDYTLTISSLQPEDF |
| | | ATYFCQQGNTLPFTFGCGTKLEIK |
| 209 | HFR1-1 | QVQLVQSGAEVKKPGSSVKVSCKASGYTFT |
| 210 | HFR1-2 | EVQLVESGGGLVKPGGSLRLSCAASGYTFT |
| 211 | HFR1-3 | EVQLQQSGPELVKPGASVKVSCKASGYTFT |
| 212 | HFR1-4 | EVQLQQSGPEVKKPGASVKVSCKASGYTFT |
| 213 | HFR1-5 | QVQLQQSGSELKKPGASVKVSCKASGYTFT |
| 214 | HFR1-6 | EIQLQQSGSELKKPGASVKVSCKASGYTFT |
| 215 | HFR1-7 | QVQLVQSGAEVKKPGASVKVSCKASGYTFT |
| 216 | HFR1-8 | QVQLVQSGAEVVKPGASVKMSCKASGYTFT |
| 217 | HFR2-1 | WVRQAPGQGLEWIG |
| 218 | HFR2-2 | WVRQAPGKGLEWIG |
| 219 | HFR2-3 | WVRQPPGQGLEWIG |
| 220 | HFR2-4 | WVRQAPGQGLEWMG |
| 221 | HFR2-5 | WVKQAPGQGLEWIG |
| 222 | HFR3-1 | RVTITADESTSTAYMELSSLRSEDTAVYYC |
| | | TS |
| 223 | HFR3-2 | RFTISRDDSKNTLYLQMNSLKTEDTAVYYC |
| | | TS |
| 224 | HFR3-3 | RVTITADKSTSTAYMELSSLTSEDTAVYYC |
| | | TS |
| 225 | HFR3-4 | RVTITADKSTSTAYMELSSLRSEDTAVYYC |
| | | TS |
| 226 | HFR3-5 | RVTMTRDTSTSTVYMELSSLRSEDTAVYYC |
| | | AS |
| 227 | HFR3-6 | RVTMTSDKSTSTAYMELSSLRSEDTAVYYC |
| | | TS |
| 228 | HFR3-7 | RATLTSDKSTSTAYMELSSLRSEDTAVYYC |
| | | TS |
| 229 | HFR3-8 | RVTITADKSTSTAYMELSSLRSEDTAVYYC |
| | | AR |
| 230 | HFR4-2 | WGQGTTLTVSS |
| 231 | LFR1-1 | AIQMTQSPSSLSASVGDRVTITC |
| 232 | LFR1-2 | DIVMTQSPDSLAVSLGERATINC |
| 233 | LFR1-3 | NIQMTQSPSAMSASVGDRVTITC |
| 234 | LFR1-4 | DIQMTQSPSTLSASVGDRVTITC |
| 235 | LFR1-5 | AIVMTQSPATLSVSPGERVTLSC |
| 236 | LFR1-6 | NIVMTQSPATLSVSPGERATLSC |
| 237 | LFR1-7 | EIVMTQSPATLSLSPGERATLSC |
| 238 | LFR1-8 | DIVMTQSPATLSVSPGERVTLSC |
| 239 | LFR1-9 | DIVMTQSPSSLSASVGDRVTLTC |
| 240 | LFR1-10 | EIVMTQSPATLSVSPGERVTLSC |
| 241 | LFR2-1 | WYQQKPGKAPKLLIK |
| 242 | LFR2-2 | WFQQKPGKAPKLLIK |
| 243 | LFR2-3 | WFQQKPGKVPKLLIK |
| 244 | LFR2-4 | WYQQKPDQSPKLLIK |
| 245 | LFR2-5 | WYQQKPDQSPRLLIK |
| 246 | LFR2-6 | WYQQKPGQPPKLIY |
| 247 | LFR2-7 | WYQQKPGQAPRLLIK |
| 248 | LFR3-1 | GVPSRFSGSGSGTDFTLTISSLQPEDFATY |
| | | YCQ |
| 249 | LFR3-2 | GVPSRFSGSGSGTEFTLTISSLQPEDFATY |
| | | YCQ |
| 250 | LFR3-3 | GVPSRFSGSGSGTEFTLTISSLQPDDFATY |
| | | YCQ |
| 251 | LFR3-4 | GVPSRFSGSGSGRDFTLTISSLQPEDFAVY |
| | | YCQ |
| 252 | LFR3-5 | GVPSRFSGSGSGRDFTLTISSLEPEDFAVY |
| | | YCQ |
| 253 | LFR3-6 | GVPSRFSGSGSGTEFTLTISSLQPEDFAVY |
| | | YCQ |
| 254 | LFR3-7 | GVPSRFSGSGSGSDFTLTISSLQPEDFATY |
| | | YCQ |
| 255 | LFR3-8 | GVPSRFSGSGSGSDFTLKISSVEAEDVGVY |
| | | YCQ |
| 256 | LFR3-9 | GIPSRFSGSGSGSDFTLTISSLQPEDFATF |
| | | YCQ |
| 257 | LFR3-10 | GIPSRFSGSGSGSDFTLTISSVQPEDFATF |
| | | FCQ |
| 258 | LFR3-11 | GIPARFSGSGSGSEFTLTISSVQSEDFAVF |
| | | FCQ |
| 259 | LFR4-1 | FGPGTKVDIK |
| 260 | LFR4-2 | FGQGTRLEIK |
| 261 | LFR4-3 | FGQGTKVEIK |
| 262 | LFR4-4 | FGGGTKLEIK |
| 263 | LFR4-5 | FGGGTKVEIK |
| 264 | 23A0198 HCDR1 | SYGIT |
| 265 | 23A0198 HCDR2 | ENYPRSGNTYYNEKFKG |
| 266 | 23A0198 HCDR3 | AEFISTVVAPYYYALDY |
| 267 | 23A0198 LCDR1 | KASKKVTIYGSTSALH |
| 268 | 23A0198 LCDR2 | NLAKPES |
| 269 | 23A0198 LCDR3 | LQNKEVPYT |
| 270 | TLA0015 VH | EIQLVESGGGLIQPGGSLRISCAVSGFSLS |
| | | SYGVDWVRQAPGKCLEWLGVIWGFGGTNYN |
| | | SALKSRLTISKDNSKNTVYLQMNSLRAEDT |
| | | AVYYCASGNFDAMDYWGQGTLVTVSS |
| 271 | TLA0015 VL | DIQMTQSPSSLSASVGDRVTITCRASQDIS |
| | | NYLNWYQQKPGKAVKLLIYYTSRLHSGVPS |
| | | RFSGSGSGTDYTLTISSLQPEDFATYFCQQ |
| | | GNTLPFTFGCGTKLEIK |
| 272 | HFR4-1 | WGQGTLVTVSS |
| 273 | LFR3-12 | GVPDRESGSGSGTDFTLTISSLQAEDVAVY |
| | | YC |

1-36. (canceled)

37. An isolated antigen binding protein, comprising: a first binding domain and a second binding domain, wherein the first binding domain specifically binds to TNF-like protein A (TL1A), and the second binding domain specifically binds to IL-23p19, wherein the first binding domain comprises a heavy chain variable region (VH), and the VH comprises an HCDR3 comprising an amino acid sequence of SEQ ID NO: 14.

38. The isolated antigen binding protein according to claim 37, wherein HCDR3 comprises an amino acid sequence of any one of SEQ ID NOs: 15-18.

39. The isolated antigen binding protein according to claim 37, wherein the VH comprises an HCDR2 comprising an amino acid sequence of SEQ ID NO: 5.

40. The isolated antigen binding protein according to claim 39, wherein the HCDR2 comprises an amino acid sequence of any one of SEQ ID NOs: 6-13.

41. The isolated antigen-binding protein according to claim 39, wherein the VH comprises an HCDR1 comprising the amino acid sequence of SEQ ID NO:1.

42. The isolated antigen-binding protein according to claim 41, wherein the HCDR1 comprises an amino acid sequence of any one of SEQ ID NOs:2-4.

43. The isolated antigen-binding protein according to claim 37, wherein the VH of the first binding domain comprises the HCDR1 as set forth in SEQ ID NO:2, the HCDR2 as set forth in SEQ ID NO:6, and the HCDR3 as set forth in SEQ ID NO:15.

44. The isolated antigen-binding protein according to claim 43, wherein the first binding domain further comprises a light chain variable region (VL), and the VL of the first binding domain comprising at least one light chain complementary determining region (LCDR), which comprises an amino acid sequence selected from the amino acid sequences shown in SEQ ID NO:42, SEQ ID NO:43 and SEQ ID NO:44.

45. The isolated antigen-binding protein according to claim 37, wherein the first binding domain comprises the VH as set forth in SEQ ID NO:270.

46. The isolated antigen-binding protein according to claim 37, wherein the first binding domain comprises the VL as set forth in SEQ ID NO:271.

47. The isolated antigen-binding protein according to claim 37, wherein the VH of the second binding domain comprises the HCDR1 as set forth in SEQ ID NO: 264, the HCDR2 as set forth in SEQ ID NO: 265, and the HCDR3 as set forth in SEQ ID NO: 266.

48. The isolated antigen-binding protein according to claim 47, wherein the VL of the second binding domain comprises the LCDR1 as set forth in SEQ ID NO: 267, the LCDR2 as set forth in SEQ ID NO: 268, and the LCDR3 as set forth in SEQ ID NO: 269.

49. The isolated antigen-binding protein according to claim 37, wherein the second antigen binding domain comprises a VL as set forth in SEQ ID NO: 166 and/or the second antigen binding domain comprises a VH as set forth in SEQ ID NO: 148.

50. The isolated antigen-binding protein according to claim 37, wherein the antigen-binding protein comprises the scFv of the first antigen binding domain or the scFv of the second antigen binding domain.

51. The isolated antigen-binding protein according to claim 50, wherein the scFv of the first antigen-binding domain comprises a sequence as set forth in SEQ ID NO: 133.

52. The isolated antigen-binding protein according to claim 37, wherein the VL and VH of the second binding domain comprises the VH as set forth in SEQ ID NO:148 and VL as set forth in SEQ ID NO:166.

53. The isolated antigen-binding protein according to claim 37, wherein the first heavy chain is selected from the sequence shown in any one of SEQ ID NOs: 183-208 and/or the first light chain is selected from the sequence shown in any one of SEQ ID NOs: 175-182.

54. The isolated antigen-binding protein according to claim 37, wherein the first antigen-binding domain comprises HCDR1 as set forth in SEQ ID NO: 2, HCDR2 as set forth in SEQ ID NO: 6, HCDR3 as set forth in SEQ ID NO: 15, LCDR1 as set forth in SEQ ID NO: 42, LCDR2 as set forth in SEQ ID NO: 43, and LCDR3 as set forth in SEQ ID NO: 44; and the second antigen-binding domain comprises HCDR1 as set forth in SEQ ID NO: 264, HCDR2 as set forth in SEQ ID NO: 265, HCDR3 as set forth in SEQ ID NO: 266, LCDR1 as set forth in SEQ ID NO: 267, LCDR2 as set forth in SEQ ID NO: 268, and LCDR3 as set forth in SEQ ID NO: 269.

55. The isolated antigen-binding protein according to claim 54, wherein the first antigen-binding domain comprises a VH as set forth in SEQ ID NO: 270 and a VL as set forth in SEQ ID NO: 271; and the second antigen-binding domain comprises a VH as set forth in SEQ ID NO: 148 and a VL as set forth in SEQ ID NO: 166.

56. The isolated antigen-binding protein according to claim 55, wherein the first antigen-binding domain comprises an scFv as set forth in SEQ ID NO: 133; and the second antigen-binding domain comprises the VH as set forth in SEQ ID NO: 148 and the VL as set forth in SEQ ID NO: 166.

57. The isolated antigen-binding protein according to claim 55 comprising a light chain as shown in SEQ ID NO: 182 and heavy chain as shown in SEQ ID NO: 207.

58. An isolated nucleic acid molecule encoding an antigen-binding protein comprising: a first binding domain and a second binding domain, wherein the first binding domain specifically binds to TNF-like protein A (TL1A), and the second binding domain specifically binds to IL-23p19, wherein the first binding domain comprises a heavy chain variable region (VH), and the VH comprises an HCDR3 comprising an amino acid sequence of SEQ ID NO: 14.

59. A host cell comprising an isolated nucleic acid according to claim 58.

60. A pharmaceutical composition comprising an isolated antigen-binding protein according to claim 37, and a pharmaceutically acceptable carrier.

61. A method for preparing an isolated antigen-binding protein, comprising culturing a host cell comprising one or more nucleic acids encoding an isolated antigen binding protein, comprising a first binding domain and a second binding domain, wherein the first binding domain specifically binds to TNF-like protein A (TL1A), and the second binding domain specifically binds to IL-23p19, wherein the first binding domain comprises a heavy chain variable region (VH), and the VH comprises an HCDR3 comprising an amino acid sequence of SEQ ID NO: 14, and recovering the isolated antigen-binding protein.

62. A method for treating a TL1A or IL-23 mediated disease or condition in a subject in need thereof, the metho comprising: administering an isolated antigen binding protein to the subject, an antigen binding protein comprising a first binding domain and a second binding domain, wherein the first binding domain specifically binds to TNF-like protein A (TL1A), and the second binding domain specifically binds to IL-23p19, wherein the first binding domain comprises a heavy chain variable region (VH), and the VH comprises an HCDR3 comprising an amino acid sequence of SEQ ID NO: 14, thereby treating the subject.

63. The method according to claim 62, wherein the TL1A or IL-23 mediated disease or condition is an inflammatory disease selected from one of the following: allergy, ankylosing spondylitis, asthma, atopic dermatitis, autoimmune diseases or disorders, cancer, celiac disease, chronic obstructive pulmonary disease (COPD), chronic peptic ulcer, cystic fibrosis, diabetes (e.g., type 1 diabetes and type 2 diabetes), glomerulonephritis, gout, hepatitis (e.g., active hepatitis), an immune-mediated disease or disorder, inflammatory bowel disease (IBD) such as Crohn's disease and ulcerative colitis, myositis, osteoarthritis, pelvic inflammatory disease (PID), multiple sclerosis, neurodegenerative diseases of aging, periodontal disease (e.g., periodontitis), preperfusion injury transplant rejection, psoriasis, pulmonary fibrosis (e.g., idiopathic pulmonary fibrosis), rheumatic disease, scleroderma, sinusitis, tuberculosis.

64. The method according to claim 62, wherein the TL1A or IL-23 mediated disease or condition is an autoimmune disease selected from one of the following: achalasia, Addison's disease, Adult Stil's disease, agammaglobulinemia, alopecia areata, amyloidosis, ankylosing spondylitis, anti-GBM/anti-TBM nephritis, antiphospholipid syndrome, autoimmune angioedema, autoimmune familial autonomic dysfunction, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal and neuronal neuropathy (AMAN), Barlow disease (Balódisease), Behcet's disease, benign mucous membrane pemphigoid, bullous pemphigoid, Castleman's disease (Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatous disease (EGPA), cicatricial pemphigoid, and Cogan's syndrome), cold agglutinin disease, congenital heart block, coxsackienew myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, primary mixed cryoglobulinemia, Evans syndrome, fibromyalgia, fibrotic alveolitis, giant cell arteritis (temporal arteritis), giant cell myocarditis, glomerulonephritis, Goodpasture's syndrome, granulomatosis with polyangiitis, Graves' disease, Guillain-Barre syndrome syndrome), Hashimoto's thyroiditis, hemolytic anemia, Henoch-Schonlein purpura (HSP), herpes gravilens or pemphigoid gestationum (PG), hidradenitis suppurativa (HS) (paradoxical acne), hypogammaglobulinemia, IgA nephropathy, IgG4-associated sclerotic disease, immune thrombocytopenic purpura (ITP), Inclusion body myositis (IBM), interstitial cystitis (IC), juvenile arthritis, juvenile diabetes mellitus (type 1 diabetes), juvenile myositis (JM), Kawasaki disease, Lambert-Eaton syndrome, leukocytoclastic vasculitis, lichen planus, lichen sclerosus, lignoconjunctivitis, linear IgA disease (LAD), lupus, chronic Lyme disease (Lyme disease chronic), Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, recurrent rheumatism (PR), PandAS, paraneoplastic cerebellar degeneration (PCD), paroxysmal nocturnal hemoglobinuria (PNH), Parry Romberg syndrome, ciliary planitis (peripheral uveitis), Parsonage-Tumer syndrome), pemphigus, peripheral neuropathy, perivenous encephalomyelitis, pernicious anemia (PA), POEMS syndrome, polyarteritis nodosa, polyglandular syndrome type I, II, type III, polymyalgia rheumatica, polymyositis, post-myocardial infarction syndrome, postpericardiotomy syndrome, primary biliary cirrhosis, primary sclerosing cholangitis, progesterone dermatitis, psoriasis, psoriatic arthritis, pure red blood cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff-person syndrome (SPS), subacute bacterial endocarditis (SBE), Susac's syndrome syndrome), sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes mellitus, ulcerative colitis (UC), undifferentiated connective tissue disease (UCTD), uveitis, vasculitis, Vitiligo and Vogt-Koyanagi-Harada disease.

65. The method according to claim 62, wherein the TL1A or IL-23 mediated disease or condition is a cancer selected from one or more of the following: adenoid cystic carcinoma, adrenal carcinoma, amyloidosis, anal cancer, ataxia-telangiectasia, atypical nevus syndrome, basal cell carcinoma, cholangiocarcinoma, Birt Hogg Dube syndrome, bladder cancer, bone cancer, brain tumor, breast cancer, male breast cancer, carcinoid tumor, cervical cancer, colorectal cancer, ductal cancer, Endometrial Cancer, Esophageal Cancer, Gastric Cancer, Gastrointestinal Stromal Tumor (GIST), HER2-Positive Breast Cancer, Pancreatic Islet Cell Tumor, Juvenile Polyposis Syndrome, Kidney Cancer, Laryngeal Cancer, Leukemia—Acute Lymphoblastic Leukemia, Acute Lymphoblastic Leukemia (ALL), Acute Myeloid Leukemia AML, Adult Leukemia, Childhood Leukemia, Chronic Lymphocytic Leukemia (CLL), Chronic Myeloid Leukemia (CML), Liver Cancer, Lobular Carcinoma, Lung Cancer, Small Cell Lung Cancer (SCLC), Non-small cell lung cancer (NSCLC), Hodgkin's lymphoma (Lymphoma-Hodgkin's), non-Hodgkin's lymphoma (Lymphoma-Non-Hodgkin's), malignant glioma, melanoma, meningioma, multiple myeloma, myelodysplastic syndrome (MDS), nasopharyngeal carcinoma, neuroendocrine tumor, oral cancer, osteosarcoma, ovarian cancer, pancreatic cancer, pancreatic neuroendocrine tumor, parathyroid carcinoma, penile cancer, peritoneal cancer, Peutz-Jeghers syndrome, pituitary tumors, polycythemia vera, prostate cancer, renal cell carcinoma, retinoblastoma, salivary gland carcinoma, sarcoma, Sarcoma-Kaposi, skin cancer, small bowel cancer, stomach cancer, testicular cancer, thymoma, thyroid cancer, uterine (endometrial) cancer, vaginal cancer, and Wilms' Tumor.
